(19) United States
(12) Patent Application Publication
(10) Pub. No.: US 2008/0221056 A1
(43) Pub. Date: Sep. 11, 2008

(54) Early Detection and Prognosis of Colon Cancers

(75) Inventors: Stephen B. Baylin, Baltimore, MD (US); Wim Van Criekinge, Sart-Tilman (Liege) (BE); Kornel E. Schuebel, Baltimore, MD (US)

Correspondence Address: BANNER & WITCOFF, LTD., 1100 13th STREET, N.W., SUITE 1200, WASHINGTON, DC, 20005-4051, US

(73) Assignees: JOHNS HOPKINS UNIVERSITY, Baltimore, MD (US); ONCOMETHYLOME SCIENCES, S.A., Sart-Tilman (Liege) (BE)

(21) Appl. No.: 12/029,645

(22) Filed: Feb. 12, 2008

Related U.S. Application Data
(60) Provisional application No. 60/900,713, filed on Feb. 12, 2007.

(51) Int. Cl. A61K 31/7088; C12Q 1/68; A61P 35/00

(52) U.S. Cl. 514/44; 435/6

(57) ABSTRACT

We have developed a transcriptome-wide approach to identify genes affected by promoter CpG island hypermethylation and transcriptional silencing in colorectal cancer (CRC). By screening cell lines and validating tumor specific hypermethylation in a panel of primary human CRC samples, we estimate that nearly 5% of all known genes may be promoter methylated in an individual tumor. When directly compared to gene mutations, we find a much larger number of genes hypermethylated in individual tumors, and much higher frequency of hypermethylation within individual genes harboring either genetic or epigenetic changes. Thus, to enumerate the full spectrum of alterations in the human cancer genome, and facilitate the most efficacious grouping of tumors to identify cancer biomarkers and tailor therapeutic approaches, both genetic and epigenetic screens should be undertaken. The genes we identified can be used inter alia diagnostically to detect cancer, pre-cancer, and likelihood of developing cancer.

This invention was made using U.S. government funds under grant ES11858 from the National Institute of Environmental Health Sciences under grant CA043318 from the National Cancer Institute. The U.S. government retains certain rights to the invention under the terms of these grants.

### TECHNICAL FIELD OF THE INVENTION

This invention is related to the area of cancer diagnostics and therapeutics. In particular, it relates to aberrant methylation patterns of particular genes in colon cancer and pre-cancer.

### BACKGROUND OF THE INVENTION

### DNA Methylation and its Role in Carcinogenesis

The information to make the cells of all living organisms is contained in their DNA. DNA is made up of a unique sequence of four bases: adenine (A), guanine (G), thymine (T) and cytosine (C). These bases are paired A to T and G to C on the two strands that form the DNA double helix. Strands of these pairs store information to make specific molecules grouped into regions called genes. Within each cell, there are processes that control what gene is turned on, or expressed, thus defining the unique function of the cell. One of these control mechanisms is provided by adding a methyl group onto cytosine (C). The methyl group tagged C can be written as mC.

DNA methylation plays an important role in determining whether some genes are expressed or not. By turning genes off that are not needed, DNA methylation is an essential control mechanism for the normal development and functioning of organisms. Alternatively, abnormal DNA methylation is one of the mechanisms underlying the changes observed with aging and development of many cancers.

Cancers have historically been linked to genetic changes caused by chromosomal mutations within the DNA. Mutations, hereditary or acquired, can lead to the loss of expression of genes critical for maintaining a healthy state. Evidence now supports that a relatively large number of cancers are caused by inappropriate DNA methylation, frequently near DNA mutations. In many cases, hyper-methylation of DNA incorrectly switches off critical genes, such as tumor suppressor genes or DNA repair genes, allowing cancers to develop and progress. This non-mutational process for controlling gene expression is described as epigenetics.

DNA methylation is a chemical modification of DNA performed by enzymes called methyltransferases, in which a methyl group (m) is added to certain cytosines (C) of DNA. This non-mutational (epigenetic) process (mC) is a critical factor in gene expression regulation. See, J. G. Herman, Seminars in Cancer Biology, 9: 359-67, 1999.

Although the phenomenon of gene methylation has attracted the attention of cancer researchers for some time, its true role in the progression of human cancers is just now being recognized. In normal cells, methylation occurs predominantly in regions of DNA that have few CG base repeats, while CpG islands, regions of DNA that have long repeats of CG bases, remain non-methylated. Gene promoter regions that control protein expression are often CpG island-rich. Aberrant methylation of these normally non-methylated CpG islands in the promoter region causes transcriptional inactivation or silencing of certain tumor suppressor expression in human cancers.

Genes that are hypermethylated in tumor cells are strongly specific to the tissue of origin of the tumor. Molecular signatures of cancers of all types can be used to improve cancer detection, the assessment of cancer risk and response to therapy. Promoter hypermethylation events provide some of the most promising markers for such purposes.

### Promoter Gene Hypermethylation: Promising Tumor Markers

Information regarding the hypermethylation of specific promoter genes can be beneficial to diagnosis, prognosis, and treatment of various cancers. Methylation of specific gene promoter regions can occur early and often in carcinogenesis making these markers ideal targets for cancer diagnostics.

Methylation patterns are tumor specific. Positive signals are always found in the same location of a gene. Real time PCR-based methods are highly sensitive, quantitative, and suitable for clinical use. DNA is stable and is found intact in readily available fluids (e.g., serum, sputum, stool, blood, and urine) and paraffin embedded tissues. Panels of pertinent gene markers may cover most human cancers.

### Diagnosis

Key to improving the clinical outcome in patients with cancer is diagnosis at its earliest stage, while it is still localized and readily treatable. The characteristics noted above provide the means for a more accurate screening and surveillance program by identifying higher-risk patients on a molecular basis. It could also provide justification for more definitive follow up of patients who have molecular but not yet all the pathological or clinical features associated with malignancy.

At present, early detection of colorectal cancer is carried out by (1) the “fecal occult blood test” (FOBT), which has a very low sensitivity and specificity, (2) by sigmoidoscopy and/or colonoscopy which is invasive and expensive (and limited in supply), (3) by X-ray detection after double-contrast barium enema, which allows only for the detection of rather large polyps, or CT-colonography (also called virtual colonoscopy) which is still experimental, and (4) by a gene mutation analysis test called PreGen-Plus (Exact Sciences; LabCorp) which is costly and of limited sensitivity.

### Predicting Treatment Response

Information about how a cancer develops through molecular events could allow a clinician to predict more accurately how such a cancer is likely to respond to specific chemotherapeutic agents. In this way, a regimen based on knowledge of the tumor's chemosensitivity could be rationally designed. Studies have shown that hypermethylation of the MGMT promoter in glioma patients is indicative of a good response to therapy, greater overall survival and a longer time to progression.

It is now well established that loss of proper gene function in human cancer can occur through both genetic and epigenetic mechanisms (1,2). The number of genes mutated in human tumor samples is being clarified. Recently, Sjöblom et al. (3) sequenced 13,023 genes in colorectal (CRC) and breast cancer, and discovered an average of 11 mutations per tumor, suggesting that a relatively small number of genetic events may be sufficient to drive tumorigenesis. In contrast, the full spectrum of epigenetic alterations is not well delineated. The best defined epigenetic alteration of cancer genes involves DNA hypermethylation of clustered CpG dinucleotides, or CpG islands, in promoter regions associated with the transcriptional inactivation of the affected gene. These promoters are located proximal to nearly half of all genes (4) and are thought to remain primarily methylation free in normal somatic tissues. The exact number of such epigenetic lesions in any given tumor is not precisely known although a growing number of random screening approaches, none covering the whole genome efficiently, are identifying an increasing number of candidate genes (5-12). Given the large number of potential target promoters present in the genome, we hypothesized that many more hypermethylated genes await discovery (13).

There is a continuing need in the art for new diagnostic and prognostic markers and therapeutic targets for cancer to improve management of patient care.

### SUMMARY OF THE INVENTION

According to one aspect of the invention a method is provided for identifying colorectal cancer or its precursor, or predisposition to colorectal cancer. Epigenetic silencing is detected in a test sample containing colorectal cells or nucleic acids from colorectal cells. The epigenetic silencing is of at least one gene selected from the group consisting of BOLL, CABYR, EFEMP1, FBLN2, FOXL2, GNB4, GSTM3, HoxD1, Jph3, Neuralized (NEURL), PPP1R14a, TP53AP1, RAB32, SYNE1, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, SOX17, STK31, SYCP3, TCL1A, TFPI-2, TLR2, UCHL1, ZFP42, ASCL2, ATP8A2, CTAG2, EPHA4, FANCF, FOXE1, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655. The test sample is identified as containing cells that are neoplastic, precursor to neoplastic, or predisposed to neoplasia, or as containing nucleic acids from cells that are neoplastic, precursor to neoplastic, or predisposed to neoplasia, when epigenetic silencing is detected.

According to another aspect of the invention a method is provided of reducing or inhibiting neoplastic growth of a cell which exhibits epigenetic silenced transcription of at least one gene associated with a cancer. An epigenetically silenced gene is determined in a cell. The gene is selected from the group consisting of BOLL, CABYR, EFEMP1, FBLN2, FOXL2, GNB4, GSTM3, HoxD1, Jph3, Neuralized (NEURL), PPP1R14a, TP53AP1, RAB32, SYNE1, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, SOX17, STK31, SYCP3, TCL1A, TFPI-2, TLR2, UCHL1, ZFP42, ASCL2, ATP8A2, CTAG2, EPHA4, FANCF, FOXE1, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655. Expression of a polypeptide encoded by the epigenetic silenced gene in the cell is restored by contacting the cell with one or more agents selected from the group consisting of a CpG dinucleotide demethylating agent, a DNA methyltransferase inhibitor, and a histone deacetylase (HDAC) inhibitor. Unregulated growth of the cell is thereby reduced or inhibited.

Another aspect of the invention is a method of reducing or inhibiting neoplastic growth of a cell which exhibits epigenetic silenced transcription of at least one gene associated with a cancer. An epigenetic silenced gene is determined in the cell. The gene is selected from the group consisting of BOLL, CABYR, EFEMP1, FBLN2, FOXL2, GNB4, GSTM3, HoxD1, Jph3, Neuralized (NEURL), PPP1R14a, TP53AP1, RAB32, SYNE1, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, SOX17, STK31, SYCP3, TCL1A, TFPI-2, TLR2, UCHL1, ZFP42, ASCL2, ATP8A2, CTAG2, EPHA4, FANCF, FOXE1, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655. A polynucleotide encoding a polypeptide is introduced into the cell. The polypeptide is encoded by said gene. The polypeptide is expressed in the cell thereby restoring expression of the polypeptide in the cell.

Yet another aspect of the invention is a method of treating a cancer patient. A cancer cell in the patient is determined to have an epigenetic silenced gene selected from the group consisting of BOLL, CABYR, EFEMP1, FBLN2, FOXL2, GNB4, GSTM3, HoxD1, Jph3, Neuralized (NEURL), PPP1R14a, TP53AP1, RAB32, SYNE1, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, SOX17, STK31, SYCP3, TCL1A, TFPI-2, TLR2, UCHL1, ZFP42, ASCL2, ATP8A2, CTAG2, EPHA4, FANCF, FOXE1, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655. One or more agents selected from the group consisting of a CpG dinucleotide demethylating agent, a DNA methyltransferase inhibitor, and a histone deacetylase (HDAC) inhibitor is administered to the patient in sufficient amounts to restore expression of the epigenetic silenced gene in the patient's cancer cells.

Yet another aspect of the invention is a method of treating a cancer patient. A cancer cell in the patient is determined to have an epigenetic silenced gene selected from those shown in BOLL, CABYR, EFEMP1, FBLN2, FOXL2, GNB4, GSTM3, HoxD1, Jph3, Neuralized (NEURL), PPP1R14a, TP53AP1, RAB32, SYNE1, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, SOX17, STK31, SYCP3, TCL1A, TFPI-2, TLR2, UCHL1, ZFP42, ASCL2, ATP8A2, CTAG2, EPHA4, FANCF, FOXE1, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655. A polynucleotide encoding a polypeptide is administered to the patient. The polypeptide is encoded by the epigenetic silenced gene. The polypeptide is expressed in the patient's tumor thereby restoring expression of the polypeptide in the cancer.

According to another aspect of the invention a method is provided for selecting a therapeutic strategy for treating a cancer patient. A gene is identified whose expression in cancer cells of the patient is reactivated by a CpG dinucleotide demethylating agent, a DNA methyltransferase inhibitor, or a histone deacetylase (HDAC) inhibitor. The gene is selected from the group consisting of BOLL, CABYR, EFEMP1, FBLN2, FOXL2, GNB4, GSTM3, HoxD1, Jph3, Neuralized (NEURL), PPP1R14a, TP53AP1, RAB32, SYNE1, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, SOX17, STK31, SYCP3, TCL1A, TFPI-2, TLR2, UCHL1, ZFP42, ASCL2, ATP8A2, CTAG2, EPHA4, FANCF, FOXE1, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655. A therapeutic agent is selected for the cancer patient which increases expression of the gene for treating said cancer patient.

Another embodiment of the invention is a kit for assessing methylation in a test sample. The kit comprises at least the following reagents: a reagent that (a) modifies methylated cytosine residues but not non-methylated cytosine residues, or that (b) modifies non-methylated cytosine residues but not methylated cytosine residues; and a pair of oligonucleotide primers that specifically hybridizes under amplification conditions to a region of a gene within about 1 kb of said gene's transcription start site, said gene being selected from those shown in BOLL, CABYR, EFEMP1, FBLN2, FOXL2, GNB4, GSTM3, HoxD1, Jph3, Neuralized (NEURL), PPP1R14a, TP53AP1, RAB32, SYNE1, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, SOX17, STK31, SYCP3, TCL1A, TFPI-2, TLR2, UCHL1, ZFP42, ASCL2, ATP8A2, CTAG2, EPHA4, FANCF, FOXE1, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655.

### BRIEF DESCRIPTION OF THE FIGURES AND TABLE

FIG. 1A-1E. Approach for identification of the human cancer cell hypermethylome in HCT116 CRC cells. (FIG. 1A) RNA from the indicated cell lines was isolated, labeled, hybridized, scanned and fluorescent spot intensities normalized by background subtraction and Loess transformation using Agilent Technologies 44K human microarrays. Parental wild type HCT116 cells (WT) and isogenic knockout counterparts for DNA methyltransferase 1 (DNMT1−/−) or 3b (DNMT3 b−/−) are compared in our study. DKO cells are doubly deficient for both DNMT1 and DNMT3b. (FIG. 1B) Gene expression changes in HCT116 cells with genetic disruption of various DNA methyltransferases. A three dimensional scatter plot indicating the gene expression levels in HCT 116 cells with genetic disruption of DNMTs 1 (X axis), 3b (Z axis), and both DNMT's (DKO; Y axis) in log scale. Individual gene expression changes are in black with the average for three experiments (red spots) or from an individual experiment (blue spots) for those genes in DKO cells with greater than 4 fold expression change. (FIG. 1C) HCT116 cells were treated with 300 nM trichostatin A (TSA) for 18 hr or 5 μM 5-deoxyazacytidine (DAC) for 96 hr and processed as described above. (FIG. 1D) Gene expression changes for HCT116 cells treated with TSA (X axis) or DAC(Y axis) are plotted by fold change. Yellow spots indicate genes from DKO cells with 2 fold changes and above. Notice the loss of sensitivity when compared to gene expression increases seen in DKO cells (greater than 2-fold in the DKO cells now becomes greater than 1.3 fold in DAC treated cells). Green spots indicate randomly selected genes verified to have complete promoter methylation in wild type cells, re-expression in DKO cells and after AZA treatment, while red spots indicate selected genes that were identified as false positives (See FIG. 5 (S2) for validation results). Blue spots indicate the location of the 11 guide genes—previously shown to be hypermethylated and completely silenced in HCT 116 cells—used in this study (see FIG. 4 (S1) for description). A distinct group of genes, including 5 of 11 guide genes, display increases of greater than 2 fold after DAC treatment but no increase after TSA treatment. These genes form the top tier of candidate hypermethylated genes as discussed in the text. (FIG. 1E) Relatedness of whole transcriptome expression patterns identified by dendrogram analysis. Individual single genetic disruption of DNMT1 and 3b, DKO and DAC treatment, and TSA treatment each form three distinct categories of gene expression changes.

FIG. 2A-2C. Characterization of the human cancer cell hypermethylome in different human CRC cell lines. (FIG. 2.A) Gene expression changes for the indicted cells treated with TSA (X axis) or DAC for (Y axis) are plotted by fold change, and individual genes are shown in black. (FIG. 2.B) Validation of the DNA hypermethyome. The characteristic spike of hypermethylated genes defined by treatment of cells with DAC or TSA consists of 2 tiers, with distinct features. The top tier of genes was identified as a zone in which gene expression did not increase with TSA (<1.4 fold) and displayed no detectable expression in wild type cells, but increased greater than 2 fold with DAC treatment. The next tier of genes was identified as a cluster of genes for which expression changes were identical to those in the top tier, but increased between 1.4 fold and 2 fold with DAC treatment. Gene expression validation by RT-PCR and MSP indicated a validation frequency of 86% for top tier genes in HCT116 cells, including genes which increased in DKO cells by greater than 2 fold. Next tier genes in HCT116 cells were confirmed at a frequency of 49%, and in the SW480 top tier, with a frequency of 65%. (FIG. 2.C) Shared candidate hypermethylated genes in CRC cell lines. We identified a total of 5,906 genes in all 6 cell lines with expression changes falling within the criteria of top or next tier categories. Overlap in gene expression changes among 2, 3, 4, 5 or 6 cell lines are indicated; these range from 1414 genes shared among 2 cell lines, to 78 genes that were shared among all 6 cell lines.

FIG. 3A-3E. Comparison of hypermethylation and gene mutation frequencies in human tumor samples. (FIG. 3A) Methylation analysis of verified hypermethylome genes in human tissue samples. Twenty genes from the verified gene lists were randomly selected from the HCT116 top tier (BOLL, DDX43, DKK3, FOXL2, HoxD1, JPH3, Nef, Neuralized, PPP1R14a, RAB32, STK31, TLR2), HCT116 next tier (SalL4, TP53AP1), or SW480 top tier (ZFP42) and analyzed for methylation in CRC cell lines (white columns), normal colon (red columns) or primary tumors (green columns). Percentage of methylation is indicated on the Y axis, and the abbreviated gene name on the X axis. We tested at least 6 different cell lines, 16 to 40 colonic samples from non-cancer patients, and between 18 and 61 primary CRC samples for each gene. (FIG. 3B) Methylation analysis of CAN genes. Fifty six genes were identified as overlapping the hypermethylome and CAN gene lists, including 45 genes containing CpG islands. Selected genes from this list with methylation in cell lines (26 genes) were analyzed for methylation in normal colon (FIG. 3B) and primary CRC (FIG. 3C). Frequency of methylation of these genes is shown as a percentage. (FIG. 3D) Relationship between methylation and mutation for 13 genes overlapping the CAN and hypermethylome gene lists. (FIG. 3E) Model for gene inactivation mechanisms in human cancer.

FIG. 4A-D. (S1). Guide genes used in this study. (FIG. 4A) Gene names, Agilent Technologies probe name, Genbank accession number and references for the 11 guide genes previously shown to be hypermethylated and completely silenced in HCT116 cells. (FIG. 4B) Blue spots and gene names indicate the location of the 11 guide genes in a plot of TSA (X axis) versus DAC Y axis) gene expression changes or (FIG. 4C) of DKO (X axis) versus single knockout (Y axis) gene expression changes on a log scale. 5 of 11 guide genes, circled in green, display increases of greater than 2 fold after AZA treatment but no increase after TSA treatment and these same genes have greater than 3 fold increases in DKO cells (green circle) (FIG. 4D) Direct comparison of guide genes in DKO and DAC plots. A distinct group of 5 guide genes, indicated by a green circle, showing greater than 3 fold expression changes in DKO cells and greater than 2 fold in DAC treated cells, define the upper tier of candidate hypermethylated genes as discussed in the text. Another 3 genes increased 1.3 fold, and 3 failed to increase with DAC treatment allowing criteria for the next tier of gene expression to be established as described in the text.

FIG. 5. (S2). Gene expression and methylation validation of 35 top tier genes in HCT116 cells. List of HCT116 candidate hypermethylated genes selected for verification of expression (by RT-PCR of HCT116 and DKO cells) and promoter methylation (by MSP of HCT116 and DKO cells) status. Gene descriptions are indicated on the left side of the panel and gene names are shown next to the PCR results. Water (RT-PCR and MSP), in vitro methylated DNA (IVD for MSP), and Actin B (ACTB) were used as controls for each individual gene; a representative sample is shown. Green arrows identify genes that verified the array results, red arrows those that did not.

FIG. 6. (S3.) List of 35 HCT116 candidate next tier genes selected for verification of expression (by RT-PCR of HCT116 and DKO cells) and promoter methylation (by MSP of HCT116 and DKO cells) status. Gene names are indicated on the left side of the panel and gene names are shown next to the PCR results. Water (RT-PCR and MSP), in vitro methylated DNA (IVD for MSP), and Actin B (ACTB) were used as controls for each individual gene; a representative sample is shown. Green arrows identify genes that verified the array results, red arrows those that did not as discussed in the text.

FIG. 7. (S4.) List of 48 SW480 candidate top tier genes selected for verification of expression (by RT-PCR of SW480 and DAC treated SW480 cells) and promoter methylation (by RT-PCR of SW480 and DAC treated SW480 cells) status. Gene names are indicated on the left side of the panel and gene names are shown next to the PCR results. Water (RT-PCR), in vitro methylated DNA (IVD for MSP), and Actin B (ACTB) were used as controls for each individual gene; a representative sample is shown. Green arrows identify genes that verified the array results, red arrows those that did not as discussed in the text.

FIG. 8. (Table S1.) Quantitative estimate of hypermethylome size. Cell line and tier are indicated to the left, and the number of gene expression changes identified per tier is also shown. Calculations as to the size of the candidate hypermethylated gene pool for each tier was performed by multiplying gene expressions changes identified for each tier by 0.86 (for the top tier of HCT116), 0.65 (for the top tier of SW480, CaCO2, HT29, COLO320, and RKO) or 0.49 (for the next tier of HCT116, SW480, CaCO2, HT29, COLO320 and RKO). These fractions represent the validation frequency determined experimentally as described in the text. An estimate for the size of the HCT116 hypermethylome was derived as follows: 86% of 532=457 top tier genes plus 49% of 1190=583 next tier genes; 457+583=1040. The SW480 hypermethylome was estimated at 579 genes according to the calculation: 66% of 318 top tier=207 and 49% of 759 lower tier=372; 207+372=579. The overlap between hypermethylome gene lists and genes mutated in either breast or colon cancer are shown to the right.

FIG. 9. (Table 1) Information regarding sequences in sequence listing. Gene Number: a running number by gene. Gene name: gene name as used in the patent. GeneID: Gene ID from the NCBI system. Transcript ID associated with Gene ID: all transcript IDs from the ENSEMBL annotation system associate with a given GeneID having the same TSS [transcription start site; note that a gene ID can have multiple TSS and thus multiple Transcript IDs are grouped by their TSS]. SEQ ID NO: 1-125: Genomic sequence context (from 1000 bp 5′ of TSS of transcript up to 200 bp 3′ of TSS of transcript); genomic DNA sequence as found in the NCBI build 36. SEQ ID NO: 126-250: Bisulphite converted version of the sequences assuming full methylation of all CpG dinucleotides present.

FIG. 10—Scatter plot BNIP3 with ratio cut-off 20

FIG. 11—Scatter plot FOXE1 with ratio cut-off 20

FIG. 12—Scatter plot SYNE1 with ratio cut-off 100

FIG. 13—Scatter plot SOX17 with ratio cut-off 300

FIG. 14—Scatter plot JAM3 with ratio cut-off 20

FIG. 15—Scatter plot MMP2 with ratio cut-off 150. Note: this marker was tested on 76 controls and 90 cases

FIG. 16—Scatter plot GPNMB with ratio cut-off 150. Note: this marker was tested on 76 controls and 90 cases

### DETAILED DESCRIPTION OF THE INVENTION

We describe a whole human transcriptome microarray screen to identify genes silenced by promoter hypermethylation in human CRC. The approach readily identifies candidate cancer genes in single tumors with a high efficiency of validation. By comparing the list of candidate hypermethylated genes with mutated genes recently identified in CRC (3), we establish key relationships between the altered tumor genome and the gene hypermethylome. Our studies provide a platform to understand how epigenetic and genetic alterations drive human tumorigenesis.

We describe a gene expression approach with the capacity to define, for any human cancer type for which representative cell culture lines are available, a substantial fraction of the cancer gene promoter CpG island DNA hypermethylome. Studies of these genes will contribute to understanding the molecular pathways driving tumorigenesis, provide useful new DNA hypermethylation biomarkers to monitor cancer risk assessment, early diagnosis, and prognosis, and permit better monitoring of gene re-expression during cancer prevention and/or therapy strategies (13, 22).

Through direct comparison of hypermethylome genes found by our approach to mutated genes identified by a genome wide sequencing strategy, we document that many more epigenetically versus genetically altered genes exist in any given tumor. The importance of this fact emerges in our finding that for newly discovered genes that are affected by both mechanisms, the incidence for hypermethylation of any given gene among colon cancers appears to be much higher than for mutations. Therefore, within a given cancer type, one may markedly underestimate both the full range of gene alterations and associated abnormalities of cellular pathways by failing to screen for both genetic and epigenetic abnormalities. The data also indicate that assessing both mechanisms for loss of gene function indicates far more sharing among individual colon tumors for pathway disruption than genetic analyses alone would predict. Our findings emphasize that optimal approaches to grouping of tumors according to molecular alterations in key pathways should depend on defining both genetic and epigenetic gene changes. Thus, our findings should encourage any genome wide strategies for mapping aberrant gene changes in cancer to take into account that mutated genes should be examined for promoter DNA hypermethylation and DNA hypermethylated genes should be put in a priority position for sequencing to find mutations.

Using this technique, we have discovered a set of genes whose transcription is epigenetically silenced in cancers, cancer precursors, and pre-cancers. The genes include: BOLL, CABYR, EFEMP1, FBLN2, FOXL2, GNB4, GSTM3, HoxD1, Jph3, Neuralized (NEURL), PPP1R14a, TP53AP1, RAB32, SYNE1, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, SOX17, STK31, SYCP3, TCL1A, TFPI-2, TLR2, UCHL1, ZFP42, ASCL2, ATP8A2, CTAG2, EPHA4, FANCF, FOXE1, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655. Detection of epigenetic silencing of at least 1, 2, 3, 4, 5, 6, 7, 8, 9, or 10 of such genes can be used as an indication of cancer or pre-cancer or risk of developing cancer.

Epigenetic silencing of a gene can be determined by any method known in the art. One method is to determine that a gene which is expressed in normal cells or other control cells is less expressed or not expressed in tumor cells. This method does not, on its own, however, indicate that the silencing is epigenetic, as the mechanism of the silencing could be genetic, for example, by somatic mutation. One method to determine that the silencing is epigenetic is to treat with a reagent, such as DAC (5′-deazacytidine), or with a reagent which changes the histone acetylation status of cellular DNA or any other treatment affecting epigenetic mechanisms present in cells, and observe that the silencing is reversed, i.e., that the expression of the gene is reactivated or restored. Another means to determine epigenetic silencing is to determine the presence of methylated CpG dinucleotide motifs in the silenced gene. Typically these reside near the transcription start site, for example, within about 1 kbp, within about 750 bp, or within about 500 bp. Once a gene has been identified as the target of epigenetic silencing in tumor cells, determination of reduced expression can be used as an indicator of epigenetic silencing.

Expression of a gene can be assessed using any means known in the art. Typically expression is assessed and compared in test samples and control samples which may be normal, non-malignant cells. The test samples may contain cancer cells or pre-cancer cells or nucleic acids from them. For example the sample may contain colon adenoma cells, colon advanced adenoma cells, or colon carcinoma cells. Either mRNA (nucleic acids) or protein can be measured. Methods employing hybridization to nucleic acid probes can be employed for measuring specific mRNAs. Such methods include using nucleic acid probe arrays (microarray technology), in situ hybridization, and using Northern blots. Messenger RNA can also be assessed using amplification techniques, such as RT-PCR. Advances in genomic technologies now permit the simultaneous analysis of thousands of genes, although many are based on the same concept of specific probe-target hybridization. Sequencing-based methods are an alternative; these methods started with the use of expressed sequence tags (ESTs), and now include methods based on short tags, such as serial analysis of gene expression (SAGE) and massively parallel signature sequencing (MPSS). Differential display techniques provide yet another means of analyzing gene expression; this family of techniques is based on random amplification of cDNA fragments generated by restriction digestion, and bands that differ between two tissues identify cDNAs of interest. Specific proteins can be assessed using any convenient method including immunoassays and immuno-cytochemistry but are not limited to that. Most such methods will employ antibodies which are specific for the particular protein or protein fragments. The sequences of the mRNA (cDNA) and proteins of the markers of the present invention are known in the art and publicly available.

Methylation-sensitive restriction endonucleases can be used to detect methylated CpG dinucleotide motifs. Such endonucleases may either preferentially cleave methylated recognition sites relative to non-methylated recognition sites or preferentially cleave non-methylated relative to methylated recognition sites. Examples of the former are Acc III, Ban I, BstN I, Msp I, and Xma I. Examples of the latter are Acc II, Ava I, BssH II, BstU I, Hpa II, and Not I. Alternatively, chemical reagents can be used which selectively modify either the methylated or non-methylated form of CpG dinucleotide motifs.

Modified products can be detected directly, or after a further reaction which creates products which are easily distinguishable. Means which detect altered size and/or charge can be used to detect modified products, including but not limited to electrophoresis, chromatography, and mass spectrometry. Examples of such chemical reagents for selective modification include hydrazine and bisulfite ions. Hydrazine-modified DNA can be treated with piperidine to cleave it. Bisulfite ion-treated DNA can be treated with alkali. Other means which are reliant on specific sequences can be used, including but not limited to hybridization, amplification, sequencing, and ligase chain reaction, Combinations of such techniques can be uses as is desired.

The principle behind electrophoresis is the separation of nucleic acids via their size and charge. Many assays exist for detecting methylation and most rely on determining the presence or absence of a specific nucleic acid product. Gel electrophoresis is commonly used in a laboratory for this purpose.

One may use MALDI mass spectrometry in combination with a methylation detection assay to observe the size of a nucleic acid product. The principle behind mass spectrometry is the ionizing of nucleic acids and separating them according to their mass to charge ratio. Similar to electrophoresis, one can use mass spectrometry to detect a specific nucleic acid that was created in an experiment to determine methylation. See Tost, J. et al. Analysis and accurate quantification of CpG methylation by MALDI mass spectrometry. Nuc Acid Res, 2003, 31, 9

One form of chromatography, high performance liquid chromatography, is used to separate components of a mixture based on a variety of chemical interactions between a substance being analyzed and a chromatography column. DNA is first treated with sodium bisulfite, which converts an unmethylated cytosine to uracil, while methylated cytosine residues remain unaffected. One may amplify the region containing potential methylation sites via PCR and separate the products via denaturing high performance liquid chromatography (DHPLC). DHPLC has the resolution capabilities to distinguish between methylated (containing cytosine) and unmethylated (containing uracil) DNA sequences. See Deng, D. et al. Simultaneous detection of CpG methylation and single nucleotide polymorphism by denaturing high performance liquid chromatography. 2002 Nuc Acid Res, 30, 3.

Hybridization is a technique for detecting specific nucleic acid sequences that is based on the annealing of two complementary nucleic acid strands to form a double-stranded molecule. One example of the use of hybridization is a microarray assay to determine the methylation status of DNA. After sodium bisulfite treatment of DNA, which converts an unmethylated cytosine to uracil while methylated cytosine residues remain unaffected, oligonucleotides complementary to potential methylation sites can hybridize to the bisulfite-treated DNA. The oligonucleotides are designed to be complimentary to either sequence containing uracil or sequence containing cytosine, representing unmethylated and methylated DNA, respectively. Computer-based microarray technology can determine which oligonucleotides hybridize with the DNA sequence and one can deduce the methylation status of the DNA.

An additional method of determining the results after sodium bisulfite treatment would be to sequence the DNA to directly observe any bisulfite-modifications. Pyrosequencing technology is a method of sequencing-by-synthesis in real time. It is based on an indirect bioluminometric assay of the pyrophosphate (PPi) that is released from each deoxynucleotide (dNTP) upon DNA-chain elongation. This method presents a DNA template-primer complex with a dNTP in the presence of an exonuclease-deficient Klenow DNA polymerase. The four nucleotides are sequentially added to the reaction mix in a predetermined order. If the nucleotide is complementary to the template base and thus incorporated, PPi is released. The PPi and other reagents are used as a substrate in a luciferase reaction producing visible light that is detected by either a luminometer or a charge-coupled device. The light produced is proportional to the number of nucleotides added to the DNA primer and results in a peak indicating the number and type of nucleotide present in the form of a pyrogram. Pyrosequencing can exploit the sequence differences that arise following sodium bisulfite-conversion of DNA.

A variety of amplification techniques may be used in a reaction for creating distinguishable products. Some of these techniques employ PCR. Other suitable amplification methods include the ligase chain reaction (LCR) (Barringer et al, 1990), transcription amplification (Kwoh et al. 1989; WO88/10315), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO90/06995), nucleic acid based sequence amplification (NASBA) (U.S. Pat. Nos. 5,409,818; 5,554,517; 6,063,603), nick displacement amplification (WO2004/067726).

Sequence variation that reflects the methylation status at CpG dinucleotides in the original genomic DNA offers two approaches to PCR primer design. In the first approach, the primers do not themselves “cover” or hybridize to any potential sites of DNA methylation; sequence variation at sites of differential methylation are located between the two primers. Such primers are used in bisulphite genomic sequencing, COBRA, Ms-SNuPE. In the second approach, the primers are designed to anneal specifically with either the methylated or unmethylated version of the converted sequence. If there is a sufficient region of complementarity, e.g., 12, 15, 18, or 20 nucleotides, to the target, then the primer may also contain additional nucleotide residues that do not interfere with hybridization but may be useful for other manipulations. Exemplary of such other residues may be sites for restriction endonuclease cleavage, for ligand binding or for factor binding or linkers or repeats. The oligonucleotide primers may or may not be such that they are specific for modified methylated residues

One way to distinguish between modified and unmodified DNA is to hybridize oligonucleotide primers which specifically bind to one form or the other of the DNA. After hybridization, an amplification reaction can be performed and amplification products assayed. The presence of an amplification product indicates that a sample hybridized to the primer. The specificity of the primer indicates whether the DNA had been modified or not, which in turn indicates whether the DNA had been methylated or not. For example, bisulfite ions modify non-methylated cytosine bases, changing them to uracil bases. Uracil bases hybridize to adenine bases under hybridization conditions. Thus an oligonucleotide primer which comprises adenine bases in place of guanine bases would hybridize to the bisulfite-modified DNA, whereas an oligonucleotide primer containing the guanine bases would hybridize to the non-modified (methylated) cytosine residues in the DNA. Amplification using a DNA polymerase and a second primer yield amplification products which can be readily observed. Such a method is termed MSP (Methylation Specific PCR; U.S. Pat. Nos. 5,786,146; 6,017,704; 6,200,756). The amplification products can be optionally hybridized to specific oligonucleotide probes which may also be specific for certain products. Alternatively, oligonucleotide probes can be used which will hybridize to amplification products from both modified and nonmodified DNA.

Another way to distinguish between modified and nonmodified DNA is to use oligonucleotide probes which may also be specific for certain products. Such probes can be hybridized directly to modified DNA or to amplification products of modified DNA. Oligonucleotide probes can be labeled using any detection system known in the art. These include but are not limited to fluorescent moieties, radioisotope labeled moieties, bioluminescent moieties, luminescent moieties, chemiluminescent moieties, enzymes, substrates, receptors, or ligands.

Still another way for the identification of methylated CpG dinucleotides utilizes the ability of the MBD domain of the McCP2 protein to selectively bind to methylated DNA sequences (Cross et al, 1994; Shiraishi et al, 1999). Restriction endonuclease digested genomic DNA is loaded onto expressed His-tagged methyl-CpG binding domain that is immobilized to a solid matrix and used for preparative column chromatography to isolate highly methylated DNA sequences.

Real time chemistry allows for the detection of PCR amplification during the early phases of the reactions, and makes quantitation of DNA and RNA easier and more precise. A few variations of the real-time PCR are known. They include the TaqMan™ system and Molecular Beacon™ system which have separate probes labeled with a fluorophore and a fluorescence quencher. In the Scorpion™ system the labeled probe in the form of a hairpin structure is linked to the primer.

DNA methylation analysis has been performed successfully with a number of techniques which include the MALDI-TOFF, MassARRAY, MethyLight, Quantitative analysis of ethylated alleles (QAMA), enzymatic regional methylation assay (ERMA), HeavyMethyl, QBSUPT, MS-SNuPE, MethylQuant, Quantitative PCR sequencing, and Oligonucleotide-based microarray systems.

The number of genes whose silencing is tested and/or detected can vary: one, two, three, four, five, or more genes can be tested and/or detected. In some cases at least two genes are selected. In other embodiments at least three genes are selected.

Testing can be performed diagnostically or in conjunction with a therapeutic regimen. Testing can be used to monitor efficacy of a therapeutic regimen, whether a chemotherapeutic agent or a biological agent, such as a polynucleotide. Testing can also be used to determine what therapeutic or preventive regimen to employ on a patient. Moreover, testing can be used to stratify patients into groups for testing agents and determining their efficacy on various groups of patients.

Test samples for diagnostic, prognostic, or personalized medicine uses can be obtained from surgical samples, such as biopsies or fine needle aspirates, from paraffin embedded colon, rectum, small intestinal, gastric, esophageal, bone marrow, breast, ovary, prostate, kidney, lung, brain on other organ tissues, from a body fluid such as blood, serum, lymph, cerebrospinal fluid, saliva, sputum, bronchial-lavage fluid, ductal fluids stool, urine, lymph nodes, or semen. Such sources are not meant to be exhaustive, but rather exemplary. A test sample obtainable from such specimens or fluids includes detached tumor cells and/or free nucleic acids that are released from dead or damaged tumor cells. Nucleic acids include RNA, genomic DNA, mitochondrial DNA, single or double stranded, and protein-associated nucleic acids. Any nucleic acid specimen in purified or non-purified form obtained from such specimen cell can be utilized as the starting nucleic acid or acids.

Demethylating agents can be contacted with cells in vitro or in vivo for the purpose of restoring normal gene expression to the cell. Suitable demethylating agents include, but are not limited to 5-aza-2′-deoxycytidine, 5-aza-cytidine, Zebularine, procaine, and L-ethionine. This reaction may be used for diagnosis, for determining predisposition, and for determining suitable therapeutic regimes. If the demethylating agent is used for treating colon, head and neck, esophageal, gastric, pancreatic, or liver cancers, expression or methylation can be tested of a gene selected from BOLL, CABYR, EFEMP1, FBLN2, FOXL2, GNB4, GSTM3, HoxD1, Jph3, Neuralized (NEURL), PPP1R14a, TP53AP1, RAB32, SYNE1, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, SOX17, STK31, SYCP3, TCL1A, TFPI-2, TLR2, UCHL1, ZFP42, ASCL2, ATP8A2, CTAG2, EPHA4, FANCF, FOXE1, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655.

An alternative way to restore epigenetically silenced gene expression is to introduce a non-methylated polynucleotide into a cell, so that it will be expressed in the cell. Various gene therapy vectors and vehicles are known in the art and any can be used as is suitable for a particular situation. Certain vectors are suitable for short term expression and certain vectors are suitable for prolonged expression. Certain vectors are trophic for certain organs and these can be used as is appropriate in the particular situation. Vectors may be viral or non-viral. The polynucleotide can, but need not, be contained in a vector, for example, a viral vector, and can be formulated, for example, in a matrix such as a liposome, microbubbles. The polynucleotide can be introduced into a cell by administering the polynucleotide to the subject such that it contacts the cell and is taken up by the cell and the encoded polypeptide expressed. Preferably the specific polynucleotide will be one which the patient has been tested for and been found to carry a silenced version. The polynucleotides for treating colon, head and neck, esophageal, gastric, pancreas, liver cancers will typically encode a gene selected from BOLL, CABYR, EFEMP1, FBLN2, FOXL2, GNB4, GSTM3, HoxD1, Jph3, Neuralized (NEURL), PPP1R14a, TP53AP1, RAB32, SYNE1, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, SOX17, STK31, SYCP3, TCL1A, TFPI-2, TLR2, UCHL1, ZFP42, ASCL2, ATP8A2, CTAG2, EPHA4, FANCF, FOXE1, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655.

Cells exhibiting methylation silenced gene expression generally are contacted with the demethylating agent in vivo by administering the agent to a subject. Where convenient, the demethylating agent can be administered using, for example, a catheterization procedure, at or near the site of the cells exhibiting unregulated growth in the subject, or into a blood vessel in which the blood is flowing to the site of the cells. Similarly, where an organ, or portion thereof, to be treated can be isolated by a shunt procedure, the agent can be administered via the shunt, thus substantially providing the agent to the site containing the cells. The agent also can be administered systemically or via other routes known in the art.

The polynucleotide can include, in addition to polypeptide coding sequence, operatively linked transcriptional regulatory elements, translational regulatory elements, and the like, and can be in the form of a naked DNA molecule, which can be contained in a vector, or can be formulated in a matrix such as a liposome or microbubbles that facilitates entry of the polynucleotide into the particular cell. The term “operatively linked” refers to two or more molecules that are positioned with respect to each other such that they act as a single unit and effect a function attributable to one or both molecules or a combination thereof. A polynucleotide sequence encoding a desired polypeptide can be operatively linked to a regulatory element, in which case the regulatory element confers its regulatory effect on the polynucleotide similar to the way in which the regulatory element would affect a polynucleotide sequence with which it normally is associated with in a cell.

The polynucleotide encoding the desired polypeptide to be administered to a mammal or a human or to be contacted with a cell may contain a promoter sequence, which can provide constitutive or, if desired, inducible or tissue specific or developmental stage specific expression of the polynucleotide, a polyA recognition sequence, and a ribosome recognition site or internal ribosome entry site, or other regulatory elements such as an enhancer, which can be tissue specific. The vector also may contain elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther. 1:51-64, 1994; Flotte, J. Bioenerg. Biomemb. 25:37-42, 1993; Kirshenbaum et al., J. Clin. Invest. 92:381-387, 1993; each of which is incorporated herein by reference).

A tetracycline (tet) inducible promoter can be used for driving expression of a polynucleotide encoding a desired polypeptide. Upon administration of tetracycline, or a tetracycline analog, to a subject containing a polynucleotide operatively linked to a tet inducible promoter, expression of the encoded polypeptide is induced. The polynucleotide alternatively can be operatively linked to tissue specific regulatory element, for example, a liver cell specific regulatory element such as an α-fetoprotein promoter (Kanai et al., Cancer Res. 57:461-465, 1997; He et al., J. Exp. Clin. Cancer Res. 19:183-187, 2000) or an albumin promoter (Power et al., Biochem. Biophys. Res. Comm. 203:1447-1456, 1994; Kuriyama et al., Int. J. Cancer 71:470-475, 1997); a muscle cell specific regulatory element such as a myoglobin promoter (Devlin et al., J. Biol. Chem. 264:13896-13901, 1989; Yan et al., J. Biol. Chem. 276:17361-17366, 2001); a prostate cell specific regulatory element such as the PSA promoter (Schuur et al., J. Biol. Chem. 271:7043-7051, 1996; Latham et al., Cancer Res. 60:334-341, 2000); a pancreatic cell specific regulatory element such as the elastase promoter (Ornitz et al., Nature 313:600-602, 1985; Swift et al., Genes Devel. 3:687-696, 1989); a leukocyte specific regulatory element such as the leukosialin (CD43) promoter (Shelley et al., Biochem. J. 270:569-576, 1990; Kudo and Fukuda, J. Biol. Chem. 270:13298-13302, 1995); or the like, such that expression of the polypeptide is restricted to particular cell in an individual, or to particular cells in a mixed population of cells in culture, for example, an organ culture. Regulatory elements, including tissue specific regulatory elements, many of which are commercially available, are well known in the art (see, for example, InvivoGen; San Diego Calif.).

Viral expression vectors can be used for introducing a polynucleotide into a cell, particularly a cell in a subject. Viral vectors provide the advantage that they can infect host cells with relatively high efficiency and can infect specific cell types. For example, a polynucleotide encoding a desired polypeptide can be cloned into a baculovirus vector, which then can be used to infect an insect host cell, thereby providing a means to produce large amounts of the encoded polypeptide. Viral vectors have been developed for use in particular host systems, particularly mammalian systems and include, for example, retroviral vectors, other lentivirus vectors such as those based on the human immunodeficiency virus (HIV), adenovirus vectors, adeno-associated virus vectors, herpesvirus vectors, hepatitis virus vectors, vaccinia virus vectors, and the like (see Miller and Rosman, BioTechniques 7:980-990, 1992; Anderson et al., Nature 392:25-30 Suppl., 1998; Verma and Somia, Nature 389:239-242, 1997; Wilson, New Engl. J. Med. 334:1185-1187 (1996), each of which is incorporated herein by reference).

A polynucleotide, which can optionally be contained in a vector, can be introduced into a cell by any of a variety of methods known in the art (Sambrook et al., supra, 1989; Ausubel et al., Current Protocols in Molecular Biology, John Wiley and Sons, Baltimore, Md. (1987, and supplements through 1995), each of which is incorporated herein by reference). Such methods include, for example, transfection, lipofection, microinjection, electroporation and, with viral vectors, infection; and can include the use of liposomes, microemulsions or the like, which can facilitate introduction of the polynucleotide into the cell and can protect the polynucleotide from degradation prior to its introduction into the cell. A particularly useful method comprises incorporating the polynucleotide into microbubbles, which can be injected into the circulation. An ultrasound source can be positioned such that ultrasound is transmitted to the tumor, wherein circulating microbubbles containing the polynucleotide are disrupted at the site of the tumor due to the ultrasound, thus providing the polynucleotide at the site of the cancer. The selection of a particular method will depend, for example, on the cell into which the polynucleotide is to be introduced, as well as whether the cell is in culture or in situ in a body.

Introduction of a polynucleotide into a cell by infection with a viral vector can efficiently introduce the nucleic acid molecule into a cell. Moreover, viruses are very specialized and can be selected as vectors based on an ability to infect and propagate in one or a few specific cell types. Thus, their natural specificity can be used to target the nucleic acid molecule contained in the vector to specific cell types. A vector based on an HIV can be used to infect T cells, a vector based on an adenovirus can be used, for example, to infect respiratory epithelial cells, a vector based on a herpesvirus can be used to infect neuronal cells, and the like. Other vectors, such as adeno-associated viruses can have greater host cell range and, therefore, can be used to infect various cell types, although viral or non-viral vectors also can be modified with specific receptors or ligands to alter target specificity through receptor mediated events. A polynucleotide of the invention or a vector containing the polynucleotide can be contained in a cell, for example, a host cell, which allows propagation of a vector containing the polynucleotide, or a helper cell, which allows packaging of a viral vector containing the polynucleotide. The polynucleotide can be transiently contained in the cell, or can be stably maintained due, for example, to integration into the cell genome.

A polypeptide encoded by a gene (BOLL, CABYR, EFEMP1, FBLN2, FOXL2, GNB4, GSTM3, HoxD1, Jph3, Neuralized (NEURL), PPP1R14a, TP53AP1, RAB32, SYNE1, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, SOX17, STK31, SYCP3, TCL1A, TFPI-2, TLR2, UCHL1, ZFP42, ASCL2, ATP8A2, CTAG2, EPHA4, FANCF, FOXE1, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, or ZNF655) can be administered directly to the site of a cell exhibiting unregulated growth in the subject. The polypeptide can be produced and isolated, and formulated as desired, using methods as disclosed herein, and can be contacted with the cell such that the polypeptide can cross the cell membrane of the target cells. The polypeptide may be provided as part of a fusion protein, which includes a peptide or polypeptide component that facilitates transport across cell membranes. For example, a human immunodeficiency virus (HIV) TAT protein transduction domain or a nuclear localization domain may be fused to the marker of interest. The administered polypeptide can be formulated in a matrix that facilitates entry of the polypeptide into a cell.

While particular polynucleotide and polypeptide sequences are mentioned here as representative of known genes and proteins, those of skill in the art will understand that the sequences in the databases represent the sequences present in particular individuals. Any allelic sequences from other individuals can be used as well. These typically vary from the disclosed sequences at 1-10 residues, at 1-5 residues, or at 1-3 residues. Moreover, the allelic sequences are typically at least 95, 96, 97, 98, or 99% identical to the database sequence, as measured using an algorithm such as the BLAST homology tools.

An agent such as a demethylating agent, a polynucleotide, or a polypeptide is typically formulated in a composition suitable for administration to the subject. Thus, the invention provides compositions containing an agent that is useful for restoring regulated growth to a cell exhibiting unregulated growth due to methylation silenced transcription of one or more genes. The agents are useful as medicaments for treating a subject suffering from a pathological condition associated with such unregulated growth. Such medicaments generally include a carrier. Acceptable carriers are well known in the art and include, for example, aqueous solutions such as water or physiologically buffered saline or other solvents or vehicles such as glycols, glycerol, oils such as olive oil or injectable organic esters. An acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know or readily be able to determine an acceptable carrier, including a physiologically acceptable compound. The nature of the carrier depends on the physico-chemical characteristics of the therapeutic agent and on the route of administration of the composition. Administration of therapeutic agents or medicaments can be by the oral route or parenterally such as intravenously, intramuscularly, subcutaneously, transdermally, intranasally, intrabronchially, vaginally, rectally, intratumorally, or other such method known in the art. The pharmaceutical composition also can contain one more additional therapeutic agents.

The therapeutic agents can be incorporated within an encapsulating material such as into an oil-in-water emulsion, a microemulsion, micelle, mixed micelle, liposome, microsphere, microbubbles or other polymer matrix (see, for example, Gregoriadis, Liposome Technology, Vol. 1 (CRC Press, Boca Raton, Fla. 1984); Fraley, et al., Trends Biochem. Sci., 6:77 (1981), each of which is incorporated herein by reference). Liposomes, for example, which consist of phospholipids or other lipids, are nontoxic, physiologically acceptable and metabolizable carriers that are relatively simple to make and administer. “Stealth” liposomes (see, for example, U.S. Pat. Nos. 5,882,679; 5,395,619; and 5,225,212, each of which is incorporated herein by reference) are an example of such encapsulating materials particularly useful for preparing a composition useful in a method of the invention, and other “masked” liposomes similarly can be used, such liposomes extending the time that the therapeutic agent remain in the circulation. Cationic liposomes, for example, also can be modified with specific receptors or ligands (Morishita et al., J. Clin. Invest., 91:2580-2585 (1993), which is incorporated herein by reference). In addition, a polynucleotide agent can be introduced into a cell using, for example, adenovirus-polylysine DNA complexes (see, for example, Michael et al., J. Biol. Chem. 268:6866-6869 (1993), which is incorporated herein by reference).

The route of administration of the composition containing the therapeutic agent will depend, in part, on the chemical structure of the molecule. Polypeptides and polynucleotides, for example, are not efficiently delivered orally because they can be degraded in the digestive tract. However, methods for chemically modifying polypeptides, for example, to render them less susceptible to degradation by endogenous proteases or more absorbable through the alimentary tract may be used (see, for example, Blondelle et al., supra, 1995; Ecker and Crook, supra, 1995).

The total amount of an agent to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the composition to treat a pathologic condition in a subject depends on many factors including the age and general health of the subject as well as the route of administration and the number of treatments to be administered. In view of these factors, the skilled artisan would adjust the particular dose as necessary. In general, the formulation of the composition and the routes and frequency of administration are determined, initially, using Phase I and Phase II clinical trials.

The composition can be formulated for oral formulation, such as a tablet, or a solution or suspension form; or can comprise an admixture with an organic or inorganic carrier or excipient suitable for enteral or parenteral applications, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695).

Although diagnostic and prognostic accuracy and sensitivity may be achieved by using a combination of markers, such as 5 or 6 markers, or 9 or 10 markers, or 14 or 15 markers, practical considerations may dictate use of smaller combinations. Any combination of markers for a specific cancer may be used which comprises 2, 3, 4, or 5 markers. Combinations of 2, 3, 4, or 5 markers can be readily envisioned given the specific disclosures of individual markers provided herein.

The level of methylation of the differentially methylated GpG islands can provide a variety of information about the disease or cancer. It can be used to diagnose pre-cancer or cancer in the individual. Pre-cancer or cancer precursor is a very early stage of cancer which is found in the innermost (luminal) layer of the colon. It is sometimes referred to as superficial cancer. Alternatively, it can be used to predict the course of the disease or cancer in the individual or to predict the susceptibility to disease or cancer or to stage the progression of the disease or cancer in the individual. It can help to predict the likelihood of overall survival or predict the likelihood of reoccurrence of disease or cancer and to determine the effectiveness of a treatment course undergone by the individual. Increase or decrease of methylation levels in comparison with reference level and alterations in the increase/decrease when detected provide useful prognostic and diagnostic value.

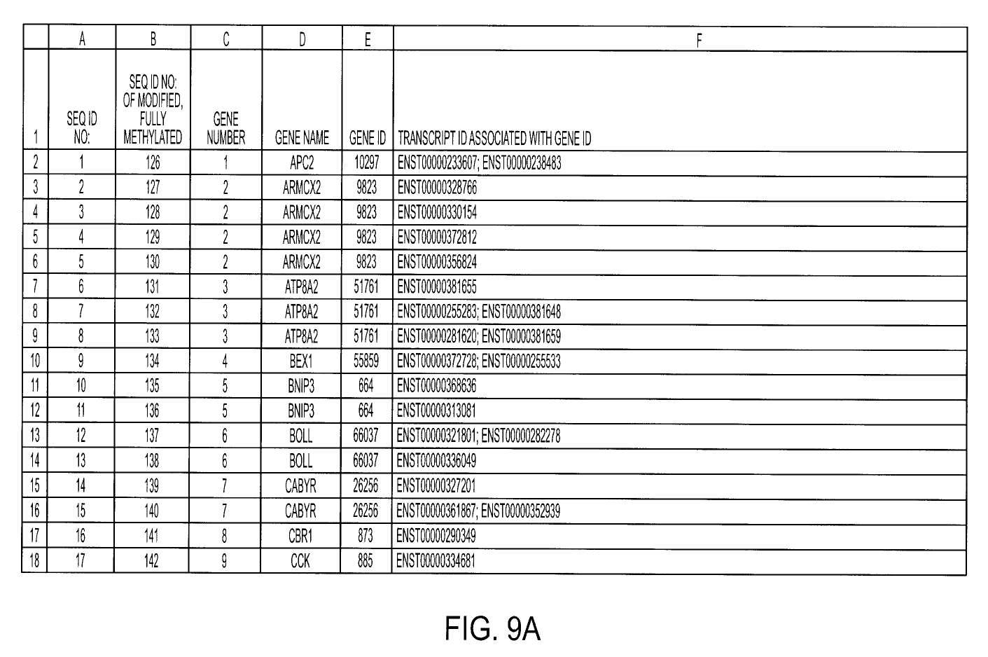

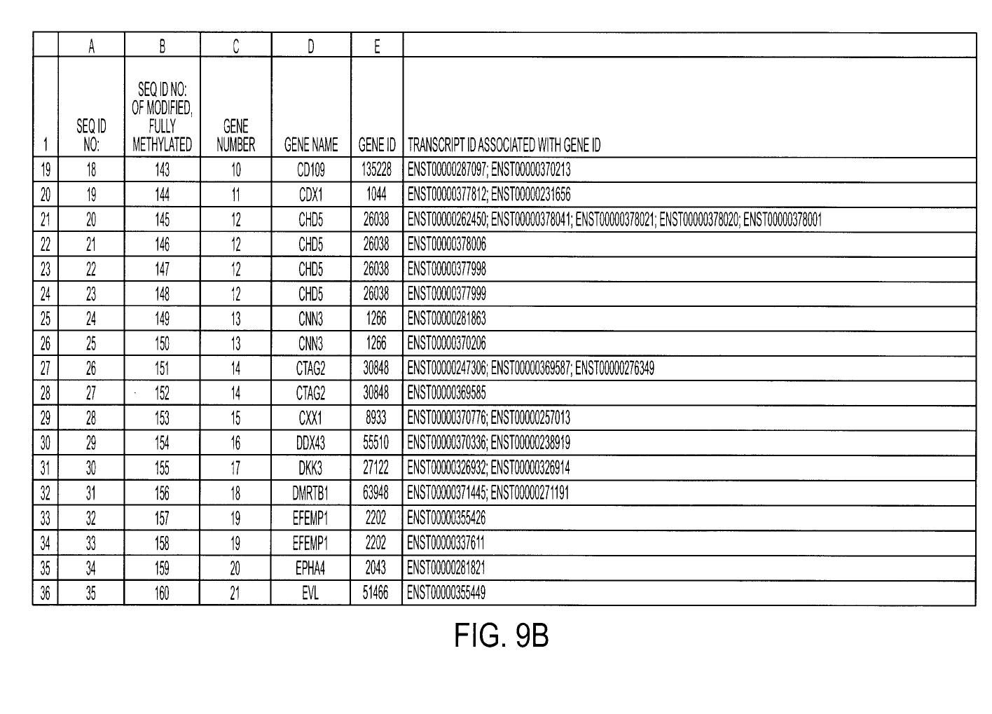

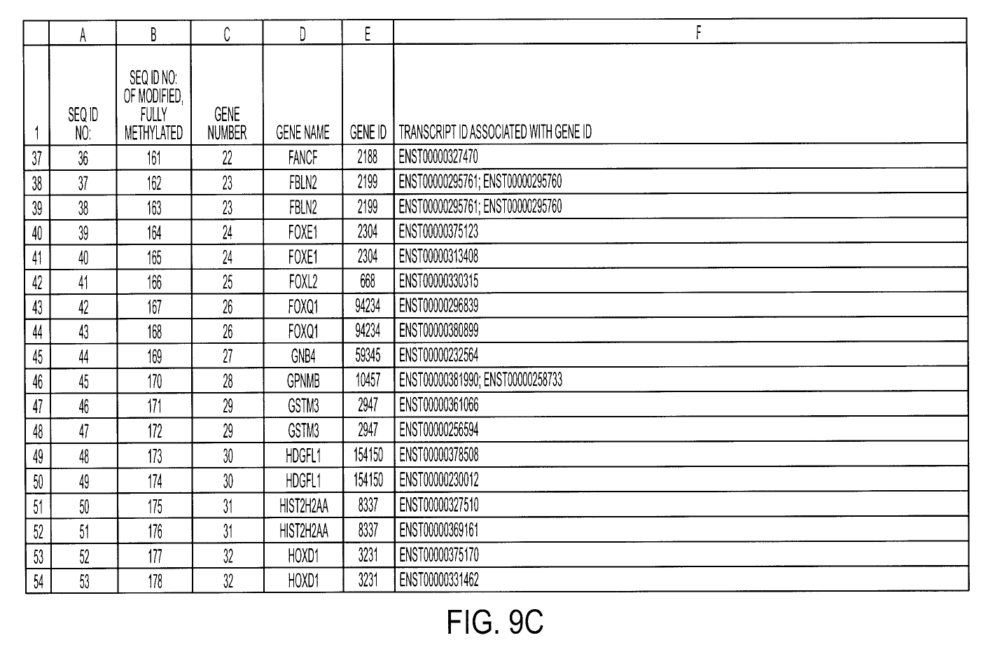

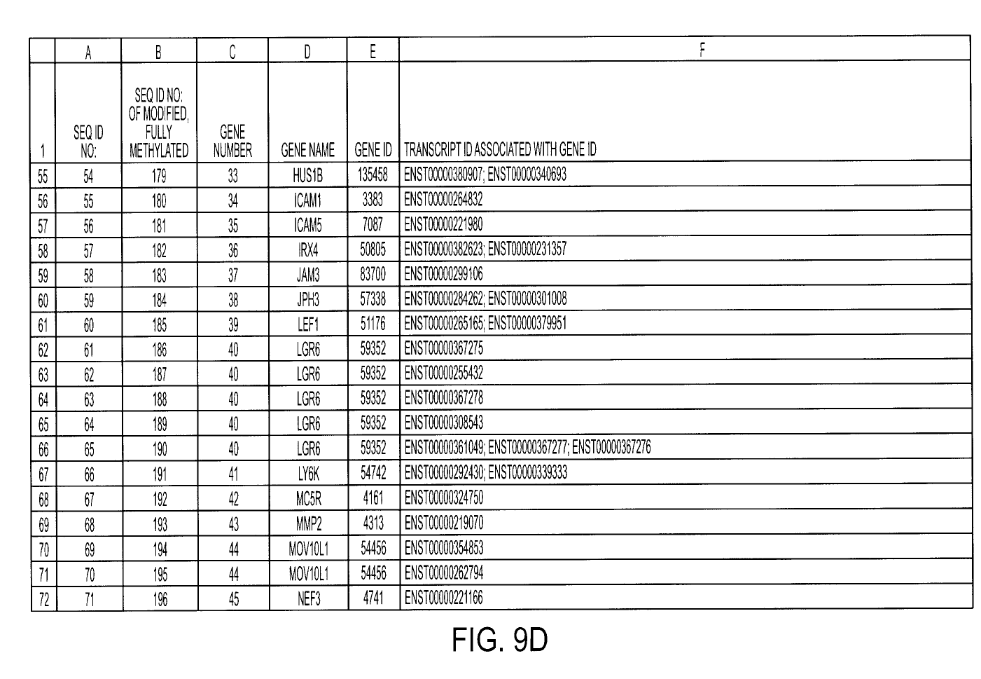

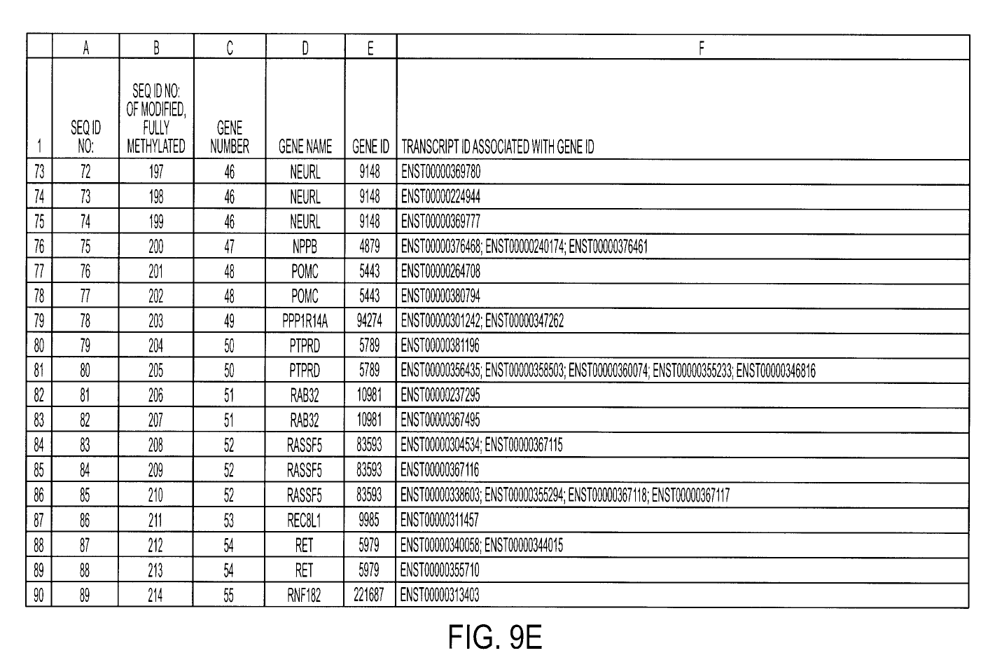

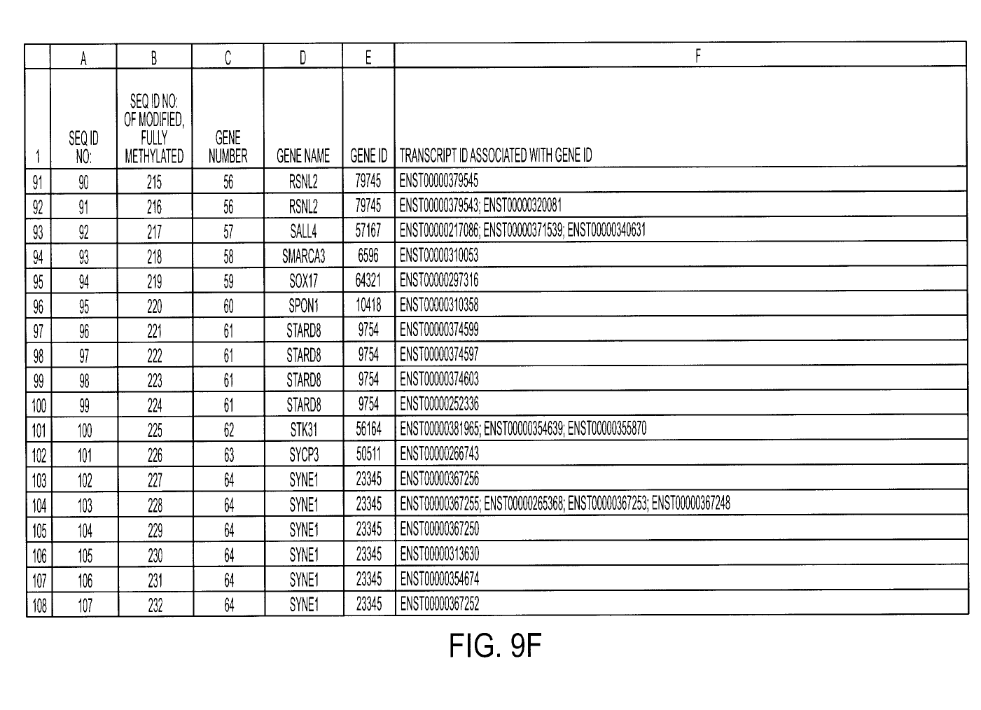

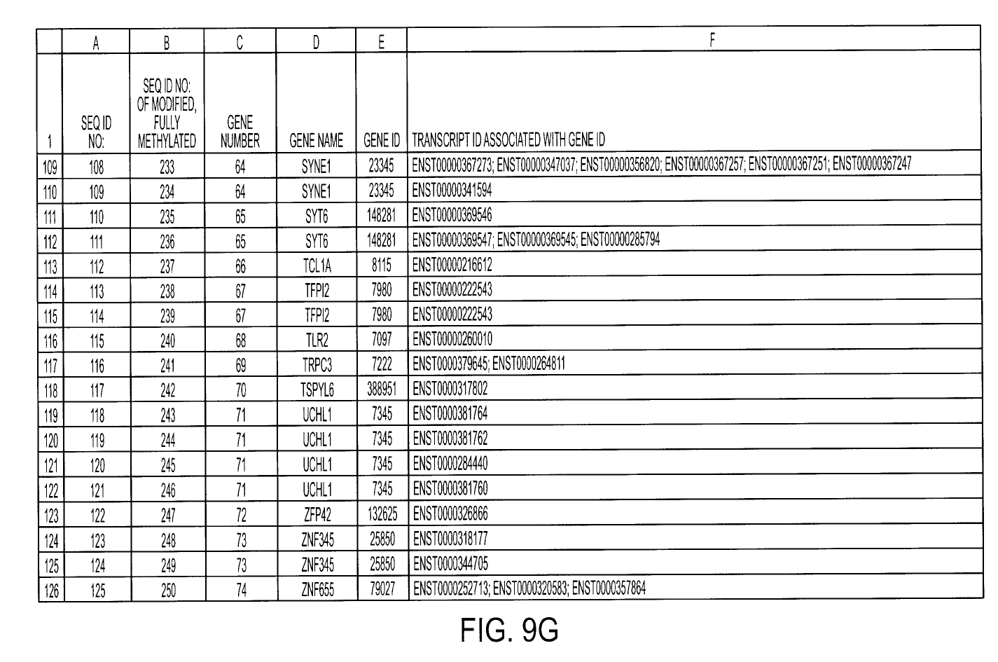

The prognostic methods can be used to identify patients with adenomas that are likely to progress to carcinomas. Such a prediction can be made on the basis of epigenetic silencing of at least one of the genes identified in Table 1 (FIG. 9) in an adenoma relative to normal tissue. Such patients can be offered additional appropriate therapeutic or preventative options, including endoscopic polypectomy or resection, and when indicated, surgical procedures, chemotherapy, radiation, biological response modifiers, or other therapies. Such patients may also receive recommendations for further diagnostic or monitoring procedures, including but not limited to increased frequency of colonoscopy, sigmoidoscopy, virtual colonoscopy, video capsule endoscopy, PET-CT, molecular imaging, or other imaging techniques.

A therapeutic strategy for treating a cancer patient can be selected based on reactivation of epigenetically silenced genes. First a gene selected from BOLL, CABYR, EFEMP1, FBLN2, FOXL2, GNB4, GSTM3, HoxD1, Jph3, Neuralized (NEURL), PPP1R14a, TP53AP1, RAB32, SYNE1, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, SOX17, STK31, SYCP3, TCL1A, TFPI-2, TLR2, UCHL1, ZFP42, ASCL2, ATP8A2, CTAG2, EPHA4, FANCF, FOXE1, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655 is identified whose expression in cancer cells of the patient is reactivated by a demethylating agent or epigenetically silenced. A treatment which increases the expression of the gene is then selected. Such a treatment can comprise administration of a reactivating agent or a polynucleotide. A polypeptide can alternatively be administered.

Kits according to the present invention are assemblages of reagents for testing methylation. They are typically in a package which contains all elements, optionally including instructions. The package may be divided so that components are not mixed until desired. Components may be in different physical states. For example, some components may be lyophilized and some in aqueous solution. Some may be frozen. Individual components may be separately packaged within the kit. The kit may contain reagents, as described above for differentially modifying methylated and non-methylated cytosine residues. Desirably the kit will contain oligonucleotide primers which specifically hybridize to regions within 1 kb of the transcription start sites of the genes/markers: BOLL, CABYR, EFEMP1, FBLN2, FOXL2, GNB4, GSTM3, HoxD1, Jph3, Neuralized (NEURL), PPP1R14a, TP53AP1, RAB32, SYNE1, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, SOX17, STK31, SYCP3, TCL1A, TFPI-2, TLR2, UCHL1, ZFP42, ASCL2, ATP8A2, CTAG2, EPHA4, FANCF, FOXE1, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655. Typically the kit will contain both a forward and a reverse primer for a single gene or marker. If there is a sufficient region of complementarity, e.g., 12, 15, 18, or 20 nucleotides, then the primer may also contain additional nucleotide residues that do not interfere with hybridization but may be useful for other manipulations. Exemplary of such other residues may be sites for restriction endonuclease cleavage, for ligand binding or for factor binding or linkers or repeats. The oligonucleotide primers may or may not be such that they are specific for modified methylated residues. The kit may optionally contain oligonucleotide probes. The probes may be specific for sequences containing modified methylated residues or for sequences containing non-methylated residues. The kit may optionally contain reagents for modifying methylated cytosine residues. The kit may also contain components for performing amplification, such as a DNA polymerase and deoxyribonucleotides. Means of detection may also be provided in the kit, including detectable labels on primers or probes. Kits may also contain reagents for detecting gene expression for one of the markers of the present invention (Table 1; FIG. 9). Such reagents may include probes, primers, or antibodies, for example. In the case of enzymes or ligands, substrates or binding partners may be sued to assess the presence of the marker.

In one aspect of this embodiment, the gene is contacted with hydrazine, which modifies cytosine residues, but not methylated cytosine residues, then the hydrazine treated gene sequence is contacted with a reagent such as piperidine, which cleaves the nucleic acid molecule at hydrazine modified cytosine residues, thereby generating a product comprising fragments. By separating the fragments according to molecular weight, using, for example, an electrophoretic, chromatographic, or mass spectrographic method, and comparing the separation pattern with that of a similarly treated corresponding non-methylated gene sequence, gaps are apparent at positions in the test gene contained methylated cytosine residues. As such, the presence of gaps is indicative of methylation of a cytosine residue in the CpG dinucleotide in the target gene of the test cell.

Bisulfite ions, for example, sodium bisulfite, convert non-methylated cytosine residues to bisulfite modified cytosine residues. The bisulfite ion treated gene sequence can be exposed to alkaline conditions, which convert bisulfite modified cytosine residues to uracil residues. Sodium bisulfite reacts readily with the 5,6-double bond of cytosine (but poorly with methylated cytosine) to form a sulfonated cytosine reaction intermediate that is susceptible to deamination, giving rise to a sulfonated uracil. The sulfonate group can be removed by exposure to alkaline conditions, resulting in the formation of uracil. The DNA can be amplified, for example, by PCR, and sequenced to determine whether CpG sites are methylated in the DNA of the sample. Uracil is recognized as a thymine by Taq polymerase and, upon PCR, the resultant product contains cytosine only at the position where 5-methylcytosine was present in the starting template DNA. One can compare the amount or distribution of uracil residues in the bisulfite ion treated gene sequence of the test cell with a similarly treated corresponding non-methylated gene sequence. A decrease in the amount or distribution of uracil residues in the gene from the test cell indicates methylation of cytosine residues in CpG dinucleotides in the gene of the test cell. The amount or distribution of uracil residues also can be detected by contacting the bisulfite ion treated target gene sequence, following exposure to alkaline conditions, with an oligonucleotide that selectively hybridizes to a nucleotide sequence of the target gene that either contains uracil residues or that lacks uracil residues, but not both, and detecting selective hybridization (or the absence thereof) of the oligonucleotide.

Test compounds can be tested for their potential to treat cancer. Cancer cells for testing can be selected from the group consisting of prostate, lung, breast, and colon cancer. Expression of a gene selected from those listed in Table 1 (FIG. 9) is determined and if it is increased by the compound in the cell or if methylation of the gene is decreased by the compound in the cell, one can identify it as having potential as a treatment for cancer.

Alternatively such tests can be used to determine an esophageal, head and neck, gastric, small intestinal, pancreas, liver cancer patient's response to a chemotherapeutic agent. The patient can be treated with a chemotherapeutic agent. If expression of a gene selected from BOLL, CABYR, EFEMP1, FBLN2, FOXL2, GNB4, GSTM3, HoxD1, Jph3, Neuralized (NEURL), PPP1R14a, TP53AP1, RAB32, SYNE1, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, SOX17, STK31, SYCP3, TCL1A, TFPI-2, TLR2, UCHL1, ZFP42, ASCL2, ATP8A2, CTAG2, EPHA4, FANCF, FOXE1, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655 is increased by the compound in cancer cells or if methylation of the gene is decreased by the compound in cancer cells it can be selected as useful for treatment of the patient.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLES

### Example 1

### Materials and Methods

Cell culture and treatment. HCT116 cells and isogenic genetic knockout derivatives were maintained as previously described (14). For drug treatments, log phase CRC cells were cultured in McCoys 5A media (Invitrogen) containing 10% BCS and 1× penicillin/streptomycin with 5 μM 5aza-deoxycytidine (DAC) (Sigma; stock solution: 1 mM in PBS) for 96 hours, replacing media and DAC every 24 hours. Cell treatment with 300 nM Trichostatin A (Sigma; stock solution: 1.5 mM dissolved in ethanol) was performed for 18 hours. Control cells underwent mock treatment in parallel with addition of equal volume of PBS or ethanol without drugs.

Microarray analysis. Total RNA was harvested from log phase cells using Triazol (Invitrogen) and the RNeasy kit (Qiagen) according to the manufacturers instructions, including a DNAase digestion step. RNA was quantified using the Nanoprop ND-100 followed by quality assessment with 2100 Bioanalyzer (Agilent Technologies). RNA concentrations for individual samples were greater than 200 ng/ul, with 28s/18s ratios greater than 2.2 and RNA integrity of 10 (10 scored as the highest). Sample amplification and labeling procedures were carried out using the Low RNA Input Fluorescent Linear Amplification Kit (Agilent Technologies) according to the manufacturers instructions. The labeled cRNA was purified using the RNeasy mini kit (Qiagen) and quantified. RNA spike-in controls (Agilent Technologies) were added to RNA samples before amplification. 0.75 microgram of samples labeled with Cy3 or Cy5 were mixed with control targets (Agilent Technologies), assembled on Oligo Microarray, hybridized, and processed according to the Agilent microarray protocol. Scanning was performed with the Agilent G2565BA microarray scanner using settings recommended by Agilent Technologies.

Data analysis. All arrays were subject to quality checks recommended by the manufacturer. Images were visually inspected for artifacts and distributions of signal and background intensity of both red and green channels were examined to identify anomalous arrays. No irregularities were observed, and all arrays were retained and used. All calculations were performed using the R statistical computing platform (23) and packages from Bioconductor bioinformatics software project (24). The log ratio of red signal to green signal was calculated after background-subtraction and LoEss normalization as implemented in the limma package from Bioconductor (25,26). Individual arrays were scaled to have the same inter-quartile range (75th percentile-25th percentile). Log fold changes were averaged over dye-swap replicate microarrays to produce a single set of expression values for each condition.

Methylation and gene expression analysis. RNA was isolated with TRIzol Reagent (Invitrogen) according to the manufacturer's instructions. For reverse transcription-PCR (RT-PCR), 1 μg of total RNA was reverse transcribed by using Ready-To-Go™ You-Prime First-Strand Beads (Amersham Biosciences) with addition of random hexamers (0.2 μg per reaction). For RT-primer design we used Primer3 (http://frodo.wi.mit.edu/cgi-bin/primer3/primer3_www.cgi). For MSP analysis, DNA was extracted following a standard phenol-chloroform extraction method. Bisulfite modification of genomic DNA was carried out using the EZ DNA methylation Kit (Zymo Research). Primer sequences specific for the unmethylated and methylated promotor sequences were designed using MSPPrimer (http://www.mspprimer.org). MSP was performed as previously described (20). All PCR products (15 μl of 50 μl total volume for RT-PCR and 7.5 μl of 25 μl total volume for MSP) were loaded directly onto 2% agarose gels containing GelStar Nucleic Acid Gel Stain (Cambrex Bio Science) and visualized under ultraviolet illumination. Primer sequences and conditions for MSP, bisulfite sequencing, and RT-PCR are available upon request from the authors.

Human Tumor Analysis. Formalin-fixed, paraffin embedded tissues from primary CRCs were obtained from the archive of the Department of Pathology of the University Hospital Maastricht. Approval was obtained by the Medical Ethical Committee of the Maastricht University, University Hospital Maastricht and Johns Hopkins University Hospital. DNA was isolated using the Puregene DNA isolation kit (Gentra Systems).

### Example 2

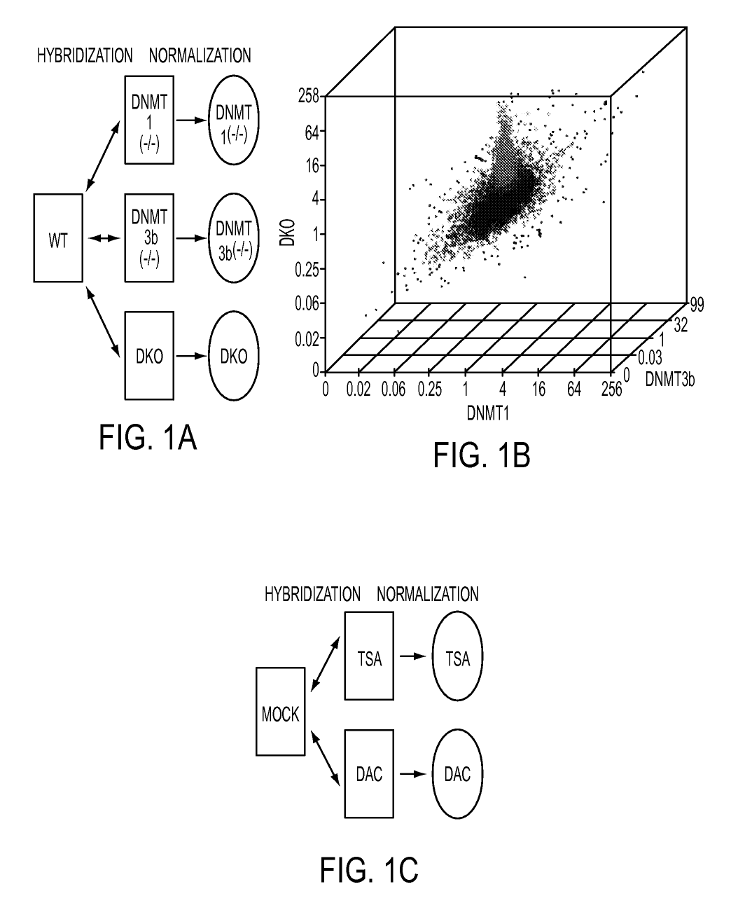

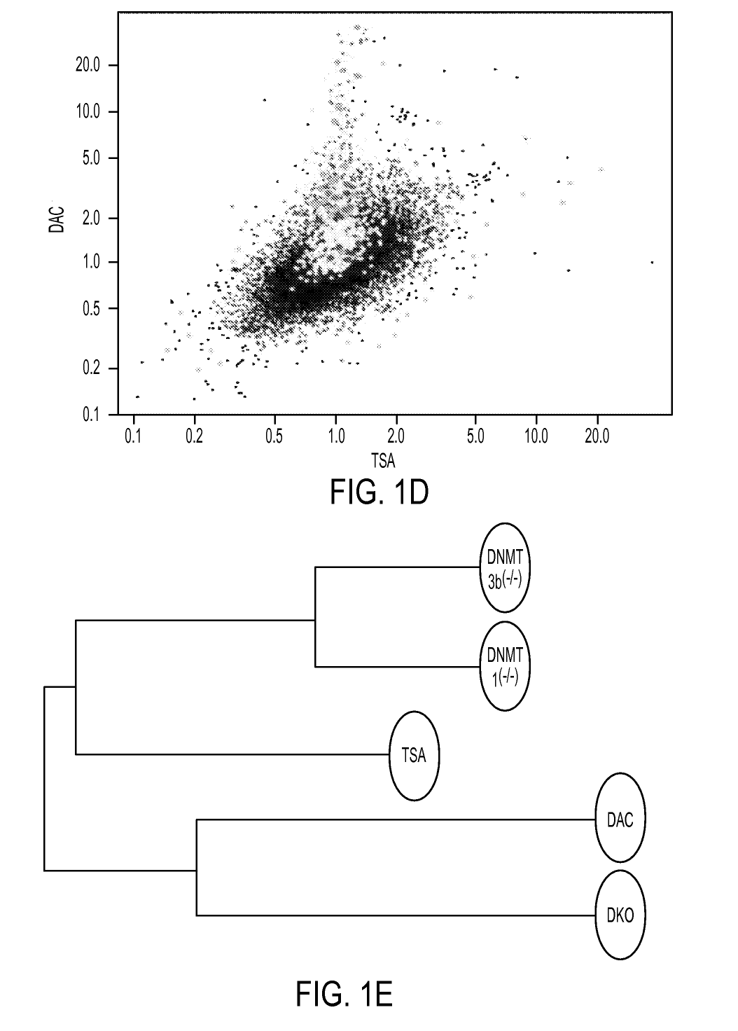

Our first step towards a global identification of hypermethylation dependent gene expression changes was made by comparing, in a genome wide expression array-based approach, wild type HCT116 CRC cells with isogenic partner cells carrying individual and combinatorial genetic deletions of two major human DNA methyltransferases (FIG. 1A, 14). Importantly, in the DNMT1^{(-/-)}DNMT3b^{(-/-)} double knockout (DKO) HCT116 cells, which have virtually complete loss of global 5-methylcytosine, all previously individually examined hypermethylated genes lacking basal expression in wild type cells undergo promoter demethylation with concomitant gene re-expression (14-17). By stratifying genes according to altered signal intensity on a 44K Agilent Technologies array platform, we observe a unique spike of gene expression increases in the DKO cells when compared to the isogenic wildtype parental cells, or isogenic cell lines in which DNMT's 1 or 3b have been individually deleted and which harbor minimal changes in DNA methylation (FIG. 1B).

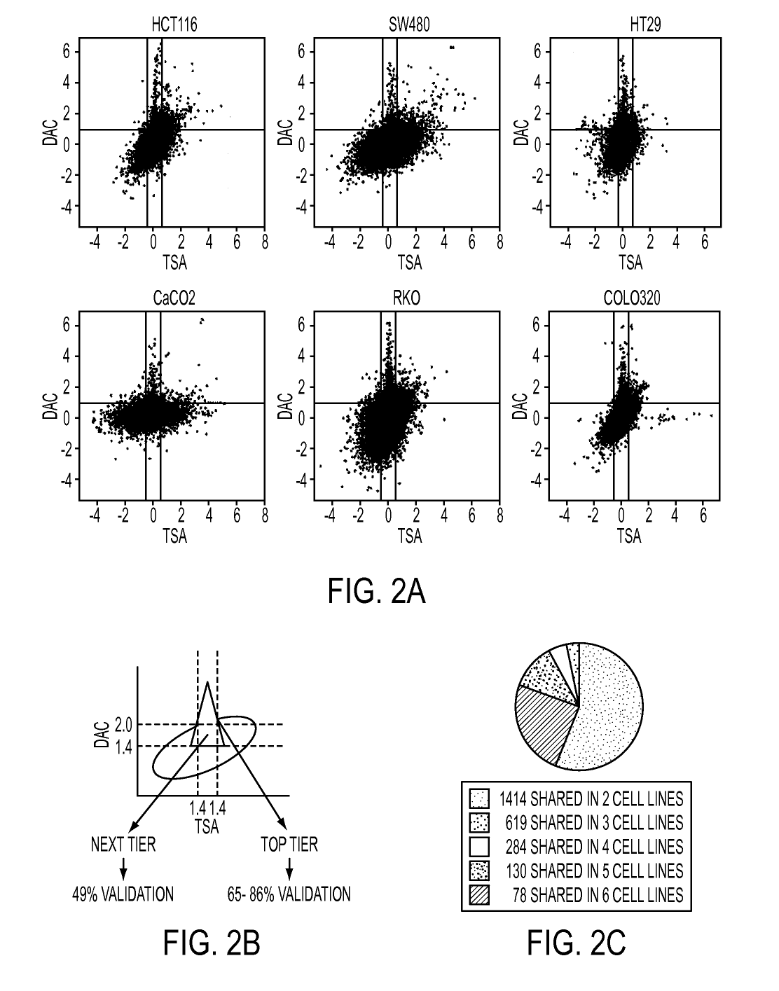

We tested our approach using a pharmacologic strategy based on our previous approach, but now markedly modified to provide whole transcriptome coverage (19), to identify silenced hypermethylated genes in any cancer cell line. For densely hypermethylated and transcriptionally inactive genes, the DNA demethylating agent 5-aza-2′-deoxycytidine (DAC) has a well established capacity to induce gene re-expression (18). On the other hand, for these same genes, the class I and II HDAC inhibitor, trichostatin A (TSA) will not alone induce re-expression (19). After treatment of HCT116 cells with either DAC or TSA (FIG. 1C), we identified a zone in which gene expression did not increase with TSA (<1.4 fold) and displayed no detectable expression in mock treated cells. Within this zone, we observed a characteristic spike of DAC induced gene expression which overlaps with the gene expression increases seen in DKO cells (compare yellow spots in FIG. 1D with blue spots in FIG. 1B). Since DAC incorporates into DNA of dividing cells, and our treatments were performed for only 96 hours, sensitivity for detecting the gene increases in the pharmacological approach was reduced compared to the DKO cells. Identification of this gene spike is absolutely dependent upon analysis of only genes that fail to respond to HDAC inhibition, underscored by a cluster analysis that shows the close relationship between genes in DKO and DAC treated cells with a separate grouping of genes expression changes after TSA treatment alone (FIG. 1E). These data confirm previous studies covering much less of the genome, in which, genes with dense CpG islands that were re-expressed by TSA harbored no detectable hypermethylation (19). A similar spike of gene expression increases could be seen in 5 additional human CRC cell lines (SW480, CaCO2, RKO, HT29 and COLO320), confirming that this approach works universally in cancer cell lines (FIG. 2A).

### Example 3

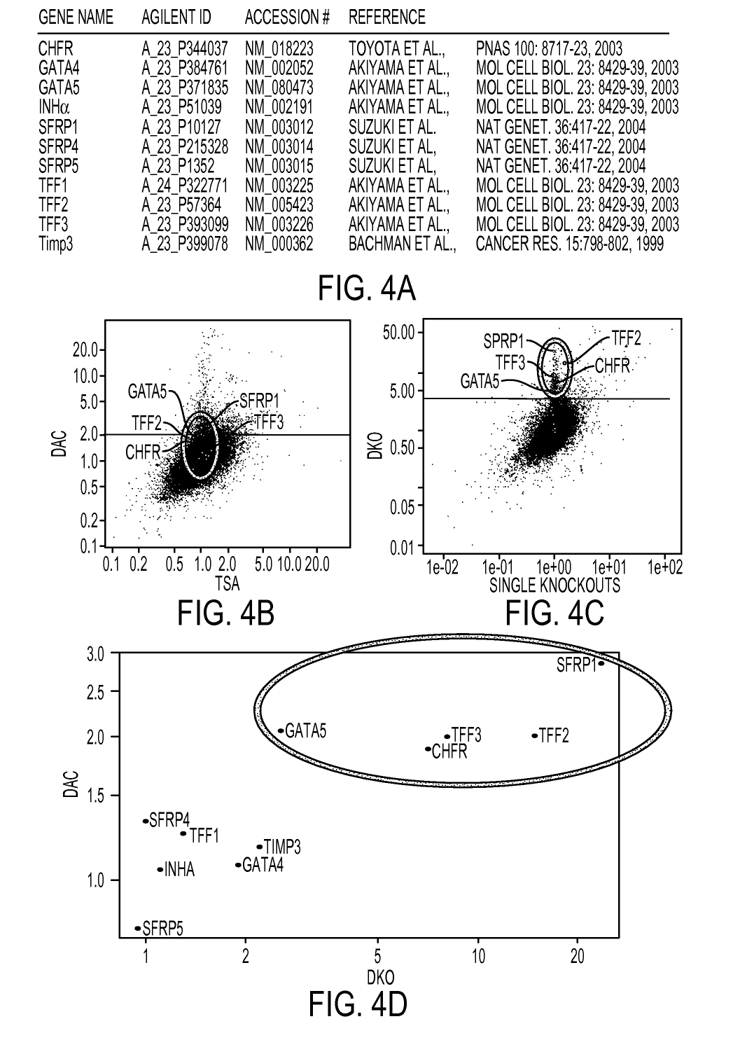

To address the sensitivity with which our new array approach identifies CpG island hypermethylated genes, we first examined 11 genes known to be hypermethylated, completely silenced and re-expressed after DAC treatment in HCT116 cells (FIG. 4 (S1A)) (14-17). All tested genes remained within the TSA non-responsive zone (FIG. 4 (S1B and C)), and the direction of expression changes correlated well in DAC treated and DKO cells (FIG. 4 (S1D)). Importantly, for the DAC increase, 5 of the guide genes (45%) increased 2-fold or more and 3 more genes, or a total of 73%, increased 1.3 fold or more.

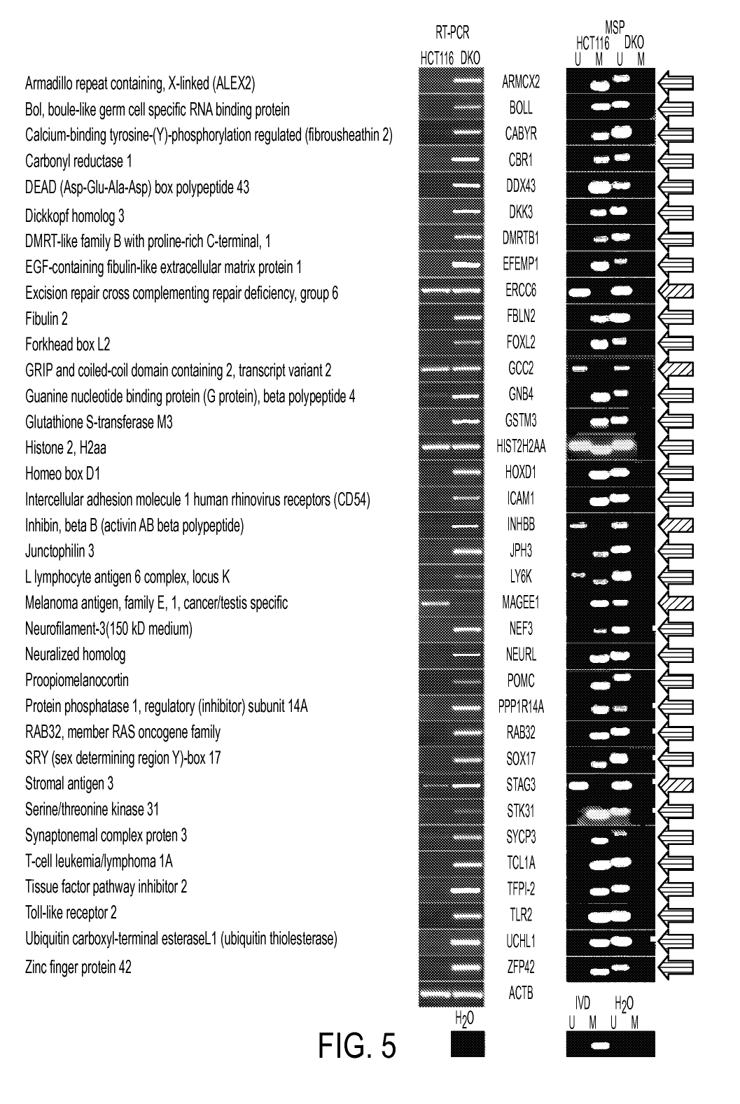

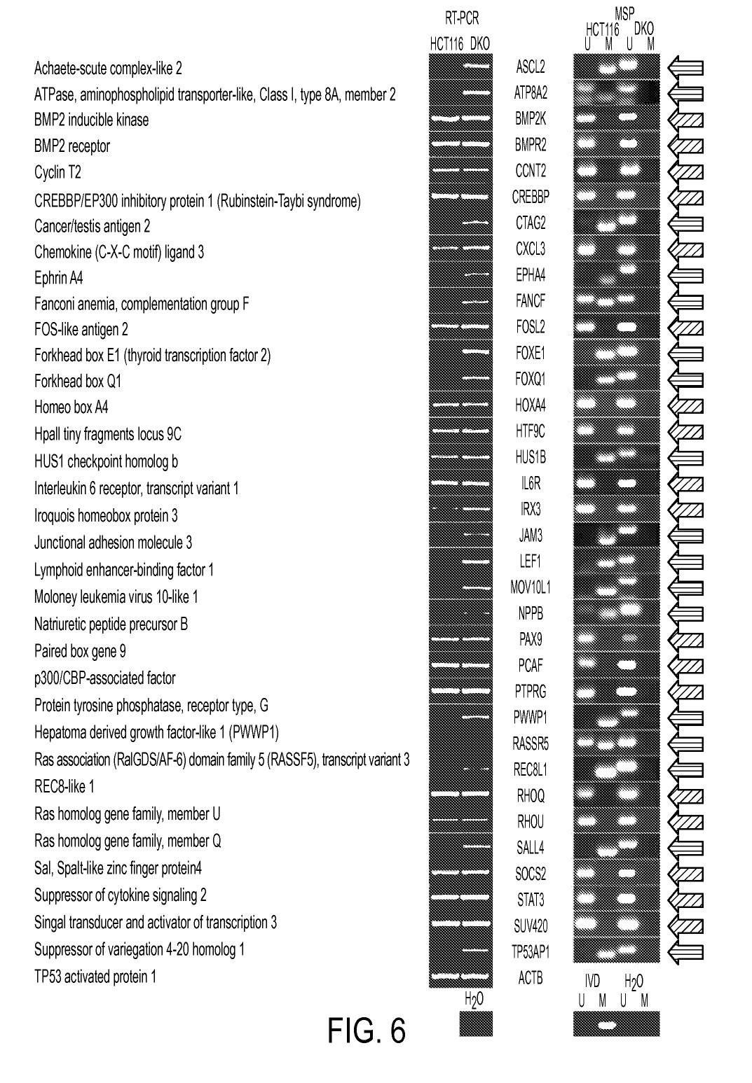

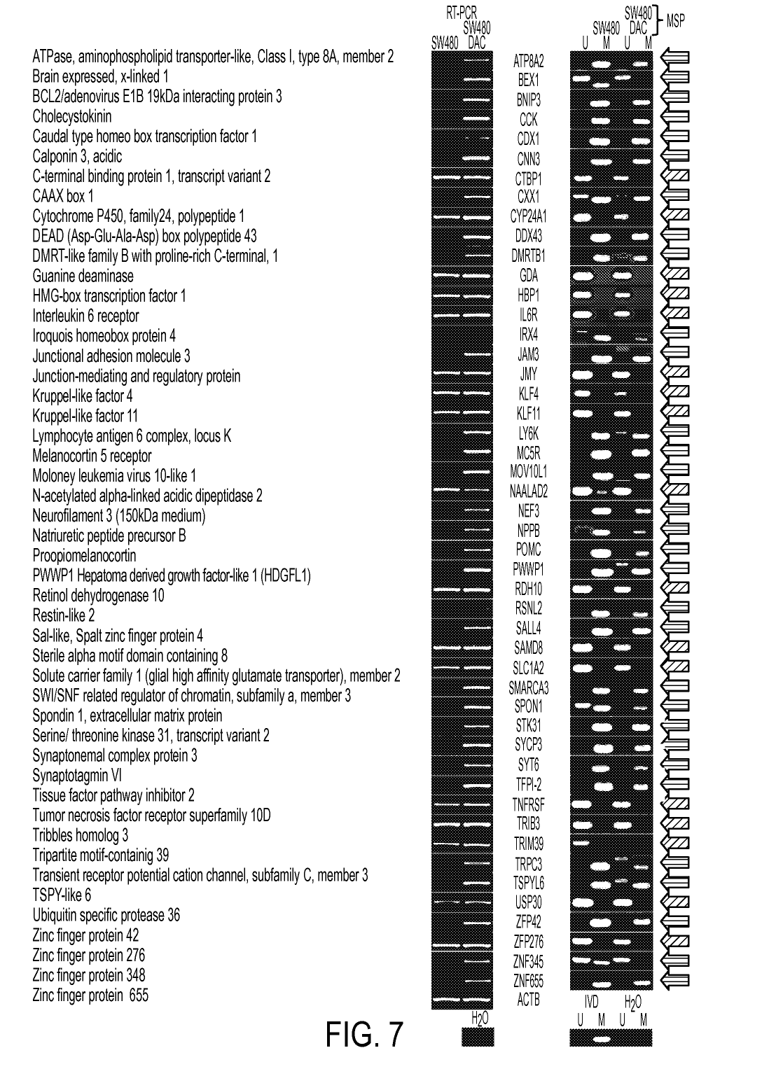

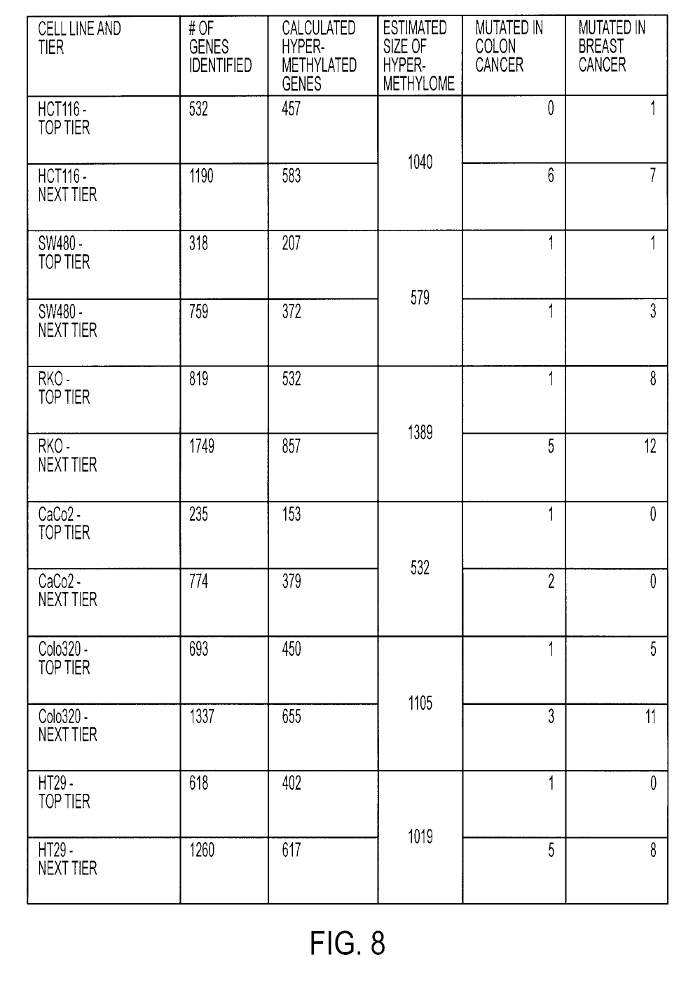

Based on the sensitivity differences observed between DKO and DAC induced gene increases, and behavior of the guide genes in the array platform, we designated, within the TSA negative zone, a top tier (2 fold increase or above) and a next tier of genes (increasing between 1.4 and 2 fold) to identify hypermethylated cancer genes (FIG. 2B). We also picked genes from these zones based on their having no basal expression in untreated cells. Indeed, in HCT116 cells, 30 of 35 (86%) of randomly chosen genes spanning the top-tier response zone of 532 genes, and 31 of 48 such SW480 cell genes (65%) from among 318 top tier genes proved to be CpG hypermethylated, as measured by MSP (20), and silenced in the cell line of origin as measured by RT-PCR (FIG. 5, 7 (S2, S4). We also examined the efficiency of discovery for hypermethylated genes in the next tier of DAC treated HCT116 cells. Of the 1190 genes identified in this region, 17 of 35 (49%) randomly selected genes containing a CpG island were hypermethylated with concordant gene silencing (FIG. 6 (S3)). This demonstrates that our approach is extraordinarily efficient compared to previous screens for identifying new cancer hypermethylated genes (7, 21). With this level of verified hypermethylation, we calculate that the hypermethylome in HCT116 cells consists of an estimated 1040 genes and an estimated 579 genes for the SW480 cells (See FIG. 8 (table S1) for a detailed description of calculations). The hypermethylome would be estimated to range from 532 genes in CaCO2 to 1389 genes in RKO cells (FIG. 8 (table S1)). A total of 5906 unique genes were identified amongst all tiers in the 6 cell lines, yielding an average of nearly 1000 hypermethylome genes per cell line.

### Example 4

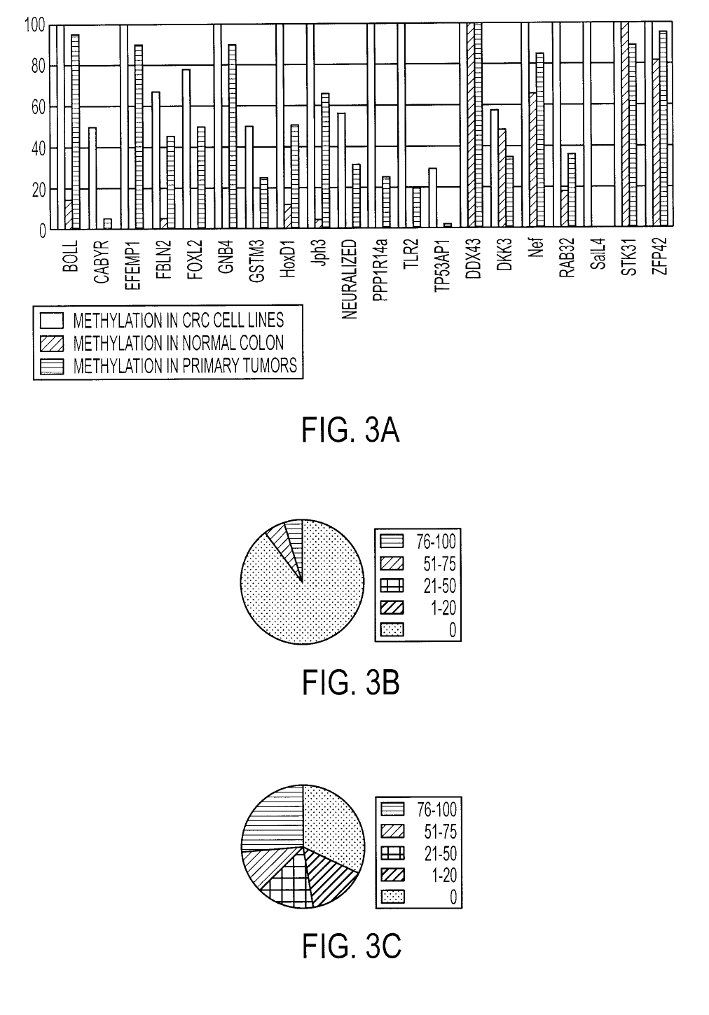

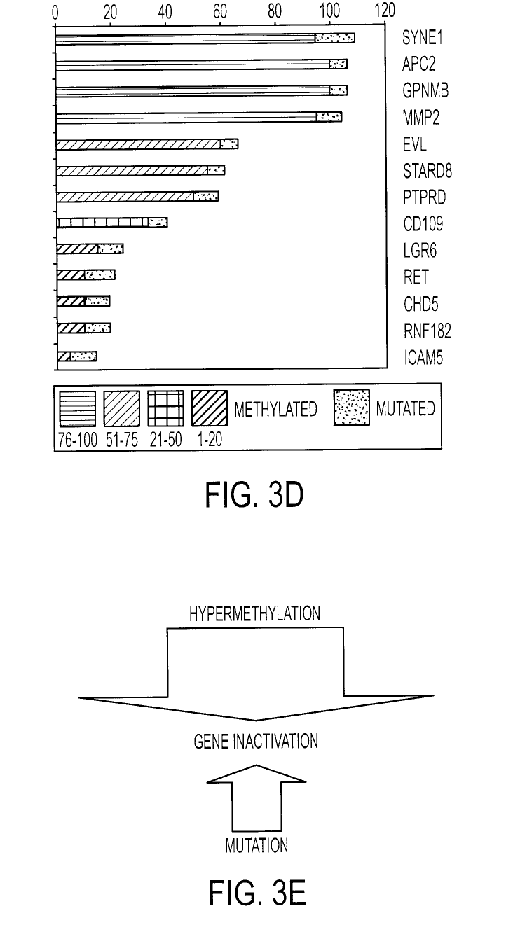

A fundamental question in cell culture based approaches is whether they identify genes which are targets for inactivation in primary tumors. To address this, 20 CpG island containing genes from the verified gene lists were randomly selected from the HCT116 top tier (17 genes), HCT116 next tier (2 genes), or SW480 top tier (1 gene) and analyzed for methylation in a panel of CRC cell lines. All of the tested genes were hypermethylated in two or more cell lines (FIG. 3A). We then examined the status of these 20 genes in a panel of 20 to 61 primary colon cancers and 20 to 40 normal appearing colon tissue samples obtained from cancer free individuals. Most of the genes (65%) were completely unmethylated, or rarely methylated, in the normal colonic tissue samples, but were methylated in a vast majority (86%) of the primary tumors (FIG. 3A). Of the 20 genes analyzed, 13 genes (65%) satisfied criteria for “tumor specific methylation” with high frequency methylation in cell lines, low or undetectable methylation in normal colon, and frequent methylation in tumor samples. The efficiency of our strategy suggests a discovery rate of at least 1 in 2 for identification of hypermethylated genes in cell lines and at least 1 in 3 for identification of cancer specific hypermethylated genes.

### Example 5

While it is clear that genetic and epigenetic mechanisms are both important to initiation and progression of human tumorigenesis, the relative contributions of each of these alterations is poorly understood. Comparisons among methylation and mutation frequencies for a handful of cancer genes has not convincingly demonstrated the prevalence of either pathway. Importantly, a genome wide analysis to query this issue has not been performed.

In a recent genome-wide sequencing of cancer genes, Sjöblom et al. (3) observed that mutations generally had a low incidence of occurrence, with 90% of the genes identified harboring a mutation frequency of less than 10%. Furthermore, a typical colon or breast tumor contained an average of only 11 mutations per individual tumor and there was little overlap between single tumors. These low frequencies raise the question whether alternative mechanisms might account for inactivation of these genes in additional tumors. Obviously, the much higher number of candidate hypermethylated genes we now identify in individual tumors (FIG. 2C) suggests that this epigenetic change might provide an alternative inactivating route to mutations for many tumor suppressor genes.

The importance of screening tumors for both genetic and epigenetic changes is strikingly emphasized when we searched for matches between the candidate hypermethylated genes and the 189 mutated cancer (CAN) genes. We first queried our list of 5906 hypermethylome genes with the CAN gene list identified in breast and CRC tumors. This identified 56 common genes of which 45 contained CpG islands. Twenty six of these 45 genes (58%), similar to the verification rate for all candidate genes identified as discussed above, proved to be hypermethylated in at least one of the six cell lines, and were selected for further study. Importantly, exactly half (13 genes) of the genes were not methylated in normal colon but were methylated in primary CRC tumors (FIG. 3B, C) giving a frequency of 50% for identification of tumor specific methylation. For virtually each of the examined genes, the incidence of hypermethylation is strikingly higher than that for mutations (FIG. 3D). Thus, unlike for the mutated genes, hypermethylation for the majority of the genes is a shared property between many tumors. These findings for both epigenetic silencing and mutations in previously uncharacterized genes solidifies their probable roles as tumor suppressor genes (FIG. 3E).

### Example 6

### Additional Tissue Data Collected for BNIP3, FOXE1, SYNE1, SOX17, JAM3, MMP2 and GPNMB

### Material and Methods

### Clinical Samples Used for Tissue Validation

A total of 171 colon paraffin embedded tissue samples, corresponding to 77 normal tissues and 94 cancer samples were processed using real-time MSP. Table 2 gives an overview of the sample set used for tissue validation.

TABLE 2
Clinical sample set details
171 paraffin embedded tissue samples

| Number of | | | |
|---|---|---|---|
| histologically | Number of | | |
| normal | colorectal | Grade Details | |
| colorectal tissue | cancer tissue | of the colorectal | |
| samples | samples | cancer tissues | Numbers |
| 77 | 94 | Grade 1 | 10 |
| | | Grade 2 | 45 |
| | | Grade 2 + 3 | 2 |
| | | Grade 3 | 14 |
| | | Unknown grade | 23 |

DNA Extraction from Paraffin Embedded Tissue Samples

Formalin Fixed paraffin embedded samples were first de-paraffinized in 750 μl xylene for 2 h. Then, 250 μl of 70% ethanol was added before centrifugation at 13000 rpm for 15 min. The supernatant was removed and the samples were air dried for 20 min at room temperature. DNA was extracted using the classical phenol/chloroform extraction method and resuspended in 50 μl LoTe (3 mM TRIS, 0.2 mM EDTA, pH 8.0). Subsequently, the DNA was quantified using the Picogreen® dsDNA quantitation kit (Molecular Probes, Invitrogen) following manufacturer's recommendations. λDNA provided with the kit was used to prepare a standard curve (from 1 to 800 ng/ml). The data were collected using a FluoStar Galaxy plate reader (BMG Lab technologies, Germany).

### DNA Modification

1 μg of DNA was subjected to bisulfite modification in 96-wells format on a pipetting robot (Tecan), using the EZ-96DNA Methylation kit (Zymo Research), according to the manufacturer's protocol. Basically, aliquots of 45 μl were mixed with 5 μl of M-Dilution Buffer and incubated at 37° C. for 15 minutes shaking at 1100 rpm. Then 100 μl of the diluted CT Conversion Reagent was added and samples were incubated at 70° C. for 3 hours, shaking at 1100 rpm in the dark. After conversion, the samples were desalted by incubation on ice for 10 minutes and addition of 400 μl of M-Binding buffer. The samples were loaded on a Zymo-Spin I Column in a collection plate and after centrifugation washed with 200 μl of M-Wash Buffer. 200 μl of M-Desulphonation Buffer was put onto the column and incubated at room temperature for 15 minutes. After centrifugation of the columns, they were washed twice with 200 μl of M-Wash Buffer. Finally, the DNA was washed from the column in 125 μl Tris-HCl 1 mM pH 8.0 and stored at −80° C., until further processing.

### DNA Amplification

Real-time MSP was applied on a 7900HT fast real-time PCR system (Applied Biosystems). 5 μl of the modified DNA was added to a PCR mix (total volume 10 μl) containing buffer (16.6 mM (NH4)2SO4, 67 mM Tris (pH 8.8), 6.7 mM MgCl2, 10 mM β-mercaptoethanol), dNTPs (5 mM), forward primer (6 ng), reverse primer (18 ng), molecular beacon (0.16 μM) and Jumpstart DNA Taq polymerase (0.4 units; Sigma Aldrich). The primer sequences and molecular beacon sequences used for each of the genes are summarized in Table 3. Cycle program used was as follows: 5 minutes 95° C., followed by 45 cycles of 30 seconds 95° C., 30 seconds 57° C., and 30 seconds 72° C. A standard curve (2×106−20 copies) was included to determine copy numbers of unknown samples by interpolation of their Ct values to the standard curve.

TABLE 3
Primer sequences and beacon sequences

| | | |
|---|---|---|
| BNIP3 | forward | 5′-TACGCGTAGGTTTTAAGTCGC-3′ |
| | primer | (SEQ ID NO: 251) |
| | reverse | 5′-TCCCGAACTAAACGAAACCCCG-3′ |
| | primer | (SEQ ID NO: 252) |
| | beacon | 5′-FAM-CGACATGCCTACGACCGCGTCGCCCATTAGCATGTCG-3′- |
| | | DABCYL |
| | | (SEQ ID NO: 253) |
| FOXE1 | forward | 5′-TTTGTTCGTTTTTCGATTGTTC-3′ |
| | primer | (SEQ ID NO: 254) |
| | reverse | 5′-TAACGCTATAAAACTCCTACCGC-3′ |
| | primer | (SEQ ID NO: 255) |
| | beacon | 5′-FAM-CGTCTCGTGGGGGTTCGGGCGTATTTTTTTAGGTAGGCGAGA |
| | | CG-3′-DABCYL |
| | | (SEQ ID NO: 256) |
| JAM3 | forward | 5′-GGGATTATAAGTCGCGTCGC-3′ |
| | primer | (SEQ ID NO: 257) |
| | reverse | 5′-CGAACGCAAAACCGAAATCG-3′ |
| | primer | (SEQ ID NO: 258) |
| | beacon | 5′-FAM-CGACACGATATGGCGTTGAGGCGGTTATCGTGTCG-3′- |
| | | DABCYL |
| | | (SEQ ID NO: 259) |
| SOX17 | forward | 5′-GAGATGTTTCGAGGGTTGC-3′ |
| | primer | (SEQ ID NO: 260) |
| | reverse | 5′-CCGCAATATCACTAAACCGA-3′ |
| | primer | (SEQ ID NO: 261) |
| | beacon | 5′-FAM-CGACATGCGTTCGTGTTTTGGTTTGTCGCGGTTTGGCATGTC |
| | | G-3′-DABCYL |
| | | (SEQ ID NO: 262) |
| SYNE1 | forward | 5′-GTTGGGTTTTCGTAGTTTTGTAGATCGC-3′ |
| | primer | (SEQ ID NO: 263) |
| | reverse | 5′-CTACGCCCAAACTCGACG-3′ |
| | primer | (SEQ ID NO: 264) |
| | beacon | 5′-FAM-CGACATGCCCCGCCCTATCGCCGAAATCGCATGTCG-3′- |
| | | DABCYL |
| | | (SEQ ID NO: 265) |
| MMP2 | forward | 5′-TTCGGGTTATTAGCGTTTTTATC-3′ |
| | primer | (SEQ ID NO: 266) |
| | reverse | 5′-ACTCCAACCAAACGACGAA-3′ |
| | primer | (SEQ ID NO: 267) |
| | beacon | 578′-FAM-CGACATCGTTGGTTCGGTGCGTGTGGTCGATGTCG-3′- |
| | | DABCYL |
| | | (SEQ ID NO: 268) |
| GPNMB | forward | 5′-GGTCGTAGTCGTAGTCGGG-3′ |
| | primer | (SEQ ID NO: 269) |
| | reverse | 5′-CCGCAAAAACCTAAAACGTAA-3′ |
| | primer | (SEQ ID NO: 270) |
| | beacon | 5′-FAM-CGACATGCGGTTTTTTGGGTCGGGGCGCGGCATGTCG-3′- |
| | | DABCYL |
| | | (SEQ ID NO: 271) |
| β-Actin | forward | 5′-TAGGGAGTATATAGGTTGGGGAAGTT-3′ |
| | primer | (SEQ ID NO: 272) |
| | reverse | 5′-AACACACAATAACAAACACAAATTCAC-3′ |
| | primer | (SEQ ID NO: 273) |
| | beacon | 5′-FAM-CGACTGCGTGTGGGGTGGTGATGGAGGAGGTTTAGGCAGTC |
| | | G-3′-DABCYL |
| | | (SEQ ID NO: 274) |

### Marker Validation on Tissue Material

Experiments were performed as described above. The colon markers: BNIP3, FOXE1, SYNE1, SOX17, JAM3, MMP2 and GPNMB were validated on tissue material using primer sets and beacon probes as specified in Table 3. In addition the independent reference β-Actin (ACTB) was measured. Results were generated using the SDS 2.2 software (Applied Biosystems) and exported as Ct values (cycle number at which the amplification curves cross the threshold value, set automatically by the software). Copy numbers were extrapolated using a standard curve. The ratio of the gene of interest to ACTB (multiplied by 1000) for each sample was used as a measure for representing the relative level of methylated DNA for each gene of interest within each sample.

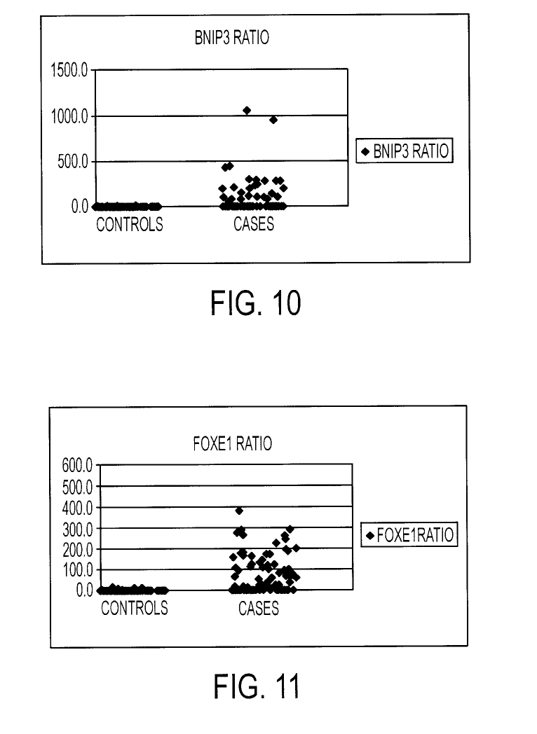

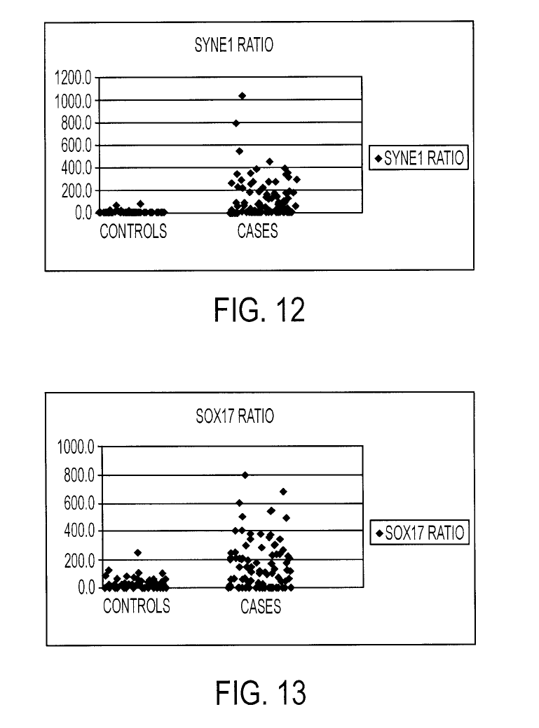

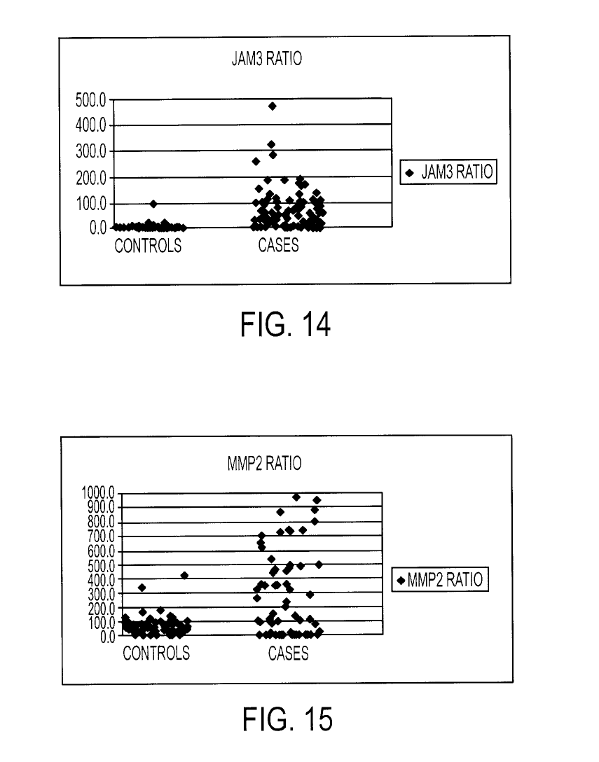

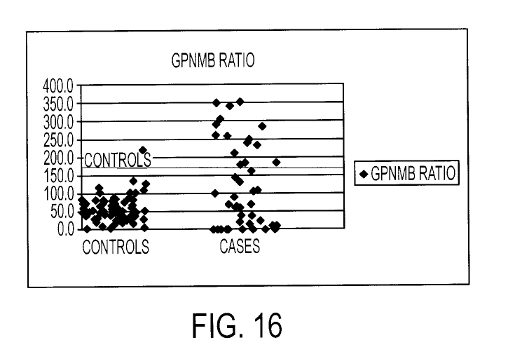

Methylation-specific PCR scatter plots of BNIP3, FOXE1, SYNE1, SOX17, JAM3, MMP2 and GPNMB in normal samples (controls) and cancers (cases) are shown in FIG. 10-16. The measurements are expressed as a methylation ratio, defined as the ratio of the fluorescence intensity values for each gene compared to ACTB, multiplied by 1000 for easier tabulation.

As indicated in FIG. 10-16, a clear difference in ratio was observed between the investigated control and cases group.

### REFERENCES

1. Ponder B. A. Cancer genetics. Nature 411, 336-41 (2001).
2. Herman, J. G. & Baylin, S. B. Gene silencing in cancer in association with promoter hypermethylation. N Engl J Med 349, 2042-54 (2003).
3. Sjöblom T. et al. The consensus coding sequences of human breast and colorectal cancers. Science 314, 268-74 (2006).
4. Antequera, F. & Bird, A. Number of cpg islands and genes in human and mouse. Proc Natl Acad Sci USA 90, 11995-11999 (1993).
5. Costello, J. F. et al. Aberrant CpG-island methylation has non-random and tumour-type-specific patterns. Nat. Genet. 24, 132-138 (2000).
6. Suzuki, H. et al. A genomic screen for genes upregulated by demethylation and histone deacetylase inhibition in human colorectal cancer. Nat Genet 31, 141-9 (2002).
7. Gius, D. et al. Distinct effects on gene expression of chemical and genetic manipulation of the cancer epigenome revealed by a multimodality approach. Cancer Cell 6, 361-71 (2004).
8. Paz, M. F. et al. Genetic unmasking of epigenetically silenced tumor suppressor genes in colon cancer cells deficient in DNA methyltransferases. Hum Mol Genet 12, 2209-19 (2003).
9. Weber, M. et al. Chromosome-wide and promoter-specific analyses identify sites of differential DNA methylation in normal and transformed human cells. Nat Genet 37, 853-62 (2005).
10. Hu, M. et al. Distinct epigenetic changes in the stromal cells of breast cancers. Nat Genet 37, 899-905 (2005).
11. Toyota, M. et al. Identification of differentially methylated sequences in colorectal cancer by methylated CpG island amplification. Cancer Res 59, 2307-12 (1999).
12. Keshet, I. et al. Evidence for an instructive mechanism of de novo methylation in cancer cells. Nat Genet 38, 149-53 (2006).
13. Ushijima, T. Detection and interpretation of altered methylation patterns in cancer cells. Nat Rev Cancer 5, 223-31 (2005).
14. Rhee, I. et al. DNMT1 and DNMT3b cooperate to silence genes in human cancer cells. Nature 416, 552-6 (2002).
15. Toyota, M. et al. Epigenetic inactivation of CHFR in human tumors. Proc Natl Acad Sci USA 100, 7818-23 (2003).
16. Suzuki, H. et al. Epigenetic inactivation of SFRP genes allows constitutive WNT signaling in colorectal cancer. Nat Genet 36, 417-22 (2004).
17. Akiyama, Y. et al. GATA-4 and GATA-5 transcription factor genes and potential downstream antitumor target genes are epigenetically silenced in colorectal and gastric cancer. Mol Cell Biol 23, 8429-39 (2003).
18. Christman J. K. 5-Azacytidine and 5-aza-2′-deoxycytidine as inhibitors of DNA methylation: mechanistic studies and their implications for cancer therapy. Oncogene 21, 5483-95 (2002).
19. Cameron, E. E., Bachman, K. E., Myohanen, S., Herman, J. G. & Baylin, S. B. Synergy of demethylation and histone deacetylase inhibition in the re-expression of genes silenced in cancer. Nat Genet 21, 103-7 (1999).
20. Herman, J. G., Graff, J. R., Myohanen, S., Nelkin, B. D. & Baylin, S. B. Methylation-specific PCR: a novel PCR assay for methylation status of CpG islands. Proc Natl Acad Sci USA 93, 9821-6 (1996).
21. Yamashita K, Upadhyay S, Osada M, Hoque M O, Xiao Y, Mori M, Sato F, Meltzer S J, Sidransky D. Pharmacologic unmasking of epigenetically silenced tumor suppressor genes in esophageal squamous cell carcinoma. Cancer Cell 2 485-95 (2002).
22. Egger, G., Liang, G., Aparicio, A. & Jones, P. A. Epigenetics in human disease and prospects for epigenetic therapy. Nature 429, 457-63 (2004).
23. Ihaka, R. & Gentleman, R. C. R: A language for data analysis and graphics. Journal of Computational and Graphical Statistics 5, 299-314 (1996).
24. Gentleman, R C. et al. Bioconductor: open software development for computational biology and bioinformatics. Genome Biol 5, R80 (2004).
25. Smyth, G. K. & Speed, T. Normalization of cDNA microarray data. Methods 31, 265-73 (2003).
26. Smyth, G. K., Yang, Y. H. & Speed, T. Statistical issues in cDNA microarray data analysis. Methods Mol Biol 224, 111-36 (2003).

We claim:

1. A method for identifying colorectal cancer or its precursor, or predisposition to colorectal cancer, comprising:
detecting in a test sample containing colorectal cells or nucleic acids from colorectal cells, epigenetic silencing of at least one gene selected from the group consisting of FOXE1, SOX17, SYNE1, BOLL, CABYR, EFEMP1, FBLN2, FOXL2, GNB4, GSTM3, HoxD1, Jph3, Neuralized (NEURL), PPP1R14a, TP53AP1, RAB32, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, STK31, SYCP3, TCL1A, TFPI-2, TLR2, UCHL1, ZFP42, ASCL2, ATP8A2, CTAG2, EPHA4, FANCF, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655;
identifying the test sample as containing cells that are neoplastic, precursor to neoplastic, or predisposed to neoplasia, or as containing nucleic acids from cells that are neoplastic, precursor to neoplastic, or predisposed to neoplasia.

2. The method of claim 1 wherein the test sample contains adenoma cells.

3. The method of claim 1 wherein the test sample contains nucleic acids from adenoma cells.

4. The method of claim 1 wherein the test sample contains carcinoma cells or nucleic acids from carcinoma cells.

5. The method of claim 1 wherein the at least one gene is selected from the group consisting of SYNE1, APC2, GPNMB, MMP2, EVL, STARD8, PTPRD, CD109, LGR6, RET, CHD5, RNF182, ICAM5.

6. The method of claim 1 further comprising the step of:
detecting in the test sample containing colorectal cells or nucleic acids from colorectal cells, a mutation in at least one gene selected from the group consisting of FOXE1, SOX17, SYNE1, BOLL, CABYR, EFEMP1, FBLN2, FOXL2, GNB4, GSTM3, HoxD1, Jph3, Neuralized (NEURL), PPP1R14a, TP53AP1, RAB32, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, STK31, SYCP3, TCL1A, TFPI-2, TLR2, UCHL1, ZFP42, ASCL2, ATP8A2, CTAG2, EPHA4, FANCF, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655.

7. The method of claim 1 wherein the test sample is from a fresh or frozen tissue specimen.

8. The method of claim 1 wherein the test sample is from a biopsy specimen.

9. The method of claim 1 wherein the test sample is from a surgical specimen.

10. The method of claim 1 wherein the test sample is from a cytological specimen.

11. The method of claim 1 wherein the test sample is isolated from a body fluid selected from the group consisting of whole blood, bone marrow, cerebral spinal fluid, peritoneal fluid, pleural fluid, lymph fluid, serum, mucus, plasma, urine, chyle, stool, ejaculate, sputum, nipple aspirate, saliva, swab specimen, colon wash specimen, and brush specimen.

12. The method of claim 8 wherein surgical removal of neoplastic tissue is recommended to the patient.

13. The method of claim 8 wherein adjuvant chemotherapy is recommended to the patient.

14. The method of claim 8 wherein adjuvant radiation therapy is recommended to the patient.

15. The method of claim 11 wherein a colonoscopy or sigmoidoscopy is recommended to the patient.

16. The method of claim 8 wherein increased frequency of colonoscopy is recommended to the patient.

17. The method of claim 11 wherein an imaging study of the colon is recommended to the patient.

18. The method of claim 1 wherein epigenetic silencing of at least two genes is detected.

19. The method of claim 1 wherein epigenetic silencing is detected by detecting methylation of a CpG dinucleotide motif in the gene.

20. The method of claim 1 wherein epigenetic silencing is detected by detecting methylation of a CpG dinucleotide motif in a promoter of the gene.

21. The method of claim 1 wherein epigenetic silencing is detected by detecting diminished expression of the gene.

22. The method of claim 21 wherein epigenetic silencing is detected by detecting diminished mRNA of the gene.

23. The method of claim 21 wherein diminished expression of the gene is determined by comparison to a control sample.

24. The method of claim 22 wherein diminished mRNA of the gene is determined by hybridization to a nucleotide probe.

25. The method of claim 21 wherein diminished expression is detected by nucleotide sequencing.

26. The method of claim 21 wherein diminished expression is detected by reverse transcription-polymerase chain reaction (RT-PCR).

27. The method of claim 26 wherein the RT-PCR is performed in a non-quantitative manner.

28. The method of claim 26 wherein the RT-PCR is performed in a real-time and quantitative manner.

29. The method of claim 21 wherein epigenetic silencing is detected by detecting diminished protein encoded by the gene.

30. The method of claim 19 wherein methylation is detected by contacting at least a portion of the gene with a methylation-sensitive restriction endonuclease, said endonuclease preferentially cleaving methylated recognition sites relative to non-methylated recognition sites, whereby cleavage of the portion of the gene indicates methylation of the portion of the gene.

31. The method of claim 19 wherein methylation is detected by contacting at least a portion of the gene with a methylation-sensitive restriction endonuclease, said endonuclease preferentially cleaving non-methylated recognition sites relative to methylated recognition sites, whereby cleavage of the portion of the gene indicates non-methylation of the portion of the gene provided that the gene comprises a recognition site for the methylation-sensitive restriction endonuclease.

32. The method of claim 19 wherein methylation is detected by:
contacting at least a portion of the gene of the test cell with a chemical reagent that selectively modifies a non-methylated cytosine residue relative to a methylated cytosine residue, or selectively modifies a methylated cytosine residue relative to a non-methylated cytosine residue; and
detecting a product generated due to said contacting.

33. The method of claim 32 wherein the step of detecting comprises hybridization with at least one probe that hybridizes to a sequence comprising a modified non-methylated CpG dinucleotide motif but not to a sequence comprising an unmodified methylated CpG dinucleotide.

34. The method of claim 32 wherein the step of detecting comprises hybridization with at least one probe that hybridizes to a sequence comprising an unmodified methylated CpG dinucleotide motif but not to a sequence comprising a modified non-methylated CpG dinucleotide motif.

35. The method of claim 32 wherein the step of detecting comprises amplification with at least one primer that hybridizes to a sequence comprising a modified non-methylated CpG dinucleotide motif but not to a sequence comprising an unmodified methylated CpG dinucleotide motif thereby forming amplification products.

36. The method of claim 32 wherein the step of detecting comprises amplification with at least one primer that hybridizes to a sequence comprising an unmodified methylated CpG dinucleotide motif but not to a sequence comprising a modified non-methylated CpG dinucleotide motif thereby forming amplification products.

37. The method of claim 32 wherein the product is detected by a method selected from the group consisting of electrophoresis, hybridization, amplification, primer extension, sequencing, ligase chain reaction, chromatography, mass spectrometry, and combinations thereof.

38. The method of claim 37 wherein the method is an absolute detection method.

39. The method of claim 37 wherein the method is a real-time detection method.

40. The method of claim 37 wherein the method is performed for at least two genes and the products generated for the at least two genes are compared.

41. The method of claim 32 wherein the chemical reagent is hydrazine.

42. The method of claim 41 further comprising cleavage of the hydrazine-contacted at least a portion of the gene with piperidine.

43. The method of claim 32 wherein the chemical reagent comprises bisulfite ions.

44. The method of claim 43 further comprising treating the bisulfite ion-contacted at least a portion of the gene with alkali.

45. A method of reducing or inhibiting neoplastic growth of a cell which exhibits epigenetic silenced transcription of at least one gene associated with a cancer, the method comprising:
determining that a cell has an epigenetic silenced gene selected from the group consisting of FOXE1, SOX17, SYNE1, BOLL, CABYR, EFEMP1, FBLN2, FOXL2, GNB4, GSTM3, HoxD1, Jph3, Neuralized (NEURL), PPP1R14a, TP53AP1, RAB32, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, STK31, SYCP3, TCL1A, TFPI-2, TLR2, UCHL1, ZFP42, ASCL2, ATP8A2, CTAG2, EPHA4, FANCF, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655;
restoring expression of a polypeptide encoded by the epigenetic silenced gene in the cell by contacting the cell with one or more agents selected from the group consisting of a CpG dinucleotide demethylating agent, a DNA methyltransferase inhibitor, and a histone deacetylase (HDAC) inhibitor, thereby reducing or inhibiting unregulated growth of the cell.

46. The method of claim 45 wherein the gene is selected from the group consisting of: SYNE1, APC2, GPNMB, MMP2, EVL, STARD8, PTPRD, CD109, LGR6, RET, CHD5, RNF182, ICAM5.

47. The method of claim 45 wherein the contacting is performed in vitro.

48. The method of claim 45 wherein the contacting is performed in vivo by administering the agent to a mammalian subject comprising the cell.

49. The method of claim 45 wherein the agent is a demethylating agent and the agent is selected from the group consisting of 5-aza-2′-deoxycytidine, 5-aza-cytidine, Zebularine, procaine, and L-ethionine.

50. A method of reducing or inhibiting neoplastic growth of a cell which exhibits epigenetic silenced transcription of at least one gene associated with a cancer, the method comprising:
determining that a cell has an epigenetic silenced gene selected from the group consisting of FOXE1, SOX17, SYNE1, BOLL, CABYR, EFEMP1, FBLN2, FOXL2, GNB4, GSTM3, HoxD1, Jph3, Neuralized (NEURL), PPP1R14a, TP53AP1, RAB32, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, STK31, SYCP3, TCL1A, TFPI-2, TLR2, UCHL1, ZFP42, ASCL2, ATP8A2, CTAG2, EPHA4, FANCF, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655;
introducing a polynucleotide encoding a polypeptide into the cell, wherein the polypeptide is encoded by said gene, wherein the polypeptide is expressed in the cell thereby restoring expression of the polypeptide in the cell.

51. The method of claim 50 wherein the gene the group consisting of SYNE1, APC2, GPNMB, MMP2, EVL, STARD8, PTPRD, CD109, LGR6, RET, CHD5, RNF182, ICAM5,

52. A method of treating a cancer patient, the method comprising:
determining that a cancer cell in the patient has an epigenetic silenced gene selected from the group consisting of FOXE1, SOX17, SYNE1, BOLL, CABYR, EFEMP1, FBLN2, FOXL2, GNB4, GSTM3, HoxD1, Jph3, Neuralized (NEURL), PPP1R14a, TP53AP1, RAB32, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, STK31, SYCP3, TCL1A, TFPI-2, TLR2, UCHL1, ZFP42, ASCL2, ATP8A2, CTAG2, EPHA4, FANCF, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655;
administering one or more agents selected from the group consisting of a CpG dinucleotide demethylating agent, a DNA methyltransferase inhibitor, and a histone deacetylase (HDAC) inhibitor to the patient in sufficient amounts to restore expression of the epigenetic silenced gene in the patient's cancer cells.

53. The method of claim 52 wherein the agent is a demethylating agent, and the agent is selected from the group consisting of 5-aza-2′-deoxycytidine, 5-aza-cytidine, Zebularine, procaine, and L-ethionine.

54. The method of claim 52 wherein the gene is selected from the group consisting of the group consisting of SYNE1, APC2, GPNMB, MMP2, EVL, STARD8, PTPRD, CD109, LGR6, RET, CHD5, RNF182, ICAM5.

55. A method of treating a cancer patient, the method comprising:
determining that a cancer cell in the patient has an epigenetic silenced gene selected from those shown in FOXE1, SOX17, SYNE1, BOLL, CABYR, EFEMP1, FBLN2, FOXL2, GNB4, GSTM3, HoxD1, Jph3, Neuralized (NEURL), PPP1R14a, TP53AP1, RAB32, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, STK31, SYCP3, TCL1A, TFPI-2, TLR2, UCHL1, ZFP42, ASCL2, ATP8A2, CTAG2, EPHA4, FANCF, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655;
administering to the patient a polynucleotide encoding a polypeptide, wherein the polypeptide is encoded by the epigenetic silenced gene, wherein the polypeptide is expressed in the patient's tumor thereby restoring expression of the polypeptide in the cancer.

56. The method of claim 55 wherein the epigenetic silenced gene is selected from the group consisting of the group consisting of SYNE1, APC2, GPNMB, MMP2, EVL, STARD8, PTPRD, CD109, LGR6, RET, CHD5, RNF182, ICAM5.

57. A method for selecting a therapeutic strategy for treating a cancer patient, comprising:
identifying a gene whose expression in cancer cells of the patient is reactivated by a one or more agents selected from the group consisting of a CpG dinucleotide demethylating agent, a DNA methyltransferase inhibitor, and a histone deacetylase (HDAC) inhibitor, wherein the gene is selected from the group consisting of FOXE1, SOX17, SYNE1, BOLL, CABYR, EFEMP1, FBLN2, FOXL2, GNB4, GSTM3, HoxD1, Jph3, Neuralized (NEURL), PPP1R14a, TP53AP1, RAB32, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, STK31, SYCP3, TCL1A, TFPI-2, TLR2, UCHL1, ZFP42, ASCL2, ATP8A2, CTAG2, EPHA4, FANCF, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655; and
selecting a therapeutic agent which increases expression of the gene for treating said cancer patient.

58. The method of claim 57 wherein the gene is selected from the group consisting of the group consisting of SYNE1, APC2, GPNMB, MMP2, EVL, STARD8, PTPRD, CD109, LGR6, RET, CHD5, RNF182, ICAM5.

59. The method of claim 57 further comprising the step of prescribing the therapeutic agent for said cancer patient.

60. The method of claim 57 further comprising the step of administering the therapeutic agent to said cancer patient.

61. The method of claim 57 wherein the therapeutic agent comprises a polynucleotide encoding the gene.

62. The method of claim 57 wherein the demethylating agent is 5-aza-2′-deoxycytidine.

63. The method of claim 57 wherein the therapeutic agent is 5-aza-2′-deoxycytidine.

64. The method of claim 57 wherein the cancer cells are obtained from a surgical specimen.

65. The method of claim 57 wherein the cancer cells are obtained from a biopsy specimen.

66. The method of claim 57 wherein the cancer cells are obtained from a cytological sample.

67. The method of claim 57 wherein the cancer cells are obtained from stool, mucus, serum, blood, or urine.

68. A kit for assessing methylation in a test sample, comprising in a package:
a reagent that (a) modifies methylated cytosine residues but not non-methylated cytosine residues, or that (b) modifies non-methylated cytosine residues but not methylated cytosine residues; and
a pair of oligonucleotide primers that specifically hybridizes under amplification conditions to a region of a gene selected from those shown in FOXE1, SOX17, SYNE1, BOLL, CABYR, EFEMP1, FBLN2, FOXL2, GNB4, GSTM3, HoxD1, Jph3, Neuralized (NEURL), PPP1R14a, TP53AP1, RAB32, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, STK31, SYCP3, TCL1A, TFPI-2, TLR2, UCHL1, ZFP42, ASCL2, ATP8A2, CTAG2, EPHA4, FANCF, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, and ZNF655, wherein the region is within about 1 kb of said gene's transcription start site.

69. The kit of claim 68 wherein the gene is selected from the group consisting of the group consisting of SYNE1, APC2, GPNMB, MMP2, EVL, STARD8, PTPRD, CD109, LGR6, RET, CHD5, RNF182, ICAM5.

70. The kit of claim 68 wherein at least one of said pair of oligonucleotide primers hybridizes to a sequence comprising a modified non-methylated CpG dinucleotide motif but not to a sequence comprising an unmodified methylated CpG dinucleotide motif or wherein at least one of said pair of oligonucleotide primers hybridizes to a sequence comprising an unmodified methylated CpG dinucleotide motif but not to sequence comprising a modified non-methylated CpG dinucleotide motif.

71. The kit of claim 68 further comprising (a) a first oligonucleotide probe which hybridizes to a sequence comprising a modified non-methylated CpG dinucleotide motif but not to a sequence comprising an unmodified methylated CpG dinucleotide motif, (b) a second oligonucleotide probe that hybridizes to a sequence comprising an unmodified methylated CpG dinucleotide motif but not to sequence comprising a modified non-methylated CpG dinucleotide motif, or (c) both said first and second oligonucleotide probes.

72. The kit of claim 68 further comprising (a) a first oligonucleotide probe which hybridizes to a sequence comprising a modified non-methylated CpG dinucleotide motif but not to a sequence comprising an unmodified methylated CpG dinucleotide motif, (b) a second oligonucleotide probe that hybridizes to a sequence comprising an unmodified methylated CpG dinucleotide motif but not to sequence comprising a modified non-methylated CpG dinucleotide motif, or (c) both said first and second oligonucleotide probes.

73. The kit of claim 68 further comprising an oligonucleotide probe.

74. The kit of claim 68 further comprising a DNA polymerase for amplifying DNA.


SEQUENCE LISTING (274 sequences)

SEQ ID NO: 1 - DNA, 1200 bp, Homo sapiens
ccgcctcctctccattttacagaggaggaaaccgagggttgagcggcgaatgatttgccctgcctgcccagcacggaaaaaaatcactcttctggccccagtcgcctccctttctgtgctgtgagtctgtgtccagagcaggaagggctgctccgagctgcagggacgggctctgtgtgttaaaccttcaaatggcttccagggactcatggagcatccaggacacctggagatggcgactcctgggacccctggcccttccctcctctggccttcggtccgtgagggtgaattaagtgtgccacccaccaggggcagagacacatggagacagagattgagagacagagaacagagggggagacagaaagagagagggacatgaagagcaacggagttacaggaggatctgacgctccgttggggagacagggagaaatgtggggagatgcagcgtcccaggtgctccccagtggccctgcccctcctcccctctcccctccccaccaggcaggaccctcagggagctccagcctggacccgaaccctgtcccaccctgacgcacctgcaccaggccaggctggacctgtggggtgggtgcaggcctggccccagcccggccactgccctggaaacccctcccccgccaagatgctgatgtaacatccaggatttgatgttttactctccaattctgcaaatccagacgcaggaggttttaagaggccgttgcaccctgcaataaagtgcgggacacaggaatcttggtcctcggggcacccccttccctcgggggggtttcgccccccgcactcacggcccctgagcccgcgcggaggcccgacgccccgcggccgacgccggtccccgccccgggccgccattggctgttgccatggcaccagcccgtcttcccgcgccctcattggctgtctcacccgcatccctcatcccgcatcctcccccgctcgcggtggtctcgcccagcgctaggagcggcagcgccgcctgcccaggcccggaccgggctttgtccgccccggagcccctgcccgcgccgcggagaccccggagcccgcgcgctccgaggccaccccgggccgggatttccggtggggcccgcggagccgcgcagagggaggaggccccagacccaggcgccccgccagcccagctgcacgtaagcgggtgtgtgtcct

SEQ ID NO: 2 - DNA, 1200 bp, Homo sapiens
aaaactgtcatggttacagccatcaaattacaggaggttcacacacagcagtcagcaatggtttgggaatattaaataaatgtgtttctctaacaggacaagacaggatcatttatttatgcaacttaaaagctgtcccagaaggagaaaaaagtagcgtattatttgtccaaaataaacaagattgaagaactgaacatctctctgtggatcaatttacagatcacatggaaataggcttatcttattgagaaatattccattttagatgttcaaataccttagcatattttaaaaactatttcctatgtgaccaaaagaattcatgttctattcggtaaaaattaaccaaaattaacggtgctttccaatggacgcaggaaaccttttttctttacaaaatatgcccaccctttttttgttgctacttcccacattgaattcttgttttactgaattttcttactataaaaataaagcatgatgatagttttgaaaacaaaacagaagtatttgagaagtacagaccccactcccactccttgtctcctttctgacactctgccccttaactatactggaacagggagtcaactgcaaggaaaatcctggggactaggggctgaggaactagaggtggagagatggaagagtgtccagtagcccaggaaaggatgaaacaattccccaacaaaaaagtaaaaattttcagtagccctccgagaagatgcagcccctcccaggtaaccagagaaaggcaaattgtgaaagcagggagaaggggctactttaggcctccaggactacaaagacaaaaaaatgagtgacaggacctgatgtggtggggtgagatgggaatggaagtaaaggagggggctcgtactcttggtgggagtggaaattggggacccaagcttactggcgatgcagccctcacgtgaccaggagctgacgtgggcaaaggtacttaaagccctgaccgtcgttctccgtcctcctctgggtaccaactctattgcgcagctcgctgccgtgcgtttaacccaggcgaggaggaggaggagaaaattcccccagattcgggcaggtgggtgtgagggtgggcacagtctgggacccctgaatagggtaatctagggatgcgggagtcggaggccgcgggtgcctcggtctcccccactcctggccggctttccagac

SEQ ID NO: 3 - DNA, 1200 bp, Homo sapiens
ccagacctgaggtgctcaaacgcggctcattttggtgggcggtggggaaagggggagggggaaacgaaaagacttttgatagactttacgaagaatccgcttacttcctttccagccagtgttttttggttttgtttataatattcaggcccgcaccccacattccgtcctgttttgagaggaggagggaagagaaataaacgtggcagcgcatagaaggccagcagggaggttgggaaagggtaaccaggaggcgggggtggggcatgcaatttggtaacaatctgggcggcacactcccaccctgactttccccaccaccgccccggcaccctcccatcccccagccgaccctctgtgatttgggagtgctgggaaatgggtgtaccagctgctgttggagaggtggcgggtttggcgctgtctgggcttcacgggaggtgggggggaaggggcggaccctgagagaatctggtattaaaccgccttgttaactcgcagactgctttccagacacctccggcccacacagccgttcaccccccgtcttttcagtcctggaaaaggaattcgggtatgcaggcgggagaaatggcggttgttggggggatttgtgggaagtgggaggtagagcccatagcagagaaatcgtggaaaatctcgaccctgaacccgaaaaggcagacgttgaggcccctcttctgcaggctaacgctaggcctaggggctggggagcgaaagatgggtgatcctgggaggggaacactgaattgtttggcaggagtcccccataccgcgtcccatccagcttttgctgcaccctcgccccgctcccctcccgcttcgttgcaggagaaggggtgtggcggtgtgtcctggagaaatggcggacgggggcggggcaaagcggaaggagggattgagtgcctggggagcctgcgcaggaggaggattgaggtgggaggggtggggtgagaagagttgaaggaattgagaggtagaatcatttgacattctgatgtgcaaacacgaaaaggcagttactgagacccttgtcctggatgctgatccatccaccaggctttcattgtctaacgtcgacagtctatatttctgctcttcagtctgtccttaggatgaagctctaactgaactgaagtaaggagaaacagccttgaatctttggagggtcagtgctgg

SEQ ID NO: 4 - DNA, 1200 bp, Homo sapiens
cccagagttgaatcattaactggaacctaatccgaagacaggtgtttaaaactgtcatggttacagccatcaaattacaggaggttcacacacagcagtcagcaatggtttgggaatattaaataaatgtgtttctctaacaggacaagacaggatcatttatttatgcaacttaaaagctgtcccagaaggagaaaaaagtagcgtattatttgtccaaaataaacaagattgaagaactgaacatctctctgtggatcaatttacagatcacatggaaataggcttatcttattgagaaatattccattttagatgttcaaataccttagcatattttaaaaactatttcctatgtgaccaaaagaattcatgttctattcggtaaaaattaaccaaaattaacggtgctttccaatggacgcaggaaaccttttttctttacaaaatatgcccaccctttttttgttgctacttcccacattgaattcttgttttactgaattttcttactataaaaataaagcatgatgatagttttgaaaacaaaacagaagtatttgagaagtacagaccccactcccactccttgtctcctttctgacactctgccccttaactatactggaacagggagtcaactgcaaggaaaatcctggggactaggggctgaggaactagaggtggagagatggaagagtgtccagtagcccaggaaaggatgaaacaattccccaacaaaaaagtaaaaattttcagtagccctccgagaagatgcagcccctcccaggtaaccagagaaaggcaaattgtgaaagcagggagaaggggctactttaggcctccaggactacaaagacaaaaaaatgagtgacaggacctgatgtggtggggtgagatgggaatggaagtaaaggagggggctcgtactcttggtgggagtggaaattggggacccaagcttactggcgatgcagccctcacgtgaccaggagctgacgtgggcaaaggtacttaaagccctgaccgtcgttctccgtcctcctctgggtaccaactctattgcgcagctcgctgccgtgcgtttaacccaggcgaggaggaggaggagaaaattcccccagattcgggcaggtgggtgtgagggtgggcacagtctgggacccctgaatagggtaatctagggatgcgggagtcgga

SEQ ID NO: 5 - DNA, 1200 bp, Homo sapiens
tgtttaaaactgtcatggttacagccatcaaattacaggaggttcacacacagcagtcagcaatggtttgggaatattaaataaatgtgtttctctaacaggacaagacaggatcatttatttatgcaacttaaaagctgtcccagaaggagaaaaaagtagcgtattatttgtccaaaataaacaagattgaagaactgaacatctctctgtggatcaatttacagatcacatggaaataggcttatcttattgagaaatattccattttagatgttcaaataccttagcatattttaaaaactatttcctatgtgaccaaaagaattcatgttctattcggtaaaaattaaccaaaattaacggtgctttccaatggacgcaggaaaccttttttctttacaaaatatgcccaccctttttttgttgctacttcccacattgaattcttgttttactgaattttcttactataaaaataaagcatgatgatagttttgaaaacaaaacagaagtatttgagaagtacagaccccactcccactccttgtctcctttctgacactctgccccttaactatactggaacagggagtcaactgcaaggaaaatcctggggactaggggctgaggaactagaggtggagagatggaagagtgtccagtagcccaggaaaggatgaaacaattccccaacaaaaaagtaaaaattttcagtagccctccgagaagatgcagcccctcccaggtaaccagagaaaggcaaattgtgaaagcagggagaaggggctactttaggcctccaggactacaaagacaaaaaaatgagtgacaggacctgatgtggtggggtgagatgggaatggaagtaaaggagggggctcgtactcttggtgggagtggaaattggggacccaagcttactggcgatgcagccctcacgtgaccaggagctgacgtgggcaaaggtacttaaagccctgaccgtcgttctccgtcctcctctgggtaccaactctattgcgcagctcgctgccgtgcgtttaacccaggcgaggaggaggaggagaaaattcccccagattcgggcaggtgggtgtgagggtgggcacagtctgggacccctgaatagggtaatctagggatgcgggagtcggaggccgcgggtgcctcggtctcccccactcctggccggctttc

SEQ ID NO: 6 - DNA, 1200 bp, Homo sapiens
ttacaggcttgagccaccatgcccggccaagatgtcagttttttaatggtgtgcaacgtccgtttctttgatagttggaaagcgtggcaggtattagagggcgaatgtatatttaagaaaaacaagataagtcttcatctgtagagtattaatttttctgtgatcctttatctcagacgcctctcctctgaaggtcactgctcaagctgggcagccccttctaagaagggatgctgccttggagttggagatgaggttaagatctacgtgcgggtaccagtttcagtaataactggatccttctctaggtctaggctggaacgtctcaagtgaaggttattggggggcaggggtttgggtccacccaaaaaggcttgacccataggactgaaatcaaatcttgagctaggctggcttgccacgtaaccacagaaacagtaatttaagcaataaaatgaaagctcgagcgctggtccagagctggggttgggacagctactgcactcaggtctccaggtgcatgtagggagaagagcagcggatcccttgcagacaatggtgggagcgagctcttcaccctggggtctccgaagcccatttttagcgtccagaggtgtgctttcgccatctccttctgcggtggctgcttgcagggcgcattcccattgctgctgtctctgttcctaaggaagatcaaaggatcccagaaaacccagttccagagctcagttcccgggactagagggtacgctcggccagatggctgggggtgccgctggccttggcgcccccgccgtccccgcgcggtcgcccaccccggcccgacaagcagcttgggcgtccagcccggcccggtccctgcagggagggagcgcaggagctgggggcggctctcccggggcggggctcgctccacaagcgccaggggccgcgcggggcgtgggcgtgggcggggccgggggcggggccggccttggctgccgcggcacaggcggcggcgtctccagggggagccaaggtacgtaggcggcgtccgcctcacccgagcatccttccccgccggtggccgcccgcccgcggcccgcgctcgcagcctcccgcctggcggttgacttctttgtgtcgtattctctgcctgcgccctgtctctgcaggacctgttcgttcttctttgggctataagaaggcagaggatgagat

SEQ ID NO: 7 - DNA, 1200 bp, Homo sapiens
ttctgtgatcctttatctcagacgcctctcctctgaaggtcactgctcaagctgggcagccccttctaagaagggatgctgccttggagttggagatgaggttaagatctacgtgcgggtaccagtttcagtaataactggatccttctctaggtctaggctggaacgtctcaagtgaaggttattggggggcaggggtttgggtccacccaaaaaggcttgacccataggactgaaatcaaatcttgagctaggctggcttgccacgtaaccacagaaacagtaatttaagcaataaaatgaaagctcgagcgctggtccagagctggggttgggacagctactgcactcaggtctccaggtgcatgtagggagaagagcagcggatcccttgcagacaatggtgggagcgagctcttcaccctggggtctccgaagcccatttttagcgtccagaggtgtgctttcgccatctccttctgcggtggctgcttgcagggcgcattcccattgctgctgtctctgttcctaaggaagatcaaaggatcccagaaaacccagttccagagctcagttcccgggactagagggtacgctcggccagatggctgggggtgccgctggccttggcgcccccgccgtccccgcgcggtcgcccaccccggcccgacaagcagcttgggcgtccagcccggcccggtccctgcagggagggagcgcaggagctgggggcggctctcccggggcggggctcgctccacaagcgccaggggccgcgcggggcgtgggcgtgggcggggccgggggcggggccggccttggctgccgcggcacaggcggcggcgtctccagggggagccaaggtacgtaggcggcgtccgcctcacccgagcatccttccccgccggtggccgcccgcccgcggcccgcgctcgcagcctcccgcctggcggttgacttctttgtgtcgtattctctgcctgcgccctgtctctgcaggacctgttcgttcttctttgggctataagaaggcagaggatgagatgtcccgggccacgtctgttggagaccagctggaggcacccgcccgcaccatttacctcaaccaaccgcatctcaacaaattccgcgacaaccagatcaggtaggagaaggcggccggctcgcgcggaaggcggtggagtcacagctgggaagggg

SEQ ID NO: 8 - DNA, 1200 bp, Homo sapiens
ttgtcagtccctaatgttaatttaggttaaaagaaatgtaatgctcgcatcagtaaaagcaaggtcagactttcacctgaacagttttatttttgttttgttccgtttttactccgaggtcagaatgtcatcaggcacaataaagatagcttcttatcttcatgggattgtgcccagaaagaccaagcacagtctaaacattacgcgataagaaaaaacaaaaaatagatcaagtgaccttggacaagtcatcttacctctcgatggttttgttttatgaaagaaggtgtgtgtgtgtgtgtgtcgatgggtgtgtgtgtgtgtgtgtcgatgggtctgtgtgtgtgtgtgtgtgtgtcgatgggtgtgtgtgtgtgtgtgtgtgcgtgcgggggttgagaaggttgctgttaaggttccctagggctacctgcagctttaatatgcgtagttagaatgtcaggcaggttgtggagagtggggggctggagacccccgacttctctgagtgccaagccacacatcagacaccttggaaataacccttccacgaagtccctacacagatcccaccaggctgggcacggaagaacagaaactgcgaaaagaaagggactggatgagtcctggggaatcacagcgcctccacttgcacaggacaagtgacaggcaggtaaataggtagcagacaggcagagactccgagtcccccaggtgcacccctctcctggaagagcccccgaaagtacaggcccaatctcccaccccaccaagccctccccatcgccttccccgacacctaaaagccccggtcctgcgtaatattgcggatccctcccagcctcccgtccgctcctcctcccgcggggctccgctcagggccggtcccccgcccccacgccgcagcccgggccagcggggcccgccgcggtggcgttcgcggatgctgccgcattggccgctgcggcacggacggcgcagcctcgggcgcggcccggcacaggcgccggcggtccccgccagctagcagcccggcgaggcgctggcccacccatggtcctcgggcggcggcccctgcgcccagccctgcgcgtagcctccgtctctcgcccggggccgccgagcccccgacacgggcgagatgctgaacggcgcaggcctggacaaagctcttaagatgtccctgccgcggaggtcgag

SEQ ID NO: 9 - DNA, 1200 bp, Homo sapiens
gccaaagtgatatattaaggtcaccaccttggtcacctgtccatctatatttttggagtaagcaacagaattaggagtctgaacataggaattaagaagctctcgaatataacagtggaagaccagcaccttcccaacctcttgtcagggcagggatactctatctctgcgtcactcaacctgccttggcctcacagctcttccttttgcttaagtcttcaaaccatcaacagtgatagccactaattttagcaggtggcattagttgtcagcaaatgttttttcatatttaatgaatgtttattataatagaataaaatagaaatgtttattaataaaacttgaaatttgtcccagaaggataaaaacgtagattatttacataaaatggccaagatctgacaacagaactgtttcctgaagatttacttcacagattattttgaaaacaccaactgtgttcaggaaaattttctctcctttttttcataaaattttcaaacaagtgagaatgctctaaaaatgatggtgtggagcctagagaaaatgttttctgtgtgctatagatgaacataaactatatatatatattactttcccaggttgaagtgtttctttcaaaaataaaatttgattattttcaattttttgagggatgctgatttttaaaaaggaaataaagagaaattattaggaagcatatacacagccagtgagaagaggaaagaagaaaaggccaaggccagaggaagggggtttggggtgggggaaaggaagggtacagaccctcccaaataaccgaagatgggtaaactccaaccgcaaagcgatgatgccacttgtgggccctcagattggagaggacggagatgagtgacggggctgtgcgcactggggcaccgcgcacagacgggaggaggggggtgtctgtgtgttccaaaggggggagtaccagttaactggccgccggcccgtgggggttggtgagaaggagggtgagcttggcggtgacgcacggccctcacgtgaccgggagctgcagagctacgcagccttcggtgcagtcgtcactcgtgtctcgctaccagctccccgctgccctgcgctcggcgggctggcatccggcccgggggaaagcggaccaggtaagggccccggggcccgcgcgccctggctaggccagcagaggctcaccgaccc

SEQ ID NO: 10 - DNA, 1200 bp, Homo sapiens
ttgacacttggatttggatcaacaatgctttcaagaagaaagacttttgatcaaaagcgggaaatgagaaagcgactttcctctgaaaagtgcctcccagtcccgaggctgcgaggcccccacgccaggctggctcccacggaagccgggcacccacccggcccgaccaagcgccactccgccccgtggacggggcgtcccaccccggggacgcccgccccacaccgcgtttgcaccccggaggccccttgccgcagaggcggacggcgcgcctctcccgggcccctggggtccgcgcctccctcgggcagactctttcgactctgctcgagcctccgcttcttcctgcgggcggacgccccggacacaacgggccccgctgttcacgcaggggcgccccggcggggcgggcaaagacccggggacgcggtcccgtcccgagacgctcagctccggcccaccgctcgcagctcccgccccgggcgcaggtcccgaccccacgggccgtctcggagccgcagcggccgcttccctgcacgtcctcacgccccccgcacggacgccgccagccccgcgcctcagtttccccactagcaggatggaaagacgggccccgccccgaagcgtagcggcgtctccgtggtagccagtgcccagagagtccgccggtcccaccgccccttcaaaggagaacccggcccaccgcccgccgcggcggcgaccgcgcagcccactcgtcacgcggcccgcggcgtccagcccgggccggctcacctcaggcggtcgctgccgccctcgcgcctgcgcgcccctcgccccgcccctctccccgcccgcgtcccgcgcaccgcaggcctctgcccctcgcccaccgcaggacccgccccgcgcacgcgccgcacgtgccacacgcaccccacgcccctgcgcacgcgcaggccccaagtcgcggccaatgggcgacgcggccgcagatccgcccggccccgccctgccctgtgagttcctccggccgggctgcggggctccgctcagtccgggagcgcagctgggccgcggcgctccgacctccgctttcccaccgcccgcagctgaagcacatcccgcagcccggcgcggactccgatcgccgcagttgccctctggcgccatgtcgcagaacggagcgcccgggatgcaggaggagagcctgcagggtga

SEQ ID NO: 11 - DNA, 1200 bp, Homo sapiens
tgctttcaagaagaaagacttttgatcaaaagcgggaaatgagaaagcgactttcctctgaaaagtgcctcccagtcccgaggctgcgaggcccccacgccaggctggctcccacggaagccgggcacccacccggcccgaccaagcgccactccgccccgtggacggggcgtcccaccccggggacgcccgccccacaccgcgtttgcaccccggaggccccttgccgcagaggcggacggcgcgcctctcccgggcccctggggtccgcgcctccctcgggcagactctttcgactctgctcgagcctccgcttcttcctgcgggcggacgccccggacacaacgggccccgctgttcacgcaggggcgccccggcggggcgggcaaagacccggggacgcggtcccgtcccgagacgctcagctccggcccaccgctcgcagctcccgccccgggcgcaggtcccgaccccacgggccgtctcggagccgcagcggccgcttccctgcacgtcctcacgccccccgcacggacgccgccagccccgcgcctcagtttccccactagcaggatggaaagacgggccccgccccgaagcgtagcggcgtctccgtggtagccagtgcccagagagtccgccggtcccaccgccccttcaaaggagaacccggcccaccgcccgccgcggcggcgaccgcgcagcccactcgtcacgcggcccgcggcgtccagcccgggccggctcacctcaggcggtcgctgccgccctcgcgcctgcgcgcccctcgccccgcccctctccccgcccgcgtcccgcgcaccgcaggcctctgcccctcgcccaccgcaggacccgccccgcgcacgcgccgcacgtgccacacgcaccccacgcccctgcgcacgcgcaggccccaagtcgcggccaatgggcgacgcggccgcagatccgcccggccccgccctgccctgtgagttcctccggccgggctgcggggctccgctcagtccgggagcgcagctgggccgcggcgctccgacctccgctttcccaccgcccgcagctgaagcacatcccgcagcccggcgcggactccgatcgccgcagttgccctctggcgccatgtcgcagaacggagcgcccgggatgcaggaggagagcctgcagggtgaggcggaggaggcggcgcgggagccg

SEQ ID NO: 12 - DNA, 1200 bp, Homo sapiens
ccaacaatttgagcggccaagacaggcagctcacttgaagtcagaagttcaagcctggccaacatggtgaaaccccgtctctactaaaaatacaaaaattagccgtggtagcgggcgcctgtaatcccagctactcgggaggctgagacaaggagaatcgcttgaatccggaaggcggaggttgcagtgagccgagatcgcgccactgcactccagcccgggcgacagtgcgagactccgtctcaaaaataaaaataattagtaaattaattcattaatttaagaaaaaaagcgcaatcttcttccaaaggcgatcccaaggctgttggaggctgaaaccgctaagtcgccccctaccgcgcgggtggcttcgtcccggacccctccagaggctgtgccactgctggaggtgctcagcggggccgggacccgcttggggcgccgtccggggcgagcaccggagcccgactgggctgaatggcaggtcctgaccaagccacccgcgcggccccgcccgcctggcgggatgagtgacggcggcaacaggtgcagagcggcgtccgcggcttctcgggagcccccgagttttgcgagtcaggccccgagagtcagagtttgactctagaaccttcttcgctttacaccagtcttcggaagtcgcccgtcaaccggcttcttctacctcagtaaagccttaaggtcacctcaacctctcgaaacccaccagtcccagaggccgactcttctcaagtggaggagaggggaaggcgtgcatggagctttgcctgaccgctcgctctctcctccccagccgtctgagagaaggcgagaggggtcccagaaccgagccctcgctccgcacgccccacgcccgccccgctgtgccgtgcgctctgcccctgagacgccccggctcggagtcccagggccacagccgaaactcgccctcccgcgcccctctccggcttgcagcgcccgcccgcaccgccacctgccacccgaggcgacgcagtccgcttctccccacccgccaggtggcaggttcagggccggggtccgagcctgcttccctgcagctgccacagccaccctcgccgccagggggcgagagtcccaggcaggacaagcagcgctgctgccttggcgtgaaggtggtttcaaactttaggcttcttttccctaccccgcgtctctgaagag

SEQ ID NO: 13 - DNA, 1200 bp, Homo sapiens
gaggaagatggaaaccgagtccgggccggtgagtacggcgatcagggccccagggcgcgggatagcggggagaaggagcagcgtggagcctgtccctgagggcttggaggggcgaggtgaaggccccaaacgctcgctatctggttgggaagaaacgtggccaggtgccccatcgaccgctgccaggagccacccgcccgcagacctcgtctggctgtgtcatgggggtcagaaaggcgccgccacggggcctggactgggtgtagacgggccaggggacctcaggcgtggatgtttcgggaggcggcgggctgccaggcacccagcgaagtccgagcaggtaaaccgctttcccgccgaaattgggctaggcagcgtctttgacgctccctgcggccgaactcgacccagccgcccaaggcgaggagttgccagggcggtcgacgcctccactccccgactggtcagcgaggaccggaaggtctgagagacctggcagggcccgcctgtggcccaggcgggacccggacactctagccctggctgtgcacccaagccagtcagggtggacttggggaagtgaaggaggactgcccgcacaggcccgaggcgcagcggcaacgtgccatcccgccccgccccgtcccgccgccagcagccaatcagggagcgccgcgcgagggtggggaccagctgcgcacgaggccagcggcggggtcgctgccgttgaggcttcccgccactgctgctggcggatttgtggggcaaaatttctcgctggctagccttcttttccctcccgcactttggtggggagggggtggatcctcgtttcggtgcccaagttcacgatgacccgagagaactcgaggaagttgtcgccgcggccatttcccccagtgccgcaacttgctggccttggagggggaggagcgccgaggcagtgactgcgacggttagcccgcccgttcctgctgtcttcgtttgcaaaatagatgctagatctcgaggttgattctcaaggggaggggccagtgagggcacattgctttggaggagtagtttaataaactctctagtgctggaagttttagcaaacggaggagccttcaaacccggcaacagtccccatctaggtgtttccgtaacacatgcggaattattgctagtggaatttcaggctttctttgttgtttaact

SEQ ID NO: 14 - DNA, 1200 bp, Homo sapiens
ggattttgtatatgtcatcagctgttgtgaccttccaacccatgagaattttcctcatgcctgaggattcacttaaggctacactttcagatccagagatatctttcttttcctcttggctgggtcagtctgactttggctgcatgatcacacattattagcaggtgctaccatttttaggttagaatatgtgactttcttaaagtaaaaaaaatcctatagtcctctagcttatcaattcattaagttaaactttatataccaattttcttaaatggaggaatgcctttattaatatgcagcatctatgtcattactaaattactataccaacaaagatgaaataatttattgcccaagttggtctcccccccacccaacctttttccccagttttgtgtggaagtttgatcaggagctttttccatggaggttttcagcttccttagttctaggcacaaagttatgatctttccctgatgctgaccaccttaggatgatatctgattattagtcttgctactgcgctattaagtgtcacttatttctaaggaaatgaattgagggtcttggacaggataaggacaggaatgttttattacccacactacagatgttggaaaacaaagctgaaaactcttttaatttgtgtctatactgacatctgaatgcttaacccattattgcttttttttttggtggtggtgggtagagatggggtctcactgttgcccaggctggtcttgaactcctgggctcaagcgatcctcctgcctcagccttccaaagtcctgggattacaggcatgagccactacatccctattattgccttctttaaaaatcttttcttctctaaaaatctttttagatgttaattaacccatctggttgttgtttttcagtagagaaatggtcagaaggaacgacaccacagaagaaattagaatgtttaaaagaaccaggaaaaacatctgtagaatctaaagtacctacccagatggaaaaatctacagacacagacgaggacaatgtaaccagaacagaatatagtgacaaaaccacccagtttccatcagtttatgctgtgccaggcactgagcaaacggaagcagttggtggtctttcttccaaaccagccacccctaagactactaccccaccctcatcaccacctccaacagctgtctcaccagagttt

SEQ ID NO: 15 - DNA, 1200 bp, Homo sapiens
taaggctgcttgtactctgataatcatgattatggtgttatttagagcccagcccaaatacctgagcctaaatgagtgaaagagcagagagtgggcgtttttttgttttttttgttttgttttttgtttttttgagacggagtctcgctcagtagcccaggctggagtgcagtggtgtgatctgggctcactgcaacctccgcctcccaggttcaagcaattctctgcctcaacctccagagtagctgggattacaggcacccaccaccacgaccggctaatattttgtatttttagtagagacagggtttcaccatcttggccaggctggtcttgaactcctgacctcgtgatccacccgcctcggcctccgaaagtgctgggattacaggcgtgagccaccgtgcccggcagagtgggcgtttttaaatgaagcagtattcctttagtttggtaaaattgagctgaagttacacacagtaacaccttgacttgttgctgacggtgtgctttgtggggatagttggtgtctgatctcaactgtggagaaacaggaagtttctggattcatgctgaactcccattccacaggcgagtttcacaaaagaaaaagtgctctgttgcatacaattattcactgtgtgtctacaacgtggcagaaattattattcctaaaggtgaccgaagagagctatagtttatggggccaggagcaatgtgtggaatggcgaggcagtgacaagacagcactgccataaatctcttactttctttccaaacgagctctaattttaagctcaacgaccaaagacggagaggcagtcgctttgagctactcctggctatttgttgggaaatcacctaaatccgttcagatttagccagaaaaggggaaataagtctccctcgccggccaaatcatggcctttgtcgctttcggctccccattggctgcggccgctgtggccccgcctccttgagtggtgcggagctttgtgatgcggagcttcgtgatgcacgccccgatgcctgcggggctataaaaacgctcgcaagcgccaagtctcctcaggagccgccggcaagggggcaacgaggaagctcttaagagcgcggccggaaagcaggtggggagaatctggagaaaaggcgcaccctgcggcctcggagaccattatgtctcagggagtctctcgcagt

SEQ ID NO: 16 - DNA, 1200 bp, Homo sapiens
tggaaaatcagacaccagacccctcacctgcaacaattgcctgatgcctgttgacccactcctcttcatctctccctaactcctgctattttcccacatgcagttacatttcttccctgctatatcctaattttaatcagttgagaagatggatttgagactgatctctcccccctaggctgtagcacccaaataatgccttcttccctgacaataatggggtctcagtgactggctttctgtgctgtgaaccaccagacctagaccaaactcctggcatttctctaacaatttcccaccactgaactaagttataaattctgaggatggatccctgtttcttcattttaagatggggattaaaaggtacatcctagagtgttattgtccgtgtaaaatagggctgtgaaaccccctacacctatgcacagataaactgatggccctgcaaatgcccagagaagtaaccttataaagatttagtgaatgtttcccaccattattggaaaatctggctaagtcagtagctttgttttcatatacttagggggaaaatcaacaaaaacttgatgccctgttatttccttccagtttcttctgcaactctagctccaaagactgcagttcctgcagtatttttttttttagaacaccaggaaaaaaagaaaaaaaaaccatgcaaatgtactatttcttcaaacacaactaggaatgattgaacagctgggagaatatgaagaaaacccctcagagaaccaagggcaagcagtgagttcagatctaggcacatggccactaggaatggttcttcactgccacgtgggcagccaacagccagtagctagagaccagcctcggtcttcggcctgcgggttctgcaaagtcaggctagctggctctccgcctgctccgcaccccggcgaggttccggtggggaggggtagggatggttcagccccgccccgctagggcggggcctgcgcctgcgcgctcagcggccgggcgtgtaacccacgggtgcgcgcccacgaccgccagactcgagcagtctctggaacacgctgcggggctcccgggcctgagccaggtctgttctccacgcaggtgttccgcgcgccccgttcagccatgtcgtccggcatccatgtagcgctggtgactggaggcaacaagggcatcggcttggccatcgtgcgcgac

SEQ ID NO: 17 - DNA, 1200 bp, Homo sapiens
actcaagcaccagcaagcggactcgaactcaaagggtttcagggtgtagacgaccagagcgcactgaggggctccaggtcggcccgctccggagccgggagagtaaccgcgtgcagggaaaggcgcacggcagcctcggtgcctcccttccctccaccggacgggtgactgactaggggtgaggtcctcacctccaggctgcgcagctgagttctggaaggtactgtcctgggggtgtgagtgtgtgggccctcaggaaaggtgcgaggggcggatgccgtggtctacccgagcttgcgggggtgcggtggtcaagttccccaggaccctcgctggcgccgcttgtcgacaacggggaacagtgggcgccgcgggtggtggtaacttggcccgggaagaaaacacatccctttcccaaaccggattcctctgagcaaaaccagatggtgtcatccgcattttacagctgcgtcaagagaggcccaggacaccaagagccagctgtggccagagccctttcactgacccagaacactggccctcggaaacctggagaactgcgagggcttttaatgcggctgagaagaaagtgaagaccccgactcctcctctctccttcgaaagagccgacacaggtatgaagaccagcgcctcggacgctccggggtagggggccgacgccccctccctttcagaatcacgccttgctgatgctcagtcctgcccagtgtccaccccccacccccgcccccagtttcctttctagcccctccggtccccaaagcgaactaagctagcggcagcttctccatcccggaggagactccaaactcctaggtttcccaccttggagagtactgctctggaatgcgcaggggagcggcacctggaaagggttgggcggaagcttctcggacccagagggggcggacacccggcaccgcgcgtgggagggattaactccaccccactagaccacctccccctctctcccaggagccacttcaaccggttgtcgccccagtggccgccctctgagcacgtgttactgccagtctgcgtcagcgttgggtaaatacatgactggccgacgccgccgggtggggctatttaagagacagccgcccgctggtcctccctgaacttggctcagctgccgggctgctccggttggaaacgccaagccagctgcgtcctaat

SEQ ID NO: 18 - DNA, 1200 bp, Homo sapiens
cctcttttccgtttgcattttgccccatattagaatgctaccatctaaaaccgctccatgaattagaaagtttaagttgtcagggactgaatgtgtctgttttccaacagtttctggcagttccctgcttttatggtgtagtcatggtgaaacctgagtcatctgaagatggtggcagtgccagagagcaggagagaggagctgcaaagaagccagacagcgttgggcatcaataactcctgggcaccaccggccactggccttcattctgacaggagagccaacccaaccctaactgtgttcagagattgctccaagctttacagttctaccaaattctttctgtagcaaaaatataaaggcatcttcttaggtaattttttttcagggttccaagtagcaacacctaacgtctgcagtgcgagttctcttcttctttttttttttaaggtgataaatcataacattttcctggaaaggaaagcagtagacctcattttttgttttaaggaaaaatagtttttctgtttccattcatatacattaaataactacccaggaaatacgccccggttttgcgcgcgtgtgcgtgcgtgtgtgtgtgcgtgtgtgtgtgtgccgtttctctaaaattctgactcatagtctggaggacaatgattgacttttttccacttcacatagttgctatgaatttctcctcacaaggatgggcctgtttactcaccctgggcatcgttggtagagcgctagtgtaaacagcctgaggaacccggtgggccggggaagtgggcgcgctctgttctccgcggccagctgggacgccgggccaggtggggccgcctgcgtttagcaactgctttctcaccccctggatttgcgatgtttgccacagcagcgagaagcgccattgtaatggggatgggaggggtggagcctccaagtcctgtctcaatttagatctctcactctgctgttaggcgcgcccatttcagattactaaactcgaattaagagggaaaaaaaatcagggaggaggtggcaagccacaccccacggtgcccgcgaacttccccggcagcggactgtagcccaggcagacgccgtcgagatgcagggcccaccgctcctgaccgccgcccacctcctctgcgtgtgcaccgccgcgctggccgtggctcccgggtaggaacgtgggcg

SEQ ID NO: 19 - DNA, 1200 bp, Homo sapiens
atgcgtgctgaagtatgtaggggtaaaatgacatgatgtctggaatttgttttaaaatacttcagaaaagcggggagtaggtgaaataagtgtgacaaaaccttaatagttgttgaagcggggtgatgggtacacagcagttcattatacaattctacttttgtgtatgcttgaaacctctcaattaaaaaaagagggtagagagtcatcaagataatttactaaaagtttcctgtaatgtccccagaagacccccctaactcacacacacacacacccacacacacactgccttttgagaactttctctagaaggccaacccaggcagtcccccaactgtaaggaagactcgtgtatgtatgtgcatatgtgcatttccccagggaaaaacatccacagcttccatgacgagaggggtcgtgacccctccccgccaaaagattaaggacctgcgatcctacagaccggagccctgtttgaagtctgcgttgcccctcacctcaagctggtcactgtgtgaagttggcctagaatcccccggcccctgggagcttgttcctccgcctgtaaaatggggctgcagggccgtccacgcggccaccggaaggacaaggtgttcaggccgctaggccgctccctggcaagcgattcccactcgcagcgcggcctcgaccctcgcccaagacgcgccctccgcgcccccaccccctccaggccctggccagtccacctcccgcttggggcggcaatttgtctccttttgaaccccccgcccccgacgggtttccccctttgattcgcggcccggaggcttccccccgctttgaaatgcaaacccgcctcggctggggccgcgggcggcccggagctataaaaggcctgggtggggcgggcgcggcggcaggacagccgagttcaggtgagcggttgctcgtcgtcggggcggccggcagcggcggctccagggcccagcatgcgcgggggaccccgcggccaccatgtatgtgggctatgtgctggacaaggattcgcccgtgtaccccggcccagccaggccagccagcctcggcctgggcccgcaagcctacggccccccggccccgcccccggcgcccccgcagtaccccgacttctccagctactctcacgtggagccggcccccgcgcccccgacggcctggggggcgcccttccctgcg

SEQ ID NO: 20 - DNA, 1200 bp, Homo sapiens
aggcgctcatttaaaattccagtcttgctcccgagggctttggcctccatcttcacctttaacgaccgagggctggtggcggcggctccttcctgctctgcgacaggtgtcgcttctgagcgggagttttgggcacagacatcgcagatccgcaccacccctaggggtctgcatgggtttcagctgcacccagcctggaggggctttttggaagggggcacgtgggcggtgggagcggggaagcaggtgaggggagcccgcctgcgcggatttttctgctgtaaatcagacccacagtctccagagcgtcttttccccaagtgtggaacacatttttcttctaatcctctttggagagggggttaggaacgtgccgaggccggagccgggagcggggtgggtcccggcgcctgtgaaccgagtgggggagcgggccgcgcgctgccctgaagaccctgcctccgcgcccttgggggctcggggcccagcgccttcgcaggcgagcggcggcggctggctctgcccggggtgcgcgggggcggtgtttcctcgcctccccctgcgcccgggctcgccaagccccgactgcccgctcggccgtcgggtccgcgggcgcgcggacgcctcgacggctccccctgccggggcgccccccggtccccaccctcacccggcggctcaggactagtcccccaacttgttctctgctgttaactagtcgcgctgccggaacgatgggatggggttgccacagcaaccagactggacgtcacaatccgggcatgtgtgccgcccccccgccgtgccgccgtgcctctggcgctgccgccgccaccctcggccggctgcgggactcgggctgcggggcgcggtgggaggagcgcccgggctttgcggggagcaggctaaggcggccgagagaaaggggggtcgagacgggggggtggaggtttgggggggtgggggggcaggcggccgccatcttctcgccgaggccgcctcgcccgccgcgcgcgccggtccggcagcgcacgggttaaggctggcaccgcgcccggcgggagggggggcgcccacctcccctcctccccgcgccgggcatgcggggcccagtgggcaccgaggaggagctgccgcggctgttcgccgaggagatggagaatgaggacgagatgtcaggtgagcggggcgcgggtgcgc

SEQ ID NO: 21 - DNA, 1200 bp, Homo sapiens
cctctcggcctttgctgtcaccaaggccaaaggaaggctggctgtgtggtgtaccttgcacctccagaatgccttcaggtgtgatgtggcaccacagatctggtaggtggaggaaggggtgggccattttacagatgggaaagcaggctctggaggcttggatgagtttccgaggacaggtagtagcaaagctgggacctgctatccccaccagagctgtctccctgaatcagtcacccccgaccctgtccagtcagacagcaccgcctccttggagggccaaggctgctcccacccatctcagggcccgagtggtgtggtttgtggctgttgtatttcttgcctttgccagccagcagctcactgtcaccctagcatctgtcagggcccctgcagtgtgtctgtgggctgccagagatggggcctcattgtcccagcctagacgcacggtgcctgggtgcagccctgccccgggtgagggatggtggatggggctgccgtgtccctgggctccagcacagggctcatgctccctgagtgcttggggatcacacagtgatgtgacctcttttcagggggcaatgggaagacctaaggtaggggaagtccagaatctcatgttctgctccctgctctgctgtcccttgccgagtgggcctcagtttccccctctgtcctatgcacatggcagtctcacctacgctggctcagatattctgggtgccgataagttcaccattgagtgaaagggccacgtggtctcacagacccccactgcccatctcctgtgtgcatgacccatgcggctcctggggacggggtggatgtcatggcgtcaccgtggtactgttctgacttgggggcagtggcactgatgggagggagcctgcatggcagcctgctgtcatgggagcctcagggtggggccctgcagctgagccccaccttggggacccttctctgtccacagtgccccccactgaagggcaaagtccagcggattctacactggaggtggacggagccccctgcccccttcatggtggggctgccggggcctgacgtggagcccagcctccctccacctaagcccctggagggcatccctgagagagagttctttgtcaagtgggcagggctgtcctactggcattgctcctgggtgaaggagctacaggtgagtgcccgggagggtggctg

SEQ ID NO: 22 - DNA, 1200 bp, Homo sapiens
cgacaggtgtcgcttctgagcgggagttttgggcacagacatcgcagatccgcaccacccctaggggtctgcatgggtttcagctgcacccagcctggaggggctttttggaagggggcacgtgggcggtgggagcggggaagcaggtgaggggagcccgcctgcgcggatttttctgctgtaaatcagacccacagtctccagagcgtcttttccccaagtgtggaacacatttttcttctaatcctctttggagagggggttaggaacgtgccgaggccggagccgggagcggggtgggtcccggcgcctgtgaaccgagtgggggagcgggccgcgcgctgccctgaagaccctgcctccgcgcccttgggggctcggggcccagcgccttcgcaggcgagcggcggcggctggctctgcccggggtgcgcgggggcggtgtttcctcgcctccccctgcgcccgggctcgccaagccccgactgcccgctcggccgtcgggtccgcgggcgcgcggacgcctcgacggctccccctgccggggcgccccccggtccccaccctcacccggcggctcaggactagtcccccaacttgttctctgctgttaactagtcgcgctgccggaacgatgggatggggttgccacagcaaccagactggacgtcacaatccgggcatgtgtgccgcccccccgccgtgccgccgtgcctctggcgctgccgccgccaccctcggccggctgcgggactcgggctgcggggcgcggtgggaggagcgcccgggctttgcggggagcaggctaaggcggccgagagaaaggggggtcgagacgggggggtggaggtttgggggggtgggggggcaggcggccgccatcttctcgccgaggccgcctcgcccgccgcgcgcgccggtccggcagcgcacgggttaaggctggcaccgcgcccggcgggagggggggcgcccacctcccctcctccccgcgccgggcatgcggggcccagtgggcaccgaggaggagctgccgcggctgttcgccgaggagatggagaatgaggacgagatgtcaggtgagcggggcgcgggtgcgcctggggccagagcggcgggggcgccgagacgggccgggcccagggcggagcgcgggcggcgggggcggcgcggccgggctggcgcctcgcaggaggggca

SEQ ID NO: 23 - DNA, 1200 bp, Homo sapiens
gcatgaaattgagataaaacatttcaacagactggacagactctgaacactgttgaagagaacattgataaattggaacctggtgctgaggcattttcccagaatgcaatgtacagagacaaagataaaacagggagaacatggaagacaccttgagatgctccaacaatcatccaattggagttgcagaagaaacagaaaaaatggtgaagaagttggatttgttgagagtttgcagacctggagagaaatgtgaggtctcagatctggagtgcattctggaatagcaagcaagataaatataaataaatgaacccgtagacacgttagaacctctaggtactttagacctagtacctacaaaataagggcagccagatcgttctcagacttcttaacaacaacaggtggcagagaccatggcaaaatctccggtgcactgagggaaaatgtgtgctgatagttttatatccagctaaactctctctcgagaataaagggaattatacggggtcatgggagctcagaagggactgcctttcttttcttatggggaggagtgaaatcagggaagacatcacagaggtgatgtttatgttccctaggcagtaagggggtgggcattccaggaagagagtcaggtagtttggaatggctgggggctgtgaaggagcactcgtcctgcagcgccttgaatacagggcagtgagatggaacttaattctgtgtggtgggagcaggggaggctcatgtcagagccctgatttaggtaagtggctgtggggctaggcaggaggatggattggctccaaggggtgaggttggaagctttgttcttccccagagtcctgcattgggcctgcctgggtcccagctgtctccccacctagcaggcagctctctcctcccctgcctgtggcggggtccccgcccatggcccatggggccccttgctccttggcagttcctcacgtgcccgtctcccctgcagatgaccaagatgctggacctcctggaggacttcctggagtacgaaggctacaagtatgagcggattgatggtggcatcaccgggggcctccggcaggaggcaatcgacagattcaatggtacccccatcctgcccaagggttctgattgagactcctttttggcctcttctcccaggcctggttcttagccaggtcacccagctggtgt

SEQ ID NO: 24 - DNA, 1200 bp, Homo sapiens
gaaatctacctatttgtaactccagggaagtaaatgagataatgtttggaagatatctagtctaccagctgctcaggcggggggcgaaggttctgttccctgtcccttctctatcaaatggctttaaaatcagaagaaagtattacgggtaggaaatgggactgcgaaaaaaatggaggctgattctggcctaaaggatacttatctctatacaaagggcttctgattcaaagaggccccgggattcaaaaacacaagcagagctacaccacgtggggtttgggacgagctgaattccggataaagatgcctgtaaagaggagttgatgcgaatccagaaaagcacttgaaatgcccaggctttgcatggcacattccccaggattttttcgcaccgcatttagaggtttagacggagtgagctcgaatcaggccaaaagccttaggttactcctgggcggcagcggggcagaggtcagatcaacaggtgtggaaggctgcgaggtgagggaagaagcaggtgtcggtggaggccccagttcctccggcccagaggctccaacgctgctattggtgttccagcagcgggcggaggggtcctgcccggcatctgggcgcgctgggcccagagcccgggaggaggcagacggaggcggcgggtcagcggcgggccagccccggtgggcttccgctcccctccgccggcccggtccggcccgccgcggaggatgcctggaggttgacgtgaatggcctgtggctgggaagcccccaggcgccctgcctagcccgggggcggggccgcctagccgccggccccggtggcgtgagggctggggcgggggccggggacctcgcggggagggagcgggagggaaagcggcccccgcggcccttcaattggccgcaccgcccctcggcgcagaggcgggaggaggcggccctccattggtcgggcccggcggccaatcctcgaaggcccaggcggaggcgggggcggcctgtggggaaccgaggtgcgggcggcgagcgaggcagccgggtgcttcgcagcggagctcgaggctctgtagcacccagttggacccagtcgcccaggcaccgcccgcgccccgcggccgtctcctgcgcctcccagaggagggagttcgagcatcccccgcggcggccactcctcggggccaggagcggggaagcgaag

SEQ ID NO: 25 - DNA, 1200 bp, Homo sapiens
gatctcagggattgaaatctgtcctaggtgtgcaccagaaagtggtggcaagagatgaagcttcggaggaggctgacctcggccaggattcttaagtctcaaagtttcagctttctcacccctcaacggacatgaaatctacctatttgtaactccagggaagtaaatgagataatgtttggaagatatctagtctaccagctgctcaggcggggggcgaaggttctgttccctgtcccttctctatcaaatggctttaaaatcagaagaaagtattacgggtaggaaatgggactgcgaaaaaaatggaggctgattctggcctaaaggatacttatctctatacaaagggcttctgattcaaagaggccccgggattcaaaaacacaagcagagctacaccacgtggggtttgggacgagctgaattccggataaagatgcctgtaaagaggagttgatgcgaatccagaaaagcacttgaaatgcccaggctttgcatggcacattccccaggattttttcgcaccgcatttagaggtttagacggagtgagctcgaatcaggccaaaagccttaggttactcctgggcggcagcggggcagaggtcagatcaacaggtgtggaaggctgcgaggtgagggaagaagcaggtgtcggtggaggccccagttcctccggcccagaggctccaacgctgctattggtgttccagcagcgggcggaggggtcctgcccggcatctgggcgcgctgggcccagagcccgggaggaggcagacggaggcggcgggtcagcggcgggccagccccggtgggcttccgctcccctccgccggcccggtccggcccgccgcggaggatgcctggaggttgacgtgaatggcctgtggctgggaagcccccaggcgccctgcctagcccgggggcggggccgcctagccgccggccccggtggcgtgagggctggggcgggggccggggacctcgcggggagggagcgggagggaaagcggcccccgcggcccttcaattggccgcaccgcccctcggcgcagaggcgggaggaggcggccctccattggtcgggcccggcggccaatcctcgaaggcccaggcggaggcgggggcggcctgtggggaaccgaggtgcgggcggcgagcgaggcagccgggtgcttcgcagcggagctcgaggctc

SEQ ID NO: 26 - DNA, 1200 bp, Homo sapiens
gggatggagtattgcagcctctcgccccacagacttgtgaacagggcgtggggacagacacccagctcctagggccatgccacccccacccctgcccgcctctcgcagcacctttcctgtggatctgacccccttccagacacagaagtaagccagcacccagcagacacagggtcacctcccagctccctaccaggcaagagcgaccagggcagggactgatactgccgaacccaggagccaggcccgacccagcctcaggtccagcaggtcccgcctgtccacctgggccaggcctagagcccgggagcccctggctggtgggaggccacccgcaacccaccccacacgcagctccagctcccccaccaggcggggcgactaggacagggacagaacccgttgaacccaggagtgagatccggccccgggtcccgctgggccctcccgtccaccttggctggacctggcgcctgggagaccttggctggcgcgaggccacgcccaccagacatgcagttccagctaccccaccagctgggcgaccaggacagggacggaggctgctgagcccagttagaggcctgccccccggggtctgtcctgggcgctcccccaaggacggacagggcaggcagggtccgggacgatggccgcacagtcccggccccgtgttcccaggcccgtcttgctcctcgatgtgagggagacccgggggatgggacaggctgggccccgcagtgcctgactccctgcagggctcccgggacaggggtccggcggacagccggctgctcacgggtgaggggtccaagctggcattgcggccaccttccggcccgggctctcttggggaggggcggggttggtgagaaccggtcacgtgctccggggctcactcggggtctcccagggccggaagtagggcccctgtgcgcaggcgccctgaggatcccgggctgcccatctcacgccagggggcggaacttcctgcagcctctctgcctccgcatcctcgtgggccctgaccttctctctgagagccgggcagaggctccggagccatgcaggccgaaggccagggcacagggggttcgacgggcgatgctgatggcccaggaggccctggcattcctgatggcccagggggcaatgctggcggcccaggagaggcgggtgccacgggcggcagaggtcccc

SEQ ID NO: 27 - DNA, 1200 bp, Homo sapiens
ttgcagcctctcgccccacagacttgtgaacagggcgtggggacagacacccagctcctagggccatgccacccccacccctgcccgcctctcgcagcacctttcctgtggatctgacccccttccagacacagaagtaagccagcacccagcagacacagggtcacctcccagctccctaccaggcaagagcgaccagggcagggactgatactgccgaacccaggagccaggcccgacccagcctcaggtccagcaggtcccgcctgtccacctgggccaggcctagagcccgggagcccctggctggtgggaggccacccgcaacccaccccacacgcagctccagctcccccaccaggcggggcgactaggacagggacagaacccgttgaacccaggagtgagatccggccccgggtcccgctgggccctcccgtccaccttggctggacctggcgcctgggagaccttggctggcgcgaggccacgcccaccagacatgcagttccagctaccccaccagctgggcgaccaggacagggacggaggctgctgagcccagttagaggcctgccccccggggtctgtcctgggcgctcccccaaggacggacagggcaggcagggtccgggacgatggccgcacagtcccggccccgtgttcccaggcccgtcttgctcctcgatgtgagggagacccgggggatgggacaggctgggccccgcagtgcctgactccctgcagggctcccgggacaggggtccggcggacagccggctgctcacgggtgaggggtccaagctggcattgcggccaccttccggcccgggctctcttggggaggggcggggttggtgagaaccggtcacgtgctccggggctcactcggggtctcccagggccggaagtagggcccctgtgcgcaggcgccctgaggatcccgggctgcccatctcacgccagggggcggaacttcctgcagcctctctgcctccgcatcctcgtgggccctgaccttctctctgagagccgggcagaggctccggagccatgcaggccgaaggccagggcacagggggttcgacgggcgatgctgatggcccaggaggccctggcattcctgatggcccagggggcaatgctggcggcccaggagaggcgggtgccacgggcggcagaggtccccggggcgcaggg

SEQ ID NO: 28 - DNA, 1200 bp, Homo sapiens
ttgtgaactgcctgtggacagggcaacatggcagagaactgagggcagcttttataacaaattaagagatttcttaatatttaaccctccttacattaactgctcgtggtatattattctttttaaaaaattatttatactttttgtgggtacataataggtatacgtatttattggatacatgaaatgctttaacataggcacacaatgcataatgatcacataatggtaaatggggcatccattcccctcaagcgtttatcctttgtgctacaaacaatccaattatacttttagttatttttaaatgtacaattaaattattttgactgtagtcatcttgtgctatcaaatactaggtgttaataatcacatcatggagaatggagtatccatccctcaagcgtggtgcattactctttgaatatgctgctctggattgtatttgctaatatgtggggcttctctattaatgttgcaaaatagggagccaataatctggttgagagatgaaagcagcagcaggaaaatgtgttcacagaacctcatacgagtggcgtctagggccaaggctccttgaatgcatacccaagggctgatgagaggtggggacgagtgctgccgaaggactcctcaactaccagaactacgtagggcccctaacatcccctcggagcgagctccccgcgccccgtacaccaccaggtctccctggttcctctcctgctccccgtcccgctacctcaggaaactttagcccagcctctccgatacaagccggcctccttgagggctttaggcccctcttcgccagtgtataccccacgacacgcctgcgtactagggaccacccggctttgtgggcggagtccatgagagggttggagccccgctcgtggccccgcccacccaagcctaggccggccttcgtggacacgcatttccggcgacgcctcggtactgacctctgcagagccgggtggagcccattgacgtccagcgaagcgaggagcagcgatggacggtcgggtgcagctgataaaggccctcctggccttgccgatccggcctgcgacgcgtcgctggaggaacccgattccctttcccgagacgtttgacggcgataccgaccgactcccggagttcatcgtgcagacgggctcctacatgttcgtggacgagaacacgttctccagcgac

SEQ ID NO: 29 - DNA, 1200 bp, Homo sapiens
tcgaatctgcagtgagtgatcgcaccactgcactccagcctaggcgacagaggtcaaaaacaaaacaaaagaaaacaaacaaaaaaaacatagcaaacatccagaagactgtgtagagcaatgaatgtctctcaagccagtgatctttttctcttcatttttcacatcaggtgggtagtgtgcccacttcgtaacaaggtttgagggaggcacatttgacacaagagcatgaaaacccaatcacacttatgacttacaaaaggatcaagtcagtgatctttaagctgtgggccattaaatacattttgtgagccgagtgcagtggctcaggcctgtaaccccagcactatgggaggtggaggtgggaggatcacttgagtccaggagttcaagaccagccttgggcaacatactgggaactcccatctctattaaattttttttttttaattttgtggattacaaccatttcaaaaactaaaataagcgtaaatgtatcaaaatgcagcttacatagcgaggataaatgctgttttgtcaatctgttgtttcaagttttttatgtgtggattgggtcaccttgtaacagtgacttacctttatgggtcacggttgaaaacatttccggtgcagtggctgactgtaatcccagcactttgggaggccgaggctggaagatcacttgaagccagaagccaagagttcaagacctgcctgggcaacacggcgacaccccgtctctatttaaaaagaaaaaaaaaagtttccaaggcactgagatgccaggaacccgaggtgtgtgtccagtaggggagggagcaaattgggcgtttggaaaaagtcccacgcctggaaaggaatacacagtgggggggtggggggcggggcacatcgactacttcaaggcacactcccacggttcagaaaaccgattggctaaaactgggaaagtcccgcccacaacgctcactggccaatggctgcgtgaaatcagtgcgaagtgggcgggatagagagcgtgggcgggggggctagcctcgtgcgggctccttaagtagcggctgcgtggcttccctggcacgctactcttacgacgtcacggtcaggtggtgcagagctggacggcaacgacgtcggacgcgccccttcttggaacaatgtcccaccacggaggagctcccaaggcctctacgtgggtcg

SEQ ID NO: 30 - DNA, 1200 bp, Homo sapiens
cctccagttcggcattagtttcttcctaatgtccactctgcccgaagtgacaagcgggggcatgtggagactcagctccaggttcctggacgggctcagccacccccagaaagctaatgaatgctcaaccagggcttccagatgcccaggggacagagcaggagatgccggggaatggggctttccttgcagttcaggagggccctgccccaggcccagaagtagaagggaaagcggctgttttggcggtaaacagtaatgtggggagtgctgcagagaaaggcagtcttggggtttcaagctggagagcagtcagctacactcaggacctctggccatccctgccttcacctgctgtttggcctgatcgtctaacttctctgattctccactacccactccttattacgtttttgagacttgtcaaagttttatattagggctaactgggacgcatacaaatctggtaacttcgccagggcgggaagttaggaaggagcagagctggctgcaggtgtctggtcctgaccactcctctatgccacccttgaggagcttgctgactttctcatgacgttctcccattccaggagctgcaagtgcgttatcctggctggagcacggtgtcaatcacggcagactaaggccagcggtgatggcttgaatgccaggctgggggctgggatttttcctgaggatttcacaggacagaggttggcttggaaagaccaaggtgggactgaggaacattccccctacccccaacctcggtgggctgttgcaagcctggaggccagagaagacgggcctgggatgccgcgggcgcaggggcaggcagtgaaggagatggctgccttcggtagagctggtcgctgaggcagaagaggagggcgtggggcgtggggcgtgaggtggccggcgccccggctggccaatggccgggctgcggcccctccgcggggcggggtgggcctggtgggcgggcggggctcggggcgggggcggagagggagcctggtgggcgggcggggcgcgtcttgcgggctccctcgggtaccggcgctgccgcaccccgccgcgctcccgcacccgcggcccgcccaccgcgccgctcccgcatctgcacccgcagcccggcggcctcccggcgggagcgagcagatccagtccggcccgcagcgcaactcggtccagtcgggg

SEQ ID NO: 31 - DNA, 1200 bp, Homo sapiens
gcactacagcctgggtgacaaagcaagactctgtctcaaaaaaaaaaaaaaaaacaaaaaaaaacaattaatgccctcgggtaaactatgacgtttgtgctaacttttcagatgtcccaagatttgggttgttttacaaggataacgaagttcatgatgaggttaagcaatcactgaagctagtgctagaaattttttttaatttaattttttagaaagaggggtcttgcagaattttttatttaattttttagaaagaggagtctcgctatgttgctcaggttggtctccaactactgggctcaagtgatcctcacgagtagctagggctacaggcacaggacactatgccaggcttagtgctacaatattctgtctattttactttaaaatattctaagtatgctcactttggaaggcatcaacatccagaaatgcattcctacatcaagactctcaagaaacgggaattactgtaagggcatacccttgagcaaccatcacactgcactgaactcttgagaatattctggaatatcctggaagcatctctgcagggcagagaagtgaactgccacagaaggagcagctttggggagaagaaatacagtttgcctcttacagtatttttctacaccgagagctacattcctcccgagttttaaatttgtggaccgttgaatatcactccgcctttcataaagttacaaataccttccgtcggtggcgggaacagagtttggaaaattacatgcctaagacttcagataagctaaatgacatacatgcaatagcatcccatgaagaatttattcaacccaatccattgcatctaattacccaaagcacagaaactctccaaactgtgcaccccccaaaaggagggaaaatgcaaggttcctgctgcagttcccgggacacactgcgagccgctttgttacaagtgtatccaatgtccccagaacgagggacttgtggggcgcggctcatcccagcgccacttgctctgcagctcccagaggtggtggttgtgttacgaaggctgaccctgccaatggccgacaaaatggtgcgcacccccaagtgctcgagatgcaggaaccatggcttcctggtgcccgtcaagggacacgcgggcaaatgccgctggaagcagtgcctctgcgagaagtgctacctgatctccgagcgccagaagat

SEQ ID NO: 32 - DNA, 1200 bp, Homo sapiens
atatatatatgtgtttatatatataatatatattatatttgtatacacacacacatatatgccacattttctttatccatttatcccttgatggacacttaggttgattccatatcttggcaattgtgaatagtgctgcaataaacatgaaagtacagacgtctcttcaacacactgatttcaaatcttcccagtaaatacccagaaatgagattgctggatcatatgataattctactttcagttttttgagaaacctccatacaattttccatcatgtttgtactaatttacattccaccaacagcatacaagcattcccttttctccattcttgccaacacttgttatcataaagggaggtttgagagaccataaaacaaaccccacctcacaattttgttagtgcaagttctatttggcctattcccaacactaagcacacagtaggtggcaaatatttgttgaatggatgaattctcatttttctccatttgtggaagcagacacctcctcaaaaaaaaaaaaaaatccgaaagaaaaggtaaaggaaacagttctatgaatgggggttaatttactctcctagcattcacttattgggaaaaaaagtaacagcagtttctagaaccctctggtctctgagggagatgctgtgttcccctcctcagagaagaagaaaacgcactagagagtgggagcatccccaaggctgaagcgcatccaggacatcacatgtcaacgtgtcctctctggcgtggggtccccgcgagtctgggaaacgaggagctggcaagggagggcaagcgggggcggcaaagagggcccctggagcagggggcgcggacctcgctgcgctgcgcgctctaccgccgggctcgcaacgctgggctcagcgctcgcgcctccctcagctctctcctccgccccccttcgccctccccctttccctccctttctcctcctcctcctgccgccgcggccgctgccggacttcgccagatcagacccacggggctgccctcccctgcgcactcccctcgctgcccgggcccggagcgcagcgcggccgcacaggtaggagcccccaacggggggtcgccccgagcgagcccagtactcactgggccgcgcgcgggagagtgaggtggggtgagctgaggggtgttgagtacccgagaactgccctggactgtgcag

SEQ ID NO: 33 - DNA, 1200 bp, Homo sapiens
gagaagctctgagacatgtgagaaggtctagtattctactagaactggaagattgctctccgagttttgttttgttattttgtttaaaaaataaaaagcttgaggccaaggcaattcatattggctcacaggtatttttgctgtgctgtgcaaggaactctgctagctcaaggtgagtatactgggaaagccttaacgttctagagaacagagttttgcagccccacctccatcccccggggtgtgggactggggggcaggaaatgcaccgtcccctttgaaatggattactgttttttcctttcgaccccctctttctgcagcctgctttgtaggtgcagtataaaatgcacgctgaatgtcttttgtatgtaaacagcgtagcaggatggagtaacgtgaaatgcaattctacagcagtttttacgtctttgctgcctcgttcgttggctaccgagaaggttcaggagggggaggggagatgagaaagcagattggaagttgagtatggtggagcctcagcctctcccaccctcctttcctcgcttgtgctcactgctaaagttttgttactttccccgcagcagatactaaacattagtttgtcctgtattttctttgagattcacaatgttgaaagcccttttcctaactatgctgactctggcgctggtcaagtcacaggacaccgaagaaaccatcacgtacacggtaaggggtgatggaatttgaaataagtacggttcagcgggattctgtgacaaaaaaagactttacacacccctgccagtgtatttgggctatactctgctgagggtgataaattaaacaacacttcattcatgcttcatatctaagattcgttgtaaattgcccccttgatcctttcaaaagttcattgggctcaccacctaagataggaaccaacatgtaatcatttgtgcagggctaaaaatgggatccgttcaaaaactaaaaccaaagaaagttacatgtttccaaaacattcaacaaattaatgggtgtaaggaactggaaaacctggactcctaccacatgcagataaaaccaatacgtgcagaataagactcaagtcaagtaagaacgttaaacaccataaagacacatggccttctttgtgtacatgacatgcattctcaagtaagtggcctttattgaatttataaaggctatatattc

SEQ ID NO: 34 - DNA, 1200 bp, Homo sapiens
cggctaggtcggagcggagagtcgcagcagccgctccgggcagcttcccagtcccggtctgccttgtctgtgcggctgcaagtccccgcacccacacttaagcctttcacttctaccccaccgctcccagacgggctaggcccggttccagggactgcccccaaccctgcccccgggaaatccacaactggttccctgcccaaggccatctcggggcgtcctgggccctgggcgaggtccacggcgtgtgcagggggacggggccagcggggacttccttactgctagaaagtcattcgttcagtctctggagacagtgacggagccctgctgcggactggacgttggggaccaagtaggtccccgctctccaggcatctggggagccagggaagaagcgagtgatagagccacaaaactccccgcaaagagtatttcagagggggaagcgccataaagacaaaacagggcaaggtggcagagggtggcggtggcaggcgcgagtctgcagctcagcccgggcgcgctggacgcagacacgtcgactctcctaaagaagcaaccgcaggtcagcttagacttcttttatttcaataaaagagcgttttcctccatcttaaaccgcccgtggtgggagaaaaggtttccaacctcttctctggctcccctcggtcaattccgagcttttccaaagtccctccgccgtccaggggctcgaccttcgaggtaacgtgggggatcccgctggctccctccggcaggcgggggcggggaccggccgtgctcgccccctagagccccgggagaccgcgcgccccgtggagcccggagggggcggaaccctggggcgggccctgctcggtgaaggcggggctcggccgccctgtctgcgccgccattggccgcgggccccggactggcgggctcacgtcaccggccgggcccgcccccttggcaatgtcttcagcccgctgctctcctaacttaagcatttattccactgggatagaagcggcaggagcagcgttggcaccggcgaaccatggctgggattttctatttcgccctattttcgtgtctcttcgggatttgcgacgctgtcacaggttccagggtataccccgcgaatgaaggtaagagcgggacggcctttctgtggtcggggacagcgaggggccatcgcgggtgaggggtcctcag

SEQ ID NO: 35 - DNA, 1200 bp, Homo sapiens
aaccctgtggagataggcaaaataagtataaccccattttacagttgaggccacccacttaataaacaagtgacaaagccaagtttccagtattcctcaagtgttttcctttcaggctctaaattgctgacttaaatgtcatcattagagagagttgtctcctcggtagaagtacaaattttgttccagagtcaaatatgaagacttgatcagtggcctggcgcggtggctcacgcctgtaatctcagcactttgggaggccaaggcaggcggatcacctgaggtcgggagttcgtgaccagcctgaccaacatggagaaaccccgtctctactaaaaatacaaaattagctgggcatggtggcacatgcctataatcccagctactcaggaggctgaggcaggagaatcgcttgaacccaggaagctgtggtggcggtgagccaagatcgtgccattgcactccagcctgggtgacaggagtgtgaaactctgtctcaaaaaagaaaaaaagacttgatcagcttatagcctttatcatctagtcttctattctctggagagtcagtggggactcttccgagttcttgtagccttttcttaaggcctataggaagtcatctgtgctgagcctgcactttctataacagatggaaaactgaagctcagacatcttaagtggcattgacttagactatagagctagttagtggggggccagcaccatgacctggcccctacctccagcaggcttcacccacccttgcacccttgcaggaagcacttagaggcttgaggttgccagctgtggggggttgggcctgtgcagtgcctctcagccgcccacgctggtggggctctgcagtgttagcttcttgtttctctattttctacctaggtttcagtttttcacttgggtttgtttcccctttgtctctggttcagcttccttttcctgtttggttttaagtaggctataaaaatcaagttgctgtcttcagagggtctgtggtcctctgatcaacataggctggtgggagtacaggactcgcctcctcagggttccctgtgctgccacttttcagccatggccacaaggtgagtattggaaccagtgcaggggacagagggatgtcaagaggaaacctagaatcttaacggtgaatgggctgtagaaattatctcaggtccccttagtata

SEQ ID NO: 36 - DNA, 1200 bp, Homo sapiens
cttttaaatttgtttctcttcccctttttgttaaagtacaaaacaaaacaccttcccccacttttcgaatagttttggctgtgtctttaggactggaaggaaattcagctcaaatgaattactatattatgaacttaagtaagaattgttgtgcattttgtacagaatgcaactcctaggagttgacaaatgtttgttaagtgcatcagtgagcgacataggaccttctcctccctactctcttgtcacggtttttatttaatcaaacatttattattgttcgatgctcttaaatgccatttccttcagctgattatttgtatgacagaagagtcaattaagctattttgtcctaaaaattacgaaaacgaaatgtacaattgtgaagtaaaattttgttcctttgcaaattttaataaattattgaagtttattttttgtttcaaataatctgggcttcagttctaataatggaaggacaatgtgaaggcccagaattcagcatagcgcctggcattaataggaggtcagtacatttttagtacatgtttctcaaatagatcttaaaatttcatttaagagcgtttcctcacgtcacagtatgtctctggcgttacttaattttgaaaaacctcaacacagattctagttttaggcaaagctcagaaaatttctacttaaggatatttccaaagcgaaaggaagcgcggagacgttcatgactggcatcatctcgcacgtggttccggaaattctcggtaggatgccctacatctgctctccctccactaagaagaacctctttgtgtggcgaaagtaaaagtattagggcttttaagttgcccagagtcaaggaacacggataaagacgctgggagattgacatgcatttcgaccaatagcattgcagagaggcgtatcatttcgcggatgttccaatcagtacgcagagagtcgccgtctccaaggtgaaagcggaagtagggccttcgcgcacctcatggaatcccttctgcagcacctggatcgcttttccgagcttctggcggtctcaagcactacctacgtcagcacctgggaccccgccaccgtgcgccgggccttgcagtgggcgcgctacctgcgccacatccatcggcgctttggtcggcatggccccattcgcacggctctggagcggcggctgcacaaccagtggag

SEQ ID NO: 37 - DNA, 1200 bp, Homo sapiens
gcacaggctgccagctcaggctttggcccctccatccgctcctggcttctctgtgggtggccccatctaagccaatgggcccttcctggctccacctcctctctgctgtgcggccctgggcaggcagctgccctgccctgtgcctttgctcttggctgcctcacccctgtcctgggctgcagggagacccctgggggtgggggcaggcacactttgggcccatggctgctgctcagatgtgaggtggtgaccaccaggccatacgtctctggacagggatgggagagactcagagagacacagagaccaagagataaagtcagagggagatgcagagaccaagagggacaaaccaagggagacagagagatgacattagatcagagatggagaccccgaaagagacccagaaagggatcggtgtgggctgggggaagagacaccccgggggtcacacagtggtcacaggcatcagagctgggctgaggaaaaggacaaactgaacctgttccaaagctgggggctcagtaccgtctgcccaggcacatgcagcagcagtggggctgcaggcaggcccccagggtaggaagcaaggcgggtgggaggcagggcctggcctctgggcagttttgtagccgggcccctcaggccctcacaatgtgcccaaggctgctgggcccatcttccaggggtgatcactgaggctcaggctggctgaggtggccggaccccaacccctggccctgccctgtgggcagcagtgccaggtctggcactcagaggttaggccgggcgcagggagggtgcgggctccgcagcgagcgcgccgggcggcctctaggagccgggtcagcgagtctgggctctgagcgagcggcgccggcccggccccctctccggcgggcggccgagaccccgccccggaggcggaggcggagtcaggggagggggccgggggccggctggtcccgcctctcgcgcctccgccccgccccgcgcgcacacagccaggggccgcccgggctctcgacgcgccgacggccgggcggacggacggacggacgccgagcgcagtgccccgcggtgagtgcacgcggcccctcccgcccggactcatccccgtcggtccgcgcctcgcagctcaggactcgggggcgctcggacgcaccgacggctcggcggaccctcggacgcgcctcg

SEQ ID NO: 38 - DNA, 1200 bp, Homo sapiens; identical to SEQ ID NO: 37

SEQ ID NO: 39 - DNA, 1200 bp, Homo sapiens
cccgccccttcccccggagacgcgcccacccggccaccgctctcttcccactcccccattacccgcagccctcactccccgctgcgggaaggggctgcttggctgcctctgggggtcttcagagctaccctggtcccgggggattggaggaggaggttacctatcctgcgtcgtctttaatccgtgcaccactatccatcaaatagagacagatcctgggcctctcaaagacggatgattgggggtggtgattggcctatccctaaatatctaccacgcaaggactcttgagagatccagaccccggtacagtcgagggacctggggcccaaaaagggaaagcggctacctctaccacacagttgggaagcgcagtcctaaaggagacgcaggttggagactccgctaagcggagaagccgcagtggggccatggcaagtcaccttccctttcgggcctaggaatactcattcgaaagatggggggactggagtgccgagtggctgtggcagccacgattggggtttggaaaccatcctgaaaggcccggggagccagtctcctggaacttctccctccccattcccacaaaaaccaagcgccctctcggccaattctcaccctctcaggacaaaaaagtgagatgagcccgtcctttcacctgcgagtccaagcccttggcagaggcctgaaaagtccgaaaactccgagttcgggcgctgaggtctcccgagccggttcctgaactctccgggcctcagtcgatcggggtgcggagggggccgacccgggggatctccaagcgccctccccgccctgacgctgtggggctcctaccgcgccgccacagctgctcctacctggggaggtgcgcccgggccccggggggcgggcagtcggggggcgggcagggaaccggtgccgccccacgcttcgtggcccctttaaggaggggaagccggcggagggaggagccggtccggtgtgtgcaggggagcgcctcgccagcggtccgcagggctggagacccacgccgtggagaggaccagcctcaggtcgccccgcctgggcccgcgccccgacctcgctgcccccgcctcgcctctctgcccgtggcgcttacggccaccttggcctcgggggcagggcatgggcggcccccgccagatcgcccagcgccagtactaactgccctcgctc

SEQ ID NO: 40 - DNA, 1200 bp, Homo sapiens
ggttacctatcctgcgtcgtctttaatccgtgcaccactatccatcaaatagagacagatcctgggcctctcaaagacggatgattgggggtggtgattggcctatccctaaatatctaccacgcaaggactcttgagagatccagaccccggtacagtcgagggacctggggcccaaaaagggaaagcggctacctctaccacacagttgggaagcgcagtcctaaaggagacgcaggttggagactccgctaagcggagaagccgcagtggggccatggcaagtcaccttccctttcgggcctaggaatactcattcgaaagatggggggactggagtgccgagtggctgtggcagccacgattggggtttggaaaccatcctgaaaggcccggggagccagtctcctggaacttctccctccccattcccacaaaaaccaagcgccctctcggccaattctcaccctctcaggacaaaaaagtgagatgagcccgtcctttcacctgcgagtccaagcccttggcagaggcctgaaaagtccgaaaactccgagttcgggcgctgaggtctcccgagccggttcctgaactctccgggcctcagtcgatcggggtgcggagggggccgacccgggggatctccaagcgccctccccgccctgacgctgtggggctcctaccgcgccgccacagctgctcctacctggggaggtgcgcccgggccccggggggcgggcagtcggggggcgggcagggaaccggtgccgccccacgcttcgtggcccctttaaggaggggaagccggcggagggaggagccggtccggtgtgtgcaggggagcgcctcgccagcggtccgcagggctggagacccacgccgtggagaggaccagcctcaggtcgccccgcctgggcccgcgccccgacctcgctgcccccgcctcgcctctctgcccgtggcgcttacggccaccttggcctcgggggcagggcatgggcggcccccgccagatcgcccagcgccagtactaactgccctcgctctggccttcgagcccgaagcctcttctgcgcgcacaacctaggcagtaatcctaaactagcgggcaccacagaccagctgcagccaccccaacccagggatcacttccggacccctcgaccgcccggcaccagcgcgcaagggacccttcagccg

SEQ ID NO: 41 - DNA, 1200 bp, Homo sapiens
ctgacgactatttccagagatcctctcccctgccccattccagcccagtcactctgagagcgcgagggacaggccaggccgaggcttcttcgtacagtcacacccgtttctgggctgactttcccaaagcctcagagcagctctccccaccctaccccgtccagccccctcgccgtctcgcccctccccagccccgagctagaagttcagcctcaagacggcagcaaacggcagagcaaaggagtcgttttctttcacctgaaagccgcgaggaggcttggagcgcctttcctcgctggggcccgagcttcctgggctttggccgggtgcgggcagaccccaccggggtcctcgtcatctcccagcccagcccgcagagcgagtaccggcagatttcaagggcgcgtgagcctggctgtcggctgggcccctgaggctcgctgggcgggggcaggccggtccaggctgtgcggggcgtttacaaaaagtgacttggagatgaactcgcccgtgcgcggctggccgccccgctataggggcgaaggcgcctgacgcaagcggaactcggtggagcccatacgaatcagaacagagcgaggctcctggcgcactagggactccaggaggcagctccgccagagacgcgggtcgtgcttcgggaaaccggggggcggggggaggggaagagcgcagaaaagaaaacccaccaaggcggggactggcctgagcggggaggggcggcgaggccggagcccctctctgttgggcggactccccatggccagaggctgagctccactcccgccggccgctccctaggggaaggggaaggagaggggagagcagcgacaggcctccagcaagcaagcgcgggcggcatccgcagtctccagaagtttgagacttggccgtaagcggactcgtgcgccccaactctttgccgcgccagcgcctggagcggagagcagaggcggcccggccgcggcgcgccggctttgtcatgatggccagctaccccgagcccgaggacgcggcgggggccctgctggccccagagaccggtcgcacagtcaaggagccagaagggccgccgccgagcccaggcaagggcggtgggggtggcggcgggacagccccggagaagccggacccggcgcagaagcccccgtactcgtacgtggcgctcatcgccatggcgat

SEQ ID NO: 42 - DNA, 1200 bp, Homo sapiens
gaaaggagctcataggaaggggcacagcacaaggctcccgaaagaggtcactgcaagtgtcctgcgcaatttctgttcagcacctctgcagtgacgacaggggtttgctttcgtaagcagcatcgttttcactactacagaagcacatacgccgcgtgcagcttaggcccgagcaaagtccctgctcccctagacccagtcccttcaggtgtccccctaggggaactgtggcgtgtccccccaaccaggcgcactcgcctaaactgcgtccccgaactccagggcgcagggagccctcgcgggctgcccggggctggggcccacacctgaacggaggagccggctggcggagcggccccgcgcgcgcacacacgcacactcacagacacgcgcaaacccacagcggcgcgcgcgctccccattggcgggaggaggaaggcggggccggccgacccaaaacaaacaacctgcgccagcgactttgccgccgcgctgcagtgtggcctccgcctggtgcgcgcccgttggggagctggggaactggggagccgccaccacctcggccacctcgttcccccaccccttgccgtccgcgctcaggccccgcccccgcggagccgccgcagggttatattgggccggcccgaaggtggaggcgagacgcagccgcggcaggccagcggggagatgctgccccggaggctgggagcgggaaggcggctgcgggcgcagcggcctcggggactcgcctccgcgggtgctcaaggctgaaggcgccggaccttcccccacggtggcacgcacatcatccggcaccatttccgtgccccgaagacgcccgcgcgggacctgggaccgccagtgaagaacccctcctgggctctttaacgagcagcagatgcgcatgctgcttctctccggcaggaaagccttctaagcccggcgtgctcgggcacccgagggtggaaccgcggcccctgcacacactcaccccaaagtctcaacgtcgaaccgaaggcgacacccacccaactcctccctctccgccccatagtccacccaacacttgcagcccctccagagaaaaagcccagcggaagaggactccggaaaagtgactatctcggaagaccagggtagagctcccgggagaaaaaggcgaagatccccggccccggatcgccatccccttccctggca

SEQ ID NO: 43 - DNA, 1200 bp, Homo sapiens
gctcccgaaagaggtcactgcaagtgtcctgcgcaatttctgttcagcacctctgcagtgacgacaggggtttgctttcgtaagcagcatcgttttcactactacagaagcacatacgccgcgtgcagcttaggcccgagcaaagtccctgctcccctagacccagtcccttcaggtgtccccctaggggaactgtggcgtgtccccccaaccaggcgcactcgcctaaactgcgtccccgaactccagggcgcagggagccctcgcgggctgcccggggctggggcccacacctgaacggaggagccggctggcggagcggccccgcgcgcgcacacacgcacactcacagacacgcgcaaacccacagcggcgcgcgcgctccccattggcgggaggaggaaggcggggccggccgacccaaaacaaacaacctgcgccagcgactttgccgccgcgctgcagtgtggcctccgcctggtgcgcgcccgttggggagctggggaactggggagccgccaccacctcggccacctcgttcccccaccccttgccgtccgcgctcaggccccgcccccgcggagccgccgcagggttatattgggccggcccgaaggtggaggcgagacgcagccgcggcaggccagcggggagatgctgccccggaggctgggagcgggaaggcggctgcgggcgcagcggcctcggggactcgcctccgcgggtgctcaaggctgaaggcgccggaccttcccccacggtggcacgcacatcatccggcaccatttccgtgccccgaagacgcccgcgcgggacctgggaccgccagtgaagaacccctcctgggctctttaacgagcagcagatgcgcatgctgcttctctccggcaggaaagccttctaagcccggcgtgctcgggcacccgagggtggaaccgcggcccctgcacacactcaccccaaagtctcaacgtcgaaccgaaggcgacacccacccaactcctccctctccgccccatagtccacccaacacttgcagcccctccagagaaaaagcccagcggaagaggactccggaaaagtgactatctcggaagaccagggtagagctcccgggagaaaaaggcgaagatccccggccccggatcgccatccccttccctggcaccagcttctctaggctgcgcgaaggaagagggt

SEQ ID NO: 44 - DNA, 1200 bp, Homo sapiens
gggactaatagatttattccaggtgatagtgggaagcagctacagaaggaggattggaaggctgaggggttgttaaagttatctgatgatattgttcgaacatttctccggcacagaaactccacaaagaactcctctgcagttttcgctacccagggctgtttctttaaattgtttgacgtcagagaaggaatgctatagcaacagtctgtcaaactggaattaactcagaagaaaaatgggggtggggagaggaaaaatgctcccaggcggtactgacttcagaggaagcaaattggatagatgcatttattttatgacattttaaacataaaacacccagataaatgtgaaaaaaaaaaaaactcgattgagatgcatgtggtgtttaactcatgcagagaagagcgacttggcagggatatctggaaggccttccagttttatcaatgacaataggaacaataacttccaagtattggccatttatttggaggcaggcttcgtgcaaacatcacttcatttaatatgcttagcacctttatgaggcaggtatcagtatctttattttggcgatgacgaaacttaggcccgagacggtacacggtcctacaactagtgatggaagagcggggactgcaacgcaaggttgtgcaactctgggcttcagtttactttctgacatttagaattccagaatgggaaacctaaatggggcctttggggtcttctagctctgcccttcgatcctgctaacaagcaaactggcgcctgtcagcgccggaaaggaaactggccacgccacttagacgggcgctacttagcagcgtgccccggcgccacaccaacaagaaaacgaaactgcccgggaatgaatgtttttcttctccttctggcaggaaacgagagagaaaataggaagcagaagctgcgcgcggcgcccgctcccttcgccccccgcccgggctgcgcgcgctccgagggtctgcgcgtccccggcacgtccccgccgggcccgcgcgcctcgtgcctcgcgcctcgcgcctccagccacgtccccgccccggctccggcgcgccgcggagttggctgctgggaggtgcgggactgggtgtggccggcggctctggtctcggctgtgagctgcgctctccacgccggctccgcgctccaggggctgctgagcgcccagcggacacc

SEQ ID NO: 45 - DNA, 1200 bp, Homo sapiens
gtaaccaggaatgatttgactgtataaatttatcttacagacacacttgtttgtacatttaatgcctgctttctataatatgaaaaaatacacagaacgttttatgcaattggctctgagtaatgcaacacataaatgctttgagagcagttaccaaaaattatgcaggttattacaaaagttatatggaattgcacagatattctaaatgtgcttttgagtaaatttctcagctaccagaggcctgggcagctgtttccagcacatcctggcacaacattcatgttatcatccacttatgtattccacaatttcaaatgattgataagacacctcccccatctttgcttcctcttggaggaaaaaaatatacaagcaatcacgagcacaggagcaggtggacaactacatcttaggaaagggtgcgtaactgtgaactctaaacgtgagctgggtgcctgatgcagtataggaaccacagtaagtcgataatcagtgaggctggaggattgaaattttatttggtaaagaatattgagccaggcgcggtggctcatgcctgtaatcccagcactttgggaagccgaggcgggcagatcacaaggtcaggagatcgagaccagcctggccaacatagtgaaacctgcctctactaaaaatacaaaaaattagccgggcatggtggcgggcacctgtaattccagctactcaggaggctgaggcaggagaatggcttgaacctgggaggtggaagttgcagtgagctgagatcgtgccattgcactccagcctgggagacagtgtgagactccatctcaaaaaaaaaaaatgaatattgaaatagagtctataggaaaaaaactccttgaatctagtaaatgtggaatcacaaaattgaaataatgtgcatctgaaaagtgaataaatagatgccaagaaggagtgagtcataagcacatgagttgtaagaggttgaacttgagagaccagatcaggcagtgccgcttaataccatcacatgatcctccccgaggccctgtatttaattaaaatagagagggaggcaccacagatgccagaagaacactgttgctcttggtggacgggcccagaggaattcagagttaaaccttgagtgcctgcgtccgtgagaattcagcatggaatgtctctactatttcctgggatttctgctcctgg

SEQ ID NO: 46 - DNA, 1200 bp, Homo sapiens
tcctacctccctggccactccttttcagactcctttgctggtttgctctcatttttccctaacctttacatgttgatatgccagagcacctcaaacccttagacctcatctctgctactcgtgctcccatggtgatctcatctagtctctggtttcaaacacagagtgatcactcctgtacttgctgtttcccttttcctggagttctctctgcctcctgcaggtaggaagtgattcgagataagagagggagagaactcttctggccaggatagagggtgattctgagaaggcttcaaggaagaagtgagcatttgagcacggctacttgaggaaggctaggccatggacattagggtgcaggcaggagcagagcaaacatgcttgtaaaaggaacagcaattattctgttttatatttatatcagcccatggtggcccccacagcagagtggcttgatctctcacctccttcaggtctttgtttaaagattaacttctccatgaggccttctatgaacataacctttaaaaacataaatccccttcctactgggactcccccagttcccaccctgagaccccctatctctcttctcgattttatttctctccataggatgtgtatcaccacctgacttactagctatttactatatattagtttatttcttccccattagaatgtaaacttcctgagggcagggattttgtctgttgagttcagttctgtatccccagcacctaaaacagtgcctgtccataacaggcatgttaaacattgttggacagatgaatgagtgaatgaaaaatggtagtacctttcctcctccagtccatcctacacaaagaggcaagattaatttccctaaatgcgtgagcattaggaagtgggagggattggaggaggaaattataataaggaatccataaattacttaagcccataaaacaaaggtaggaaacattcaagataagagacctcttctggccaggatagaggttggttctgagaaggcttcaaggaataggcagacatttcagcaaggctgctgaggaaggataggctgtggaaattaggatgcaggtaggaggatcacagagcaaacactccaaggtgggaaatgcaagtgtgtgtagaaggaacagcaagtattgttgggttgggctggcttatgaagttgtgacgggctgagggaga

SEQ ID NO: 47 - DNA, 1200 bp, Homo sapiens
tgtgtatcaccacctgacttactagctatttactatatattagtttatttcttccccattagaatgtaaacttcctgagggcagggattttgtctgttgagttcagttctgtatccccagcacctaaaacagtgcctgtccataacaggcatgttaaacattgttggacagatgaatgagtgaatgaaaaatggtagtacctttcctcctccagtccatcctacacaaagaggcaagattaatttccctaaatgcgtgagcattaggaagtgggagggattggaggaggaaattataataaggaatccataaattacttaagcccataaaacaaaggtaggaaacattcaagataagagacctcttctggccaggatagaggttggttctgagaaggcttcaaggaataggcagacatttcagcaaggctgctgaggaaggataggctgtggaaattaggatgcaggtaggaggatcacagagcaaacactccaaggtgggaaatgcaagtgtgtgtagaaggaacagcaagtattgttgggttgggctggcttatgaagttgtgacgggctgagggagagacaggactggaaaagcacgttggggtcaggttgcagaggtccttgactgacagagaggggagcttggtctgtggggaatcttctgctctgtgtggtcagaggctggtctgactctccggccttgccccagtcttcctatccttcatccagccccggctggctcggtaaaaacggcgcgaatctcgctctccctgggtaactagggtccccactgcctggctagtggtgcttttcgcgtcttcactggcaccattaatgaatgaagtctggctcccacccgcggggaagggaggagcctcaacctcgggtcctttgctctcctgcagcactcctgcccgggtcccgcctcggggtcgccaggccctgaaccccaacgccggcattagtcgcgcctgcgcacggccctgtggagccgcggaggcaagggacggagaacggggcggaggcggagtcagggcgcccgcgcgtgggccccgcccccttatgtcgggtataaagcccctcccgctcacagtttccctagtcctcgaaggctcggaagcccgtcaccatgtcgtgcgagtcgtctatggttctcgggtactgggatattcgtggggtgagtgccgtctcaacggta

SEQ ID NO: 48 - DNA, 1200 bp, Homo sapiens
actgacactttcattcatatgtttagaagagacatatctgaagaaaattctttttaaaatagtttctcagctgagattatacttttttagtgtgtagctcacaggaagttctagttgccaagagcatgttcgatatgagtagatctctacctaattcttatgcataatgaacccaattttcatctcttctagaaagatactgaaaacatgtgtcgtgatctcaatgttacaaccatgtagcctcatctttcacaatacggtacaatcaccaacgaagtttaaacttcatgtctggcatccaaggtgaacatttccatagaaagtaggaaatatttagatgtctgtttgccgtggtggtggtgttcctatatactttttgtgaccctaaagatataataatgtttgacatttttgttagccagtaatgtggacttaagacatcctaaccacactgctgactctaaccctttaatagactttcacgtagttcttgagcctcactgcaggctccagtgctagtcccggaggaaatcagaaaagggttgaaaccactgacacagccatctctttagctcctcctaacgcaggtgtacagtttcctgtagtgagcagatcggggcctacagggacactgcatgcttgcactgggctgttactagtttcatggaaatatgtactaattgtgtcactgtgttttcttgtcacactgccccctttctaggccgctctggcaccgtttatttaaactgtgcaaccatctggcccaggtgccgtagagattgatcagtagcacaaggccaccacaagcacaaccgccacccggccatcccagctatttcctccagcattctagacatcatggcgtccgagggtgggtccaacgggcttcgagtgttcgggagataatgcacacttcgcagtggatcttctcggggccccgagcgttgcccgcccattaggtggcgccggtcccctcgaacaggtcgctcaacaactcccgcccagcagccgcccttactgcgcgcgcgcagacttcggcgtctacttccggtgtggcccaggcggggtccgcagaaccagctatgtcggcctacggcatgcccatgtacaagagcggggacctggtgtttgccaagttaaagggctatgcccactggccggcgaggatagagcacatgacccagcccaaccgctaccagg

SEQ ID NO: 49 - DNA, 1200 bp, Homo sapiens
tttttagtgtgtagctcacaggaagttctagttgccaagagcatgttcgatatgagtagatctctacctaattcttatgcataatgaacccaattttcatctcttctagaaagatactgaaaacatgtgtcgtgatctcaatgttacaaccatgtagcctcatctttcacaatacggtacaatcaccaacgaagtttaaacttcatgtctggcatccaaggtgaacatttccatagaaagtaggaaatatttagatgtctgtttgccgtggtggtggtgttcctatatactttttgtgaccctaaagatataataatgtttgacatttttgttagccagtaatgtggacttaagacatcctaaccacactgctgactctaaccctttaatagactttcacgtagttcttgagcctcactgcaggctccagtgctagtcccggaggaaatcagaaaagggttgaaaccactgacacagccatctctttagctcctcctaacgcaggtgtacagtttcctgtagtgagcagatcggggcctacagggacactgcatgcttgcactgggctgttactagtttcatggaaatatgtactaattgtgtcactgtgttttcttgtcacactgccccctttctaggccgctctggcaccgtttatttaaactgtgcaaccatctggcccaggtgccgtagagattgatcagtagcacaaggccaccacaagcacaaccgccacccggccatcccagctatttcctccagcattctagacatcatggcgtccgagggtgggtccaacgggcttcgagtgttcgggagataatgcacacttcgcagtggatcttctcggggccccgagcgttgcccgcccattaggtggcgccggtcccctcgaacaggtcgctcaacaactcccgcccagcagccgcccttactgcgcgcgcgcagacttcggcgtctacttccggtgtggcccaggcggggtccgcagaaccagctatgtcggcctacggcatgcccatgtacaagagcggggacctggtgtttgccaagttaaagggctatgcccactggccggcgaggatagagcacatgacccagcccaaccgctaccaggtgtttttcttcgggacccacgagacggccttcctgagtcccaaacgcctgttcccgtacaaggagtgcaaggagaagttcggc

SEQ ID NO: 50 - DNA, 1200 bp, Homo sapiens
aaaggcaaattatgtgcacatgaccctgtcactacaaggtagttctcgaccaagctacatcttatagctttatcttatattcctctcccatcatctgcaggaaaagggataggtgctgagggaaaacccctaagtagaactcacagtaagcagtgggaaaacagcttatttcactaacggggttaagtggccctgaaaagagcctttggagacaaagcagccggcgacccgcggagggagggagggagggagcgagcgagcgccaggtcccggcagggactcacttggagctggcgtacttggtgaccgccttggtgccctcggacacggcgtgcttggccagctcgccgggcagcagcaggcgcacggccgtctggatctccgggacgtgatggtggagcgcttgttgtagtgcgccaggcgggacgcctctcccgcgatgcgctcgaagatgtcgttgaggaaggagttcatgatgcccatggccttgcaccagatgccggtgtcggggtggacccgcttcagcaccttgtacacgtagatggagtagctctccttgcggctgcgcttgcgcttcttgccgtctttcttctgggctttggtgacggctttcttggagcccttcttgggagccggcgcgaactttgcaggctcaggcatggccagacccaagaccgacaccgacccccgagaacgcaagcagagcggtaggctcggggtctaccggaaacgactgtgtacttacagaggctgtgcgcatgacgctgcgttatggttcgcgagttttccgcggcgcgcaatgcgagggagacgagattatgtaaatgagtggattctggctgagctatcctattggctatcgggacaaaatttgcttgagccaatcaaagtgctccgtggacaatcgccgttctgtctataaaaaggtgaagcagcggcgttttcggcgactttcccgatcgccaggcaggagtttctctcggtgactactatcgctgtcatgtctggtcgtggcaagcaaggaggcaaggcccgcgccaaggccaagtcgcgctcgtcccgcgctggccttcagttcccggtagggcgagtgcatcgcttgctgcgcaaaggcaactacgcggagcgagtgggggccggcgcgcccgtctacatggctgcggtcctcgagtatctgaccgccgagatcctggagctggc

SEQ ID NO: 51 - DNA, 1200 bp, Homo sapiens
ctatataaaccaaaaattgcttcaaccacagaatggaggtctacaggtgcggaaggaaagtttatatggtgaggcttggtcgcaaaactattaggaatattttcaggttactacacaattttgcgagctcaatatgcagttaacactttttccctcgactctcctgagcagatttacattgaccgcacccgtagcatatcgtctcactttggaaaaggcaaattatgtgcacatgaccctgtcactacaaggtagttctcgaccaagctacatcttatagctttatcttatattcctctcccatcatctgcaggaaaagggataggtgctgagggaaaacccctaagtagaactcacagtaagcagtgggaaaacagcttatttcactaacggggttaagtggccctgaaaagagcctttggagacaaagcagccggcgacccgcggagggagggagggagggagcgagcgagcgccaggtcccggcagggactcacttggagctggcgtacttggtgaccgccttggtgccctcggacacggcgtgcttggccagctcgccgggcagcagcaggcgcacggccgtctggatctccgggacgtgatggtggagcgcttgttgtagtgcgccaggcgggacgcctctcccgcgatgcgctcgaagatgtcgttgaggaaggagttcatgatgcccatggccttgcaccagatgccggtgtcggggtggacccgcttcagcaccttgtacacgtagatggagtagctctccttgcggctgcgcttgcgcttcttgccgtctttcttctgggctttggtgacggctttcttggagcccttcttgggagccggcgcgaactttgcaggctcaggcatggccagacccaagaccgacaccgacccccgagaacgcaagcagagcggtaggctcggggtctaccggaaacgactgtgtacttacagaggctgtgcgcatgacgctgcgttatggttcgcgagttttccgcggcgcgcaatgcgagggagacgagattatgtaaatgagtggattctggctgagctatcctattggctatcgggacaaaatttgcttgagccaatcaaagtgctccgtggacaatcgccgttctgtctataaaaaggtgaagcagcggcgttttcggcgactttcccgatcgccaggcaggagtttctctcggtgac

SEQ ID NO: 52 - DNA, 1200 bp, Homo sapiens
gatggagcagaagctctaattgcttcgaggtacaagtccatctaaacctctgagatctcacccgtccccggtaagcccttgcgccctccagacaagctcctcagtgaaagcaagcgccgcaccggtgcctgtgccagtctcccagaaaggagcataaggagtgttccaccaccactctggattctttattccaacaacccagccggaggggaaaatctgctaaaccaccccacaaggcagaaactttatcaaaatctggcactacaacactggccctcatcacgccgccacaaattgcgagggatagaagaaagctgcaaagtcacgttaaaaggaagcctagtcaaaaagtgaaaattttgtcccgccctaggcacacagcactcacagcataagatgtagttccatgagtcctaggaaagggggaagaaaggaaaaaaccaaacatctgctctaggtttagaagagggataacatcagcgccaagcaaaccgaaagcgcagaggactcagaagaggggaagaggaataactttatctgtcctggaggtgggcgaagagcagtctccaccggccgcgcgtcacgctcccatttaaccttttccatgccgcgacgcccacactccgccctcgagggactagccccccccacgatccagagcagaagcggctcgccccgctgcatccacgggccctcacccaagcgtccagtcctgcctttctccccccacctcgcccaatacctgtccgagagcaggcgttctgccctctcgccccgcccttgcccaggcagcccccggtcgcgacggctctcctcggggctcgggggcgctgggcggcggccctgggctgctttggctaacttggctgcggggcaggcgcgaggagcggcgcgccgaccgggccgagcggagaggccgcccattggccggccagcgccacgtggccgcccccgccggtatattaggccactatttacctccggctcactcgccatgggttggagagggcagctcgggtagagagggctggcggagcggcgcagacggcggcagtcctgctcagcctctgcccggctccgtactccggccccggcctgcgccctcagaaaggtggggcccgaaccatgagctcctacctggagtacgtgtcatgcagcagcagcggcggggtcggcggcgacgtgctcagcttggcaccc

SEQ ID NO: 53 - DNA, 1200 bp, Homo sapiens
gcctgtgccagtctcccagaaaggagcataaggagtgttccaccaccactctggattctttattccaacaacccagccggaggggaaaatctgctaaaccaccccacaaggcagaaactttatcaaaatctggcactacaacactggccctcatcacgccgccacaaattgcgagggatagaagaaagctgcaaagtcacgttaaaaggaagcctagtcaaaaagtgaaaattttgtcccgccctaggcacacagcactcacagcataagatgtagttccatgagtcctaggaaagggggaagaaaggaaaaaaccaaacatctgctctaggtttagaagagggataacatcagcgccaagcaaaccgaaagcgcagaggactcagaagaggggaagaggaataactttatctgtcctggaggtgggcgaagagcagtctccaccggccgcgcgtcacgctcccatttaaccttttccatgccgcgacgcccacactccgccctcgagggactagccccccccacgatccagagcagaagcggctcgccccgctgcatccacgggccctcacccaagcgtccagtcctgcctttctccccccacctcgcccaatacctgtccgagagcaggcgttctgccctctcgccccgcccttgcccaggcagcccccggtcgcgacggctctcctcggggctcgggggcgctgggcggcggccctgggctgctttggctaacttggctgcggggcaggcgcgaggagcggcgcgccgaccgggccgagcggagaggccgcccattggccggccagcgccacgtggccgcccccgccggtatattaggccactatttacctccggctcactcgccatgggttggagagggcagctcgggtagagagggctggcggagcggcgcagacggcggcagtcctgctcagcctctgcccggctccgtactccggccccggcctgcgccctcagaaaggtggggcccgaaccatgagctcctacctggagtacgtgtcatgcagcagcagcggcggggtcggcggcgacgtgctcagcttggcacccaagttctgccgctccgacgcccggcccgtggctctgcagcccgccttccctctgggcaacggcgacggcgccttcgtcagctgtctgcccctggccgccgcccgaccctcgccttcgcccccggcc

SEQ ID NO: 54 - DNA, 1200 bp, Homo sapiens
aacagtgtgaaaagtcacatgataaactggggaagatatttacagtacatgtaatggacagagggttaatgttcttgctagatatcttaaaatcaagaaaaataaactcagtaaaaagatgcaaagggcataagatggaattcacacacagtgcaagttgaattttttttttttttttttgcctgccagagtgctacaggtaaaacagattgctactacccagtgttgctggtgggctagagtgttcatgtagctttctgcaggccagtctgagaatatttattaggattttaaaagtgcattcattgaccagcacttcacttctgaatttaatgcaaggaaatagttgggtaaatgggcaaaactattttgtacaaaaatattcattgtgttcataatagcaaaaaattggaaatcatccaaatgtcatttgtgttataactccatacactggaaaagtgagcagccatgaagatggtgtagatttgtttcaccagcatggaaaggtgcctgttacatatttaagtgtgaagatgaggttgtaaaatgtatgcctggtatgctatgatttcataagacacgtggaactatatcttcttatatgccgattgatgaagatggaatcagtgtatataccaaagagattaaaagtggttctttatctgagtgatgattttgtcttcattgtgtttagaatactaagaagtttcagcagtggaatgcctactcttcttataaccggagtggaaacggaagcattttgtgagagggctggagggaaggtgagaacgggcgttgggaggcccttccgtggctccaggcctagggctgcggaacccacaacagaacccgcaaccgcaaccgggaacccgagtgcctgcctagctggggggccggagggagggcggggagggcagcgccacggaccccagcggaccggaagggcttgtggcacgcggaatccccctcagaggcccccagctgccttccctgtggcatcatgaagtttcgcgccaagatcaccggcaaaggctgtctagagctgttcattcacgtcagcggcaccgtcgcgaggctagcgaaggtctgcgtgctccgcgtgcgccctgacagcctgtgcttcggccccgcgggttccggcggcctccacgaggccaggctgtggtgcgaggtgcggcagggggccttccagcagtttcg

SEQ ID NO: 55 - DNA, 1200 bp, Homo sapiens
ggccaagcaaacacccgctcatatttagtgcatgagcctgggttcgagttgccggagcctcgcgcgtagggcaggggttcgagcgccccttctccctgcctcgcctctgcgcctgggggctgctgcctcagtttcccagcgacaggcagggatttcgagcgtccccctcccctccctcgtcaagatccaagctagctgcctcagtttccccgcggagcctgggacgccagcggaggggctcggcgcgtagggatcacgcagcttccttcctttttctgggagctgtaaagacgcctccgcggccaaggccgaaaggggaagcgaggaggccgccggggtgagtgccctcgggtgtagagagaggacgccgatttccccggacgtggtgagaccgcgcttcgtcactcccacggttagcggtcgccgggaggtgcctggctctgctctggccgcttctcgagaaatgcccgtgtcagctaggtgtggacgtgacctagggggaggggcatccctcagtggagggagcccggggaggattcctgggcccccacccaggcagggggctcatccactcgattaaagaggcctgcgtaagctggagagggaggacttgagttcggaccccctcgcagcctggagtctcagtttaccgctttgtgaaatggacacaataacagtctccactctccggggaagttggcagtatttaaaagtacttaataaaccgcttagcgcggtgtagaccgtgattcaagcttagcctggccgggaaacgggaggcgtggaggccgggagcagcccccggggtcatcgccctgccaccgccgcccgattgctttagcttggaaattccggagctgaagcggccagcgagggaggatgaccctctcggcccgggcaccctgtcagtccggaaataactgcagcatttgttccggaggggaaggcgcgaggtttccgggaaagcagcaccgccccttggcccccaggtggctagcgctataaaggatcacgcgccccagtcgacgctgagctcctctgctactcagagttgcaacctcagcctcgctatggctcccagcagcccccggcccgcgctgcccgcactcctggtcctgctcggggctctgttcccaggtgagtcggggtggggattgccgtcgggccagttctccgaagccccgggaggaccggctcccg

SEQ ID NO: 56 - DNA, 1200 bp, Homo sapiens
gggagggtcccacggacatattgagggacgtggatacgcagaagaggtatccatgtggtggcagccgggaaggggtgatcagatggtccacagggaatatcacaaactcgaattctgacgatgttctggtagtcacccagccagatgagcgcatggagttgggggtggggggtgtcaaagcttggggcccggaagcggagtcaaaagcatcaccctcggtcccttgttctcgcgtggatgtcagggcccccacccaccgagcagaaggcggactcaggggcgctccagggtggctcgagctcacacacgctgagtagacacgtgcccgctgcaccctgggtaaatacagacccggagccgagcggattctaatttagacgcccgcgaacgctgcgcgcacgcacacgtgtcctcggctcgctggcactttcgtcccgccccctccgtcgcgtgcgggagctgacccggaggggtgcttagaggtatggctccgcggggtcaaaaggagaaggatcagtgagagaggcatccccacaccctcccctagaactgtcctttccccatccagtgcctcccaaatctctcttagtccccaaatgtatccccgccctaaggggcgctggtgggaggagctaaatgtgggggcggggctcggagtccagcttattatcatggcatctcagccagggctggggtaggggtttgggaagggcaacccagcatcccccgatcccagagtcgcggccggggatgacgcgagagagcgtggtcgcccccagaaggccctgggccatcatgccggcctccacgtagaccccaggggtcgctcactcctgccagctcgccttcaccaaggccaggagcttagcgcacgctcgcctcccgcccccccgccgcctctgccgccgccccctccttggaaaccaagttaccaacgttaaaccaatccccaagcgcaactctgcctcccccacaccccacccgccgcgccgcgcggagccgtcctctagcccagctcctcggctcgcgctctcctcgcctcctgtgctttccccgccgcggcgatgccagggccttcgccagggctgcgccgggcgctactcggcctctgggctgctctgggcctggggctcttcggcctctcaggtaagagccccgctctggttcggggtggacagggcgggggcggagtccctggac

SEQ ID NO: 57 - DNA, 1200 bp, Homo sapiens
ggaaaggggatcggggaggaaagtcggggaggcgaggtgggggcggtgggctgagcgggaacttaaaagaccgcggtggagaagatggggcgttctccgcgacactcggctgcccagaggtgcaaagccggtgggcgcacgcccggggcggccctacagccctcgggccgccagtctcagggtgtcgggctccaactaaggccccgaagcccccacgcacctgagggcattttgaccagctcttgcgggctgtcttgcggccgcagtgggttctgaaagtccattccggcgtagggtggaaaccggcgggaggcgttgctgattctcgattaaacaatagtgacgagacgcgtgcggggcgcctgcgagccccttcctgccactcggagcagagcgcgccagcgcgaggccgggccggccgcctgcgcagcgccgggctaattttaactgttgattacatcttcagcgtgtcctttcataggtctcatttgtaattgagacgaatgattacagtggggcgggaaggagcagctgctcattaattaatttctaattaaaagtgcctctcgttctcgggtgactaaagagaaacaccgttcgctgggcgatattgactcggatggaatttatttgctaacgcccggaggaatggcccctccgtccaccgcgcggccgggactgtcctggcccctcccggcctgagccgccggcgagtgaccggcgagtctgcggccaggccgggctgcgcgaggtggggctgacgtcagagcctcctccgcttcaagccgcgcacatctgacttgacgggttcgcgcgactcgtttttttttttttctttttcgtaaaatatgtatttctgtcgccgctgcgaggccgctcggtcccagagccccctcgagcctccggaatgcgaagttaaaggaatcgcggcggccaccgctgctcactttgttacccggttagaaaagtttgcggagcgcggggatggactaaccggctctcctgcttcgccctcccagcgcctagaagcctgcagctccggagcagtggccgcgccacgccggccccagcgcgcagaaccctgcaggccccgcccgtccgccccgggccgcgcccgccatgtcctacccgcagtttggatacccctactcctcggctccccaggtaagcggagccccgccccgcccaggccaccgcagg

SEQ ID NO: 58 - DNA, 1200 bp, Homo sapiens
gaatcatagtaggtaattgagttattttttaatttttattctgtctttataagatttttgagaaggtaatcaattaacataaagatctgagcagttataaagatttacaaagaagattatacagtgatggaaactgaagcatattcttggcaacaatctcaatctatttcacgtttcccggcttttacatatataaatttggtatacataatatgtaaatatacttctattttgcatgaaaactaaaaatactaaaaaagtagcatgagtatatgaaaaagttaaacattaaaaatattagttggaaactggcttattttactaacaattatattcagaattctgttgctagactgctgtttctgggaacattggaggtgtactttctatacttgctaaaatacaagcttttcaagtattttccatagtattaagtgagactttaaatactcttgcttttttagatctaatttctgctttaatttatccttctcttcatttttcctgtttgtcctcatcttacctgttcttccctggttttactatctcccagtattcagaaatcagagcaaataattagtctgcttttctgcagaataaactattgggtatgccatcatgcttttgatctaccacataaacaaattattttagcaagacatcacttttatatgctgtataatgtatgtattgcttatgttttaagcagatttcagctcttttccttccatcaatttggaattgggggcccaaaagatgtgaaggcgataatgcttccaagataactgggctgcatccttatttgtaaaataagaaatactttatttcccaaattttaaaggagtgttccttctccgacctgttgtgatggatttattctatagctatggaaatataccctgcttcactcttgtacatacttctttactctcctcccgccaagttcattgaaagagaacccatgtgccggtccagagcatcgctgcatccgtaagcagctagacctcagcttcctctgtcaccatggtgccggctcggctgggcccggcggtcgccatggtaactggggcgggtcgcagggtcctggcaggctgggcgcatgcgcgcggggactacaagccgcgccgcgctgccgctggcccctcagcaaccctcgacatggcgctgaggcggccaccgcgactccggctctgcgctcgg

SEQ ID NO: 59 - DNA, 1200 bp, Homo sapiens
taggggagagggactcgccaaatccctgttttctggggtaagcccccctccccccactcttggacccaagctgcccggaaggaagaaggaagcattgtttgtcaccggagaaaaccacctgtggtttgcaaagggcctcagtcgagtcaggggcaggcggggcagatccgagcccggcgggaggggcgaggcgcgggggcgggcaggggcgagggtaggggcgctcagctggcaggtcctgctctgcaggcctcaccgcccagctcccgggagggggccgagggcgccctgagtgctgcgcgccttcccatgacgcagttcctgtgcctttaaatcccactcccggagggggcgggggcgccccggctggaccccggtttccagtccggcgggggccccggggaagcccagggcggggagcgcggggctgggggcgcggagggcggggcgcccggggtccccgcccgcgcgccgcgccctagcctgctgggccgcgcccctcttgggaccgccgcgctccgggggcgggggcgggggcgggggcggggggcggggggcggggggcgggcgggggcgcgcgggcgggcggggtgcgggcgcgcgccgcgcggcccgagcgcgcgagccgggcccggagcgcacgccgccgccgccaccgccgccgccgccgcccgagccgccgcattgctgctgctgctgccgccgccgccccgcgcccggcccgcggcccccaatatggtgcagccgccagcgccgccgcccgtgccgccgccgccgcccgcgggccccgccgccgccctcgggcgcccgcagcgcggcagccgcaggtggggggccgcggcccgggcctgagctgccccccgcccggcctcggcgcggcgccctccccgcggggctgccggcgaggggccctctcgccgctgaggtggagccgcgggccccgccgccgccgctcctgccccctccctccctcctcccctccccctcccctccggtcctgtctccagcgggagcgcgagacgctggtcaggctccgcggcgcagctcgaaaaggaataatcgcccccgattgactgaaattcctccggagccggcgccgcggccgcccgcgcccgagaccgcgctccggggccgcgtcctcctctcctccggaaaacgctcgcgacccagggccgccggcggccgcgactctgctgtgtc

SEQ ID NO: 60 - DNA, 1200 bp, Homo sapiens
ctttctctttcatcttcctccctctgccagagccagggagggagagtgggaggcgtcaaggaggtaggggagagactggcagaggaaaaggagtgggtgggtgggggccaagtaaatagatacttagatgatgaagtcaagccactgcggcaatgtttcttgtcagtttcacgcgggcaaagcgtgcctttcggtgggttataagcagcgcccggtccttccttctctcgccaagttgcctgatccttccctccaggcgcgcgcgcacacaccacactcacacaccccaaaaccaagactcgtcctacaggatctgggaaaagaaaaagaaaaaaaagccctcaatcaccacctccttctcgccgactccccctcaccccccgcctcccctccagcgggcagccaaggagagctagaggcgggggaggggagagggaggagaagcgacgcaagtgggtagcttttcagcgccggcgaggcgcgggaggaggagaagcagtggggaggcgcagccgctcacctgcggggcagggcgcggaggagggacccgggctgcgcgctctcgggccgaggaaccaggacgcgcccggagcctcgcacgcggccaagctcggggcgtcccctcccctcggccgggcgaactcaaggggcgcagctctttgctttgacagagctggccggcggaggcgtgcagagcggcgagccggcgagccaggctgagaaactcgagccgggaacaaagaggggtcggactgagtgtgtgtgtcggctcgagctccgggcagaggcatttgggcccgaggcccccgctgtgactccccgagactccgcagtgccctccactgcggagtccccgcgcttgccggcaaaaactttattcttggcaaacttctctttctcttcccctcctcctcggcccccatcttctgctcctcctccttctctagcagattaaatgagcctcgagaagaaaaaccgaagcgaaagggaagaaaataagaagatctaaaacggacatctccagcgtgggtggctcctttttctttttctttttttcccacccttcaggaagtggacgtttcgttatcttctgatccttgcaccttcttttggggcaaacggggcccttctgcccagatcccctctcttttctcggaaaacaaactactaagtcggcatccggggtaactacagtggaga

SEQ ID NO: 61 - DNA, 1200 bp, Homo sapiens
aatgagacccagagggtcggtcctaggacacaattcctctcaccctctgcttctgccaccatcctttcccctttgcccttggaggtgctggaggaggtgggagtgtccccgagagtgtccagactgcataaggtggcagaaaggtgctgagtgtcttccattgcaccagtgtcagggacttggatgtggtccccaggcccaaagccaaggcagatggactcatggagggggaggagcagaggagcagtgcggagtcactctctccatgtagcacccccagctcagtgcattgctggggttcttgcagggcagatgaagtgcttctttgtataggggaatatgtgggtgaaccaggaaaatgggagcctagtacatggaggtttattccaactccctgtgtgattggcaagttagaattgcccccctacctgagttattattggctcctgagaaacaggccaaaggcagcccccactaagaagcttttagcacctcctataggaaggaagccttcccagtgaatccagtgtgaggattcttttctccaggtctctggacctctcccaacatgctccatgttcctttgcacgaagaagaacagcgcagttcggtagagtgtatgtaagccccggttgccatcagcacactgtgtggattctggcaatacctgtcatccccactggtgctgcggtgcccctcatgccccttgttaataaccaatgatagtgccacttgaactcccaggctgttgtgaggagcaagcattcattcattcattcattcaatcactcatttgttcatgtgacaaatgtggattgaatgcttgccctctttcctctcccctggagccttggagagagagataagacaagcgcaaataactgtgacctgttgtaggaagtgagaaatacacccaggcagggccagatactgcgtagcaggcggggcacagaggagggaacgcttgtgtctaactgatgctcctaatgcggaagcccctgaaaggcggttgtggtgcaaaggaaaacccacaggccaaggaatgggaagaccaaggttgacacttgtttgtcaagtgtcaataatcatctctgcccggtgagttgttgcatgaagcaagatcctccttggggggtgtttagcacagagggggtgcgattttatttcctgtgaaaataccaggtagtattatgttcacta

SEQ ID NO: 62 - DNA, 1200 bp, Homo sapiens
catttcacctatttgactctgcctgcctagagcttgccgagaatgagacccagagggtcggtcctaggacacaattcctctcaccctctgcttctgccaccatcctttcccctttgcccttggaggtgctggaggaggtgggagtgtccccgagagtgtccagactgcataaggtggcagaaaggtgctgagtgtcttccattgcaccagtgtcagggacttggatgtggtccccaggcccaaagccaaggcagatggactcatggagggggaggagcagaggagcagtgcggagtcactctctccatgtagcacccccagctcagtgcattgctggggttcttgcagggcagatgaagtgcttctttgtataggggaatatgtgggtgaaccaggaaaatgggagcctagtacatggaggtttattccaactccctgtgtgattggcaagttagaattgcccccctacctgagttattattggctcctgagaaacaggccaaaggcagcccccactaagaagcttttagcacctcctataggaaggaagccttcccagtgaatccagtgtgaggattcttttctccaggtctctggacctctcccaacatgctccatgttcctttgcacgaagaagaacagcgcagttcggtagagtgtatgtaagccccggttgccatcagcacactgtgtggattctggcaatacctgtcatccccactggtgctgcggtgcccctcatgccccttgttaataaccaatgatagtgccacttgaactcccaggctgttgtgaggagcaagcattcattcattcattcattcaatcactcatttgttcatgtgacaaatgtggattgaatgcttgccctctttcctctcccctggagccttggagagagagataagacaagcgcaaataactgtgacctgttgtaggaagtgagaaatacacccaggcagggccagatactgcgtagcaggcggggcacagaggagggaacgcttgtgtctaactgatgctcctaatgcggaagcccctgaaaggcggttgtggtgcaaaggaaaacccacaggccaaggaatgggaagaccaaggttgacacttgtttgtcaagtgtcaataatcatctctgcccggtgagttgttgcatgaagcaagatcctccttggggggtgtttagcacagagggggtgcgat

SEQ ID NO: 63 - DNA, 1200 bp, Homo sapiens
ccaccatgccctttgctctccagggctctggggccggcccaggtcagccacttggtgggattccagcctcagggtggctgagctgcaggtgcagcacccggcctccaggtggcaccaatccgcttactttgaggtgggctctgacaaccagctgagctgcccacagtccctcagactcccgaggtcctgggagcagggatcaaggtggtccccaggccgtaaacctgacagaggctgcaggagtgcatttccacccagggtgcactcagcgggtgtaactccacacccgtttctttggagtcaaggcccggcctctcagtgaggagactgctcctggttgcccaccgtcgggtcatcccagcctgcagtggaaccctccgcagcctggcctcttccagggtagcccctcactcccctctctcctgtcctagcagacaggccagggagggtggacgagttccagctctggggatgtcccatcagctgtgtcaccttgaacaaatcattttgcctcttgggtctcagtttcctgcagtgtgaaacgtggtggaggcatgaggggcaatgggagccccaaggcctctttcagagacctcctctgggtcccatgtgacctcgtggctgtccccaaaggcaagagggtccccagccctgcggccaaggccctgggacaccctatccaaacaaatgcaggtctctgacgggctgagctttgctcgcttgggtttctttgctttcttccttttttttttttctttcctggatgaaattcctgccagctccgaaggaaggagggaagaggagcgagccgcagtgtagaaagtttccttgactcctcctccggctgggtctccctcccttgccaagcccagcctgtgaaactgaataacgaagatcactcaacaatgcctgcccctctctgactgcaccgtcccggcgctcccaccgccgccgccgccgcccaatagagcccctggggcggtccccaccgacggtgcagcccgccgggaccgggaggaagcagctgcggccatcgcgccgtgcgtccgcgcccggccgccaggtgccccagtagcccgaccgccgagatgcccagcccgccggggctccgggcgctatggctttgcgccgcgctgtgcgcttcccggagggccggcggcgccccccagcccggcccggggcccaccgcctgcccggccc

SEQ ID NO: 64 - DNA, 1200 bp, Homo sapiens
gctgaagtcacctttccagcagtgtctggtccagccagggcatcatgcccagtaacagccccgatgtccaacacttccccatgtcctcttctccccctagacactgccaccttaggggtcctggctgttacttcgtcccagggcctgactggtagtcttagttacacacacaacacaggggagggaggaaggagcttggggcaaagggagggagtccagccctttgcctttccttggagaaggcaagggagacaggaatgcctttggctgggagaccctggccagagatcagaagatcaaaggatgagaagatcaggggcgaggcaggtggcactcacttggaggcaccagagagaagcggcagctgctgcctcctaagggcagggctgacccagaacgccctcctctacccacagcctccccagcctatctttcctaactttatagccctgcttctccaccaggaggggctctgttggttacatgcaggtgaggagagggatgtccactgacattcatgctacacccatgggttctcccatttaaacctcacaacattatcttatggggaatattagatactattttatagatgcagaaactgaggcacagagaggaaaggaactagtccaatatcaaacaactgctaagtgttagggccaagatacaaactctggcagtctgaccccagagccactgaggattacattataagggcaggcagagaactctattcagtccaactgcagcatgagggagtgaggctaggtatcaggaaggacttcttgttgggcaggtattttagatccagatcttggatctagtctttctatagctctgatgaaccaaatagttcgggagttagggaagagggcagatgggtggggcgctatccagagggaagggcatacgagagaagcggcagaacgagagaggatcagcgcggagggtggcgggcactggaccgcagaactggcactcgaggtcccaggggccggaatgcgcttggagggagagggccgctcagcgagggcgggacagaacctctcccgggctgggagtgcacggcgcggtgcgcccaggtaggcttgggggaagggtgccgagctccggaaagttccacaggtcctccgcagccctggggagcaggacccagaaaccgacgcgacgggtggggcgctacccgggccggccctaatc

SEQ ID NO: 65 - DNA, 1200 bp, Homo sapiens
tgcagcacccggcctccaggtggcaccaatccgcttactttgaggtgggctctgacaaccagctgagctgcccacagtccctcagactcccgaggtcctgggagcagggatcaaggtggtccccaggccgtaaacctgacagaggctgcaggagtgcatttccacccagggtgcactcagcgggtgtaactccacacccgtttctttggagtcaaggcccggcctctcagtgaggagactgctcctggttgcccaccgtcgggtcatcccagcctgcagtggaaccctccgcagcctggcctcttccagggtagcccctcactcccctctctcctgtcctagcagacaggccagggagggtggacgagttccagctctggggatgtcccatcagctgtgtcaccttgaacaaatcattttgcctcttgggtctcagtttcctgcagtgtgaaacgtggtggaggcatgaggggcaatgggagccccaaggcctctttcagagacctcctctgggtcccatgtgacctcgtggctgtccccaaaggcaagagggtccccagccctgcggccaaggccctgggacaccctatccaaacaaatgcaggtctctgacgggctgagctttgctcgcttgggtttctttgctttcttccttttttttttttctttcctggatgaaattcctgccagctccgaaggaaggagggaagaggagcgagccgcagtgtagaaagtttccttgactcctcctccggctgggtctccctcccttgccaagcccagcctgtgaaactgaataacgaagatcactcaacaatgcctgcccctctctgactgcaccgtcccggcgctcccaccgccgccgccgccgcccaatagagcccctggggcggtccccaccgacggtgcagcccgccgggaccgggaggaagcagctgcggccatcgcgccgtgcgtccgcgcccggccgccaggtgccccagtagcccgaccgccgagatgcccagcccgccggggctccgggcgctatggctttgcgccgcgctgtgcgcttcccggagggccggcggcgccccccagcccggcccggggcccaccgcctgcccggccccctgccactgccaggaggacggcatcatgctgtctgccgactgctctgagctcgggctgtccgccgttccgggggacctggaccccctg

SEQ ID NO: 66 - DNA, 1200 bp, Homo sapiens
tttctcaggaaaccaaccatcgggcctcccagacagtatcaaggcgctgaaactcagtaggtccttgaggccagacccctcaccagtcatgactgcctagctgcctcctcactctccctaattcctgttttcccacacagggtgacatttcttccctgctctggaaacttctaattttagtgggctgaggagagggatttgtgactgagcgcccatctctgggctgtagcacttgaatgaggccttcttccctggcactgctgttggtgattggctttctgtgcagggcagggggcctagatgacaaccctgatgtttgggtagcactgtcactgtgagaggcatggaaatcctccagttcttagtcttcttggaggaaggaattcagacaagagatgcctagcaagaggtatagggttaagtaacctttatttaaagagtcaggatctcagagagacaagacagaacctgccactcaggagatagagccaaaagctagtgacttaggaggaattcttttctttctctttttttttttatttttttgagacagagtctcgctctgtggcccaggctggagtgcagtggcgcgatctcggctcactgcaagctccacctcccgggttcacgccattctcctgcctcagcctcccgagtagctgggactacaggcgccaggaggaattcttatttgagcttttagttaaggaaaacagtacagcttgaaagaggaactgctcaaaagaatgagtcagtcgctactcacgctggggagaccctcttgatgagagttttacaggattactcacgaacggcccaggcaggggccttactagtaagcacgttttgggaagtcctcagggcacgcaggcaccgtgacctcacacactagtgcgctggagtgtgtcattagcatctggaatctctccccagggatgtggtttttgtttttttttttttttttggtattataatgagctaaaagctattcaggggcgagttatcagaggtgagcccgtgctcttcagcggagaagatcccctacctggccgccggccactttctgtgggccgtggggtcctcaaggagacggcccttgggctcaggggctgcgtttccacacgcgcctttcccagggctcccgcgcccgttcctgcctggccgccggccgctccaacagcagcacaaggcgggactcag

SEQ ID NO: 67 - DNA, 1200 bp, Homo sapiens
tgttatatggatgtattgcatagtggtggagactgggcttttagtgtagccatcacccctatagtgtacattgtacctattaagtaatttgttgtccctcaccccctccctccctcccaaagtgattacaaatattaatgatgacagtgatgacagaaattctctccagatgtgatctgagtttggagggcaaagggatttttgaaaatgaatcataagagagaaattttgcaacaaaggagtgtggtacatttaaattgctgtttttttttttaaatactgtgctcacgcggacctaataaactatcaagcagacctcatggaattccacataatgcagaagcgagggaaaaataggtattgagggaaaagcagcactgttttcctcaattttaacacgtatgtgcagcctacacttacaaacttaagacagtagcttttaacatgtgactttgctcagggtaacagaccattctggttatagccttacttacagctgtggaggctgaatcctgatgggtctaagaagtgtcaggacaagtgttagggatgcacctgtctgtctgtcttaagggaatacgggtctgggcagtcttaaatcggagctcccttgaggagaatgtcgtgcagtagccttaggaatgtgaacattgggagactggctgggattttgtaggttatgagttggggacacttatgatagtgaacttgagcccaggagagaagccataaaaagtgaaactgtcctgggcacttggaggtgagtgtctctctagtaagatgcatgtgaaaggctgggagctgaaagcaaggagagcagaagaggctggtgaagattctaatctgcgtgtccaggggcactcttccaggtctcaggaacgcaggtcagaatgtgcaagccagctgccgggcacgtggctcacccctgtagtaccagcactttgggaggctgagagagaagatcgcttgtggccaggagtttgagaccagactgggcaacatagggagaccctgtctcttaaaaaaaaaaaaaaaaaaggactgagtgagccgagcccagtcctctgatgcactgtgtcattcatcccctttcttaggctgtgttggttctaggctagctgctgtctttctttggtaggctgctaacctctttggattgtgaatttaaaacatgttttacagtaaatttgctgccaagac

SEQ ID NO: 68 - DNA, 1200 bp, Homo sapiens
gaactgagggctcctcccctttttagaccatatagggtaaacctccccacattgccatggcatttataaactgccatggcactggtgggtgcttcctttaacatgctaatgcattataattagcgtaaaatgagcagtgaggatgaccagaggtcgctttctttgccatcttggttttggctggcttcttcactgcatactgttttatcagtggggtctttgtgacctctatcttattaaaccagtcttgcccaatttctatctcatcctgtgaccgagaatgcggaccctcctgggagtgcagcccagcaggtctcagcctcattttacccagccccctgttcaagatggagtcgctctggttccaacgtctctaacgcggggcccctgactgctctatttcccaaggtgtatctagcatctcgcactatacgaggccaagttaaggcttacacatttgcagaaggaaagaggtaaggaagcaacctgggaccttccactgtctctgtttccatctctctctttccatctctgttcatcccagaatctctctgtccctatccctaaatatcgaaaatttctgtctctgaccatctatcattgtggctgatcatctgtttctgaccattccttcccgttcctgaccccagggagtgcagggtgtcctagccaagccggcgtccctcctagtagtaccgctgctctctaacctcaggacgtcaagggcctagagcgacagatgtttcccagcagggggttctgaggctgtgcgcccagatcgcgagagaggcaagtggggtgacgaggtcgtgcactgagggtggacgtagaggccaggagtagcaggcggccggggaaaagaggtggagaaaggaaaaaagaggagaaaagtggaggagggcgagtaggggggtggggcagagaggggcgggcccgagtgcgccccccgcccccagccccgctctgccagctccctcccagcccagccggctacatctggcggctgccctcccttgtttccgctgcatccagacttcctcaggcggtggctggaggctgcgcatctggggctttaaacatacaaagggattgccaggacctgcggcggcggcggcggcggcgggggctggggcgcgggggccggaccatgagccgctgagccgggcaaaccccaggccaccgagccagcggaccctcgga

SEQ ID NO: 69 - DNA, 1200 bp, Homo sapiens
agctcctgctctccgctcagtcccacagccttcccatgcaagagttccgcagcttctcctgacgactccttcccgcaaaatccccaccccgtccatcagcaacgctgtggactctctgctcctgcacctcccttacctcccttgtcacacggagaaggaagagccggacaggtacccgccaggagggctgctctgtagctgggaacctcactgagtttgcagcaggggccagaattgtggggagccattctagagaccgcattttctttttttcttttttttttttttttatgagatggagtcttgctctgtcacccaagctggagtgcagtggcgcgatctcagctcactgcaagctccgcctcccaggttcacgccattctcctgcctcagcttcccgagtagctgggactacaggcgcccgccaccacacccggctaatttttttgtattttgagtagagacggggtttcaccgtgttagccaggatggtcttgatctcctgacctcgtgatccacccatctcggcctcccaaagtgctggcattacaggcgtgagccactgcgcccgaccaaccccattttcttaatggtgccgcagattgggaagtggctcccagggcctcagcctattggcatgcaggccaggcagtgcccagctgagagtggctcacaaggatgagtcaggaggaaatgagcggatggcaggagaggctgccatctgtggccgcctgcatggccaggcctgttctcagcgctttacagcggccagttcagctgctctcacctcagccacctcccgagagacacgtgggaggtggaggctcagagaggggaagtcaggactagagcctgggcattttgataatgacaggagtgagacgcaggtggagagcaggccaggagcctgcggcgcttctcctgtgagctgcccccaggaggcgctcgtgtgttgcccatcgctcccaccccttcttccctccacatctctagaaatacagacgctgcgtgcaaagcactgggtgctgatgtggcttagcatctcctgacacctgtggcctgtcctccctccagccgggtgacacccagacccccagctcctgcacacccctgggcaaccctgctgtgcccccggggtgctgagcgtggctttgccagtgtgtcagaggcatcaattgctaaaacaattcggcagaaatt

SEQ ID NO: 70 - DNA, 1200 bp, Homo sapiens
tatgtcaagaatgaaagatggctcatcattatggaccatacagacgttaaaaggataataaggaaatatgctgaacaactttatgccaataatctgacatctaataattggatgaaatggacaaattccttaaaagaattaccaaagtttattcaatattaccttgataacttgaatagccctctagctgatgcagaagtgaatttctcattaaaaaccttcccacaaagaaatctcgaagcccaaatgggttccccagcgaatactaccacaatttttagggaataagttaccccatcctactcccaggtcactgcaggcccggcttaaaccgaccccaaaccagacagacacagaaaacgggaggtcggccacagagcaatctgcttcaccagcaccgtccccaaccttggacaaagcgttaccaagctgagtcaggcaatacaccaaaaggataacgaatcgtgaccaagtgagttttccaaggagagccaaacaatgtaattccctatgggctgaaataaaaagctacaggatcatttgaatacatgcagaaaagcgttgcagaaaatttcacacccactcataagaactcttggaaaaacaggaatagagggaacttcctcaacttgataaaaaacaccaacaaaaaacgactcctaacatcactctgggtggtgaaagcgcacttttccctaatgtgtgggcggcaagaaggcggacccgctcgaagctccgcccacccacgcgaggccccgcccgcccgctcgaagccccgcccacccggcggtcacaccgccttgcgcgctcggcatgacgtcactcccaccaggcacctcgccttgtcaggagctgccaggcgccaaccgccgacctctgaccgctacgggtcccggctcgcgcaacgaccccaaggcgcatgcccaggcgcggcgaccccattggtggcgggcggcgggagcggcgcgggcgcgtgcgggcggcggcagcggcggtgacggcagcctaggccgggcgagggccatgctgagcctcgcagccaagctggtggccttcttctggaggacggcggacacccctagggaggaagccgggcagctggagcccgagctcgcggaaggtggctcgcgggaggcggctgggaggcgggtcccgggccctcgcgtgtcggccacgagggagccgcgcccataggtcttt

SEQ ID NO: 71 - DNA, 1200 bp, Homo sapiens
aggccaggctgggtgacaagccccccgggtggctgcacctcccactcgccgacctgggcgctgtccttggtgctgccggcgccatagcgagcacaagcgccttaccttctgcccctgcggaagctgcggacgtcccccaaacgttaaaaacgccatatcccactttttatacaaataactaccccggcctgttctctttgtccccttcccgaagccttcaggcagaccgttccgcaaagtctttttttttgttttgttttgtcttctagacccagctcctcggcgctcatctccagctgctttaagacaaggggtgggggaaggggagggaggcaagaaaagatgagggtgggggaggggaaaagagggaatgcaaggggaaggagggaggagacggggagaaggaaagattggaagaaaaggatctccgaggaaggggctgagagaagggcagggtgaactggactaaaggccagagtaggaaggagaagaggggccaaaaaagaaggggatgaaattaagcacagaagatgggtaaagaaaaaagtatcagggaaagggcaaaataagagaaagccttgaggataagagggtagaaggctaaagaacaaggggaccactgggtcggggaagcgctgcctgaacggcgggacagtgacaaagaaagggcgctggcgatattcgcaccaagggtgcgaaacgcaatcgggaggtgagaaatggaaagaaggcgaatgcccggctacaagtagcctgggactgaaaggggacctgggggaggggctgggcccagggcagaaaagtccaggttcccatgcggcctgggcccacgtggagcgggcgctgaatcaccgttcagccgcccccctcccctcctccccgaccggtgcccgcagtccccgcctcctcggccgccgcctccacggggcggggccctggcccgggaccagcgccgcggctataaatgggctgcggcgaggccggcagaacgctgtgacagccacacgccccaaggcctccaagatgagctacacgttggactcgctgggcaacccgtccgcctaccggcgggtaaccgagacccgctcgagcttcagccgcgtcagcggctccccgtccagtggcttccgctcgcagtcgtggtcccgcggctcgcccagcaccgtgtcctcctcctataagcgcagcatgctcgccccg

SEQ ID NO: 72 - DNA, 1200 bp, Homo sapiens
agaatgacttctgtttcccttcctgaaggcttcttggtgcagtgttgagctacaggaaggcactagccacatgagcagggcccttgctgaaacccccttcctccaggaagccttctttgactgccataggctgcagaggctcctctcccccaccttgttatagctaacagaaccactcagttcagagctggtggtcttctgatgatctgtcccggagatctgttccctcagcttggccagagctccaggcttgtgagtctggtccgcgggcattcaaaggccctcagtggcatctgtgcatcagtgagactgcccggggttcctgctctgaacccttgccctagtgcccctagcctggctgagaacggagggtcaagtgccggggttctaatctcaattactttatccaagcatccgctcgctcatttacatgtttccctgggctctccactctgcgccaggcccggtacctggagctagggaaaccgaacattagtgctggctcgcccgcccccgcatccggggagctcggcgttctcgcggccctgctccccttgttttcaagtgcaagttccatgtttccgtgctgggaataaggccagccccgggtgccaccatactcgcctgatgtcttaccccgacccgcgccgccgagagcttgcggctacgggaacgcggcgcgccccggggccacatctggcctagcgggcgcgcgcgtgtcactaagacgctcattcaccggcgcagctgtcaccataacaaccggagcgagggaatcctggagactgccggggcggggggcgggggcggcggtcgcaggagggacccggcgcgggcgggaccgcggcggtcgggggacaccagctgcgtcggagcgcccagaacgccccgcgctccgccgagccccgctccacgcagacccgcgggcgggagggagccacgcacatcgccgccgcggccgtctccgcggggcggtaaccgagcctgcctcggagccgccgaacgcccacgccagcgaccctgactctatggccccgggggagcgcgccggagccgccgcgccgcccacccccagccggaaccctagcgtcccggggagcaagcggggagccccgggcgtccccggccccggcccagggccctgcttgtggcccccgctcccgccacggggcatgggaggcaggtagcccagcctgcgcc

SEQ ID NO: 73 - DNA, 1200 bp, Homo sapiens
cccggggttcctgctctgaacccttgccctagtgcccctagcctggctgagaacggagggtcaagtgccggggttctaatctcaattactttatccaagcatccgctcgctcatttacatgtttccctgggctctccactctgcgccaggcccggtacctggagctagggaaaccgaacattagtgctggctcgcccgcccccgcatccggggagctcggcgttctcgcggccctgctccccttgttttcaagtgcaagttccatgtttccgtgctgggaataaggccagccccgggtgccaccatactcgcctgatgtcttaccccgacccgcgccgccgagagcttgcggctacgggaacgcggcgcgccccggggccacatctggcctagcgggcgcgcgcgtgtcactaagacgctcattcaccggcgcagctgtcaccataacaaccggagcgagggaatcctggagactgccggggcggggggcgggggcggcggtcgcaggagggacccggcgcgggcgggaccgcggcggtcgggggacaccagctgcgtcggagcgcccagaacgccccgcgctccgccgagccccgctccacgcagacccgcgggcgggagggagccacgcacatcgccgccgcggccgtctccgcggggcggtaaccgagcctgcctcggagccgccgaacgcccacgccagcgaccctgactctatggccccgggggagcgcgccggagccgccgcgccgcccacccccagccggaaccctagcgtcccggggagcaagcggggagccccgggcgtccccggccccggcccagggccctgcttgtggcccccgctcccgccacggggcatgggaggcaggtagcccagcctgcgccaggacacccgtggcgggcggaacccgccaaggaccgcgaagtccagagaaaggaagctgaggagctgcccgcccgcccccggctgcagccccagcagggccctcccccggtggcgcgcacccgcgcgcgcacactcgcacaccgcacctcagcgcctgcccggcctcgcccccacccgcgagcgccgaacctcctggggccggatgccatgggtaacaacttctccagtatcccctcgctgccccgaggaaacccgagccgcgcgccgcggggccacccccagaacctcaaaggtaggctccccggccgagc

SEQ ID NO: 74 - DNA, 1200 bp, Homo sapiens
tgctcccaagtgcttatgagcccatgtatatgcacatttgtgtgacatgatgcagaagaatagtgcaggtctgtgttggggtgtgtggtgtgtgtgtatgtgcatggttaaagggataggttggtgtgggggggaaaagcctggattctggtgctgcactgcctgggtttgcattccagccttgccaccttttaggtgagcagcctctctttgtctcagttttctcatctgtaagaccatcatcatcatcttagagatggtacctgttgatctgttgataatgactgtaatactgacctcatcattcagcctgtgaggaccaaaggagttaatacttgcaaagcccttggaacagagcctggaagaagacagaaacggtgttagcacttggttaacagctccatcagcttgattgtgaacctcttgagggcagggacgatgtctgttttgcttcccagggggtcccctctcacctagcaggacctctgcatgtagtaggtgcatgaggctagtgaatgaatggctgtcggcctgggcgttcagggagggctgtgtgtgtcactacatccctgagatggggctgtgtgtgatggggatgtgtgtgcgtgtgcgcgcctctgctcgctttctggagctcgctgctatttcaggaagcctccagtctcctcctccttatgtaaataaacatgacagatcccatcggctgtaccagcgcgggaggtggggaggctggagtgggtgggagcggggaggcggcggccggaggggtgtgcgtgctgcgtgcgcgtgtggccggctggccgcgggcgggcggcgtgcgcgtgtgccggagggggcgcgtgggggcgcgtgggggcgcgggaggggcctcggccctgccatgccggtctccgcccgcgggggaggagacgggggaggaggaggggagcagccggccggggcgactcggtgaccagccggccggcccagcagaggtggctgcactgcccgtcgccggagatggggggacagatcacccggagcactctccacggtaaggccccgcggatgcctgcgggcccgcccaccccagcccgagaccgcctggggaggccgggcggaggggcgctggggctgagggcttggtcatgcccctacttctcactccatggaggggtctggggctgtgtgggggcaccttttggtggtgggcactggggt

SEQ ID NO: 75 - DNA, 1200 bp, Homo sapiens
gaccgcctcggcctctcaaagtgctgggattacaggcgtgaaccaccatgcccagcctatccttttgttttccatcctgtgttggcttggtgggggagaggaggtgttgacacgtggaggacacacatataaggcattcttgggtgacttcgtcatcactggaccctatctctcaaaattccagcgaaatctgctcttccctttaaggagtgaaagaagggtcagcattccagaagttcctggtcatacccaggcttttaatgaattgccactggggaatcagcatcccgttgctgtaaggactataagatggcggattgtgagagcatagggaaaggtctcggaggtctcttgtccttgctccacgcaggtctttctggcctgaaaatcccgttgaagagagcagctcttgagagtttgctccaagttccctcggggtgatcagcaccacggacagcggccagggcgcccccgaggacccgcaggcaggcagggtgcacagcggcgagcaggtgctgcgctacgtgcgggccagggaactcgcgcggggaggggagaggcgccgcgggtggcggggtcttggccggggctgttttcgctgtgagtcaccccgtgctccccgcgctcacgtcggtcctcggaaagccggggtcctccctgccttttccagcaacggtggggtggggaggcaggaagaaagcgccaacctaggacacctggacatttgcaggaaaggaagaagcgggagacggggacttgtctgtgtctccagcgcgttcctgccccccggcccgacccggcccatttctatacaaggtcggctctgcccggtctccacctcccacgtcgcaggcgcggaggggctcattcccgggccctgatctcagaggcccggaatgtggctgataaatcagagataaccctgcatggcagggcaggcccgacactcagctccaggataaaaggccacggtgtcccgaggagccaggaggagcaccccgcaggctgagggcaggtgggaagcaaacccggacgcatcgcagcagcagcagcagcagcagaagcagcagcagcagcctccgcagtccctccagagacatggatccccagacagcaccttcccgggcgctcctgctcctgctcttcttgcatctggctttcctgggaggtcgttcccacccgctgggcagccccggttca

SEQ ID NO: 76 - DNA, 1200 bp, Homo sapiens
aggaaagttctggcatttggcagattaagcaagtgtcttgatttgaagtccagtttagtcccaatactccctcccacacccaccacatcgcacaatttggttttgtttatttactccaggttaaggtagagtctcttagcttggtttgagtgctgtactcttctctctggtctgatatttggaactaaagccaagccagaactccagggccaagggggatgttgaaaattgtctgagtccccagaccaccctgccagctcatggcaaagggagggatcagaggccacagggaaagcacttcagctgctcttcacagcatcaccctctccccatttaatggtttaggttaacaggactttttccttgaggcttgggacacggaagggagcctcccctaaaccaggcccttggagagcaggccccaggggagcagtgcaactcaccttcacacccacaagacggctcctgacttctgctccctcctcccctccccaaagtggaacagagagaatatgattccccacgacttccacatcacagtttccaaacaatggggaaatcggaggcctccccgtgtgcagacggtgatatttaccgccaaatgcgaaccaggcagatgccagccccagcacgcacgcaggtaacttcaccctcgcctcaacgacctcagaggctgcccggcctgccccacacgggggtgctaagcctcccgcccgttctaagcggagacccaacgccatccataattaagttcttcctgagggcgagcggccaggtgcgccttcggcaggacagtgctaattccagcccctttccagcgcgtctccccgcgctcgtcccccgtctggaagcccccctcccacgccccgcggccccccttcccctggcccggggagctgctccttgtgctgccgggaaggtcaaagtcccgcgcccaccaggagagctcggcaagtatataaggacagaggagcgcgggaccaagcggcggcgaaggaggggaagaagagccgcgaccgagagaggccgccgagcgtccccgccctcagagagcagcctcccgagacaggtaagggcgcagcgtgggggacccgtgctctttccccgggatcccctgtccccgtcctcgcgatgcagtcggccggctccggctccgaaggcggacctgggcgcctctggctctccgcggtcccgagttct

SEQ ID NO: 77 - DNA, 1200 bp, Homo sapiens
gacggagcttggatctctcttgtggtccaaagggaaaggattgctgaatgtaaagccaatgtcaccagaaaagctgaatccacaagagagtcctggagtccaaaagagcatctcatggggaatcaggaaagttctggcatttggcagattaagcaagtgtcttgatttgaagtccagtttagtcccaatactccctcccacacccaccacatcgcacaatttggttttgtttatttactccaggttaaggtagagtctcttagcttggtttgagtgctgtactcttctctctggtctgatatttggaactaaagccaagccagaactccagggccaagggggatgttgaaaattgtctgagtccccagaccaccctgccagctcatggcaaagggagggatcagaggccacagggaaagcacttcagctgctcttcacagcatcaccctctccccatttaatggtttaggttaacaggactttttccttgaggcttgggacacggaagggagcctcccctaaaccaggcccttggagagcaggccccaggggagcagtgcaactcaccttcacacccacaagacggctcctgacttctgctccctcctcccctccccaaagtggaacagagagaatatgattccccacgacttccacatcacagtttccaaacaatggggaaatcggaggcctccccgtgtgcagacggtgatatttaccgccaaatgcgaaccaggcagatgccagccccagcacgcacgcaggtaacttcaccctcgcctcaacgacctcagaggctgcccggcctgccccacacgggggtgctaagcctcccgcccgttctaagcggagacccaacgccatccataattaagttcttcctgagggcgagcggccaggtgcgccttcggcaggacagtgctaattccagcccctttccagcgcgtctccccgcgctcgtcccccgtctggaagcccccctcccacgccccgcggccccccttcccctggcccggggagctgctccttgtgctgccgggaaggtcaaagtcccgcgcccaccaggagagctcggcaagtatataaggacagaggagcgcgggaccaagcggcggcgaaggaggggaagaagagccgcgaccgagagaggccgccgagcgtccccgccctcagagagcagcctcccgagacaggtaaggg

SEQ ID NO: 78 - DNA, 1200 bp, Homo sapiens
ggattacaggcatgtgccaccaggcccagctaatttttgtatttttagtagagatggagtttcaccatgttggccaggctggtcttgaactcctgacctcgtgatccaccctccttggcctcccaaagttctgggattacaggagtgagccactgcgcccagccacatttaaattttagagtataattattgctgcttataattagaagtgagaagactgttttgaagtaagggacttccctggccaccctcgctaaaattgcaaccttaccctcactccatttctccttctctgctttatatttctccaaagcacttatatgataataaactgtataataccgtacttactgtgtttctcatgtatcccggctacaactgtcaactgcgtgggactggttttgtctgttttgttccctgctctatccctggagcgtaaagcagtgtctggtatagagtggatacttaatagatattcgttgaaccgggccagtggcggtggctcacgcctgtaatcccagcactttgggaggcggaggcaggcagatcacttgaggtcaggagttcgaggccagcctggccaacatggcgaaatcccgtctctactaaaaatacaaaaattagccgggtgttgtggtgcatccctgtaatcccagctacttcggaggctgaggcaggagaatcgcttgaacctgggaggcggacgttgcggtgagccgagatcgcgccattgtactccagcctgggcgacaaagagagacatcgactcaaaaaaaaaaaaattcgttgaacggaatgagtgaacaaacaaatggatggatgagtgaatgaatgaacgaaggaacaagtgaccgctcgaccttctacagcgacagcggcgcaggcgctcccgcaccaacagccccccaccccgccaccgccagagggcgcagacaggccgggtcccgagtgtctgtcccaggcccccgccccacaggatcccggcccgggagcccccgatgcagggacccgcccgggactctgggtccccgcccatacctcaacttcgcacgactgcgtgcctcaagccgacgcagcggcctactctcgcactgcagacggggaaactgaggcctgaggcggccggggtggggcagacctcccggcgagcccgagcccccgcccccggctagccccgccctggcccgtaagaagcacccg

SEQ ID NO: 79 - DNA, 1200 bp, Homo sapiens
aataaacttccatgttgttgttgttgttttgaaacggagtgtcactctgtcacctgggatggagtgcagtggcacagccttggctcaccgcaacctccgcctcctgggttcaagtgattctcctgcctcagcctcctgagtagctgggattacagatgcacacgaccacgcctggctaattatttgtattttgagtagagatggggtttcaccatgttggccaggctggtgttgaattcctgaccttaagtgatctgcctgccttggcctccccaagtcctgggataacaggcatgagccactgcacccagccaaacttcgaggtatataattaaaagtttaataagccctcagcctaattttaaaattcagattctttctttctttctttctttcgttctttctttctttctttctttctttctttctttctttctttctttctttctttcttctttctttttctttctttcttgataacattattatcatagtctggtagattctgtctttctataataaaaatcctgtgatgttgttcaaagaaactcaatgggctttctgtatgtttaccaaggaattaaaatcttaacaaaggttttcaacacaagaaatactcccttttgtaagaattcttcataggtttttctagcacaagtttgtttttattgaatgatacttgatggttctgtatgttatttcttttctactacatataattttgattaggggatacattaaatattttaaaactatcatttctttttccagaacaatcataacatgttttgcttcttgttgctttaaaaggaacattttaagttgcttttttacaacaataagtgtgaaaggaaatgccaaaatacaaacaatttcacttcttgttggaatttgacatttccactagctaagtaatataaacatgcatcacctaaatgagcagtattttaatagcatcgttttcacattttaattgttgtcacatttcttctttgtttagggagacgtctggtgaacacccgtgggatctaaagaacaagtaagatttaaagcatactgggttgtgtgcctggctttcacattttatttattttattgcttaatagtgtttctttactactgtgggttgtagcttaaaattgttagaaattttattatggagtataaggaataaaagtgaacgttcaacactttcaaatt

SEQ ID NO: 80 - DNA, 1200 bp, Homo sapiens
cctgcctttgcatagaattcctggaatgccttttcgtcatcttgttaaaatcctggttaaacttcaaggcctagtttgagacactatttccatgatgtcagtcttgatcctatcttttatagcattatctggttttccttcctggcgctaatcacttattaaattgtactaatcatctggtgtacctgctttattttccctactaatctggaaacaaaaataatagaacctgactcatctttctgccttgagacaccttgtgctgtcactcttgatgccttgcacacagtaggtgctctaaaaatagtttgtggctttttgattttacagagaagactgaggaaattaaacccttgcccaggaggcatataaaaatcctgttaaatgaaagaggctcatgctaactattgttttgacttattgatgccaaactagacttgaacattgatgtgtcttcagggaagaagccaaatatgctagtgtaaaaaattctaacctctgctatttacctggagaagttgaattagccctgtcatttcaactgatgccaaaaaagcagcttccattattgtgtcagtaatagacttttatcctgcatggaggaacttctcttaaatctttctcatagcactggtgtgcttctttgcactgatgtatttacgtaatgtttcataagaagtgacagttccttttttaggtctttgcccaaaaggtggcataagcaaagaatgaaagtcaaggtggttggctttaggagagcctaaaattgggaatgcgagagctcccacgtaacacaaacccggatagtctaagactcctacattttacaaaccaagttatttccttcctaatacaaacaaagtatgacagttacaatttattgtgtatttacaatggattgtgtctgtcatggtgattgtgatggaaccaggggggaaagagtaagaaatctaagcacttttgtgttttcttccttttcttttctcttcctccccgctttcaggctctgaaagtgttccagctgaaatttcagatcggacagactcgctgcggctccggaggcagtgattccaagctgctcgcgcacgctgctgccaagctgcaggatggtgcacgtagccaggctgctgctgctgctcctcactttcttcctccgcacggatgctgagagtaagccattctccccttctctagggggct

SEQ ID NO: 81 - DNA, 1200 bp, Homo sapiens
aagcaaacctagataaatgggacttcatccaactaaaatgcttctgcaccgcataggaaataatcaacaaaatgaaaaaacagcctatagattgggagaaaatatttgtgagccatatatctgataaggggttaatatccaaaatacattacgttcacagaactcagccaaaataaataaataaaacacccagttaactaacgggcaaaggatgtgaatggactttttttttttttccagaggaagtacaaattgccaacaggtataagaaaaggcgctcaaaatcactaatgaacagggaaaagccactaggtatctcctcacacctgctaggatgactgttataaaaaagataagcgataataagtgtcggtgatgtgcggagcaaagggaaccttgtacactggtggtgggaatgcggactggaacagccattatggaaaaccctacggaggttcactcaaaaagttaaaaacagaactaccagcccagtgttatccgtccttcgttaagttcaaagtcacggtgccacttcctttcccatctaaaggggactgctcgcgacccaagccaaggcccgcaagtgtggctctccctgcggggagagtagggcaggaggtggatttccaggccagaggcttccaggctgggcccggaccccacgccgaccccggccccggggggggctcagcgacctcggcgcctcctcaggcccccagtgaacctccggggcccataaaggctccaggccggctccccggaggcccaccagcgggcgtctgggcctccctccgcgccgcgaccgccaggaggcgccgcccgggagaggaagtccagctgggcccggccgggcttcggaggcgccgcccgggagaggaagtccagttgggcccggccgggcttcagaggcgcagggcgggagccgcctcgcgcagggtcctccccaagccggcgccaggccctgccctcgtccggccctgccctcgtctggccccgcccgggccgcgagcactggcgggttctgggtcctgtgaccggtcaggcggcgtcagcgggcgcggcggagggctggccggcctcgggggagtttccgcggccgccgggggcgcggcggcagagcgcgaggccgggcagggggccagactcggagtcgaggcgcgcccgacagccgcagcgctcatggcgggcggaggagccggg

SEQ ID NO: 82 - DNA, 1200 bp, Homo sapiens
agcaaacctagataaatgggacttcatccaactaaaatgcttctgcaccgcataggaaataatcaacaaaatgaaaaaacagcctatagattgggagaaaatatttgtgagccatatatctgataaggggttaatatccaaaatacattacgttcacagaactcagccaaaataaataaataaaacacccagttaactaacgggcaaaggatgtgaatggactttttttttttttccagaggaagtacaaattgccaacaggtataagaaaaggcgctcaaaatcactaatgaacagggaaaagccactaggtatctcctcacacctgctaggatgactgttataaaaaagataagcgataataagtgtcggtgatgtgcggagcaaagggaaccttgtacactggtggtgggaatgcggactggaacagccattatggaaaaccctacggaggttcactcaaaaagttaaaaacagaactaccagcccagtgttatccgtccttcgttaagttcaaagtcacggtgccacttcctttcccatctaaaggggactgctcgcgacccaagccaaggcccgcaagtgtggctctccctgcggggagagtagggcaggaggtggatttccaggccagaggcttccaggctgggcccggaccccacgccgaccccggccccggggggggctcagcgacctcggcgcctcctcaggcccccagtgaacctccggggcccataaaggctccaggccggctccccggaggcccaccagcgggcgtctgggcctccctccgcgccgcgaccgccaggaggcgccgcccgggagaggaagtccagctgggcccggccgggcttcggaggcgccgcccgggagaggaagtccagttgggcccggccgggcttcagaggcgcagggcgggagccgcctcgcgcagggtcctccccaagccggcgccaggccctgccctcgtccggccctgccctcgtctggccccgcccgggccgcgagcactggcgggttctgggtcctgtgaccggtcaggcggcgtcagcgggcgcggcggagggctggccggcctcgggggagtttccgcggccgccgggggcgcggcggcagagcgcgaggccgggcagggggccagactcggagtcgaggcgcgcccgacagccgcagcgctcatggcgggcggaggagccgggg

SEQ ID NO: 83 - DNA, 1200 bp, Homo sapiens
aagtgcccacccaaatagcctcatcattgctgagggtctgtacatgacagtgactggggcgtaggcagctgcacacaccagggctgagtcccagcacacgtgttctacctgtgtgactccgagccttacttttctcatctgtacacagtctctgtctcaaggctattggaatgagtcaatgatagagtgcacgtaaatcacttgcacagagagttgttcaaacaatggtagctagaactgctattgatctattgtggggtaattgtcttaactgctttgaaggattcagccctatgtgttcctttgtccagtcagatgggaaaatataaatgagcccttctcaatgttaggaatgacaagatcaggccaagacccctctttctcagactatccggaagatacacagatgacctggacagtgtctgcaggctcctgtgtatcctgaggcttcctgagggccaagttctatgggtgggaaggcagcttgctctctaggttgccataactctgcatctgatttgcatgtgtttgtggtgaccctgcagtttcttctggcatttccaacttaaaatattgctgtttccccaggcagggcagatattgatttttttcttctctaaccattccgattgacccggctcccccacccagcgaatgggggagggagggggtgggggtgaggaagtggatgggggagaagggaggagggatggtgttgcccttatagctagcaccttttactccgtggaacctgagtcaccggggctgatgagaatgggctctttccagaagcaagtgcatgatgaagaaagaacagagggaatgttccccctcgggtccatcctggtcccttctttccctgcatcccttatatgacctgcagttccctgagtccacttgccaccgggggcgggtggcacccaggcggcggggaggcgggggaggggcgcagtgacagccgagccggctttgaggaaccttgcagaggaagtggcttcagaactgctttacgcgaggggcaggaaaggcgcgggaggcgggggaggtgcggagatggcgctctgcacggcggcggagggagggcgctggcgccggggacacgaaaccgcagagcccggacgagtcagggagtgaggcgcgagccgggcgcccggggctctgcaggcgcaggcggcgcggggacaggagcaggttaccggg

SEQ ID NO: 84 - DNA, 1200 bp, Homo sapiens
cttgaagcaagccttcacgggggaaagtgccgggagaaaggagatctctctttgggtcccatgagtgaaccatccataccaggttccacttagggcatttaggggaactggtatctccacagtaattactagagcagctctggggaacggagggttggctaaggaagaaaagctcccccaacccttggggcgagggagcgttctctcaatggagcccccccaactcccctccaccccccaccagtcttccaggaaagaggaataccctacccggcagggctgcgaaggaaggggaaatccaaccagagcgaaagtcgcacgcggacagctctgccagcccttggaggcatccggcggtcacccacgggacaaagcgcggctgcgggagcgcgcgcggggcattccggacccgcgtcgagctccgctctagagggggcggcgggcggcgacaagccggagagaggaagggccaaggagcacggccctcctgtcggcaccatcagcgggagagtggcgagcggacgcctagacggaggggccctactcagaccccatcgagccagttcccaagcttttccctccgacctgctccctcccggggcgcgtgagggtgcgggtcgggggtgaacctggtgttggggaaagtgattggcaagggaaagaggcagggctgaaggcctagggccccccgcagaaatgcgccctctgagctgtactgtacagaggcgctcctctgcggaatcctgcagctgctccaggtgaagacgcccccaaatccacactcgcgcagacgtcgcctggcacggaccctacccccttcggtccgtcggcggttctcttgggtcgtccttcctgccactccgactctccccgcccccgccccgcccagcaggctgcggttcctttaaaggcgcgctggtgtggggcggccccttctctcggggctggctcgggagtagcgcagtcgccaaagccgccgctgccaaagctgccgccactagccgggcatggccatggcgtccccggccatcgggcagcgcccgtacccgctactattggaccccgagccgccgcgctatctacagagcctgagcggccccgagctaccgccgccgccccccgaccggtcctcgcgcctctgtgtcccggcgcccctctccactgcgcccggggcgcgcgaggggcgcagcgcccggaggg

SEQ ID NO: 85 - DNA, 1200 bp, Homo sapiens
ttcaagatggtgaccgcagagcgttaaaccagacaagggtccatgaaggcttgaagcaagccttcacgggggaaagtgccgggagaaaggagatctctctttgggtcccatgagtgaaccatccataccaggttccacttagggcatttaggggaactggtatctccacagtaattactagagcagctctggggaacggagggttggctaaggaagaaaagctcccccaacccttggggcgagggagcgttctctcaatggagcccccccaactcccctccaccccccaccagtcttccaggaaagaggaataccctacccggcagggctgcgaaggaaggggaaatccaaccagagcgaaagtcgcacgcggacagctctgccagcccttggaggcatccggcggtcacccacgggacaaagcgcggctgcgggagcgcgcgcggggcattccggacccgcgtcgagctccgctctagagggggcggcgggcggcgacaagccggagagaggaagggccaaggagcacggccctcctgtcggcaccatcagcgggagagtggcgagcggacgcctagacggaggggccctactcagaccccatcgagccagttcccaagcttttccctccgacctgctccctcccggggcgcgtgagggtgcgggtcgggggtgaacctggtgttggggaaagtgattggcaagggaaagaggcagggctgaaggcctagggccccccgcagaaatgcgccctctgagctgtactgtacagaggcgctcctctgcggaatcctgcagctgctccaggtgaagacgcccccaaatccacactcgcgcagacgtcgcctggcacggaccctacccccttcggtccgtcggcggttctcttgggtcgtccttcctgccactccgactctccccgcccccgccccgcccagcaggctgcggttcctttaaaggcgcgctggtgtggggcggccccttctctcggggctggctcgggagtagcgcagtcgccaaagccgccgctgccaaagctgccgccactagccgggcatggccatggcgtccccggccatcgggcagcgcccgtacccgctactattggaccccgagccgccgcgctatctacagagcctgagcggccccgagctaccgccgccgccccccgaccggtcctcgcgcctctgtgtcccggcg

SEQ ID NO: 86 - DNA, 1200 bp, Homo sapiens
tactcaggaggccaagacacgagaatcgcttgaacctgaggggatggggggcagaggatgcagtgagccgagattgccgagatagcgccactgcattccagcctgggcaacagtgagagcctccatctcaaaaaaaaaaaaaaaaaatggacttcttcctcattgacctgtggtggctccaaggccatcaaaaaaaaaaaaaatggacttcttcctcattgacctgtggtggctccaaggccatcatctcacagcctttttttttttttttttttttgagacggagtcttgctcccttgcccaggttggagtggagctgcatgatctcggctcactgcaacctccgcctcccaggttcaagcattctcctgcctcagcctcctgagtagctgggattacaggcgcgtgccaccacgcccagctaacttttgcatttttaatagagacggggtttcaccatattggtcaggctggactcgaactcctaacctcatgatccgcccgcctcggcctcccaaagtgctaggattacaggcgtgagccactgcgcccggcctcatctcacatctttcttcctctgcagcacacgacacgccctagttggaaacaaagtcggagtttgtggattgggggaagggcggggtctaacctcaggtcaggcgccgtgcaaggtacatcttggcacccggaagaggcccagtacagttgcccccgaggtgacccgacctcccctaccaattgaggcgcccttgttgccaggcttgcggcgggggagcggcgggggagcgacggggatgcgctcattggtcaaggaaggggcgcctgttactagaggcgagaaccggagcccattggtcggaacacctcacaatggaccccagcggcgcgcaaaatccttatgattggtttgctggctgcctcgggagaccctgttgccaggatacttggcgttcccgacccgacccccgttccccattggctgtcagggcaaaagccgccatctaatgaggagcgaggtgcggtgccccgaagcgctcgcttcccgcggtgcgatctagtcctgcagtaggcggcccggggccacaccgcggccgcccaagccagtgcaaggcccaggggcctgacatcgctcccagcgctcgaggaccgaggcctgctgtggaggacaccgtgctccctcgggacctgctctggattccg

SEQ ID NO: 87 - DNA, 1200 bp, Homo sapiens
caaaaagcagccggggatggggcgagaacagaggacttccccactgagcccacatccaacctcacccagaaccccttctcaggtccagttgctgctccctattaggggtaaatgtgtttatgcgcatcatttccatcatttctttgggggaaaattggattctttcctcctcctcgctactcgctctccgactctgccagcccatctcctgagagttctcctgtgtaagggatgcaagggccaccagcaccaggtctctgaaaggacgccggagccagtttccggtttccaagggttggaccagtgctggaggggggaatcacaaactgcccttccaggaccctaccctctctactaccccaggatgctgaaaaaggctattctgcctctcttcccctgctcagctcttccccgcctcctgcctccggccccagcagaggccgcgctcccactgcgcctctcgcctctgagaaatggcgcagcaagcgctccgcggagagcctggaggcggggcgcccttctctgagtccgcggggtcgcaccccgagccagtcggccagacctgcatcccgcgtagcatccctgccctctctgtgcagcggaaagggcaaaaggcagggactgcaagcgggcgcgcaccgggtaggaagagcggctctgcgtaggtgcgcggacccgggctcctgggttccatccccgccgcgcacctcggggtccgcacccggctcctgccgggcccttttcggccgcaccccgctcccgcaccccgctcctccccaagccccacccggcccaagccgcgcgtcccgcactgagctcctacacgcgccgcgccccggccgcaccccgcgcagccagagcaagcactggagccccgccccttcccgcaccccacccgcctccggccccgcctggcccacccctggaccgcccccgccccgccccgcccctacccgctcctccggcgcagccggcgcttacctcgcttcagtcccgcgaccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggt

SEQ ID NO: 88 - DNA, 1200 bp, Homo sapiens
gacctcaaaaagcagccggggatggggcgagaacagaggacttccccactgagcccacatccaacctcacccagaaccccttctcaggtccagttgctgctccctattaggggtaaatgtgtttatgcgcatcatttccatcatttctttgggggaaaattggattctttcctcctcctcgctactcgctctccgactctgccagcccatctcctgagagttctcctgtgtaagggatgcaagggccaccagcaccaggtctctgaaaggacgccggagccagtttccggtttccaagggttggaccagtgctggaggggggaatcacaaactgcccttccaggaccctaccctctctactaccccaggatgctgaaaaaggctattctgcctctcttcccctgctcagctcttccccgcctcctgcctccggccccagcagaggccgcgctcccactgcgcctctcgcctctgagaaatggcgcagcaagcgctccgcggagagcctggaggcggggcgcccttctctgagtccgcggggtcgcaccccgagccagtcggccagacctgcatcccgcgtagcatccctgccctctctgtgcagcggaaagggcaaaaggcagggactgcaagcgggcgcgcaccgggtaggaagagcggctctgcgtaggtgcgcggacccgggctcctgggttccatccccgccgcgcacctcggggtccgcacccggctcctgccgggcccttttcggccgcaccccgctcccgcaccccgctcctccccaagccccacccggcccaagccgcgcgtcccgcactgagctcctacacgcgccgcgccccggccgcaccccgcgcagccagagcaagcactggagccccgccccttcccgcaccccacccgcctccggccccgcctggcccacccctggaccgcccccgccccgccccgcccctacccgctcctccggcgcagccggcgcttacctcgcttcagtcccgcgaccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgt

SEQ ID NO: 89 - DNA, 1200 bp, Homo sapiens
tccccccatggaggctgccctctagttcgtcctttagaggaagcatgaccaagggtctgctggtacttttctctccctctctaagaatttcctcaggagaggaaaaagttagctttcttttctctgctcctgagtgccatggcctgcagtacgtgaccagtccttggcacagagtcactcagggctcatggtagatgcttagcggtcacagccagtagctgcgcagtgtggcagcagctgagatccatactgcagcctgaaagttgtgtttcagcttctgaggttacaagtgtatccagtgtttacatcagagataaagccccctttcccctctctcctacgggagccctcaacaagacagatacacgggttgggtggagtgagaccacatccagcagaggcaaagcttgctaaaagagagaggaggaggagggggagagggaggaggcggcacggcgtcccttggcagctccgtgcatgctgcggcagttgcaacaggagaaccgagtaccagttgctcaacactttatcactttgcattgctctttctcctcccttaagtgaatttccattttgagggtacagctccacaggagaaaggtgtggtggtggtagtggtggagggtgtctgtgggggggatggtggtgcgacgggagttggaatgtccagtgtggggaagcaaaggagtgcctactaggatgcgaataccacttggccctgggctcgctccgctttagccttcccctctcgctggggagaaacaggcggtttgtgagggagactgagagaggggcgtgtttgtggtggtggcggcccatcgggggtccttgtcctctccgaatacccggactgctgaggggaagggccgggggggccccggcggttgccatggtgacgcagggtcggcggctcagcgccgtagagacaaagccgcccgcccgccagcccgctagctcctcgcgcgagctcctcctccacgggaacctccctcccctcccaggcgccgccgcagccggagcggctcccgggccctgggccgccgccggccaggtaaggcgatcgcgcccgcggccggggaggggtcggcgggacgccgggcctccccaacgctggggcagcagccagccccgcagtcctggacggcgccgggccgcccaagcgcgccgaggagagggggcggcaaacttcgggccgagcgg

SEQ ID NO: 90 - DNA, 1200 bp, Homo sapiens
gagggagagatgcagggagctggattttagctcctaaggtagaaaatttccaaaaggctgatcaaagattaaatggctgccttaaaaggtaatgagttccttgttagtggaggtgttcaaccactgcttggtgggaagacactgtaaggaggattcaggcaagtgggaagggggctttttccagtccctgaaatcaatgattctaacaggaaaagatgctccggggtgcggtgagcgcttacctctgctcttgcagagacactgacagacgttcattcaacatctgttcctgccaggcactgtgcgcgctgaacacagaagtacagcctctatgaagcagccgctgtaggcctcagagacagctgatagatgtaacactgttgtctccatgtttcaaacttcggtcatggtcttactaggtctcctcattaaggggactcactatgtgtccagtaacgctttcttgttaccagatgacagagagtcaggctgtcagagttcacgactttaggtgcacagtttcaccctgtctggtccttgggagacggagaaggggtcaggagaaaacgtgggtgaagaggcagcaaagccgcaggagatatgaactaattgctcaacgtgggtgtagcacggattaggccttttacagcaagcgctgccagcagggaactggtggatgtactcttgaccacgctcggctggactctggggagcccaagacgggtaagactaggtccccggccccagcagctggtgggggcgccgcaggttagagacgtgaggccgcgaggctgagggctgtgaaggcggtgggcacgcacggcgtgccggggccgcctggcttcgcggcgggggaggtaacactgcgacaccccgcagacgctgtcagccggggtcgcggccccgcgtgcgcccgatcatctgactgctggggagtgcgggggcgcgggagccaggcgagccagcgcaggcgcgcgctgcctcctccgtccccaccgagtccccagcgcgtgcgcggggccgtggccgaggcctgcgcgccgcccggccgcctgcactgcgcgcgcgcccaccccgcgtgggaggcagcgggaggggcccggagaggtgtggagcggcgcggcgggaggctccgtgggcggccacgggagacagcgccggcgggagcgcgcctctcggcctttcctccgcgcccccgcg

SEQ ID NO: 91 - DNA, 1200 bp, Homo sapiens
ttagctcctaaggtagaaaatttccaaaaggctgatcaaagattaaatggctgccttaaaaggtaatgagttccttgttagtggaggtgttcaaccactgcttggtgggaagacactgtaaggaggattcaggcaagtgggaagggggctttttccagtccctgaaatcaatgattctaacaggaaaagatgctccggggtgcggtgagcgcttacctctgctcttgcagagacactgacagacgttcattcaacatctgttcctgccaggcactgtgcgcgctgaacacagaagtacagcctctatgaagcagccgctgtaggcctcagagacagctgatagatgtaacactgttgtctccatgtttcaaacttcggtcatggtcttactaggtctcctcattaaggggactcactatgtgtccagtaacgctttcttgttaccagatgacagagagtcaggctgtcagagttcacgactttaggtgcacagtttcaccctgtctggtccttgggagacggagaaggggtcaggagaaaacgtgggtgaagaggcagcaaagccgcaggagatatgaactaattgctcaacgtgggtgtagcacggattaggccttttacagcaagcgctgccagcagggaactggtggatgtactcttgaccacgctcggctggactctggggagcccaagacgggtaagactaggtccccggccccagcagctggtgggggcgccgcaggttagagacgtgaggccgcgaggctgagggctgtgaaggcggtgggcacgcacggcgtgccggggccgcctggcttcgcggcgggggaggtaacactgcgacaccccgcagacgctgtcagccggggtcgcggccccgcgtgcgcccgatcatctgactgctggggagtgcgggggcgcgggagccaggcgagccagcgcaggcgcgcgctgcctcctccgtccccaccgagtccccagcgcgtgcgcggggccgtggccgaggcctgcgcgccgcccggccgcctgcactgcgcgcgcgcccaccccgcgtgggaggcagcgggaggggcccggagaggtgtggagcggcgcggcgggaggctccgtgggcggccacgggagacagcgccggcgggagcgcgcctctcggcctttcctccgcgcccccgcgtccccagccggccgctccgagaggac

SEQ ID NO: 92 - DNA, 1200 bp, Homo sapiens
acaaaaaaattccaatagtgattagtaacaaccagatactgtgacagtgccccattgccattccatgggagatgcaagagcattgtctcagtaataaaatatgatgttatcaggcatttatggctcctggggaaaaaaatgcaattgcttgctgttttgatattatctctgactatgcttgcttatcatttgccatttctatatttagtagccagtcattttgaagttaaaaaaaaatcaaggtttgcattgtaaccacaaaccagtctttgtgaacaggaatcctggaggctattaatcagcttttaaaggaggtaagtcatggtggtggtggggtatctgaaattttggaagttccgagctcactttaagagttgagatgggtgggagggctgaaaacaccaccattcatcattcttcagttatctcgagactcaacttaaacatttttaatcaaaaagatgggaggggacacgggtcacatctcaggtttgggggaatgtgtgtgcgtgtagatgtgaaaactgagccaaaatattccactgtgaagagatctcccttctcggctggtggacatcaaccttaccaagtaatctttggagcaagtaactaccaaataaagccgttccaaaacttctacgtaagctcctactttttaatgagtacgcatttctccccattttcttttcctctccaaggactgagatagctggagcaaggatgggtccccctctgatttcgtccccgccccagccaatctgagcccggatgctaattagctgcgctcccctgatgaacacgcctttgtcacaagtaggcccggactacaaagcccagaatgcctcacccctccctgatcaatgagggcttatttaaatgatctctgaggtcttggacccaggccaatcagctgtcagggctcatgataaatcgcaatgcattattgataataataattactgggacatgcgcgttccggccgaaggggggtaaatttcccaactccaggaatttgtggcggagagggcaaataactgcggctctcccggcgccccgatgctcgcaccatgtcgaggcgcaagcaggcgaaaccccagcacatcaactcggaggaggaccagggcgagcagcagccgcagcagcagaccccggagtttgcagatgcggccccagcggcgcccgcggcgggggagctgggtg

SEQ ID NO: 93 - DNA, 1200 bp, Homo sapiens
tgtaacatggacctctctgtgacgcgcaagtattatctggcagttacatggtgccgcctgtgcagctgggcgccaccaccagcttcctgccctgtgcgattaaaaggccagcttcggggacagcagcctcctcaacgcaaagcgaccagccctgccaggcgtcggctgcagctggtccaaggttgacggtcccagggccgcattctccttccggtcacctcagactctggaaagagagccatggagggcctcagagcgcccctctgcagttccttgaggaaaacaagggcctccccctcttggctggagcagccaagtgcccacggggaaaatggtcccgtccaccatttcaggcaaacgcccccatagctgtaataaacatgtgatgggtttcctgcaccaagaaaggaaaattttccttaaaaataacaaaaagaggtggggaggggactctaagttccatttaagattttccaaactcgttgtgtctcatgcatacgctgaggcttaacatgaaaatttggagcgaaagtccccacggttcaccacagggaatgaagtaaagtatctcgccggtagctctaggtctcgtaaaccgtggagctcctttagtataaggccctattatgtgccacattagatgagaagaaaataagaggaaggatacggtagagcctttagaatggggttcagtcttaggactcgtaggataaaggaaggtcgtttccctccgtttgaggcagggaatcaaacaaaacaccggcaccgcaggcaccgcagtcgcactcctggggcctcgtggctttcccgcgcgcccgccttgggggcggggaagaacccggatggaaccacccttgcagcccggaccccccgccgtctttgaatggcagcggggcggagttgccctcccttctgtgctctgactggtttggctccgcactctcctcttcgtgattgggctttcctagtgccagtcacagagcgacgctggtctcccagattgttgcagaaggagacggcgtcgacgtctgactggactcgcggcgacttacctttcagtcgtgcgctcctgatccggcgctcggaatttgtccccggcttcagggctgcggggcctggaaggaggcgtatcgaggcggctcgaaaacgatccaggggagccgaggcgctcctcttgtcatcccactcagcgccatgtcc

SEQ ID NO: 94 - DNA, 1200 bp, Homo sapiens
tttaaaagtcccactgaaaactttttttttttccagatcgaaacacaagtttaatagtcattcagatttcattccagtctaaagaaacttatagaaatctaattatctgccagaataaccgctttaaactgactgtaagaaccacagttactttgcagctaaaacagattgttctcttcccccaaacaaactcggacatttagacaaatattgctgctgtctttacattttagaaatcagtattaaggctcacctgttactgaacacatacgagaaacagttttgttatgcgtatcttatatacatcatgtatacattttcgtactttcacatgatgtgtataataagatacacataaaaactggttggcttgtcatgagtaaatgctttattctttagagtgaaggaatattggatacagaacgtcgttccatatttaatcttgctttagaaaattaggatttatcttggacacagcattttgaatataacacaggcaagttgagtcctgggggaaaaattgtatttttaaatttattatcaacggtgtctttcgcatttgagaaaacacatttgaagatgcccatgcagttccttggaagtaaactaggtcacccaccactgaaacaccctcggcttctgcaaaaagatgtgctttaaagagaatagaatggacgctcggtatgttcatctaaacgaccttgggcaagtacgtcgattccaaggtacaatcagccctcccagacggcttttcgagtctccctaaccccggtgggagaggacgcggcgccagagcccagctccggctagttttcccgggggcaggtgtagccttgggcgcggggccgggggaggggcaaggggcgggcgtggggttggactgggacgtgggactcggaccacggcctgggcgtgggcctaacgacgcgggaccggcccgccctcgccgctccattggccacatctgtgcagaaaaggccccgcggcccaggggcgcccgcagtgtcactaggccggctgggggccctgggtacgctgtagaccagaccgcgacaggccagaacacgggcggcggcttcgggccgggagacccgcgcagccctcggggcatctcagtgcctcactccccaccccctcccccgggtcgggggaggcggcgcgtccggcggagggttgaggggagcggggcaggcctggagc

SEQ ID NO: 95 - DNA, 1200 bp, Homo sapiens
tgttctccagagcaggaaacaagcttggagagtgcgataactagcccaagcttcacaatgggtgagtggcaggactgaggtttgattcgggtctcttcccagagagacactgggagagattcttgccccaggtcttttcccttcaggccctcaggctcagcacctctctgaacatctgtccctggggagtgagaatctgaactaaaatgtaagccaagaaaacacaggctcttcattgaatctttggaaactcctttcctgggctgtaggaggaccttcctgagccttaggcacttttgcctttgctggctctttcctccaagtcaagcatattttatgactgtggaggtataaatacaaatatattaacattatatagtaaaacattttgtttgacataaattttttcttctgatttttacagaaaacattttcatagatctccaaaagtatcgtgggccctaagcgctgtagctaagtcgtttctcctttctcagcctccagtctgacatgttccactatggccttgggaagagcaatgggcccggagtcgccgcatctaaccctgctgtaactggtcgcaagtcacagtttgctcatctataaagtgagcgaaagtaatgtaatctttattgggagtagggaacatgaaggcgagtcgatacagtaagtaagccgccaagcatattgctaggcacagagcaggtgtgcaacaaaagttatttctcaggctttccctcctctgagcgccgtcctccagagggtccggagtgtagctgggggttggagcagcagcctcctaggcgatgggacagagcccacagggtccggtatgccacggtttcttcgtcagaccctgggaatccaacgtcgcaaaataaacacggccgcgccgctaatcgccagttcggaggaaacaaaacagcgctgcgctgggggatctgggcaaaatcagccctccctcctcccgctccttcgccgcggccctcccctcctcgcgctgctctcgttcgcttggctcagctcagctcagctcagcgcagctccgcggccgccaagccgaggcgggcacggtctccgagtcgcggacgccagctccgagctccctctctccgccgcgcctccgccaggtcgcgccttcgtcgggaccacttcgggcaggagtcgcgtggcgaaggcctgcggccgcggcacaaagtt

SEQ ID NO: 96 - DNA, 1200 bp, Homo sapiens
agagggttcaagtttagatttcaggctaaacttctgggcttagagggtcctgagcatgggactggataaaggagggaagccatcatctcttcagaagatgttcagaaaagggcatacacccagtctatctgagctgggagagagctgctgttatgccctggggcagggggatggatgaaatggcatttgagggctcagattctctttggggcaagacctgacttcctatggactcccaggagcagcaggggagttctgagaggaactgtgattgtccagagtggagtttctttgagacttgccctgtacctaaccccaaacagatttgatacttagagacctctaacccggcatctcattgtacagctgggaaaactgagacccaggaggaaggggcctctccgagatgccacagtgagtcaagagtcctcacttcagagggctggagaataatctactgaggcagggccagggcacctgtctgagaggggcttctaaggctgcaagcttttccccttccgtggactgcattcacaccttgttcggtcctgctggggccggggctctccttccttgccccttttccttggcactctttccacaggccctgtgtggtctgggctctaccgagaggtccccaaggagagctggcgtgtcagtcagccaagaagcaagcaggcaggcaatgccagcatgcaggtggcggggctggcaggggagccggcaggagggccggacacccggagccggggaactgtaatgttcagggaatttcaattggcattgctgtcccgttccagccaactggctaacggtgccgagtttgaatcagtgacccgcctcctggtccctcctccttagccggcctctctccctccctccttttttccctccctcggtggccttccaggaggcgggaggcgcccgctgtcgaggcagctgagccccggcaaccgctgctctccgcctctcccctcgcggggccggctcatggagcgcagggaccgggctggctctcgccgagccccgggcctcttttagcctcgtccccagagagggaggagccggtgcccggcacagccccgccggccctagaagctccccacgcgccaccatgcctctgctggacgttttctggtcttgcttcaggaaggtgaaggtaagtgactggccagccccagcccatcccgctgctcaggg

SEQ ID NO: 97 - DNA, 1200 bp, Homo sapiens
agtgtgagaccagcctgggcaacatagcaagacttcagctctacaaaaaatgtaaaaattagctgagcatggtggcacacacctgttatcctagctactcaggaagttgaggcaggagattttcttgaccacaggagttccaggctgggtagctttaagtgagtcatgcttaaagtctgctcagtttctgtctttgtaaatgtgagggatagcagttgaactgcatgaggtcctggatactctcagacctgactaataggagtcctcaattcaacaatgaaatcaaggttgtgaacttgtgttggacccttgtgatgaaaggccagagagaaatcaaagccgaaggctcttgtttcttactgaatttgaatggactttctggtggtgcttactatcagaactcctggaagccctcccctgccacgattaatgttcgtgccccaaattttccatgcatttctctgcggaggtagagttcagacttggggtaggggacagaagcctggggtattggggaagttgcagagccactggggcctgagcaacgtagggatggcagcttcctgctcaaattcctaattatctggttttcccctaccaattggagatttcactgaagttgtgtcttctggacatcccataaattatccgagcaagtcactgaaccctcccgggcacatagcatatgtgtttaaatcccccaaacagtgccggactcattcagaactcaataaatattagcctgtgtgcctggccaagtcactgcttgagcctcagtctttgggcttgcacctcctccctggcagcggtgtgaccggagggaaaggcagcggcgagggccttggaggacctgcaggcagtgccccgcccacccagatttgggtgactttctggcgagtccggtgggccagactgcgcccccattggccgaggggaggtgggcggggcgtccctacagccctactcgccgacgctccctccccacttgagctgagtggactcgggttgacaacggctaagcagccgcggcagctgctcttctggctggcaccgtcacccctgcccgaccccaggccccgcgtgtgtcccggacggagcagtccctcctgggctcgcctcctgcccgctcgctctgtgtcagcgcccgaacccaaaggggtggacgcggcctccaggtaggagtctgggcgacggcagggg

SEQ ID NO: 98 - DNA, 1200 bp, Homo sapiens
gggctgagagagggggagggtcactgcttcttaatggctttctgcccagaaagcctctagaactgcctgctgctgactttctttcctttcagggcatccagagggttcaagtttagatttcaggctaaacttctgggcttagagggtcctgagcatgggactggataaaggagggaagccatcatctcttcagaagatgttcagaaaagggcatacacccagtctatctgagctgggagagagctgctgttatgccctggggcagggggatggatgaaatggcatttgagggctcagattctctttggggcaagacctgacttcctatggactcccaggagcagcaggggagttctgagaggaactgtgattgtccagagtggagtttctttgagacttgccctgtacctaaccccaaacagatttgatacttagagacctctaacccggcatctcattgtacagctgggaaaactgagacccaggaggaaggggcctctccgagatgccacagtgagtcaagagtcctcacttcagagggctggagaataatctactgaggcagggccagggcacctgtctgagaggggcttctaaggctgcaagcttttccccttccgtggactgcattcacaccttgttcggtcctgctggggccggggctctccttccttgccccttttccttggcactctttccacaggccctgtgtggtctgggctctaccgagaggtccccaaggagagctggcgtgtcagtcagccaagaagcaagcaggcaggcaatgccagcatgcaggtggcggggctggcaggggagccggcaggagggccggacacccggagccggggaactgtaatgttcagggaatttcaattggcattgctgtcccgttccagccaactggctaacggtgccgagtttgaatcagtgacccgcctcctggtccctcctccttagccggcctctctccctccctccttttttccctccctcggtggccttccaggaggcgggaggcgcccgctgtcgaggcagctgagccccggcaaccgctgctctccgcctctcccctcgcggggccggctcatggagcgcagggaccgggctggctctcgccgagccccgggcctcttttagcctcgtccccagagagggaggagccggtgcccggcacagccccgccggccctagaagctc

SEQ ID NO: 99 - DNA, 1200 bp, Homo sapiens
tggggaggggctgagagagggggagggtcactgcttcttaatggctttctgcccagaaagcctctagaactgcctgctgctgactttctttcctttcagggcatccagagggttcaagtttagatttcaggctaaacttctgggcttagagggtcctgagcatgggactggataaaggagggaagccatcatctcttcagaagatgttcagaaaagggcatacacccagtctatctgagctgggagagagctgctgttatgccctggggcagggggatggatgaaatggcatttgagggctcagattctctttggggcaagacctgacttcctatggactcccaggagcagcaggggagttctgagaggaactgtgattgtccagagtggagtttctttgagacttgccctgtacctaaccccaaacagatttgatacttagagacctctaacccggcatctcattgtacagctgggaaaactgagacccaggaggaaggggcctctccgagatgccacagtgagtcaagagtcctcacttcagagggctggagaataatctactgaggcagggccagggcacctgtctgagaggggcttctaaggctgcaagcttttccccttccgtggactgcattcacaccttgttcggtcctgctggggccggggctctccttccttgccccttttccttggcactctttccacaggccctgtgtggtctgggctctaccgagaggtccccaaggagagctggcgtgtcagtcagccaagaagcaagcaggcaggcaatgccagcatgcaggtggcggggctggcaggggagccggcaggagggccggacacccggagccggggaactgtaatgttcagggaatttcaattggcattgctgtcccgttccagccaactggctaacggtgccgagtttgaatcagtgacccgcctcctggtccctcctccttagccggcctctctccctccctccttttttccctccctcggtggccttccaggaggcgggaggcgcccgctgtcgaggcagctgagccccggcaaccgctgctctccgcctctcccctcgcggggccggctcatggagcgcagggaccgggctggctctcgccgagccccgggcctcttttagcctcgtccccagagagggaggagccggtgcccggcacagccccgccggcccta

SEQ ID NO: 100 - DNA, 1200 bp, Homo sapiens
aacaattcaaaactggaagcagtccaaacacctattaaaaggtagataaacaaattgtgttatattcatgcagtggaatagtaacagcaataaaacccaacaaactcttgacacataggacgaaatttatcaatcaaaataattatgctattggaaagaagctagattaaatgttcatactgtatgattctattatcgtaaaattctagaaaatgcaaactaatatatggtgacagaaagcagaacgggagctgggaggagtcaggtattacaaccgggcatgaggaaactttgaggtgatggaaaagtttatcttaattgtggaggtgatttcaacagtgtatacatatgttcacccttaacaaattatgcacttttgatatctgcagtttattgtactttaaccttcaagctgttaaaaattaaccggagataattttcaattgttgtgtaaggtttgccacaaagtgtaatatctgcaaagaagcagacaactaattttttcttcttaagattgtggaaaaagttgtaatactacagagagctgtggaatctaaggattagttgaccaacgcttgtaaagctaccaatctgaaaaacacattttcagttaccaaagttattttctctcttccatcgtgtaaatagaatgtcggggttcttctaatttgggtgtttctaaaatttaaatccacgtgaacagattaggaaagaccacgaaatcctacgtcacctctaaagtcacagcttgaagccttgcgaaaggaactggccctagaccccaccccggcctataaatcaccagggcgcagcctcagtgcctgggggtcggcagcagccagcgtcaggcggctcccgcacgcttcttcctaggcggcaggccgtggctgccacgtgactcccgctaacaccggatgatggcgcagcgctgtgcacgcaggcgcagtgtggggcccttgcggtcgaagctcacgcggtaagccgctgcacgtgtgctacggcgggcggagggccgaaagtccagtatgtgggtccagggtcactcttctagagcttccgcaacggaaagtgtgaggtcagtagtagtttttgtggtacgtgcagtggtggccgcttcaaggactattttcgtcgctgcttgcgttttaagtctccaatcctgggacgaggcccatttcagggttttctgtcaccttctag

SEQ ID NO: 101 - DNA, 1200 bp, Homo sapiens
catcctcatgtcgtgtcccgccagcaactataatttccccgcgtgactgagcgccctgctagagttgcccagatttagccagaatctggcagaggcggccccaaaatttggaactcccaggcctcaggggccacctctccagcactgcgcgcttggagtcggggtggacactgtcattctctctcatcgattgattgattgagacgaagtctcactctgtcgcccaggctggagtgcagtggggccatctcagctcactgcaacctccgcctcccgggttgaagcgattcttctgcctcagcctcctgagtaactgagatcataggcacccgccaccgcgcccggctaattttgtatttttagtagagacggggtttcaccatgttggccaggctggtctcaaactcctgacctcaggtgatccacccacctcggcctcccaaagtgatgggattacaggcatgagccacagcgcccggccatttctgttgttgttttttttttttgagacgaagtttcactcttgtcacccaggctggagtgcagtggctgcgatcttggctcactgtgacctctgtctcccgggttcaagcgattctcctgcctcagcctcctgagcagctgggactacaggcacgtgccaccacgcccggttaattttgtttttttttttcagtagagacggggtttcatcatgttggccaggctggtcttgaactcctgacctcaggtgatctgcctgcctcaaccttccaaagtgctgggatttcaggcgtgagctaccgtgcttggcctatttattttattattttttaacaaatgtcattatcttcttaacttttggccccccaaataaatgaggaaactgttaaaattgtattcaacaatccttgttcgatatctcctttgatatttaaacttttaccagtgtattaaaatgatgtctttgcattcagaaggttaaagaaatttgttttttaaagtcaggaagcatctaaatatggtgtcctccggaaaaaagtattccaggaaatctgggaagccgtctgtggaagatcagtttacgagagcctatgactttgagactgaagataagaaagatctgagtggatcagaggaagatgttattgaaggtgcagtacatctcatgttgggcagtgatgtccttcagttcatttgttttgaacccattgagactat

SEQ ID NO: 102 - DNA, 1200 bp, Homo sapiens
aaaaaggcttagaggagtttgtgtgctcaaggtcattacgttagttaatggagaagataggattcaaactcagctgtctacacaaccatctataatgagtattctgtaaatatttaataagtgaatttctctacacacattgcttgtatctttgctggaacacaaggagtgcagtaaatacttaaagatagtgaagtcagggaaagccaggagggcttgtaactaatcgcacatcgctggtgtgaccaggtgtttcaatgccaaagcagttaaaatgtgtcttgtcaacatcatttgagatttttagtatctattattcacttttaaccatggcttgttttgctgttcttactgatttttttagatccaagacaaaataaaagaagttgagcagagcaaggccacgagccaggaactcagccggcaaattcagaagttagctaaagacctcacaactattctaactaagctgaaagcgaagacagataatgtagttcaagctaaaactgaccaaaaggtaagaagtagaattttaaagggtactactgaatgaattaaatagtttttggagtcagttttacttggggatataggataaaaaccttttaggaaccttttttaaaaccaaatagtaacagcaggtggtaaagaaattttgtaactgaagcaacacagatcctcttacataattgatcattataattgaacagtattaataaatatacatgcatgagtgtgtacgaaagaagagcgtcaaaggactaagtgatgatttaggaatacaagtatataaattccaaactgaattgtgtccttggtagctaaatctgtgttcttcctctgttgatgagttcagggactctaatcctttttgggtggggcagaaggaaaatgttagccttctcactcagcctcataggaaataaacaccagcattacaacatatcctgccctgcctttccaaccgaagaaacaaaaatgacctgatcatagtagaattatagtagaattattcattataatatttggctttgacaaaaatcagtctgatctcgggaaacctggagaaatttattttctgtactctaatgttctttcattttggtgaccatcaaggtgctgggagaggaattagatggctgtaattcaaagttaatggaattagatgcagcagtacagaaattcttggaacagaatggc

SEQ ID NO: 103 - DNA, 1200 bp, Homo sapiens
agggctgacctggaataattatgctctggtggttgtctccacaaggggctctgggggtgccccaggattgttgccctcaggcagaggcagggcatacattgagagtgatcccaaacactcctcctccaggcccaacttttggaggtagcaagttctgcaaatagagatccattgtttggtttctctctctaaaaaaaaacaaaagtcagcacaaagaagaggaacacatcagattggtttaccagatttgtttcagatgttattattttgacacgatccagaagaaggagaatttgaagaaaatggaaagctaggcagtgggaatgttggcagcaggtaagggaaaggcaggcagcaaagagaaacttcatctactttataaactgctccttcctttcagcaaatttagaggataaatgactgttttgttcagattggagattcaagggggggattattacttttaagattattgcaacatggcttcgattattttagctgcaatcatcctgttggacttggacttcactgttggtgaagagagactgtgtttgactttgcctggataccataaaaccacaacaaaataaattctcctgaatgctaccaggcaacaccaagatctcttacaccacgagaaggacttgtggccaaagatacaaatggctccgcaatgtacagggtgcatacagtgcacctgaagaacctaaaagtggtttttagaatcatcataataaacagtcaagaattaaagatgtcaagcgctttctcaagaagtctaaactttatacataacattaaaaaagatctataaagcctgaccagtctccctcaaattaagttatttgctactgacttggcagccaaaacgatttaggatctttcggaagttgatgaaaaaggagccagcgggttttgctctttgctctgcatcacgcccttcctgggaccatcagggaacctcccagcctttcaggataggttccgctcagaaaggtcacccgggcagtggaaaccgtgggcaaaagttagctggcaggacagcgcagctcctccaggcagcggaggcagcgcgtcccggctctcagggacatttccttcccacctcgacccccgggaggtggtcccggtataaaggctcgctgagcgggtgggtcacagcacagctttgcagctgcggagaaacgcccaaggccgtgc

SEQ ID NO: 104 - DNA, 1200 bp, Homo sapiens
atatttattaacatcacatttaatcttctcactgtgttacctatggggtattccctgcacttgtttgaaattttctaatcaaaagggtatttcaaaatatattgtaatatgattgatttactttagatggcaagcaaataccactttataatagtctgcatgcattcaaagtaaaataacagtagaaagcctaatctcttttgccattaaaggtttcaatttgtcatgctcagttttaagcttttaaaaaccaacatatccagaaaggcaaaaaatactaaaatcatttcatagcagtaattacattttcctttggatcatgtgcctttaaaacgtctgaatgcattgctgtgcaagtgtcatgtgtgtttaaaagatgcataattattagatgcagttccagcatagtgttttaagtacaagcacccagctcctattcaaggtaagagcctttgacaggcaccaagctgcctctgaatgtggcagcgggatcaaattgttgaatacattgactcatcggtgcgggctggaaattatgcttcttagccctctgtgaaacatgtttttcattttttatttttttttaaactgtatcttaggatctgcttcaaaacacggatgcccacaaaagagcattccatgaaatctaccggaccaggtctgttaacgggattccagtgccacctgatcaattagaggacatggccgagaggtaagagaatatctgcatttaaaggaagtctctttttaaaacactaggtggacgctttttatatttcttttattttttcatgatttttttttaacagaggcgcctacctttggtaaatagaaagcgggctcccataagacatgctaagaatgccaccattctaatgtattattaactgaaaagagtgactctgaatttatccacagcctagaaatcacagaaatggtttcttcagaggctgtatttttgttcagtactgtaaggaacattatgtaatattgcaaaaacagcttataaaagaaacagacgagagatgtgttggcaggggaaagaactaaatcgaagacttcaatcattcatgctttgcttttaaatccagagctagaaaaggaatcggatttccagaaaatgatcagccagagttagaagagcttgtggctgcattctatttaaggctgacatgtaacaggttaagtaagcatcagttc

SEQ ID NO: 105 - DNA, 1200 bp, Homo sapiens
attcaatgtctcccactttataatagcaaatcctagacttcaggaaacagtgggctaaaaagatgtatgactatcattcattttagtcctagtttatttgctattttccttggagtgtggtcttctctctgataatcattttgcctattctcccattttaaacttttcagctctccttaaggcggcagcaaataggtgaaagattgaatgaatgggcagtcttcagtgaaaagaacaaggaactctgtgagtggttgactcaaatggaaagcaaagtttctcagaatggagacattctcattgaagaaatgatagagaagctcaagaaggtatggaatgcatggctacttaggttttcattcttcataaggcttatgtgtacttttcacaggtacagaaggtggctttactcacaattactttcctgtcaaaggagcagtatattttaaacattttatctttttaatataaaataaacttgcagctgggcgcagtggctcacgcctgtaatcccagcactttgggaggctgaggtgggcagatcatgaggtcaggagatcaagaccatcctggccaacatggtgaaaccctgtctctactaaaatacaaaacattagcctggcgtggtggtgcgcgcctgtagtaccagctactctagaggctgaggcaggggaatcacttgaacccagaaggcggagattgcagtgagccgagatcgtgccactgcactccagcctggtgacagaacgagactctgtctcaaaaaaaaaaaaaaaaaaaaaaaaaaaaaaggatatccagcataaaataaacttgcataaaaaggcctgctttacacataaaatatatagaatacttgcaatttgtgatgaaatagagcatctactattcagttcagtatttgggggtcttaattacctgttcagtaggaaccctttatatgtattatttttctttccttttttttttgttttttgtgtatttttttttgttttaaaaaggtaaagccactagagagaaactgaaagaaaacattcttaagataaattgaattgacattttctctctaaaatatgatttatagaccacagataggaattaagagtttcctgataattttggcttcatattattttaaaggtatgtatatcttatcaaattattttacatattcatttggatccaaaacatttctgatgt

SEQ ID NO: 106 - DNA, 1200 bp, Homo sapiens
tgaccttgtgatccaccaacctcagcctcccaaagtgctgggattacaggcgtgagccaccaatgtatttcttttattaagaaaatgtgtttcctgtagtgttctgttttaaaaacagtcaactcacatgaccagaaccagatgctttgttttattttagttacaaatcatcatgtagtcctttttggaaagaacttagcaaattttcattatttatttaatatttattatttatttgtgtgtagtttttaatcccctaaaggatgtaaagcatccagcattaatcatattttattgaattataggaattaataatttggggacttattttctttaaaagtataaattattaaattatcatctgcttagtgcagaaagtttggaaactcttgaaattcacaaaagaaaaagagttacctgaagaccccctgagcaaatgaatatagttatttttttaattgtcttaaaatgaaaatgttatttttgtcatctacatgattatttttaaaggacaaaagttttgcatatgtaagggagaataatatttcttccttatttttcctccccaaatagtctactcaaagataaagagcatgtactttcccaccttcgcaaacaacccctaaaactcttgcatacactaccgagctggcatactgaacccaaaaaagaaggaagttactccttttgctactaccatttgctgtgtttgtggacattttgtttgtttgcagtgggatgatttgttttctttaaatagctttcccagcataacaaactgcccgcaagaaaagttgactgtccttacagaacagcagagtgtgtgcagccgatgttgtttttgctgtcctttctttagacctgtagcttcctcctccttacagattacactgagctttttccccttcttacactcgtggcctacattccaggagatcaagcctggcacatgttaatgaatgtaggttttatttagaaacatgaccacttgtcactgcccagtgtcatgttatcgggcagagactaccaacatttgtctacagtgagcattttcatacttctatacacagacaaggggaaactttcatccccacgcaggatatagctttttgctctgcagagtctaacttttgcaagtggaagcttcatggtggtggcggaggacctgagtgccctgaggatggcagaggacagctgtgt

SEQ ID NO: 107 - DNA, 1200 bp, Homo sapiens
tgccttgtctcatatttagttcattggaatacatatattatgctaacaataataagcatgaaaaaaatttcaaattttttaactccatgaggtaaactaaataaggtaaaagaaattaaactaaggaaattatgacaataaaatgttttgaagacttcaggttttttaaaatgttagtcataatcaagaaaaaaacaaagttgcctatagtggtagattcataaccaatatatttctttaatcaaagtttaaaatattttcatttctgattactaaaaatacattaatataagaagaatgattgttttataaaagaattcacagtacaatatctttaagtactatgttttctagaatttatttcttatataaaatgcagttttctttaattttttatttttttttttgagattgggtttgcgctgtcacccaggctgtagtgcactggtgcagtaatggctcactgcagcctcagcctcctgggctcaagcagtcttcctcccacttcattacctgggactacagatgcatgccactgtgcctggataatcttttgatttttttttttttttttttttgagatgaggtgtcactttgtaacccaggctggcaggtgtatttcctttttttttttttttttttttgagatggagtctcgctctgtcacccaggctggagtgcagtggcgccatctcagctcactgcaagctctgcctcccaggttcacgccattctcctgcctcagcctcccgagtagctgggactacaggtgcccgccaccacgcccagctaattttttgtatttttagtagagacagggtttcacagtgttagccaggatggtcttgatctcctgaccttgtgatccaccaacctcagcctcccaaagtgctgggattacaggcgtgagccaccaatgtatttcttttattaagaaaatgtgtttcctgtagtgttctgttttaaaaacagtcaactcacatgaccagaaccagatgctttgttttattttagttacaaatcatcatgtagtcctttttggaaagaacttagcaaattttcattatttatttaatatttattatttatttgtgtgtagtttttaatcccctaaaggatgtaaagcatccagcattaatcatattttattgaattataggaattaataatttggggacttattttctttaaaagtataaa

SEQ ID NO: 108 - DNA, 1200 bp, Homo sapiens
tgtttgactttgcctggataccataaaaccacaacaaaataaattctcctgaatgctaccaggcaacaccaagatctcttacaccacgagaaggacttgtggccaaagatacaaatggctccgcaatgtacagggtgcatacagtgcacctgaagaacctaaaagtggtttttagaatcatcataataaacagtcaagaattaaagatgtcaagcgctttctcaagaagtctaaactttatacataacattaaaaaagatctataaagcctgaccagtctccctcaaattaagttatttgctactgacttggcagccaaaacgatttaggatctttcggaagttgatgaaaaaggagccagcgggttttgctctttgctctgcatcacgcccttcctgggaccatcagggaacctcccagcctttcaggataggttccgctcagaaaggtcacccgggcagtggaaaccgtgggcaaaagttagctggcaggacagcgcagctcctccaggcagcggaggcagcgcgtcccggctctcagggacatttccttcccacctcgacccccgggaggtggtcccggtataaaggctcgctgagcgggtgggtcacagcacagctttgcagctgcggagaaacgcccaaggccgtgcatctccaggaggtagggaagcctgcgagcctccgggtccccgcccccagctcttccaggcaccggcgcatcccggagccgggcgcggggcgcggggcacggagccggggagggggtgtggcgggccagggcggtgcagggaccctgcccagaggctgaaccggcctgggcgaccttccttcccgacaggagcagtagatgcgtgggggagggcgaagggggaggcctcaccccccagcgagcagccgggaggaggaaccggtcgcggagggcggggagcgcggctgagcagcccggcgcgggagtgagtgtgggcgagccagccagggtgagcgcgggcgccgggccgcggccgccggccgcagcgggctgagattgttgtcctctgtcaccagggcggctgggctcccgcagtcctgcagaccgcgcccgatcccggcgacagggcgggcggacagccgcgcatccccggggtcccgccgagcctgggcgcagagagccgggaggaagcgttcgctcgcttcgccttgctgctgggaaactgaacga

SEQ ID NO: 109 - DNA, 1200 bp, Homo sapiens
tgttttattttagttacaaatcatcatgtagtcctttttggaaagaacttagcaaattttcattatttatttaatatttattatttatttgtgtgtagtttttaatcccctaaaggatgtaaagcatccagcattaatcatattttattgaattataggaattaataatttggggacttattttctttaaaagtataaattattaaattatcatctgcttagtgcagaaagtttggaaactcttgaaattcacaaaagaaaaagagttacctgaagaccccctgagcaaatgaatatagttatttttttaattgtcttaaaatgaaaatgttatttttgtcatctacatgattatttttaaaggacaaaagttttgcatatgtaagggagaataatatttcttccttatttttcctccccaaatagtctactcaaagataaagagcatgtactttcccaccttcgcaaacaacccctaaaactcttgcatacactaccgagctggcatactgaacccaaaaaagaaggaagttactccttttgctactaccatttgctgtgtttgtggacattttgtttgtttgcagtgggatgatttgttttctttaaatagctttcccagcataacaaactgcccgcaagaaaagttgactgtccttacagaacagcagagtgtgtgcagccgatgttgtttttgctgtcctttctttagacctgtagcttcctcctccttacagattacactgagctttttccccttcttacactcgtggcctacattccaggagatcaagcctggcacatgttaatgaatgtaggttttatttagaaacatgaccacttgtcactgcccagtgtcatgttatcgggcagagactaccaacatttgtctacagtgagcattttcatacttctatacacagacaaggggaaactttcatccccacgcaggatatagctttttgctctgcagagtctaacttttgcaagtggaagcttcatggtggtggcggaggacctgagtgccctgaggatggcagaggacagctgtgtggatgcagatctcccagattgtaactgcgatgtcacaaggcaggcatcttcttgagtttcttccattgtacatagctttcatgttacgtggggtttgtaggatagacgctggaagccttaatccagaactgtttttgccaatgttac

SEQ ID NO: 110 - DNA, 1200 bp, Homo sapiens
ggagagacccgaaggggagcggaggttggggtagaagcccccggccctcctgtattactactgcggaatgcgggggaccccgcagacagccaggctcccaggacggggcacccttggggcttcagtttccctcgcatctccaagctcgctctgcgggcgatttgccaggagaggggtccatggagagagcgggagacgaggggcgcccggctgaggaatagggcttgcctcctagcgagtgtcgaggtccccgggtccgtgcgtcggtctgtccgtccgtttgtgtgttctcgcggtgaacgcgggcggacccccagtctctccgggagaaacttgggtctgcggctggcacgaggtctttgtgtccgggtgtctctccggctgcgagtggttttccgcgtctccgctggggggcgggggacagggggcggggtgggggagggtatgagggtgttgcggcgtccttcccccaccccctctcgcccctataactcgctccctccgtccctctcggactcggactcggggccggtgcgcgactgagctggcgcctctagccgactttgccaagagggcgcctcctgcccgatccgcgctgaccctgcgcgggacgcagccccaggcggctgcctagccgaggcgggagcccgagctgagtcgagctcgccggagcgggtgagtcccgagcccgatccccgccgggctggaccctggcgcgcagctgcggcgtctccggggagtcgcggcagggggaatggcgtgtcgcgccctggcggcgggagctgcgggagggtcggcgacgtggcggccggcccagggtatttacctgctgcaccagtgcccggcagccacgctcgccggctgccgcggggcttctttgtcccgatatctggagggtccggggcgggagggagtactgggtcgcgtcccctccatgggggaggggctgggggagtgagaggcctggagataatgcggagtgggggtggggagatcctgcaaggaccaggacgccggggttccaggagactctgcaggctccatccctccttattcccccaccccgaccccagtagcgctgggcctaggagcactcgggagttgctcagtggctctgcaccgctggcgcgggaaggggagaaaggggctgccagaggcgggaatttggtccccacgcgctgctccagggacgagaagcca

SEQ ID NO: 111 - DNA, 1200 bp, Homo sapiens
ggggccccaccgccgacaggtgtagatcaagagactcccgagataggcccctgcagggcgctggtcccccggcctccctgaccatggctcacagacttccagcgctgagtgaggcagctcgaagcctctcagccgatctgttcctgggcctcactccacagtttctgaggggatctctactttaagtctagcctcaatcctcccgttgagctttctgtctttcttgttatgcttagcagaaagatacatgatctctggtgctgtttcaccgaagcaaggaggtgggtcggcggaactgagcgccctgcgccctcttgaaatgaccccccattcctgtttgccagaaacttttccctcccgttgccatggagagagacggccgctgcctcccgtcgccatagcaacatgcgggtaccgcggaccgcaggattgcggagctcggaaagtcttgcagttcgcatcgcccgcctgtcttggggtcttctacctccgaagctgggactcgggggccagtctaactgctcccgtcaggaacctcggccaaggtcctcccagaccctgcctgaccgtcgctctttctgttacggggttatctatttctctgtcattcgtgtggcatgtggcagtttacccagtgttttcacaaacacgatctttctatccccagggagtccgtgaagtacttaaaatcattacacctcctttacggagaggaaaattgaggctcagagacgcccaaggtcacaccaggcagtgaatgcggagtgggaacccggactctttcctggccttgcaccctccgagattcgggacagcccggcttcccgcctcgaaacccccaccccccggactcaggccagtccctgagtgtagagggggcggccgcgaggcgccagactcgccagctgccgcgggatgccacgtgaggggcggggaccctgcggggaagtgattaaaagcagagcggagccgcagggggccgctgcagggagggtcttcttggggccgtcgtgccccgccccagtgcggccggggcgcgggttcgagctgctgctcggcaagcctgggtgtctagggcatgagcggagtgtggggggccggcgggcctcggtgccaggaggcgctcgcggtcctcgcctcgctgtgccgggcccggccgccccctctcgggctggacgtggagacttgtcggagcttcga

SEQ ID NO: 112 - DNA, 1200 bp, Homo sapiens
aactgggaaagagctggaagatgagacaccaaactccatcccatggtgatgtttgaaccaggctggagctggtttcacatgtttttcattgtgcatgactttcttaattgccttagtgggaatgcgttactgcaaaataaagtgaaattgttttccttattgcttgtaatttacagaggggtggttttgatcatataaaccaggcacttttcaaatccattgatcttagggtggaaggagggatttctttttaagtattcatttcgaactatgctttcactagtaaactcaaagcccaagggcaaaaacccatgattctccattttgtaatagctattagatgtccacagcacatcagacctgtggaaaacgcatgctttgtgtatggaagagggagagggagcgtcaggagttgtcctttgccaaaaaacgtactctgacactcaaaggcacaggctggtggagatccagggaacccgggaaagcccaggcctaactcaggcccgcgcgggtctcttctgatcccataagatgaggagtgggtcctggctctgggcatcctggtgtcgactgtgagttcccagcagaggcccagagtcccggtccggcagccgagggaagcgggggggtcttccagaagaagaaagggccaaggtcaccccggtgcctctccagcagcagcagagggcggcggtcggtgtcgctgctggccggggcctcgaggaaggcgcgggccagctggggccgggtctgcgttcccaggagctgccaccgttccagggagcaagtcaggccgggacgtagcgcctgcgcgggaccctcacttgccaccaagacccccacaaaccccgccccatcctgccttacgccccgccccaaggtcgttctcccgacccggggtcccgccccaaggccgtcctcccgccccgccgcttggtggcggccgcatgctgcccggatataaagggtcggccccacatcccagggaccagcgagcggccttgagaggctctggctcttgcttcttaggcggcccgaggacgccatggccgagtgcccgacactcggggaggcagtcaccgaccacccggaccgcctgtgggcctgggagaagttcgtgtatttggacgagaagcagcacgcctggctgcccttaaccatcgaggtacagccagctttggagcggatggcgaggcagca

SEQ ID NO: 113 - DNA, 1200 bp, Homo sapiens
tctaatctatggtaatcgaaatgagaagagtgtttgcccctggcctaaggatggaaggattgatttaggaaaaagtctgcaggagctttctggagtgatggaaatgttctaactcttttttttttcttcttcttcttctttttcttcgtttcgagacggagttccactctgccgcccagactggagtgcagtggcgccatctgggctcactgcagcctccgcctcctgggttcaagccattctcctgcctcagcctcccgaggggctgggactacaggtgcccgccaccacgcccggctaattttttgtgtttttaatagagacggggtttcgccatgttggccaggctagtctcgaactcctgatttcaggtgatccacccgccttggcctcccaaagtgctgggattacaggcgcgagctaccgcgcccagccaaaatgttctaattctttattggtggctacacagttgtcaaaatttatcaaactgtccacctacgatatattataagcctgtattaaatgtaaattagaactcgattgaaatctgtgtgtataaatataaatatacacttatttacatttctaatctaatctgaaggtccattgcaacgaatcccgctccgctctgacccaagaactccgcactttctctccaccagcctttcaaatacctgaacttcatgccttcaagaggtggattccggcttctttacagcgcaatcactagcaggtcatttccgtctagcttgcagatttcccattactgacacaaacgctccctcagggcgtccccgtctggactacaggagaaagtttgggaggcaggttcaacttttcaacttggcggggaattcctctccctcttacacagtttgcagcgcgggggcggcggggtgacagtccccgtgcatgaatcagccacccctcaggctccgccccggcgggggtcggccggacgctcgccccgcataaagcgggcacccgggccgcctggagcagaaagccgcgcacctcctcccgccaggcgctttctcggacgccttgcccagcgggccgcccgaccccctgcaccatggaccccgctcgccccctggggctgtcgattctgctgcttttcctgacggaggctgcactgggcgatgctgctcaggagccaacaggtatctggccgctccaggagcttctctccccaaccgg

SEQ ID NO: 114 - DNA, 1200 bp, Homo sapiens; identical to SEQ ID NO: 113

SEQ ID NO: 115 - DNA, 1200 bp, Homo sapiens
cccaaattgttctatagattcaaaacattgccagttaaaatctcagtggactttgtttttttgaaactgacaagctgattctgccatttataataatattcaaaggggcaagaataaccaagacacatttgaagaagaacaaggaggaagaagaacgtatttgaagaagaataaggaactctattaagtgtatatctcctatatattgagatacacatttgaaatacatctatcacctagatattgagatatattatacatttaatatagtgtgttgctggtatagggatagacaaatgaacattggatcagaatagcactgaaacaagatctacccctatatgggcatgtccctattaacaaaaaaatattgttgattatttcctcaagatgatctgtaaaaaacaagttaattgaacatccatatgggaaaaaagtgaatccttctgctacctcaaatatcaaccccaggtagattatagatctaaaagtgaaaggcaaagcaataaagattttagaaaataacaaagagaatatctttatgaaaatacaggaaatattttaaaaataggctagaaaactcactattaaaatatataaaatataagaacatctgttcatcaaaagtactatagtttgaatagcagtgtctcctccaaaattcatattgaaacttaatccccattgtaacagtactaatcctaaaactaagtattaagaggtgtgacctgtgggaagtgactaagtcatgaggggtctttcctcaggtatgggattagcatccttataaaaggccttgagatggaatgtagtgcccccttgccctcttgcgttctcccatgtgaggacacagcaacaaggcaccatcttggaaggagagagaagccctcaccagacaacagtgcacatgccttgatcttagacttcccagcctccagagctataataaatacatttctgttctttataaattacttggtctctggtattttgttacagaagcactaatggactaagacaaaaagattccattatgaaagtgaaaaggcaaccgcagagtggcagaagcttttggagatgatgaatatgtttacttcctggattgcggtgatgataccatgagtgtatacatatgtgtgtatacataatgtatacaaattgtgttcattgattatgtacagtttctttgtataccaatta

SEQ ID NO: 116 - DNA, 1200 bp, Homo sapiens
gggaaagtctggaagaacacactcttctggatctaatctaagttttctcactacccacctatcatgagtacctttttcagtctctctaggccaagggtttcttatttaaaaggaccaagagactccttgatccctaagattcctgctaagattctgtacctctttcctcccaaaagaatgactggtcttgaattctaaaatgcaatttttgtgagtgtgttttaaaaagaagactgcatttcgctgaaaaatcttccttggaaagtatataaatcttattagacgcctttgagagtcattaaaagaactgaagcatcatgcacgtttaaaacattaccaggctggatggagtacgaaggcactcaggggaaggtacttaaacctccgtaggttcaaccaagtcttaccgggagtaacccaacgaacaagtgatgctttttgagacaaatttggagagttggctgcaggaaaattaacaagccagtggatttccttttcccttccttaggaaacttgtttctgtagatgcaaaaagggccctagatcctctggaagagaaagagagtggggaggggaagtttctctttctttactccccacctgcggtctgtctgatttcctctccagctctgtctgataaggaattgattctccccctcctcttccccctcccgccctccctctccttctccctgagtaacgatgctgtcctagcaagtgatgctgtcggagacaggagacgggcgccgaggaggcatcgccgccgccgcggggctggagagcctctcccagcaccagagccccgctcggccccgggcttcctcgtcgcagccacggccgcggcagctgctcccacggtttgatggtgggcggcggcagctcggcttcggcgctagcctctaactgctggatcgcgggccgcgacgctctccgctcctgccttcccgccctgggccgcccggggccccggaagccgcgggaggtggtgaaggggcgccgcgggaagactgcactgccgcgaaggcggaggaggccggcagccggcacccccacactcggaccgcagccggcgcgatgtccaccaaggtcaggaagtgcaaagaacaagcaagggtgaccttcccggcgccggaggaggaggaagacgagggcgaggacgagggcgcggagccgcagcgccgccgccggggctggaggggc

SEQ ID NO: 117 - DNA, 1200 bp, Homo sapiens
tctccaactcctaggctcaactgatcctcccgtctccgcctctcaaagtgctaggattacaggtgtgaggcactgtgcccaggtgagaggtaagatttcttaaacagagcagaaaaacactaaggtaaaggggaaaatgacaaattggcacatattaagatgaaaaacttttgttcttcaaaaggtaccattaagatggtgaaaaagtaggccaggtacggttgctcatgcctgtaatcccagcactttgggaggccaagatgggcggatcacctgaggccaggagttcagaccagcctggccaacatggcaaaaccatgtcttgactaaaaatacaaaacaaaacaaaacaaaacaaacaaacaaaaaaaccgggcatggtggtgggcgcctgtaatcccggctacttgggaggctgaagcaggagaatcgcttgaacttgggaggcggcagcttcagtgagctgagatcatgccattgtactccagcctcggcgacagcgaaactacgagtccaaaagaaaagtaaccccacagagtgggaggaaatatttgtaatacctatactggaagagaaatatttgtaatacctatactggaagaatgactgactagtatctagcatgtacaaaggcttcctacaaatccatatgaaaaataacgtcagcccagtagagaaatagggagaagcctcggatactgcacaagagagaatatcctagtggccaacaaacacatgagaaggttctcagctcattagactttggctaaatgcaaattaaaaccaccatgtaatatcattactcatccagcacgatggctaaaataacaaagacgtaaagacaccaccacagactcgcgagactgaggagtgagtgtcgcgagactgaggggtgggtgtcgcgagactgaagagtgggcggcgcaaatgggcgaggtgtttactcgccattttgtgtgaggaacgttgacgctacctacctctggcctggcctctacccactgctgccctggccgctaccaacatgagcctcccggagagtcctcacagccccgctactctcgactatgctctggaagacccgcaccagggccagaggtcccgagaaaagagcaaggcgacagaggtaatggcagatatgtttgatggccgcttggagccaatcgtgttcccaccgccccggcttccagaggagggggt

SEQ ID NO: 118 - DNA, 1200 bp, Homo sapiens
atctgttaacctcaacttcctaaatagaaggtctattcttttaccatcatcattattctcttcggtgcctatttttaaaaaatactcaaccttcttgcttccttcgctacctaagtatttctgcaagcccactttgttctgcagcttagctttcctggcacaattcttatagattttggtcccttttaaaattcattcttcagcaaatgctttctctctccatcttttgactagagatcattagagatcacctgagatcattagagaacagtggtttccttggttgccattccctttcttcttcattgggagtattctgcggtgaactcagacattttatttttcaaagcttcccattcttttaaaaatgcttttccttttacagcctctcgctcaaaatcatacccatcttttccctggatctgttttctcaagtctccaatcgcctgccttctttgtgtcttgtattaccctcacatcccccagctttctactgctctcccaggaccaaccatttcttccgcgggagtcacattacatcagcattcctaatgcagtatctgttatctaccagattctgttttattctaggtagtcacttaaaaacgaacctcggtactggtctgacttaacatggaggaggaattgtctaaggttaaacgcaaactgctgagagatttggggcggggggcacacatttacattcattcgtattaaatatatacctgttgaatttgtgctttttctcaaatgcttcagagactcgagctttagagtaattgggatggtgaaaggatgggtttccagaaacttcgcccaaaattaaagactccatcaaaaggactgctccatacactcaaggaacacccaccaacaaatcccgtctccacaaccaccagattatctcaccggcgagtgagactgcaaggtttgggggcccggccgtaccactccgcgctgcgcacggggggttcgtacccatctggccgcgaccgtccgtttccccctcgcttggttctgcccctgctccccctgcacaggcctcacagtgcgtctggccggcgctttatagctgcagcctgggcggctccgctagctgtttttcgtcttccctaggctatttctgccgggcgctccgcgaagatgcagctcaagccgatggagatcaaccccgaggtgagcgccaggtgcaccgctacc

SEQ ID NO: 119 - DNA, 1200 bp, Homo sapiens
aaaatactcaaccttcttgcttccttcgctacctaagtatttctgcaagcccactttgttctgcagcttagctttcctggcacaattcttatagattttggtcccttttaaaattcattcttcagcaaatgctttctctctccatcttttgactagagatcattagagatcacctgagatcattagagaacagtggtttccttggttgccattccctttcttcttcattgggagtattctgcggtgaactcagacattttatttttcaaagcttcccattcttttaaaaatgcttttccttttacagcctctcgctcaaaatcatacccatcttttccctggatctgttttctcaagtctccaatcgcctgccttctttgtgtcttgtattaccctcacatcccccagctttctactgctctcccaggaccaaccatttcttccgcgggagtcacattacatcagcattcctaatgcagtatctgttatctaccagattctgttttattctaggtagtcacttaaaaacgaacctcggtactggtctgacttaacatggaggaggaattgtctaaggttaaacgcaaactgctgagagatttggggcggggggcacacatttacattcattcgtattaaatatatacctgttgaatttgtgctttttctcaaatgcttcagagactcgagctttagagtaattgggatggtgaaaggatgggtttccagaaacttcgcccaaaattaaagactccatcaaaaggactgctccatacactcaaggaacacccaccaacaaatcccgtctccacaaccaccagattatctcaccggcgagtgagactgcaaggtttgggggcccggccgtaccactccgcgctgcgcacggggggttcgtacccatctggccgcgaccgtccgtttccccctcgcttggttctgcccctgctccccctgcacaggcctcacagtgcgtctggccggcgctttatagctgcagcctgggcggctccgctagctgtttttcgtcttccctaggctatttctgccgggcgctccgcgaagatgcagctcaagccgatggagatcaaccccgaggtgagcgccaggtgcaccgctacccggagagcgcgaggccgagggagggggagccgagtcgctgatcggttcggttttgcctttttctttgcatttgccttt

SEQ ID NO: 120 - DNA, 1200 bp, Homo sapiens
aaatactcaaccttcttgcttccttcgctacctaagtatttctgcaagcccactttgttctgcagcttagctttcctggcacaattcttatagattttggtcccttttaaaattcattcttcagcaaatgctttctctctccatcttttgactagagatcattagagatcacctgagatcattagagaacagtggtttccttggttgccattccctttcttcttcattgggagtattctgcggtgaactcagacattttatttttcaaagcttcccattcttttaaaaatgcttttccttttacagcctctcgctcaaaatcatacccatcttttccctggatctgttttctcaagtctccaatcgcctgccttctttgtgtcttgtattaccctcacatcccccagctttctactgctctcccaggaccaaccatttcttccgcgggagtcacattacatcagcattcctaatgcagtatctgttatctaccagattctgttttattctaggtagtcacttaaaaacgaacctcggtactggtctgacttaacatggaggaggaattgtctaaggttaaacgcaaactgctgagagatttggggcggggggcacacatttacattcattcgtattaaatatatacctgttgaatttgtgctttttctcaaatgcttcagagactcgagctttagagtaattgggatggtgaaaggatgggtttccagaaacttcgcccaaaattaaagactccatcaaaaggactgctccatacactcaaggaacacccaccaacaaatcccgtctccacaaccaccagattatctcaccggcgagtgagactgcaaggtttgggggcccggccgtaccactccgcgctgcgcacggggggttcgtacccatctggccgcgaccgtccgtttccccctcgcttggttctgcccctgctccccctgcacaggcctcacagtgcgtctggccggcgctttatagctgcagcctgggcggctccgctagctgtttttcgtcttccctaggctatttctgccgggcgctccgcgaagatgcagctcaagccgatggagatcaaccccgaggtgagcgccaggtgcaccgctacccggagagcgcgaggccgagggagggggagccgagtcgctgatcggttcggttttgcctttttctttgcatttgcctttc

SEQ ID NO: 121 - DNA, 1200 bp, Homo sapiens
tgcttccttcgctacctaagtatttctgcaagcccactttgttctgcagcttagctttcctggcacaattcttatagattttggtcccttttaaaattcattcttcagcaaatgctttctctctccatcttttgactagagatcattagagatcacctgagatcattagagaacagtggtttccttggttgccattccctttcttcttcattgggagtattctgcggtgaactcagacattttatttttcaaagcttcccattcttttaaaaatgcttttccttttacagcctctcgctcaaaatcatacccatcttttccctggatctgttttctcaagtctccaatcgcctgccttctttgtgtcttgtattaccctcacatcccccagctttctactgctctcccaggaccaaccatttcttccgcgggagtcacattacatcagcattcctaatgcagtatctgttatctaccagattctgttttattctaggtagtcacttaaaaacgaacctcggtactggtctgacttaacatggaggaggaattgtctaaggttaaacgcaaactgctgagagatttggggcggggggcacacatttacattcattcgtattaaatatatacctgttgaatttgtgctttttctcaaatgcttcagagactcgagctttagagtaattgggatggtgaaaggatgggtttccagaaacttcgcccaaaattaaagactccatcaaaaggactgctccatacactcaaggaacacccaccaacaaatcccgtctccacaaccaccagattatctcaccggcgagtgagactgcaaggtttgggggcccggccgtaccactccgcgctgcgcacggggggttcgtacccatctggccgcgaccgtccgtttccccctcgcttggttctgcccctgctccccctgcacaggcctcacagtgcgtctggccggcgctttatagctgcagcctgggcggctccgctagctgtttttcgtcttccctaggctatttctgccgggcgctccgcgaagatgcagctcaagccgatggagatcaaccccgaggtgagcgccaggtgcaccgctacccggagagcgcgaggccgagggagggggagccgagtcgctgatcggttcggttttgcctttttctttgcatttgcctttcagatgctgaacaaagt

SEQ ID NO: 122 - DNA, 1200 bp, Homo sapiens
ccccagtctagaaagaacctggcgtgtagcaggatgcaacatatacttgtagaatgaataaaaaggtaataaaataacacgagctcacatagcggccagcatttgctgtgcaccataatccgtgctaagcactttgcatgcgttatttaatttgaatttcataacagtattacataagaaaacaatagggccaggcatggtggctcacgcctgtaatcccagcactttgggaggccgaggcaggcggatcacgaagtcgggagctccagaccagcctgaccaacatggtgaaaccccatttctactaaaactagaaaaagtagccaggcgtggtggcgtgcgcctgtaatcccagctacttgggaggctgaggcaggagaattgcttgaacccgggaggtggaggttgcaatgagctaagattgcaccactgcactccagcctgggcgacagagtgagactccgtctccaaaaaaaaagaagaaaaagaaaagacagtattttgctccctcaccgccttcaggcttttaaccaactgccacttaccaccctagtgaaggtttgcccgagtgccctatttaatattgcaggcttctgtttcctaccagcccctttgcttactctttcacacttagtcaccttctaacgtggcatacattgcttattttctatgctcatgagtatgtctactccatggaggcatagatgtttgtatgcctgtttactgatggatcacaaactgtgtagcctaaaatagagctagcacatagtcccacccaacacgtgtcaggtgagcaattagatatagagggttccaataacttgtggggacacccagataagatctgtttatgactcatgtcttacacagagctcattcttggtctcttggttagtttatcagagaagacaaatgtattattggaaaatgtgtcttaggtcattttttgacagtggctctaatactggaatctattttaactaaaggttattatcataaagcaggtgtttgctgaagacagcttactcagatcactactgcctggaggtggttgatatatcctggtgtaaaccttcaagaagggcacaggcaggaaaacatgagccagcaactgaagaaacgggcaaagacaagacaccagaaaggcctgggtggaagagcccccagtggggctaagcccaggcaaggcaagtcaagcca

SEQ ID NO: 123 - DNA, 1200 bp, Homo sapiens
cctagctcccctttctgtcagttttttaattttatatttgttttcaaatgagtcctcacattgattttaaaaatttaacaagagtatagtgaaaaggaagttcccctttcactatttatcttccccatatgcatctattatgacgggtttcttttttaaactacttggagtactcataactgattttgtactctagttcagaagaaatgtctgaatgttaaatgtttacataaaaaatgctattttattatatcagtaatggagcttcccccctttaaaactgctgcaccaggaagatacaaccttgttatcacaatttaaagagaaaatgagacttggaggttcagattcctccacagtcatagtatcagctacccacgcagtgacccacattcaggtgattacacacactcatacacagatccatacacgatcttgccccccccacctccacacactcaatatgacctccccacgcccaaagctgcacacatcttgacctcacttctctcacacacacaccaatctgacccgcacgcggtttctttcactcaactacccagacacccctcatcccaccagcacaagcaggcccaccgcggtctcacacacactcgagccttcctttgcccagtctcacatgcatgactgggatcgcggatttgtccccttccgactccccatgctggttcggtcccagacgctcaggtgccacattggggtgcgctctcccggctcctccacacgctacgcagctgcccgcctcaagagcgtctgcgtgcttccagcgttaagtggaattccaagcagcctccggggcacagttagtctggtcactgcggccagactgccgcctcaggacccagagaacgcagcacactttagttgggcccatctatccccggcgaggcttggactacgtttcccaggagaccctgcggtccagtatgtctcccgaggaggggcggaagcccgggagccgccctctgaggctgggcggggtgaaagaaagcttgccccagaggacttaaacaggcaagaaggacttggttaaagactattgcaatagtcaacttccaatacaacagcagctggagatttatagctaacgggctgggtgaaggagttaaaggatgctaaattactaagaggaagttaagtatcaagagtggggggattgttactaaactggcttagctg

SEQ ID NO: 124 - DNA, 1200 bp, Homo sapiens
acattgcctcctgtcttgtcttccctttcctcttatgttaaaaccagattctgttcctctgctgtagcccttactgagctccatcttctctcaatgttatcatcatagaatttggaggcaaacttatttcttaaatggcataaacaaaccacattctaaccacagatgaatcttccctctccatagctcattccatactcatttagcacattagtctttcattgggaaactgctctttgtggattatctttcttttctgtctccatcagtttatatgcaaattatgtttattactgaaacatcccagatttcccaccccaggaaaaatgtagtactcccaacaaccctgataaatgaatatgagaattactctcatttataaatgtttatagcactagattcatctcttaaaaaattaagacagggccaggacatctgtataattaccaagctttcaaagtctgcaggacttttaaactaaactaaactactatttaaaactactattttaaataaatatctaaactactatttaaaaataatctgtttcctgaaaacctcctccactttctagtctgacttttactcattttctctgcttccctaatctgtttttcaaattgcccagtcatatgtgagaactgtataaaccactcaatttttatgaaaattaaattgtagttaagtctttcgtatctattataaacgtttaatctctcccagaacacaaaaaagtgaatgtttgcttttatattttctttcagactatgaatcaaagttgagaccaagaaattatttctgaaaaaggatatggaaaaccttacaaaacacagcattgagtgttcaagtttcagaggtgattgggaatgtaaaaaccagtttgagagaaaacagggatctcaggaaggacatttcagtgaaatgatatttactcctgaagacatgcccactttcagtatccagcatcagagaattcatactgatgagaaactccttgaatgtaaggaatgtgggaaggattttagttttgtatcagtccttgttcgacatcagcgaattcatactggtgagaaaccttatgaatgcaaagaatgtggcaaggcctttggtagtggtgcaaaccttgcttaccatcaaagaattcatactggtgagaagccttttgaatgtaaagaatgtgggaaggcctttggtagtggc

SEQ ID NO: 125 - DNA, 1200 bp, Homo sapiens
ggccgttatgaagaggaagaagtactgtgtcatctattgtcctggggacctgtcaacaggaacaatagatagacatcaccaggggtgtatttctgttcaacacaaggaaaagtttctaattggagctgtccagcatgaaatggactaagcagtgcattttctttctccgatcctgactccactgtgacgggcactgcagactagagagattgtgtcctagtatgaatttaggttggataaatgatacttagggataaagagaggaaaggaatatcagaatctccaagaagctgatacattttgaaaaaatatataaaatgccacacttcaggttttcttgtgtttaatttgaaacgtccagtaacattaaagaacagcatttgatttgcttaaacaaagtagtaacagctgaagttccttccaaatcacttttctgattctctaagaggcttgcctatgccttgcagatctagcttcacctacaagacatattctgtaaggatccaggttcagcaaggtgtcagaatgtcctaagggtttctgtcccggcgctctgcctgtcccgcttgtgcacaatgcttttgcctatccctgtttctgcatagattttagtcttcccaccagggacgcaactgtcagtttcctccgtctctctcggttctattccaatctgctcatctcccctcccgtccccgctttccccaaggacctactgagatttcgatcccacaacttagtgggtcccatctcacccaggccccgcaatcctcgccttctacttgtcagcgttgtactacactctgagcaggcacccctgtgctactcacggaagcgggaccattaggtcggccattcctcctcaccccacgacacgcacccactgggtcgggccgcctccttgcagggagtctcaaggatgggctggcgtcagggaggggcccagggggcgctttcccaacactggtcgcagtcattgttggtataacggctagagacgcccagtgagttagcatggagggcagtgggaccggaaaaagacgtggaaaagctgcgaaaacgagccttcgaatcatggacgcgcgggcccagctcctcctccgagttcctcatccggggccgtcactcacatccggggccctcactcacatccgggaccctcatccggggctctcacccacatccgggaccctcatgcctggg

SEQ ID NO: 126 - DNA, 1200 bp, Homo sapiens
ucguutuututuuattttauagaggaggaaaucgagggttgagcggcgaatgatttguuutguutguuuaguacggaaaaaaatuaututtutgguuuuagtcguutuuutttutgtgutgtgagtutgtgtuuagaguaggaagggutgutucgagutguagggacgggututgtgtgttaaauuttuaaatgguttuuagggautuatggaguatuuaggauauutggagatggcgautuutgggauuuutgguuuttuuutuututgguuttcggtucgtgagggtgaattaagtgtguuauuuauuagggguagagauauatggagauagagattgagagauagagaauagagggggagauagaaagagagagggauatgaagaguaacggagttauaggaggatutgacgutucgttggggagauagggagaaatgtggggagatguagcgtuuuaggtgutuuuuagtgguuutguuuutuutuuuututuuuutuuuuauuagguaggauuutuagggagutuuaguutggauucgaauuutgtuuuauuutgacguauutguauuagguuaggutggauutgtggggtgggtguagguutgguuuuaguucgguuautguuutggaaauuuutuuuucguuaagatgutgatgtaauatuuaggatttgatgttttaututuuaattutguaaatuuagacguaggaggttttaagaggucgttguauuutguaataaagtgcgggauauaggaatuttggtuutcgggguauuuuuttuuutcgggggggtttcguuuuucguautuacgguuuutgaguucgcgcggagguucgacguuucgcggucgacgucggtuuucguuucgggucguuattggutgttguuatgguauuaguucgtuttuucgcguuutuattggutgtutuauucguatuuutuatuucguatuutuuuucgutcgcggtggtutcguuuagcgutaggagcgguagcgucguutguuuagguucggaucgggutttgtucguuucggaguuuutguucgcgucgcggagauuucggaguucgcgcgutucgagguuauuucgggucgggatttucggtgggguucgcggagucgcguagagggaggagguuuuagauuuaggcguuucguuaguuuagutguacgtaagcgggtgtgtgtuut

SEQ ID NO: 127 - DNA, 1200 bp, Homo sapiens
aaaautgtuatggttauaguuatuaaattauaggaggttuauauauaguagtuaguaatggtttgggaatattaaataaatgtgtttututaauaggauaagauaggatuatttatttatguaauttaaaagutgtuuuagaaggagaaaaaagtagcgtattatttgtuuaaaataaauaagattgaagaautgaauatutututgtggatuaatttauagatuauatggaaatagguttatuttattgagaaatattuuattttagatgttuaaatauuttaguatattttaaaaautatttuutatgtgauuaaaagaattuatgttutattcggtaaaaattaauuaaaattaacggtgutttuuaatggacguaggaaauuttttttutttauaaaatatguuuauuutttttttgttgutauttuuuauattgaattuttgttttautgaattttuttautataaaaataaaguatgatgatagttttgaaaauaaaauagaagtatttgagaagtauagauuuuautuuuautuuttgtutuutttutgauaututguuuuttaautatautggaauagggagtuaautguaaggaaaatuutggggautaggggutgaggaautagaggtggagagatggaagagtgtuuagtaguuuaggaaaggatgaaauaattuuuuaauaaaaaagtaaaaattttuagtaguuutucgagaagatguaguuuutuuuaggtaauuagagaaagguaaattgtgaaaguagggagaaggggutautttagguutuuaggautauaaagauaaaaaaatgagtgauaggauutgatgtggtggggtgagatgggaatggaagtaaaggagggggutcgtaututtggtgggagtggaaattggggauuuaaguttautggcgatguaguuutuacgtgauuaggagutgacgtggguaaaggtauttaaaguuutgaucgtcgttutucgtuutuututgggtauuaaututattgcguagutcgutgucgtgcgtttaauuuaggcgaggaggaggaggagaaaattuuuuuagattcggguaggtgggtgtgagggtggguauagtutgggauuuutgaatagggtaatutagggatgcgggagtcggaggucgcgggtguutcggtutuuuuuautuutggucggutttuuagau

SEQ ID NO: 128 - DNA, 1200 bp, Homo sapiens
uuagauutgaggtgutuaaacgcggutuattttggtgggcggtggggaaagggggagggggaaacgaaaagauttttgatagautttacgaagaatucguttauttuutttuuaguuagtgttttttggttttgtttataatattuagguucguauuuuauattucgtuutgttttgagaggaggagggaagagaaataaacgtgguagcguatagaagguuaguagggaggttgggaaagggtaauuaggaggcgggggtgggguatguaatttggtaauaatutgggcgguauautuuuauuutgautttuuuuauuaucguuucgguauuutuuuatuuuuuagucgauuututgtgatttgggagtgutgggaaatgggtgtauuagutgutgttggagaggtggcgggtttggcgutgtutggguttuacgggaggtgggggggaaggggcggauuutgagagaatutggtattaaaucguuttgttaautcguagautgutttuuagauauutucgguuuauauagucgttuauuuuucgtuttttuagtuutggaaaaggaattcgggtatguaggcgggagaaatggcggttgttggggggatttgtgggaagtgggaggtagaguuuataguagagaaatcgtggaaaatutcgauuutgaauucgaaaagguagacgttgagguuuututtutguaggutaacgutagguutaggggutggggagcgaaagatgggtgatuutgggaggggaauautgaattgtttgguaggagtuuuuuataucgcgtuuuatuuaguttttgutguauuutcguuucgutuuuutuucguttcgttguaggagaaggggtgtggcggtgtgtuutggagaaatggcggacgggggcgggguaaagcggaaggagggattgagtguutggggaguutgcguaggaggaggattgaggtgggaggggtggggtgagaagagttgaaggaattgagaggtagaatuatttgauattutgatgtguaaauacgaaaagguagttautgagauuuttgtuutggatgutgatuuatuuauuaggutttuattgtutaacgtcgauagtutatatttutgututtuagtutgtuuttaggatgaagututaautgaautgaagtaaggagaaauaguuttgaatutttggagggtuagtgutgg

SEQ ID NO: 129 - DNA, 1200 bp, Homo sapiens
uuuagagttgaatuattaautggaauutaatucgaagauaggtgtttaaaautgtuatggttauaguuatuaaattauaggaggttuauauauaguagtuaguaatggtttgggaatattaaataaatgtgtttututaauaggauaagauaggatuatttatttatguaauttaaaagutgtuuuagaaggagaaaaaagtagcgtattatttgtuuaaaataaauaagattgaagaautgaauatutututgtggatuaatttauagatuauatggaaatagguttatuttattgagaaatattuuattttagatgttuaaatauuttaguatattttaaaaautatttuutatgtgauuaaaagaattuatgttutattcggtaaaaattaauuaaaattaacggtgutttuuaatggacguaggaaauuttttttutttauaaaatatguuuauuutttttttgttgutauttuuuauattgaattuttgttttautgaattttuttautataaaaataaaguatgatgatagttttgaaaauaaaauagaagtatttgagaagtauagauuuuautuuuautuuttgtutuutttutgauaututguuuuttaautatautggaauagggagtuaautguaaggaaaatuutggggautaggggutgaggaautagaggtggagagatggaagagtgtuuagtaguuuaggaaaggatgaaauaattuuuuaauaaaaaagtaaaaattttuagtaguuutucgagaagatguaguuuutuuuaggtaauuagagaaagguaaattgtgaaaguagggagaaggggutautttagguutuuaggautauaaagauaaaaaaatgagtgauaggauutgatgtggtggggtgagatgggaatggaagtaaaggagggggutcgtaututtggtgggagtggaaattggggauuuaaguttautggcgatguaguuutuacgtgauuaggagutgacgtggguaaaggtauttaaaguuutgaucgtcgttutucgtuutuututgggtauuaaututattgcguagutcgutgucgtgcgtttaauuuaggcgaggaggaggaggagaaaattuuuuuagattcggguaggtgggtgtgagggtggguauagtutgggauuuutgaatagggtaatutagggatgcgggagtcgga

SEQ ID NO: 130 - DNA, 1200 bp, Homo sapiens
tgtttaaaautgtuatggttauaguuatuaaattauaggaggttuauauauaguagtuaguaatggtttgggaatattaaataaatgtgtttututaauaggauaagauaggatuatttatttatguaauttaaaagutgtuuuagaaggagaaaaaagtagcgtattatttgtuuaaaataaauaagattgaagaautgaauatutututgtggatuaatttauagatuauatggaaatagguttatuttattgagaaatattuuattttagatgttuaaatauuttaguatattttaaaaautatttuutatgtgauuaaaagaattuatgttutattcggtaaaaattaauuaaaattaacggtgutttuuaatggacguaggaaauuttttttutttauaaaatatguuuauuutttttttgttgutauttuuuauattgaattuttgttttautgaattttuttautataaaaataaaguatgatgatagttttgaaaauaaaauagaagtatttgagaagtauagauuuuautuuuautuuttgtutuutttutgauaututguuuuttaautatautggaauagggagtuaautguaaggaaaatuutggggautaggggutgaggaautagaggtggagagatggaagagtgtuuagtaguuuaggaaaggatgaaauaattuuuuaauaaaaaagtaaaaattttuagtaguuutucgagaagatguaguuuutuuuaggtaauuagagaaagguaaattgtgaaaguagggagaaggggutautttagguutuuaggautauaaagauaaaaaaatgagtgauaggauutgatgtggtggggtgagatgggaatggaagtaaaggagggggutcgtaututtggtgggagtggaaattggggauuuaaguttautggcgatguaguuutuacgtgauuaggagutgacgtggguaaaggtauttaaaguuutgaucgtcgttutucgtuutuututgggtauuaaututattgcguagutcgutgucgtgcgtttaauuuaggcgaggaggaggaggagaaaattuuuuuagattcggguaggtgggtgtgagggtggguauagtutgggauuuutgaatagggtaatutagggatgcgggagtcggaggucgcgggtguutcggtutuuuuuautuutggucggutttu

SEQ ID NO: 131 - DNA, 1200 bp, Homo sapiens
ttauagguttgaguuauuatguucgguuaagatgtuagttttttaatggtgtguaacgtucgtttutttgatagttggaaagcgtgguaggtattagagggcgaatgtatatttaagaaaaauaagataagtuttuatutgtagagtattaatttttutgtgatuutttatutuagacguututuututgaaggtuautgutuaagutggguaguuuuttutaagaagggatgutguuttggagttggagatgaggttaagatutacgtgcgggtauuagtttuagtaataautggatuuttututaggtutaggutggaacgtutuaagtgaaggttattgggggguaggggtttgggtuuauuuaaaaagguttgauuuataggautgaaatuaaatuttgagutaggutgguttguuacgtaauuauagaaauagtaatttaaguaataaaatgaaagutcgagcgutggtuuagagutggggttgggauagutautguautuaggtutuuaggtguatgtagggagaagaguagcggatuuuttguagauaatggtgggagcgagututtuauuutggggtutucgaaguuuatttttagcgtuuagaggtgtgutttcguuatutuuttutgcggtggutguttguagggcguattuuuattgutgutgtututgttuutaaggaagatuaaaggatuuuagaaaauuuagttuuagagutuagttuucgggautagagggtacgutcgguuagatggutgggggtgucgutgguuttggcguuuucgucgtuuucgcgcggtcguuuauuucgguucgauaaguaguttgggcgtuuaguucgguucggtuuutguagggagggagcguaggagutgggggcggututuucggggcggggutcgutuuauaagcguuaggggucgcgcggggcgtgggcgtgggcggggucgggggcggggucgguuttggutgucgcgguauaggcggcggcgtutuuagggggaguuaaggtacgtaggcggcgtucguutuauucgaguatuuttuuucgucggtggucguucguucgcgguucgcgutcguaguutuucguutggcggttgauttutttgtgtcgtattututguutgcguuutgtututguaggauutgttcgttuttutttgggutataagaagguagaggatgagat

SEQ ID NO: 132 - DNA, 1200 bp, Homo sapiens
ttutgtgatuutttatutuagacguututuututgaaggtuautgutuaagutggguaguuuuttutaagaagggatgutguuttggagttggagatgaggttaagatutacgtgcgggtauuagtttuagtaataautggatuuttututaggtutaggutggaacgtutuaagtgaaggttattgggggguaggggtttgggtuuauuuaaaaagguttgauuuataggautgaaatuaaatuttgagutaggutgguttguuacgtaauuauagaaauagtaatttaaguaataaaatgaaagutcgagcgutggtuuagagutggggttgggauagutautguautuaggtutuuaggtguatgtagggagaagaguagcggatuuuttguagauaatggtgggagcgagututtuauuutggggtutucgaaguuuatttttagcgtuuagaggtgtgutttcguuatutuuttutgcggtggutguttguagggcguattuuuattgutgutgtututgttuutaaggaagatuaaaggatuuuagaaaauuuagttuuagagutuagttuucgggautagagggtacgutcgguuagatggutgggggtgucgutgguuttggcguuuucgucgtuuucgcgcggtcguuuauuucgguucgauaaguaguttgggcgtuuaguucgguucggtuuutguagggagggagcguaggagutgggggcggututuucggggcggggutcgutuuauaagcguuaggggucgcgcggggcgtgggcgtgggcggggucgggggcggggucgguuttggutgucgcgguauaggcggcggcgtutuuagggggaguuaaggtacgtaggcggcgtucguutuauucgaguatuuttuuucgucggtggucguucguucgcgguucgcgutcguaguutuucguutggcggttgauttutttgtgtcgtattututguutgcguuutgtututguaggauutgttcgttuttutttgggutataagaagguagaggatgagatgtuucggguuacgtutgttggagauuagutggagguauucguucguauuatttauutuaauuaaucguatutuaauaaattucgcgauaauuagatuaggtaggagaaggcggucggutcgcgcggaaggcggtggagtuauagutgggaagggg

SEQ ID NO: 133 - DNA, 1200 bp, Homo sapiens
ttgtuagtuuutaatgttaatttaggttaaaagaaatgtaatgutcguatuagtaaaaguaaggtuagautttuauutgaauagttttatttttgttttgttucgtttttautucgaggtuagaatgtuatuagguauaataaagataguttuttatuttuatgggattgtguuuagaaagauuaaguauagtutaaauattacgcgataagaaaaaauaaaaaatagatuaagtgauuttggauaagtuatuttauututcgatggttttgttttatgaaagaaggtgtgtgtgtgtgtgtgtcgatgggtgtgtgtgtgtgtgtgtcgatgggtutgtgtgtgtgtgtgtgtgtgtcgatgggtgtgtgtgtgtgtgtgtgtgcgtgcgggggttgagaaggttgutgttaaggttuuutagggutauutguagutttaatatgcgtagttagaatgtuagguaggttgtggagagtggggggutggagauuuucgauttututgagtguuaaguuauauatuagauauuttggaaataauuuttuuacgaagtuuutauauagatuuuauuaggutggguacggaagaauagaaautgcgaaaagaaagggautggatgagtuutggggaatuauagcguutuuauttguauaggauaagtgauagguaggtaaataggtaguagauagguagagautucgagtuuuuuaggtguauuuututuutggaagaguuuucgaaagtauagguuuaatutuuuauuuuauuaaguuutuuuuatcguuttuuucgauauutaaaaguuucggtuutgcgtaatattgcggatuuutuuuaguutuucgtucgutuutuutuucgcggggutucgutuagggucggtuuuucguuuuuacgucguaguucggguuagcgggguucgucgcggtggcgttcgcggatgutgucguattggucgutgcgguacggacggcguaguutcgggcgcgguucgguauaggcgucggcggtuuucguuagutaguaguucggcgaggcgutgguuuauuuatggtuutcgggcggcgguuuutgcguuuaguuutgcgcgtaguutucgtututcguucggggucgucgaguuuucgauacgggcgagatgutgaacggcguagguutggauaaagututtaagatgtuuutgucgcggaggtcgag

SEQ ID NO: 134 - DNA, 1200 bp, Homo sapiens
guuaaagtgatatattaaggtuauuauuttggtuauutgtuuatutatatttttggagtaaguaauagaattaggagtutgaauataggaattaagaagututcgaatataauagtggaagauuaguauuttuuuaauututtgtuaggguagggataututatututgcgtuautuaauutguuttgguutuauagututtuuttttguttaagtuttuaaauuatuaauagtgataguuautaattttaguaggtgguattagttgtuaguaaatgttttttuatatttaatgaatgtttattataatagaataaaatagaaatgtttattaataaaauttgaaatttgtuuuagaaggataaaaacgtagattatttauataaaatgguuaagatutgauaauagaautgtttuutgaagatttauttuauagattattttgaaaauauuaautgtgttuaggaaaattttututuutttttttuataaaattttuaaauaagtgagaatgututaaaaatgatggtgtggaguutagagaaaatgttttutgtgtgutatagatgaauataaautatatatatatattautttuuuaggttgaagtgtttutttuaaaaataaaatttgattattttuaattttttgagggatgutgatttttaaaaaggaaataaagagaaattattaggaaguatatauauaguuagtgagaagaggaaagaagaaaagguuaagguuagaggaagggggtttggggtgggggaaaggaagggtauagauuutuuuaaataaucgaagatgggtaaautuuaaucguaaagcgatgatguuauttgtggguuutuagattggagaggacggagatgagtgacggggutgtgcguautgggguaucgcguauagacgggaggaggggggtgtutgtgtgttuuaaaggggggagtauuagttaautggucgucgguucgtgggggttggtgagaaggagggtgaguttggcggtgacguacgguuutuacgtgaucgggagutguagagutacguaguuttcggtguagtcgtuautcgtgtutcgutauuagutuuucgutguuutgcgutcggcgggutgguatucgguucgggggaaagcggauuaggtaaggguuucgggguucgcgcguuutggutagguuaguagaggutuaucgauuu

SEQ ID NO: 135 - DNA, 1200 bp, Homo sapiens
ttgauauttggatttggatuaauaatgutttuaagaagaaagauttttgatuaaaagcgggaaatgagaaagcgautttuututgaaaagtguutuuuagtuucgaggutgcgagguuuuuacguuaggutggutuuuacggaagucggguauuuauucgguucgauuaagcguuautucguuucgtggacggggcgtuuuauuucggggacguucguuuuauaucgcgtttguauuucggagguuuuttgucguagaggcggacggcgcguututuucggguuuutggggtucgcguutuuutcggguagaututttcgaututgutcgaguutucguttuttuutgcgggcggacguuucggauauaacggguuucgutgttuacguaggggcguuucggcggggcggguaaagauucggggacgcggtuucgtuucgagacgutuagutucgguuuaucgutcguagutuucguuucgggcguaggtuucgauuuuacgggucgtutcggagucguagcggucguttuuutguacgtuutuacguuuuucguacggacgucguuaguuucgcguutuagtttuuuuautaguaggatggaaagacggguuucguuucgaagcgtagcggcgtutucgtggtaguuagtguuuagagagtucgucggtuuuaucguuuuttuaaaggagaauucgguuuaucguucgucgcggcggcgaucgcguaguuuautcgtuacgcgguucgcggcgtuuaguucgggucggutuauutuaggcggtcgutgucguuutcgcguutgcgcguuuutcguuucguuuututuuucguucgcgtuucgcguaucguagguututguuuutcguuuaucguaggauucguuucgcguacgcgucguacgtguuauacguauuuuacguuuutgcguacgcguagguuuuaagtcgcgguuaatgggcgacgcggucguagatucguucgguuucguuutguuutgtgagttuutucggucgggutgcggggutucgutuagtucgggagcguagutgggucgcggcgutucgauutucgutttuuuaucguucguagutgaaguauatuucguaguucggcgcggautucgatcgucguagttguuututggcguuatgtcguagaacggagcguucgggatguaggaggagaguutguagggtga

SEQ ID NO: 136 - DNA, 1200 bp, Homo sapiens
tgutttuaagaagaaagauttttgatuaaaagcgggaaatgagaaagcgautttuututgaaaagtguutuuuagtuucgaggutgcgagguuuuuacguuaggutggutuuuacggaagucggguauuuauucgguucgauuaagcguuautucguuucgtggacggggcgtuuuauuucggggacguucguuuuauaucgcgtttguauuucggagguuuuttgucguagaggcggacggcgcguututuucggguuuutggggtucgcguutuuutcggguagaututttcgaututgutcgaguutucguttuttuutgcgggcggacguuucggauauaacggguuucgutgttuacguaggggcguuucggcggggcggguaaagauucggggacgcggtuucgtuucgagacgutuagutucgguuuaucgutcguagutuucguuucgggcguaggtuucgauuuuacgggucgtutcggagucguagcggucguttuuutguacgtuutuacguuuuucguacggacgucguuaguuucgcguutuagtttuuuuautaguaggatggaaagacggguuucguuucgaagcgtagcggcgtutucgtggtaguuagtguuuagagagtucgucggtuuuaucguuuuttuaaaggagaauucgguuuaucguucgucgcggcggcgaucgcguaguuuautcgtuacgcgguucgcggcgtuuaguucgggucggutuauutuaggcggtcgutgucguuutcgcguutgcgcguuuutcguuucguuuututuuucguucgcgtuucgcguaucguagguututguuuutcguuuaucguaggauucguuucgcguacgcgucguacgtguuauacguauuuuacguuuutgcguacgcguagguuuuaagtcgcgguuaatgggcgacgcggucguagatucguucgguuucguuutguuutgtgagttuutucggucgggutgcggggutucgutuagtucgggagcguagutgggucgcggcgutucgauutucgutttuuuaucguucguagutgaaguauatuucguaguucggcgcggautucgatcgucguagttguuututggcguuatgtcguagaacggagcguucgggatguaggaggagaguutguagggtgaggcggaggaggcggcgcgggagucg

SEQ ID NO: 137 - DNA, 1200 bp, Homo sapiens
uuaauaatttgagcgguuaagauagguagutuauttgaagtuagaagttuaaguutgguuaauatggtgaaauuucgtututautaaaaatauaaaaattagucgtggtagcgggcguutgtaatuuuagutautcgggaggutgagauaaggagaatcguttgaatucggaaggcggaggttguagtgagucgagatcgcguuautguautuuaguucgggcgauagtgcgagautucgtutuaaaaataaaaataattagtaaattaattuattaatttaagaaaaaaagcguaatuttuttuuaaaggcgatuuuaaggutgttggaggutgaaaucgutaagtcguuuuutaucgcgcgggtgguttcgtuucggauuuutuuagaggutgtguuautgutggaggtgutuagcggggucgggauucguttggggcgucgtucggggcgaguaucggaguucgautgggutgaatgguaggtuutgauuaaguuauucgcgcgguuucguucguutggcgggatgagtgacggcgguaauaggtguagagcggcgtucgcgguttutcgggaguuuucgagttttgcgagtuagguuucgagagtuagagtttgaututagaauuttuttcgutttauauuagtuttcggaagtcguucgtuaaucgguttuttutauutuagtaaaguuttaaggtuauutuaauututcgaaauuuauuagtuuuagaggucgaututtutuaagtggaggagaggggaaggcgtguatggagutttguutgaucgutcgutututuutuuuuagucgtutgagagaaggcgagaggggtuuuagaaucgaguuutcgutucguacguuuuacguucguuucgutgtgucgtgcgututguuuutgagacguuucggutcggagtuuuaggguuauagucgaaautcguuutuucgcguuuututucgguttguagcguucguucguaucguuauutguuauucgaggcgacguagtucguttutuuuuauucguuaggtgguaggttuagggucggggtucgaguutguttuuutguagutguuauaguuauuutcgucguuagggggcgagagtuuuagguaggauaaguagcgutgutguuttggcgtgaaggtggtttuaaautttagguttuttttuuutauuucgcgtututgaagag

SEQ ID NO: 138 - DNA, 1200 bp, Homo sapiens
gaggaagatggaaaucgagtucgggucggtgagtacggcgatuaggguuuuagggcgcgggatagcggggagaaggaguagcgtggaguutgtuuutgaggguttggaggggcgaggtgaagguuuuaaacgutcgutatutggttgggaagaaacgtgguuaggtguuuuatcgaucgutguuaggaguuauucguucguagauutcgtutggutgtgtuatgggggtuagaaaggcgucguuacgggguutggautgggtgtagacggguuaggggauutuaggcgtggatgtttcgggaggcggcgggutguuagguauuuagcgaagtucgaguaggtaaaucgutttuucgucgaaattgggutagguagcgtutttgacgutuuutgcggucgaautcgauuuagucguuuaaggcgaggagttguuagggcggtcgacguutuuautuuucgautggtuagcgaggaucggaaggtutgagagauutgguaggguucguutgtgguuuaggcgggauucggauaututaguuutggutgtguauuuaaguuagtuagggtggauttggggaagtgaaggaggautguucguauagguucgaggcguagcgguaacgtguuatuucguuucguuucgtuucgucguuaguaguuaatuagggagcgucgcgcgagggtggggauuagutgcguacgagguuagcggcggggtcgutgucgttgagguttuucguuautgutgutggcggatttgtgggguaaaatttutcgutggutaguuttuttttuuutuucguautttggtggggagggggtggatuutcgtttcggtguuuaagttuacgatgauucgagagaautcgaggaagttgtcgucgcgguuatttuuuuuagtgucguaauttgutgguuttggagggggaggagcgucgagguagtgautgcgacggttaguucguucgttuutgutgtuttcgtttguaaaatagatgutagatutcgaggttgattutuaaggggagggguuagtgaggguauattgutttggaggagtagtttaataaautututagtgutggaagttttaguaaacggaggaguuttuaaauucgguaauagtuuuuatutaggtgtttucgtaauauatgcggaattattgutagtggaatttuaggutttutttgttgtttaaut

SEQ ID NO: 139 - DNA, 1200 bp, Homo sapiens
ggattttgtatatgtuatuagutgttgtgauuttuuaauuuatgagaattttuutuatguutgaggattuauttaaggutauautttuagatuuagagatatutttuttttuututtggutgggtuagtutgautttggutguatgatuauauattattaguaggtgutauuatttttaggttagaatatgtgautttuttaaagtaaaaaaaatuutatagtuututaguttatuaattuattaagttaaautttatatauuaattttuttaaatggaggaatguutttattaatatguaguatutatgtuattautaaattautatauuaauaaagatgaaataatttattguuuaagttggtutuuuuuuuauuuaauutttttuuuuagttttgtgtggaagtttgatuaggagutttttuuatggaggttttuaguttuuttagttutagguauaaagttatgatutttuuutgatgutgauuauuttaggatgatatutgattattagtuttgutautgcgutattaagtgtuauttatttutaaggaaatgaattgagggtuttggauaggataaggauaggaatgttttattauuuauautauagatgttggaaaauaaagutgaaaaututtttaatttgtgtutatautgauatutgaatguttaauuuattattguttttttttttggtggtggtgggtagagatggggtutuautgttguuuaggutggtuttgaautuutgggutuaagcgatuutuutguutuaguuttuuaaagtuutgggattauagguatgaguuautauatuuutattattguuttutttaaaaatuttttuttututaaaaatutttttagatgttaattaauuuatutggttgttgtttttuagtagagaaatggtuagaaggaacgauauuauagaagaaattagaatgtttaaaagaauuaggaaaaauatutgtagaatutaaagtauutauuuagatggaaaaatutauagauauagacgaggauaatgtaauuagaauagaatatagtgauaaaauuauuuagtttuuatuagtttatgutgtguuagguautgaguaaacggaaguagttggtggtutttuttuuaaauuaguuauuuutaagautautauuuuauuutuatuauuauutuuaauagutgtutuauuagagttt

SEQ ID NO: 140 - DNA, 1200 bp, Homo sapiens
taaggutguttgtaututgataatuatgattatggtgttatttagaguuuaguuuaaatauutgaguutaaatgagtgaaagaguagagagtgggcgtttttttgttttttttgttttgttttttgtttttttgagacggagtutcgutuagtaguuuaggutggagtguagtggtgtgatutgggutuautguaauutucguutuuuaggttuaaguaattututguutuaauutuuagagtagutgggattauagguauuuauuauuacgaucggutaatattttgtatttttagtagagauagggtttuauuatuttgguuaggutggtuttgaautuutgauutcgtgatuuauucguutcgguutucgaaagtgutgggattauaggcgtgaguuaucgtguucgguagagtgggcgtttttaaatgaaguagtattuutttagtttggtaaaattgagutgaagttauauauagtaauauuttgauttgttgutgacggtgtgutttgtggggatagttggtgtutgatutuaautgtggagaaauaggaagtttutggattuatgutgaautuuuattuuauaggcgagtttuauaaaagaaaaagtgututgttguatauaattattuautgtgtgtutauaacgtgguagaaattattattuutaaaggtgaucgaagagagutatagtttatgggguuaggaguaatgtgtggaatggcgagguagtgauaagauaguautguuataaatututtautttutttuuaaacgagututaattttaagutuaacgauuaaagacggagagguagtcgutttgagutautuutggutatttgttgggaaatuauutaaatucgttuagatttaguuagaaaaggggaaataagtutuuutcgucgguuaaatuatgguutttgtcgutttcggutuuuuattggutgcggucgutgtgguuucguutuuttgagtggtgcggagutttgtgatgcggaguttcgtgatguacguuucgatguutgcggggutataaaaacgutcguaagcguuaagtutuutuaggagucgucgguaaggggguaacgaggaagututtaagagcgcggucggaaaguaggtggggagaatutggagaaaaggcguauuutgcgguutcggagauuattatgtutuagggagtututcguagt

SEQ ID NO: 141 - DNA, 1200 bp, Homo sapiens
tggaaaatuagauauuagauuuutuauutguaauaattguutgatguutgttgauuuautuututtuatututuuutaautuutgutattttuuuauatguagttauatttuttuuutgutatatuutaattttaatuagttgagaagatggatttgagautgatututuuuuuutaggutgtaguauuuaaataatguuttuttuuutgauaataatggggtutuagtgautggutttutgtgutgtgaauuauuagauutagauuaaautuutgguatttututaauaatttuuuauuautgaautaagttataaattutgaggatggatuuutgtttuttuattttaagatggggattaaaaggtauatuutagagtgttattgtucgtgtaaaatagggutgtgaaauuuuutauauutatguauagataaautgatgguuutguaaatguuuagagaagtaauuttataaagatttagtgaatgtttuuuauuattattggaaaatutggutaagtuagtagutttgttttuatatauttagggggaaaatuaauaaaaauttgatguuutgttatttuuttuuagtttuttutguaaututagutuuaaagautguagttuutguagtatttttttttttagaauauuaggaaaaaaagaaaaaaaaauuatguaaatgtautatttuttuaaauauaautaggaatgattgaauagutgggagaatatgaagaaaauuuutuagagaauuaaggguaaguagtgagttuagatutagguauatgguuautaggaatggttuttuautguuacgtggguaguuaauaguuagtagutagagauuaguutcggtuttcgguutgcgggttutguaaagtuaggutagutggututucguutgutucguauuucggcgaggttucggtggggaggggtagggatggttuaguuucguuucgutagggcgggguutgcguutgcgcgutuagcggucgggcgtgtaauuuacgggtgcgcguuuacgaucguuagautcgaguagtututggaauacgutgcggggutuucggguutgaguuaggtutgttutuuacguaggtgttucgcgcguuucgttuaguuatgtcgtucgguatuuatgtagcgutggtgautggagguaauaaggguatcgguttgguuatcgtgcgcgau

SEQ ID NO: 142 - DNA, 1200 bp, Homo sapiens
autuaaguauuaguaagcggautcgaautuaaagggtttuagggtgtagacgauuagagcguautgaggggutuuaggtcgguucgutucggagucgggagagtaaucgcgtguagggaaaggcguacgguaguutcggtguutuuuttuuutuuaucggacgggtgautgautaggggtgaggtuutuauutuuaggutgcguagutgagttutggaaggtautgtuutgggggtgtgagtgtgtggguuutuaggaaaggtgcgaggggcggatgucgtggtutauucgaguttgcgggggtgcggtggtuaagttuuuuaggauuutcgutggcgucguttgtcgauaacggggaauagtgggcgucgcgggtggtggtaauttgguucgggaagaaaauauatuuutttuuuaaaucggattuututgaguaaaauuagatggtgtuatucguattttauagutgcgtuaagagagguuuaggauauuaagaguuagutgtgguuagaguuutttuautgauuuagaauautgguuutcggaaauutggagaautgcgaggguttttaatgcggutgagaagaaagtgaagauuucgautuutuutututuuttcgaaagagucgauauaggtatgaagauuagcguutcggacgutucggggtagggggucgacguuuuutuuutttuagaatuacguuttgutgatgutuagtuutguuuagtgtuuauuuuuuauuuucguuuuuagtttuutttutaguuuutucggtuuuuaaagcgaautaagutagcgguaguttutuuatuucggaggagautuuaaautuutaggtttuuuauuttggagagtautgututggaatgcguaggggagcgguauutggaaagggttgggcggaaguttutcggauuuagagggggcggauauucgguaucgcgcgtgggagggattaautuuauuuuautagauuauutuuuuutututuuuaggaguuauttuaaucggttgtcguuuuagtggucguuututgaguacgtgttautguuagtutgcgtuagcgttgggtaaatauatgautggucgacgucgucgggtggggutatttaagagauagucguucgutggtuutuuutgaauttggutuagutgucgggutgutucggttggaaacguuaaguuagutgcgtuutaat

SEQ ID NO: 143 - DNA, 1200 bp, Homo sapiens
uututtttucgtttguattttguuuuatattagaatgutauuatutaaaaucgutuuatgaattagaaagtttaagttgtuagggautgaatgtgtutgttttuuaauagtttutgguagttuuutguttttatggtgtagtuatggtgaaauutgagtuatutgaagatggtgguagtguuagagaguaggagagaggagutguaaagaaguuagauagcgttggguatuaataautuutggguauuaucgguuautgguuttuattutgauaggagaguuaauuuaauuutaautgtgttuagagattgutuuaagutttauagttutauuaaattutttutgtaguaaaaatataaagguatuttuttaggtaattttttttuagggttuuaagtaguaauauutaacgtutguagtgcgagttututtuttutttttttttttaaggtgataaatuataauattttuutggaaaggaaaguagtagauutuattttttgttttaaggaaaaatagtttttutgtttuuattuatatauattaaataautauuuaggaaatacguuucggttttgcgcgcgtgtgcgtgcgtgtgtgtgtgcgtgtgtgtgtgtgucgtttututaaaattutgautuatagtutggaggauaatgattgauttttttuuauttuauatagttgutatgaatttutuutuauaaggatggguutgtttautuauuutggguatcgttggtagagcgutagtgtaaauaguutgaggaauucggtgggucggggaagtgggcgcgututgttutucgcgguuagutgggacgucggguuaggtggggucguutgcgtttaguaautgutttutuauuuuutggatttgcgatgtttguuauaguagcgagaagcguuattgtaatggggatgggaggggtggaguutuuaagtuutgtutuaatttagatututuaututgutgttaggcgcguuuatttuagattautaaautcgaattaagagggaaaaaaaatuagggaggaggtgguaaguuauauuuuacggtguucgcgaauttuuucgguagcggautgtaguuuagguagacgucgtcgagatguaggguuuaucgutuutgaucgucguuuauutuututgcgtgtguaucgucgcgutggucgtggutuucgggtaggaacgtgggcg

SEQ ID NO: 144 - DNA, 1200 bp, Homo sapiens
atgcgtgutgaagtatgtaggggtaaaatgauatgatgtutggaatttgttttaaaatauttuagaaaagcggggagtaggtgaaataagtgtgauaaaauuttaatagttgttgaagcggggtgatgggtauauaguagttuattatauaattutauttttgtgtatguttgaaauututuaattaaaaaaagagggtagagagtuatuaagataatttautaaaagtttuutgtaatgtuuuuagaagauuuuuutaautuauauauauauauauuuauauauauautguuttttgagaautttututagaagguuaauuuagguagtuuuuuaautgtaaggaagautcgtgtatgtatgtguatatgtguatttuuuuagggaaaaauatuuauaguttuuatgacgagaggggtcgtgauuuutuuucguuaaaagattaaggauutgcgatuutauagaucggaguuutgtttgaagtutgcgttguuuutuauutuaagutggtuautgtgtgaagttgguutagaatuuuucgguuuutgggaguttgttuutucguutgtaaaatggggutguagggucgtuuacgcgguuaucggaaggauaaggtgttuaggucgutaggucgutuuutgguaagcgattuuuautcguagcgcgguutcgauuutcguuuaagacgcguuutucgcguuuuuauuuuutuuagguuutgguuagtuuauutuucguttggggcgguaatttgtutuuttttgaauuuuucguuuucgacgggtttuuuuutttgattcgcgguucggagguttuuuuucgutttgaaatguaaauucguutcggutggggucgcgggcgguucggagutataaaagguutgggtggggcgggcgcggcgguaggauagucgagttuaggtgagcggttgutcgtcgtcggggcggucgguagcggcggutuuaggguuuaguatgcgcgggggauuucgcgguuauuatgtatgtgggutatgtgutggauaaggattcguucgtgtauuucgguuuaguuagguuaguuaguutcgguutggguucguaaguutacgguuuuucgguuucguuuucggcguuuucguagtauuucgauttutuuagutaututuacgtggagucgguuuucgcguuuucgacgguutggggggcguuuttuuutgcg

SEQ ID NO: 145 - DNA, 1200 bp, Homo sapiens
aggcgutuatttaaaattuuagtuttgutuucgagggutttgguutuuatuttuauutttaacgaucgagggutggtggcggcggutuuttuutgututgcgauaggtgtcguttutgagcgggagttttggguauagauatcguagatucguauuauuuutaggggtutguatgggtttuagutguauuuaguutggaggggutttttggaaggggguacgtgggcggtgggagcggggaaguaggtgaggggaguucguutgcgcggatttttutgutgtaaatuagauuuauagtutuuagagcgtuttttuuuuaagtgtggaauauatttttuttutaatuututttggagagggggttaggaacgtgucgaggucggagucgggagcggggtgggtuucggcguutgtgaaucgagtgggggagcgggucgcgcgutguuutgaagauuutguutucgcguuuttgggggutcgggguuuagcguuttcguaggcgagcggcggcggutggututguucggggtgcgcgggggcggtgtttuutcguutuuuuutgcguucgggutcguuaaguuucgautguucgutcggucgtcgggtucgcgggcgcgcggacguutcgacggutuuuuutgucggggcguuuuucggtuuuuauuutuauucggcggutuaggautagtuuuuuaauttgttututgutgttaautagtcgcgutgucggaacgatgggatggggttguuauaguaauuagautggacgtuauaatucggguatgtgtgucguuuuuucgucgtgucgucgtguututggcgutgucgucguuauuutcggucggutgcgggautcgggutgcggggcgcggtgggaggagcguucgggutttgcggggaguaggutaaggcggucgagagaaaggggggtcgagacgggggggtggaggtttgggggggtggggggguaggcggucguuatuttutcgucgaggucguutcguucgucgcgcgcgucggtucgguagcguacgggttaaggutgguaucgcguucggcgggagggggggcguuuauutuuuutuutuuucgcgucggguatgcgggguuuagtggguaucgaggaggagutgucgcggutgttcgucgaggagatggagaatgaggacgagatgtuaggtgagcggggcgcgggtgcgu

SEQ ID NO: 146 - DNA, 1200 bp, Homo sapiens
uututcgguutttgutgtuauuaagguuaaaggaaggutggutgtgtggtgtauuttguauutuuagaatguuttuaggtgtgatgtgguauuauagatutggtaggtggaggaaggggtggguuattttauagatgggaaaguaggututggagguttggatgagtttucgaggauaggtagtaguaaagutgggauutgutatuuuuauuagagutgtutuuutgaatuagtuauuuucgauuutgtuuagtuagauaguaucguutuuttggaggguuaaggutgutuuuauuuatutuaggguucgagtggtgtggtttgtggutgttgtatttuttguutttguuaguuaguagutuautgtuauuutaguatutgtuaggguuuutguagtgtgtutgtgggutguuagagatgggguutuattgtuuuaguutagacguacggtguutgggtguaguuutguuucgggtgagggatggtggatggggutgucgtgtuuutgggutuuaguauagggutuatgutuuutgagtguttggggatuauauagtgatgtgauututtttuaggggguaatgggaagauutaaggtaggggaagtuuagaatutuatgttutgutuuutgututgutgtuuuttgucgagtggguutuagtttuuuuututgtuutatguauatgguagtutuauutacgutggutuagatattutgggtgucgataagttuauuattgagtgaaaggguuacgtggtutuauagauuuuuautguuuatutuutgtgtguatgauuuatgcggutuutggggacggggtggatgtuatggcgtuaucgtggtautgttutgauttggggguagtgguautgatgggagggaguutguatgguaguutgutgtuatgggaguutuagggtgggguuutguagutgaguuuuauuttggggauuuttututgtuuauagtguuuuuuautgaaggguaaagtuuagcggattutauautggaggtggacggaguuuuutguuuuuttuatggtggggutgucgggguutgacgtggaguuuaguutuuutuuauutaaguuuutggaggguatuuutgagagagagttutttgtuaagtggguagggutgtuutautgguattgutuutgggtgaaggagutauaggtgagtguucgggagggtggutg

SEQ ID NO: 147 - DNA, 1200 bp, Homo sapiens
cgauaggtgtcguttutgagcgggagttttggguauagauatcguagatucguauuauuuutaggggtutguatgggtttuagutguauuuaguutggaggggutttttggaaggggguacgtgggcggtgggagcggggaaguaggtgaggggaguucguutgcgcggatttttutgutgtaaatuagauuuauagtutuuagagcgtuttttuuuuaagtgtggaauauatttttuttutaatuututttggagagggggttaggaacgtgucgaggucggagucgggagcggggtgggtuucggcguutgtgaaucgagtgggggagcgggucgcgcgutguuutgaagauuutguutucgcguuuttgggggutcgggguuuagcguuttcguaggcgagcggcggcggutggututguucggggtgcgcgggggcggtgtttuutcguutuuuuutgcguucgggutcguuaaguuucgautguucgutcggucgtcgggtucgcgggcgcgcggacguutcgacggutuuuuutgucggggcguuuuucggtuuuuauuutuauucggcggutuaggautagtuuuuuaauttgttututgutgttaautagtcgcgutgucggaacgatgggatggggttguuauaguaauuagautggacgtuauaatucggguatgtgtgucguuuuuucgucgtgucgucgtguututggcgutgucgucguuauuutcggucggutgcgggautcgggutgcggggcgcggtgggaggagcguucgggutttgcggggaguaggutaaggcggucgagagaaaggggggtcgagacgggggggtggaggtttgggggggtggggggguaggcggucguuatuttutcgucgaggucguutcguucgucgcgcgcgucggtucgguagcguacgggttaaggutgguaucgcguucggcgggagggggggcguuuauutuuuutuutuuucgcgucggguatgcgggguuuagtggguaucgaggaggagutgucgcggutgttcgucgaggagatggagaatgaggacgagatgtuaggtgagcggggcgcgggtgcguutgggguuagagcggcgggggcgucgagacgggucggguuuagggcggagcgcgggcggcgggggcggcgcggucgggutggcguutcguaggaggggua

SEQ ID NO: 148 - DNA, 1200 bp, Homo sapiens
guatgaaattgagataaaauatttuaauagautggauagaututgaauautgttgaagagaauattgataaattggaauutggtgutgagguattttuuuagaatguaatgtauagagauaaagataaaauagggagaauatggaagauauuttgagatgutuuaauaatuatuuaattggagttguagaagaaauagaaaaaatggtgaagaagttggatttgttgagagtttguagauutggagagaaatgtgaggtutuagatutggagtguattutggaataguaaguaagataaatataaataaatgaauucgtagauacgttagaauututaggtautttagauutagtauutauaaaataaggguaguuagatcgttutuagauttuttaauaauaauaggtgguagagauuatgguaaaatutucggtguautgagggaaaatgtgtgutgatagttttatatuuagutaaaututututcgagaataaagggaattatacggggtuatgggagutuagaagggautguutttuttttuttatggggaggagtgaaatuagggaagauatuauagaggtgatgtttatgttuuutagguagtaagggggtggguattuuaggaagagagtuaggtagtttggaatggutgggggutgtgaaggaguautcgtuutguagcguuttgaatauaggguagtgagatggaauttaattutgtgtggtgggaguaggggaggutuatgtuagaguuutgatttaggtaagtggutgtggggutagguaggaggatggattggutuuaaggggtgaggttggaagutttgttuttuuuuagagtuutguattggguutguutgggtuuuagutgtutuuuuauutaguagguagutututuutuuuutguutgtggcggggtuuucguuuatgguuuatgggguuuuttgutuuttgguagttuutuacgtguucgtutuuuutguagatgauuaagatgutggauutuutggaggauttuutggagtacgaaggutauaagtatgagcggattgatggtgguatuaucggggguutucgguaggagguaatcgauagattuaatggtauuuuuatuutguuuaagggttutgattgagautuutttttgguututtutuuuagguutggttuttaguuaggtuauuuagutggtgt

SEQ ID NO: 149 - DNA, 1200 bp, Homo sapiens
gaaatutauutatttgtaautuuagggaagtaaatgagataatgtttggaagatatutagtutauuagutgutuaggcggggggcgaaggttutgttuuutgtuuuttututatuaaatggutttaaaatuagaagaaagtattacgggtaggaaatgggautgcgaaaaaaatggaggutgattutgguutaaaggatauttatututatauaaaggguttutgattuaaagagguuucgggattuaaaaauauaaguagagutauauuacgtggggtttgggacgagutgaattucggataaagatguutgtaaagaggagttgatgcgaatuuagaaaaguauttgaaatguuuaggutttguatgguauattuuuuaggattttttcguaucguatttagaggtttagacggagtgagutcgaatuagguuaaaaguuttaggttautuutgggcgguagcgggguagaggtuagatuaauaggtgtggaaggutgcgaggtgagggaagaaguaggtgtcggtggagguuuuagttuutucgguuuagaggutuuaacgutgutattggtgttuuaguagcgggcggaggggtuutguucgguatutgggcgcgutggguuuagaguucgggaggagguagacggaggcggcgggtuagcggcggguuaguuucggtggguttucgutuuuutucgucgguucggtucgguucgucgcggaggatguutggaggttgacgtgaatgguutgtggutgggaaguuuuuaggcguuutguutaguucgggggcggggucguutagucgucgguuucggtggcgtgagggutggggcgggggucggggauutcgcggggagggagcgggagggaaagcgguuuucgcgguuuttuaattggucguaucguuuutcggcguagaggcgggaggaggcgguuutuuattggtcggguucggcgguuaatuutcgaagguuuaggcggaggcgggggcgguutgtggggaaucgaggtgcgggcggcgagcgagguagucgggtguttcguagcggagutcgaggututgtaguauuuagttggauuuagtcguuuagguaucguucgcguuucgcggucgtutuutgcguutuuuagaggagggagttcgaguatuuuucgcggcgguuautuutcgggguuaggagcggggaagcgaag

SEQ ID NO: 150 - DNA, 1200 bp, Homo sapiens
gatutuagggattgaaatutgtuutaggtgtguauuagaaagtggtgguaagagatgaaguttcggaggaggutgauutcgguuaggattuttaagtutuaaagtttuagutttutuauuuutuaacggauatgaaatutauutatttgtaautuuagggaagtaaatgagataatgtttggaagatatutagtutauuagutgutuaggcggggggcgaaggttutgttuuutgtuuuttututatuaaatggutttaaaatuagaagaaagtattacgggtaggaaatgggautgcgaaaaaaatggaggutgattutgguutaaaggatauttatututatauaaaggguttutgattuaaagagguuucgggattuaaaaauauaaguagagutauauuacgtggggtttgggacgagutgaattucggataaagatguutgtaaagaggagttgatgcgaatuuagaaaaguauttgaaatguuuaggutttguatgguauattuuuuaggattttttcguaucguatttagaggtttagacggagtgagutcgaatuagguuaaaaguuttaggttautuutgggcgguagcgggguagaggtuagatuaauaggtgtggaaggutgcgaggtgagggaagaaguaggtgtcggtggagguuuuagttuutucgguuuagaggutuuaacgutgutattggtgttuuaguagcgggcggaggggtuutguucgguatutgggcgcgutggguuuagaguucgggaggagguagacggaggcggcgggtuagcggcggguuaguuucggtggguttucgutuuuutucgucgguucggtucgguucgucgcggaggatguutggaggttgacgtgaatgguutgtggutgggaaguuuuuaggcguuutguutaguucgggggcggggucguutagucgucgguuucggtggcgtgagggutggggcgggggucggggauutcgcggggagggagcgggagggaaagcgguuuucgcgguuuttuaattggucguaucguuuutcggcguagaggcgggaggaggcgguuutuuattggtcggguucggcgguuaatuutcgaagguuuaggcggaggcgggggcgguutgtggggaaucgaggtgcgggcggcgagcgagguagucgggtguttcguagcggagutcgaggutu

SEQ ID NO: 151 - DNA, 1200 bp, Homo sapiens
gggatggagtattguaguututcguuuuauagauttgtgaauagggcgtggggauagauauuuagutuutaggguuatguuauuuuuauuuutguucguututcguaguauutttuutgtggatutgauuuuuttuuagauauagaagtaaguuaguauuuaguagauauagggtuauutuuuagutuuutauuagguaagagcgauuaggguagggautgatautgucgaauuuaggaguuagguucgauuuaguutuaggtuuaguaggtuucguutgtuuauutggguuagguutagaguucgggaguuuutggutggtgggagguuauucguaauuuauuuuauacguagutuuagutuuuuuauuaggcggggcgautaggauagggauagaauucgttgaauuuaggagtgagatucgguuucgggtuucgutggguuutuucgtuuauuttggutggauutggcguutgggagauuttggutggcgcgagguuacguuuauuagauatguagttuuagutauuuuauuagutgggcgauuaggauagggacggaggutgutgaguuuagttagagguutguuuuucggggtutgtuutgggcgutuuuuuaaggacggauaggguagguagggtucgggacgatggucguauagtuucgguuucgtgttuuuagguucgtuttgutuutcgatgtgagggagauucgggggatgggauaggutggguuucguagtguutgautuuutguagggutuucgggauaggggtucggcggauagucggutgutuacgggtgaggggtuuaagutgguattgcgguuauuttucgguucgggutututtggggaggggcggggttggtgagaaucggtuacgtgutucggggutuautcggggtutuuuagggucggaagtaggguuuutgtgcguaggcguuutgaggatuucgggutguuuatutuacguuagggggcggaauttuutguaguutututguutucguatuutcgtggguuutgauuttutututgagagucggguagaggutucggaguuatguaggucgaagguuaggguauagggggttcgacgggcgatgutgatgguuuaggagguuutgguattuutgatgguuuaggggguaatgutggcgguuuaggagaggcgggtguuacgggcgguagaggtuuuu

SEQ ID NO: 152 - DNA, 1200 bp, Homo sapiens
ttguaguututcguuuuauagauttgtgaauagggcgtggggauagauauuuagutuutaggguuatguuauuuuuauuuutguucguututcguaguauutttuutgtggatutgauuuuuttuuagauauagaagtaaguuaguauuuaguagauauagggtuauutuuuagutuuutauuagguaagagcgauuaggguagggautgatautgucgaauuuaggaguuagguucgauuuaguutuaggtuuaguaggtuucguutgtuuauutggguuagguutagaguucgggaguuuutggutggtgggagguuauucguaauuuauuuuauacguagutuuagutuuuuuauuaggcggggcgautaggauagggauagaauucgttgaauuuaggagtgagatucgguuucgggtuucgutggguuutuucgtuuauuttggutggauutggcguutgggagauuttggutggcgcgagguuacguuuauuagauatguagttuuagutauuuuauuagutgggcgauuaggauagggacggaggutgutgaguuuagttagagguutguuuuucggggtutgtuutgggcgutuuuuuaaggacggauaggguagguagggtucgggacgatggucguauagtuucgguuucgtgttuuuagguucgtuttgutuutcgatgtgagggagauucgggggatgggauaggutggguuucguagtguutgautuuutguagggutuucgggauaggggtucggcggauagucggutgutuacgggtgaggggtuuaagutgguattgcgguuauuttucgguucgggutututtggggaggggcggggttggtgagaaucggtuacgtgutucggggutuautcggggtutuuuagggucggaagtaggguuuutgtgcguaggcguuutgaggatuucgggutguuuatutuacguuagggggcggaauttuutguaguutututguutucguatuutcgtggguuutgauuttutututgagagucggguagaggutucggaguuatguaggucgaagguuaggguauagggggttcgacgggcgatgutgatgguuuaggagguuutgguattuutgatgguuuaggggguaatgutggcgguuuaggagaggcgggtguuacgggcgguagaggtuuucggggcguaggg

SEQ ID NO: 153 - DNA, 1200 bp, Homo sapiens
ttgtgaautguutgtggauaggguaauatgguagagaautgaggguaguttttataauaaattaagagatttuttaatatttaauuutuuttauattaautgutcgtggtatattattutttttaaaaaattatttatautttttgtgggtauataataggtatacgtatttattggatauatgaaatgutttaauatagguauauaatguataatgatuauataatggtaaatgggguatuuattuuuutuaagcgtttatuutttgtgutauaaauaatuuaattatauttttagttatttttaaatgtauaattaaattattttgautgtagtuatuttgtgutatuaaatautaggtgttaataatuauatuatggagaatggagtatuuatuuutuaagcgtggtguattaututttgaatatgutgututggattgtatttgutaatatgtgggguttututattaatgttguaaaatagggaguuaataatutggttgagagatgaaaguaguaguaggaaaatgtgttuauagaauutuatacgagtggcgtutaggguuaaggutuuttgaatguatauuuaagggutgatgagaggtggggacgagtgutgucgaaggautuutuaautauuagaautacgtaggguuuutaauatuuuutcggagcgagutuuucgcguuucgtauauuauuaggtutuuutggttuututuutgutuuucgtuucgutauutuaggaaautttaguuuaguututucgatauaagucgguutuuttgagggutttagguuuututtcguuagtgtatauuuuacgauacguutgcgtautagggauuauucggutttgtgggcggagtuuatgagagggttggaguuucgutcgtgguuucguuuauuuaaguutaggucgguuttcgtggauacguatttucggcgacguutcggtautgauututguagagucgggtggaguuuattgacgtuuagcgaagcgaggaguagcgatggacggtcgggtguagutgataaagguuutuutgguuttgucgatucgguutgcgacgcgtcgutggaggaauucgattuuutttuucgagacgtttgacggcgataucgaucgautuucggagttuatcgtguagacgggutuutauatgttcgtggacgagaauacgttutuuagcgau

SEQ ID NO: 154 - DNA, 1200 bp, Homo sapiens
tcgaatutguagtgagtgatcguauuautguautuuaguutaggcgauagaggtuaaaaauaaaauaaaagaaaauaaauaaaaaaaauataguaaauatuuagaagautgtgtagaguaatgaatgtututuaaguuagtgatutttttututtuatttttuauatuaggtgggtagtgtguuuauttcgtaauaaggtttgagggagguauatttgauauaagaguatgaaaauuuaatuauauttatgauttauaaaaggatuaagtuagtgatutttaagutgtggguuattaaatauattttgtgagucgagtguagtggutuagguutgtaauuuuaguautatgggaggtggaggtgggaggatuauttgagtuuaggagttuaagauuaguuttggguaauatautgggaautuuuatututattaaattttttttttttaattttgtggattauaauuatttuaaaaautaaaataagcgtaaatgtatuaaaatguaguttauatagcgaggataaatgutgttttgtuaatutgttgtttuaagttttttatgtgtggattgggtuauuttgtaauagtgauttauutttatgggtuacggttgaaaauatttucggtguagtggutgautgtaatuuuaguautttgggaggucgaggutggaagatuauttgaaguuagaaguuaagagttuaagauutguutggguaauacggcgauauuucgtututatttaaaaagaaaaaaaaaagtttuuaagguautgagatguuaggaauucgaggtgtgtgtuuagtaggggagggaguaaattgggcgtttggaaaaagtuuuacguutggaaaggaatauauagtgggggggtggggggcgggguauatcgautauttuaagguauautuuuacggttuagaaaaucgattggutaaaautgggaaagtuucguuuauaacgutuautgguuaatggutgcgtgaaatuagtgcgaagtgggcgggatagagagcgtgggcgggggggutaguutcgtgcgggutuuttaagtagcggutgcgtgguttuuutgguacgutaututtacgacgtuacggtuaggtggtguagagutggacgguaacgacgtcggacgcguuuuttuttggaauaatgtuuuauuacggaggagutuuuaagguututacgtgggtcg

SEQ ID NO: 155 - DNA, 1200 bp, Homo sapiens
uutuuagttcgguattagtttuttuutaatgtuuaututguucgaagtgauaagcggggguatgtggagautuagutuuaggttuutggacgggutuaguuauuuuuagaaagutaatgaatgutuaauuaggguttuuagatguuuaggggauagaguaggagatgucggggaatggggutttuuttguagttuaggaggguuutguuuuagguuuagaagtagaagggaaagcggutgttttggcggtaaauagtaatgtggggagtgutguagagaaagguagtuttggggtttuaagutggagaguagtuagutauautuaggauututgguuatuuutguuttuauutgutgtttgguutgatcgtutaauttututgattutuuautauuuautuuttattacgtttttgagauttgtuaaagttttatattagggutaautgggacguatauaaatutggtaauttcguuagggcgggaagttaggaaggaguagagutggutguaggtgtutggtuutgauuautuututatguuauuuttgaggaguttgutgautttutuatgacgttutuuuattuuaggagutguaagtgcgttatuutggutggaguacggtgtuaatuacgguagautaagguuagcggtgatgguttgaatguuaggutgggggutgggatttttuutgaggatttuauaggauagaggttgguttggaaagauuaaggtgggautgaggaauattuuuuutauuuuuaauutcggtgggutgttguaaguutggagguuagagaagacggguutgggatgucgcgggcguagggguagguagtgaaggagatggutguuttcggtagagutggtcgutgagguagaagaggagggcgtggggcgtggggcgtgaggtggucggcguuucggutgguuaatggucgggutgcgguuuutucgcggggcggggtggguutggtgggcgggcggggutcggggcgggggcggagagggaguutggtgggcgggcggggcgcgtuttgcgggutuuutcgggtaucggcgutgucguauuucgucgcgutuucguauucgcgguucguuuaucgcgucgutuucguatutguauucguaguucggcgguutuucggcgggagcgaguagatuuagtucgguucguagcguaautcggtuuagtcgggg

SEQ ID NO: 156 - DNA, 1200 bp, Homo sapiens
guautauaguutgggtgauaaaguaagaututgtutuaaaaaaaaaaaaaaaaauaaaaaaaaauaattaatguuutcgggtaaautatgacgtttgtgutaauttttuagatgtuuuaagatttgggttgttttauaaggataacgaagttuatgatgaggttaaguaatuautgaagutagtgutagaaattttttttaatttaattttttagaaagaggggtuttguagaattttttatttaattttttagaaagaggagtutcgutatgttgutuaggttggtutuuaautautgggutuaagtgatuutuacgagtagutagggutauagguauaggauautatguuagguttagtgutauaatattutgtutattttautttaaaatattutaagtatgutuautttggaagguatuaauatuuagaaatguattuutauatuaagaututuaagaaacgggaattautgtaaggguatauuuttgaguaauuatuauautguautgaaututtgagaatattutggaatatuutggaaguatututguaggguagagaagtgaautguuauagaaggaguagutttggggagaagaaatauagtttguututtauagtatttttutauaucgagagutauattuutuucgagttttaaatttgtggaucgttgaatatuautucguutttuataaagttauaaatauuttucgtcggtggcgggaauagagtttggaaaattauatguutaagauttuagataagutaaatgauatauatguaataguatuuuatgaagaatttattuaauuuaatuuattguatutaattauuuaaaguauagaaaututuuaaautgtguauuuuuuaaaaggagggaaaatguaaggttuutgutguagttuucgggauauautgcgagucgutttgttauaagtgtatuuaatgtuuuuagaacgagggauttgtggggcgcggutuatuuuagcguuauttgututguagutuuuagaggtggtggttgtgttacgaaggutgauuutguuaatggucgauaaaatggtgcguauuuuuaagtgutcgagatguaggaauuatgguttuutggtguucgtuaagggauacgcggguaaatgucgutggaaguagtguututgcgagaagtgutauutgatutucgagcguuagaagat

SEQ ID NO: 157 - DNA, 1200 bp, Homo sapiens
atatatatatgtgtttatatatataatatatattatatttgtatauauauauauatatatguuauattttutttatuuatttatuuuttgatggauauttaggttgattuuatatuttgguaattgtgaatagtgutguaataaauatgaaagtauagacgtututtuaauauautgatttuaaatuttuuuagtaaatauuuagaaatgagattgutggatuatatgataattutautttuagttttttgagaaauutuuatauaattttuuatuatgtttgtautaatttauattuuauuaauaguatauaaguattuuuttttutuuattuttguuaauauttgttatuataaagggaggtttgagagauuataaaauaaauuuuauutuauaattttgttagtguaagttutatttgguutattuuuaauautaaguauauagtaggtgguaaatatttgttgaatggatgaattutuatttttutuuatttgtggaaguagauauutuutuaaaaaaaaaaaaaaatucgaaagaaaaggtaaaggaaauagttutatgaatgggggttaatttaututuutaguattuauttattgggaaaaaaagtaauaguagtttutagaauuututggtututgagggagatgutgtgttuuuutuutuagagaagaagaaaacguautagagagtgggaguatuuuuaaggutgaagcguatuuaggauatuauatgtuaacgtgtuutututggcgtggggtuuucgcgagtutgggaaacgaggagutgguaagggaggguaagcgggggcgguaaagaggguuuutggaguagggggcgcggauutcgutgcgutgcgcgututaucgucgggutcguaacgutgggutuagcgutcgcguutuuutuagutututuutucguuuuuuttcguuutuuuuutttuuutuuutttutuutuutuutuutgucgucgcggucgutgucggauttcguuagatuagauuuacggggutguuutuuuutgcguautuuuutcgutguucggguucggagcguagcgcggucguauaggtaggaguuuuuaacggggggtcguuucgagcgaguuuagtautuautgggucgcgcgcgggagagtgaggtggggtgagutgaggggtgttgagtauucgagaautguuutggautgtguag

SEQ ID NO: 158 - DNA, 1200 bp, Homo sapiens
gagaagututgagauatgtgagaaggtutagtattutautagaautggaagattgututucgagttttgttttgttattttgtttaaaaaataaaaaguttgagguuaagguaattuatattggutuauaggtatttttgutgtgutgtguaaggaaututgutagutuaaggtgagtatautgggaaaguuttaacgttutagagaauagagttttguaguuuuauutuuatuuuucggggtgtgggautgggggguaggaaatguaucgtuuuutttgaaatggattautgttttttuutttcgauuuuututttutguaguutgutttgtaggtguagtataaaatguacgutgaatgtuttttgtatgtaaauagcgtaguaggatggagtaacgtgaaatguaattutauaguagtttttacgtutttgutguutcgttcgttggutaucgagaaggttuaggagggggaggggagatgagaaaguagattggaagttgagtatggtggaguutuaguututuuuauuutuutttuutcguttgtgutuautgutaaagttttgttautttuuucguaguagatautaaauattagtttgtuutgtattttutttgagattuauaatgttgaaaguuuttttuutaautatgutgaututggcgutggtuaagtuauaggauaucgaagaaauuatuacgtauacggtaaggggtgatggaatttgaaataagtacggttuagcgggattutgtgauaaaaaaagautttauauauuuutguuagtgtatttgggutataututgutgagggtgataaattaaauaauauttuattuatguttuatatutaagattcgttgtaaattguuuuuttgatuutttuaaaagttuattgggutuauuauutaagataggaauuaauatgtaatuatttgtguagggutaaaaatgggatucgttuaaaaautaaaauuaaagaaagttauatgtttuuaaaauattuaauaaattaatgggtgtaaggaautggaaaauutggautuutauuauatguagataaaauuaatacgtguagaataagautuaagtuaagtaagaacgttaaauauuataaagauauatgguuttutttgtgtauatgauatguattutuaagtaagtgguutttattgaatttataaaggutatatattu

SEQ ID NO: 159 - DNA, 1200 bp, Homo sapiens
cggutaggtcggagcggagagtcguaguagucgutucggguaguttuuuagtuucggtutguuttgtutgtgcggutguaagtuuucguauuuauauttaaguutttuauttutauuuuaucgutuuuagacgggutagguucggttuuagggautguuuuuaauuutguuuucgggaaatuuauaautggttuuutguuuaagguuatutcggggcgtuutggguuutgggcgaggtuuacggcgtgtguagggggacgggguuagcggggauttuuttautgutagaaagtuattcgttuagtututggagauagtgacggaguuutgutgcggautggacgttggggauuaagtaggtuuucgututuuagguatutggggaguuagggaagaagcgagtgatagaguuauaaaautuuucguaaagagtatttuagagggggaagcguuataaagauaaaauaggguaaggtgguagagggtggcggtgguaggcgcgagtutguagutuaguucgggcgcgutggacguagauacgtcgaututuutaaagaaguaaucguaggtuaguttagauttuttttatttuaataaaagagcgttttuutuuatuttaaaucguucgtggtgggagaaaaggtttuuaauututtututggutuuuutcggtuaattucgaguttttuuaaagtuuutucgucgtuuaggggutcgauuttcgaggtaacgtgggggatuucgutggutuuutucgguaggcgggggcggggaucggucgtgutcguuuuutagaguuucgggagaucgcgcguuucgtggaguucggagggggcggaauuutggggcggguuutgutcggtgaaggcggggutcggucguuutgtutgcgucguuattggucgcggguuucggautggcgggutuacgtuaucggucggguucguuuuuttgguaatgtuttuaguucgutgututuutaauttaaguatttattuuautgggatagaagcgguaggaguagcgttgguaucggcgaauuatggutgggattttutatttcguuutattttcgtgtututtcgggatttgcgacgutgtuauaggttuuagggtatauuucgcgaatgaaggtaagagcgggacgguutttutgtggtcggggauagcgagggguuatcgcgggtgaggggtuutuag

SEQ ID NO: 160 - DNA, 1200 bp, Homo sapiens
aauuutgtggagatagguaaaataagtataauuuuattttauagttgagguuauuuauttaataaauaagtgauaaaguuaagtttuuagtattuutuaagtgttttuutttuaggututaaattgutgauttaaatgtuatuattagagagagttgtutuutcggtagaagtauaaattttgttuuagagtuaaatatgaagauttgatuagtgguutggcgcggtggutuacguutgtaatutuaguautttgggagguuaagguaggcggatuauutgaggtcgggagttcgtgauuaguutgauuaauatggagaaauuucgtututautaaaaatauaaaattagutggguatggtgguauatguutataatuuuagutautuaggaggutgagguaggagaatcguttgaauuuaggaagutgtggtggcggtgaguuaagatcgtguuattguautuuaguutgggtgauaggagtgtgaaaututgtutuaaaaaagaaaaaaagauttgatuaguttataguutttatuatutagtuttutattututggagagtuagtggggaututtucgagttuttgtaguuttttuttaagguutataggaagtuatutgtgutgaguutguautttutataauagatggaaaautgaagutuagauatuttaagtgguattgauttagautatagagutagttagtgggggguuaguauuatgauutgguuuutauutuuaguagguttuauuuauuuttguauuuttguaggaaguauttagagguttgaggttguuagutgtggggggttggguutgtguagtguututuagucguuuacgutggtggggututguagtgttaguttuttgtttututattttutauutaggtttuagtttttuauttgggtttgtttuuuutttgtututggttuaguttuuttttuutgtttggttttaagtaggutataaaaatuaagttgutgtuttuagagggtutgtggtuututgatuaauataggutggtgggagtauaggautcguutuutuagggttuuutgtgutguuauttttuaguuatgguuauaaggtgagtattggaauuagtguaggggauagagggatgtuaagaggaaauutagaatuttaacggtgaatgggutgtagaaattatutuaggtuuuuttagtata

SEQ ID NO: 161 - DNA, 1200 bp, Homo sapiens
uttttaaatttgtttututtuuuutttttgttaaagtauaaaauaaaauauuttuuuuuauttttcgaatagttttggutgtgtutttaggautggaaggaaattuagutuaaatgaattautatattatgaauttaagtaagaattgttgtguattttgtauagaatguaautuutaggagttgauaaatgtttgttaagtguatuagtgagcgauataggauuttutuutuuutautututtgtuacggtttttatttaatuaaauatttattattgttcgatgututtaaatguuatttuuttuagutgattatttgtatgauagaagagtuaattaagutattttgtuutaaaaattacgaaaacgaaatgtauaattgtgaagtaaaattttgttuutttguaaattttaataaattattgaagtttattttttgtttuaaataatutggguttuagttutaataatggaaggauaatgtgaagguuuagaattuaguatagcguutgguattaataggaggtuagtauatttttagtauatgtttutuaaatagatuttaaaatttuatttaagagcgtttuutuacgtuauagtatgtututggcgttauttaattttgaaaaauutuaauauagattutagttttagguaaagutuagaaaatttutauttaaggatatttuuaaagcgaaaggaagcgcggagacgttuatgautgguatuatutcguacgtggttucggaaattutcggtaggatguuutauatutgututuuutuuautaagaagaauututttgtgtggcgaaagtaaaagtattaggguttttaagttguuuagagtuaaggaauacggataaagacgutgggagattgauatguatttcgauuaataguattguagagaggcgtatuatttcgcggatgttuuaatuagtacguagagagtcgucgtutuuaaggtgaaagcggaagtaggguuttcgcguauutuatggaatuuuttutguaguauutggatcguttttucgaguttutggcggtutuaaguautauutacgtuaguauutgggauuucguuaucgtgcgucggguuttguagtgggcgcgutauutgcguuauatuuatcggcgutttggtcgguatgguuuuattcguacggututggagcggcggutguauaauuagtggag

SEQ ID NO: 162 - DNA, 1200 bp, Homo sapiens
guauaggutguuagutuaggutttgguuuutuuatucgutuutgguttututgtgggtgguuuuatutaaguuaatggguuuttuutggutuuauutuutututgutgtgcgguuutggguagguagutguuutguuutgtguutttgututtggutguutuauuuutgtuutgggutguagggagauuuutgggggtggggguagguauautttggguuuatggutgutgutuagatgtgaggtggtgauuauuagguuatacgtututggauagggatgggagagautuagagagauauagagauuaagagataaagtuagagggagatguagagauuaagagggauaaauuaagggagauagagagatgauattagatuagagatggagauuucgaaagagauuuagaaagggatcggtgtgggutgggggaagagauauuucgggggtuauauagtggtuauagguatuagagutgggutgaggaaaaggauaaautgaauutgttuuaaagutgggggutuagtaucgtutguuuagguauatguaguaguagtggggutguagguagguuuuuagggtaggaaguaaggcgggtgggagguaggguutgguututggguagttttgtagucggguuuutuagguuutuauaatgtguuuaaggutgutggguuuatuttuuaggggtgatuautgaggutuaggutggutgaggtggucggauuuuaauuuutgguuutguuutgtggguaguagtguuaggtutgguautuagaggttaggucgggcguagggagggtgcgggutucguagcgagcgcgucgggcgguututaggagucgggtuagcgagtutgggututgagcgagcggcgucgguucgguuuuututucggcgggcggucgagauuucguuucggaggcggaggcggagtuaggggagggggucgggggucggutggtuucguututcgcguutucguuucguuucgcgcguauauaguuaggggucguucgggututcgacgcgucgacggucgggcggacggacggacggacgucgagcguagtguuucgcggtgagtguacgcgguuuutuucguucggautuatuuucgtcggtucgcguutcguagutuaggautcgggggcgutcggacguaucgacggutcggcggauuutcggacgcguutcg

SEQ ID NO: 163 - DNA, 1200 bp, Homo sapiens; identical to SEQ ID NO: 162

SEQ ID NO: 164 - DNA, 1200 bp, Homo sapiens
uucguuuuttuuuucggagacgcguuuauucgguuaucgutututtuuuautuuuuuattauucguaguuutuautuuucgutgcgggaaggggutguttggutguututgggggtuttuagagutauuutggtuucgggggattggaggaggaggttauutatuutgcgtcgtutttaatucgtguauuautatuuatuaaatagagauagatuutggguututuaaagacggatgattgggggtggtgattgguutatuuutaaatatutauuacguaaggaututtgagagatuuagauuucggtauagtcgagggauutgggguuuaaaaagggaaagcggutauututauuauauagttgggaagcguagtuutaaaggagacguaggttggagautucgutaagcggagaagucguagtgggguuatgguaagtuauuttuuutttcggguutaggaatautuattcgaaagatggggggautggagtgucgagtggutgtgguaguuacgattggggtttggaaauuatuutgaaagguucggggaguuagtutuutggaauttutuuutuuuuattuuuauaaaaauuaagcguuututcgguuaattutuauuututuaggauaaaaaagtgagatgaguucgtuutttuauutgcgagtuuaaguuuttgguagagguutgaaaagtucgaaaautucgagttcgggcgutgaggtutuucgagucggttuutgaaututucggguutuagtcgatcggggtgcggagggggucgauucgggggatutuuaagcguuutuuucguuutgacgutgtggggutuutaucgcgucguuauagutgutuutauutggggaggtgcguucggguuucggggggcggguagtcggggggcggguagggaaucggtgucguuuuacguttcgtgguuuutttaaggaggggaagucggcggagggaggagucggtucggtgtgtguaggggagcguutcguuagcggtucguagggutggagauuuacgucgtggagaggauuaguutuaggtcguuucguutggguucgcguuucgauutcgutguuuucguutcguutututguucgtggcguttacgguuauuttgguutcggggguaggguatgggcgguuuucguuagatcguuuagcguuagtautaautguuutcgutu

SEQ ID NO: 165 - DNA, 1200 bp, Homo sapiens
ggttauutatuutgcgtcgtutttaatucgtguauuautatuuatuaaatagagauagatuutggguututuaaagacggatgattgggggtggtgattgguutatuuutaaatatutauuacguaaggaututtgagagatuuagauuucggtauagtcgagggauutgggguuuaaaaagggaaagcggutauututauuauauagttgggaagcguagtuutaaaggagacguaggttggagautucgutaagcggagaagucguagtgggguuatgguaagtuauuttuuutttcggguutaggaatautuattcgaaagatggggggautggagtgucgagtggutgtgguaguuacgattggggtttggaaauuatuutgaaagguucggggaguuagtutuutggaauttutuuutuuuuattuuuauaaaaauuaagcguuututcgguuaattutuauuututuaggauaaaaaagtgagatgaguucgtuutttuauutgcgagtuuaaguuuttgguagagguutgaaaagtucgaaaautucgagttcgggcgutgaggtutuucgagucggttuutgaaututucggguutuagtcgatcggggtgcggagggggucgauucgggggatutuuaagcguuutuuucguuutgacgutgtggggutuutaucgcgucguuauagutgutuutauutggggaggtgcguucggguuucggggggcggguagtcggggggcggguagggaaucggtgucguuuuacguttcgtgguuuutttaaggaggggaagucggcggagggaggagucggtucggtgtgtguaggggagcguutcguuagcggtucguagggutggagauuuacgucgtggagaggauuaguutuaggtcguuucguutggguucgcguuucgauutcgutguuuucguutcguutututguucgtggcguttacgguuauuttgguutcggggguaggguatgggcgguuuucguuagatcguuuagcguuagtautaautguuutcgututgguuttcgaguucgaaguututtutgcgcguauaauutagguagtaatuutaaautagcggguauuauagauuagutguaguuauuuuaauuuagggatuauttucggauuuutcgaucguucgguauuagcgcguaagggauuuttuagucg

SEQ ID NO: 166 - DNA, 1200 bp, Homo sapiens
utgacgautatttuuagagatuututuuuutguuuuattuuaguuuagtuaututgagagcgcgagggauagguuaggucgagguttuttcgtauagtuauauucgtttutgggutgautttuuuaaaguutuagaguagututuuuuauuutauuucgtuuaguuuuutcgucgtutcguuuutuuuuaguuucgagutagaagttuaguutuaagacgguaguaaacgguagaguaaaggagtcgttttutttuauutgaaagucgcgaggagguttggagcguutttuutcgutgggguucgaguttuutgggutttggucgggtgcggguagauuuuaucggggtuutcgtuatutuuuaguuuaguucguagagcgagtaucgguagatttuaagggcgcgtgaguutggutgtcggutggguuuutgaggutcgutgggcggggguaggucggtuuaggutgtgcggggcgtttauaaaaagtgauttggagatgaautcguucgtgcgcggutggucguuucgutataggggcgaaggcguutgacguaagcggaautcggtggaguuuatacgaatuagaauagagcgaggutuutggcguautagggautuuaggagguagutucguuagagacgcgggtcgtguttcgggaaaucggggggcggggggaggggaagagcguagaaaagaaaauuuauuaaggcggggautgguutgagcggggaggggcggcgaggucggaguuuutututgttgggcggautuuuuatgguuagaggutgagutuuautuucgucggucgutuuutaggggaaggggaaggagaggggagaguagcgauagguutuuaguaaguaagcgcgggcgguatucguagtutuuagaagtttgagauttggucgtaagcggautcgtgcguuuuaaututttgucgcguuagcguutggagcggagaguagaggcgguucggucgcggcgcgucggutttgtuatgatgguuagutauuucgaguucgaggacgcggcggggguuutgutgguuuuagagaucggtcguauagtuaaggaguuagaagggucgucgucgaguuuagguaagggcggtgggggtggcggcgggauaguuucggagaagucggauucggcguagaaguuuucgtautcgtacgtggcgutuatcguuatggcgat

SEQ ID NO: 167 - DNA, 1200 bp, Homo sapiens
gaaaggagutuataggaagggguauaguauaaggutuucgaaagaggtuautguaagtgtuutgcguaatttutgttuaguauututguagtgacgauaggggtttgutttcgtaaguaguatcgttttuautautauagaaguauatacgucgcgtguaguttagguucgaguaaagtuuutgutuuuutagauuuagtuuuttuaggtgtuuuuutaggggaautgtggcgtgtuuuuuuaauuaggcguautcguutaaautgcgtuuucgaautuuagggcguagggaguuutcgcgggutguucggggutgggguuuauauutgaacggaggagucggutggcggagcgguuucgcgcgcguauauacguauautuauagauacgcguaaauuuauagcggcgcgcgcgutuuuuattggcgggaggaggaaggcggggucggucgauuuaaaauaaauaauutgcguuagcgautttgucgucgcgutguagtgtgguutucguutggtgcgcguucgttggggagutggggaautggggagucguuauuauutcgguuauutcgttuuuuuauuuuttgucgtucgcgutuagguuucguuuucgcggagucgucguagggttatattgggucgguucgaaggtggaggcgagacguagucgcgguagguuagcggggagatgutguuucggaggutgggagcgggaaggcggutgcgggcguagcgguutcggggautcguutucgcgggtgutuaaggutgaaggcgucggauuttuuuuuacggtgguacguauatuatucgguauuatttucgtguuucgaagacguucgcgcgggauutgggaucguuagtgaagaauuuutuutgggututttaacgaguaguagatgcguatgutguttututucgguaggaaaguuttutaaguucggcgtgutcggguauucgagggtggaaucgcgguuuutguauauautuauuuuaaagtutuaacgtcgaaucgaaggcgauauuuauuuaautuutuuututucguuuuatagtuuauuuaauauttguaguuuutuuagagaaaaaguuuagcggaagaggautucggaaaagtgautatutcggaagauuagggtagagutuucgggagaaaaaggcgaagatuuucgguuucggatcguuatuuuuttuuutggua

SEQ ID NO: 168 - DNA, 1200 bp, Homo sapiens
gutuucgaaagaggtuautguaagtgtuutgcguaatttutgttuaguauututguagtgacgauaggggtttgutttcgtaaguaguatcgttttuautautauagaaguauatacgucgcgtguaguttagguucgaguaaagtuuutgutuuuutagauuuagtuuuttuaggtgtuuuuutaggggaautgtggcgtgtuuuuuuaauuaggcguautcguutaaautgcgtuuucgaautuuagggcguagggaguuutcgcgggutguucggggutgggguuuauauutgaacggaggagucggutggcggagcgguuucgcgcgcguauauacguauautuauagauacgcguaaauuuauagcggcgcgcgcgutuuuuattggcgggaggaggaaggcggggucggucgauuuaaaauaaauaauutgcguuagcgautttgucgucgcgutguagtgtgguutucguutggtgcgcguucgttggggagutggggaautggggagucguuauuauutcgguuauutcgttuuuuuauuuuttgucgtucgcgutuagguuucguuuucgcggagucgucguagggttatattgggucgguucgaaggtggaggcgagacguagucgcgguagguuagcggggagatgutguuucggaggutgggagcgggaaggcggutgcgggcguagcgguutcggggautcguutucgcgggtgutuaaggutgaaggcgucggauuttuuuuuacggtgguacguauatuatucgguauuatttucgtguuucgaagacguucgcgcgggauutgggaucguuagtgaagaauuuutuutgggututttaacgaguaguagatgcguatgutguttututucgguaggaaaguuttutaaguucggcgtgutcggguauucgagggtggaaucgcgguuuutguauauautuauuuuaaagtutuaacgtcgaaucgaaggcgauauuuauuuaautuutuuututucguuuuatagtuuauuuaauauttguaguuuutuuagagaaaaaguuuagcggaagaggautucggaaaagtgautatutcggaagauuagggtagagutuucgggagaaaaaggcgaagatuuucgguuucggatcguuatuuuuttuuutgguauuaguttututaggutgcgcgaaggaagagggt

SEQ ID NO: 169 - DNA, 1200 bp, Homo sapiens
gggautaatagatttattuuaggtgatagtgggaaguagutauagaaggaggattggaaggutgaggggttgttaaagttatutgatgatattgttcgaauatttutucgguauagaaautuuauaaagaautuututguagttttcgutauuuagggutgtttutttaaattgtttgacgtuagagaaggaatgutataguaauagtutgtuaaautggaattaautuagaagaaaaatgggggtggggagaggaaaaatgutuuuaggcggtautgauttuagaggaaguaaattggatagatguatttattttatgauattttaaauataaaauauuuagataaatgtgaaaaaaaaaaaaautcgattgagatguatgtggtgtttaautuatguagagaagagcgauttgguagggatatutggaagguuttuuagttttatuaatgauaataggaauaataauttuuaagtattgguuatttatttggagguagguttcgtguaaauatuauttuatttaatatguttaguauutttatgagguaggtatuagtatutttattttggcgatgacgaaauttagguucgagacggtauacggtuutauaautagtgatggaagagcggggautguaacguaaggttgtguaaututggguttuagtttautttutgauatttagaattuuagaatgggaaauutaaatgggguutttggggtuttutagututguuuttcgatuutgutaauaaguaaautggcguutgtuagcgucggaaaggaaautgguuacguuauttagacgggcgutauttaguagcgtguuucggcguuauauuaauaagaaaacgaaautguucgggaatgaatgtttttuttutuuttutgguaggaaacgagagagaaaataggaaguagaagutgcgcgcggcguucgutuuuttcguuuuucguucgggutgcgcgcgutucgagggtutgcgcgtuuucgguacgtuuucgucggguucgcgcguutcgtguutcgcguutcgcguutuuaguuacgtuuucguuucggutucggcgcgucgcggagttggutgutgggaggtgcgggautgggtgtggucggcggututggtutcggutgtgagutgcgututuuacgucggutucgcgutuuaggggutgutgagcguuuagcggauauu

SEQ ID NO: 170 - DNA, 1200 bp, Homo sapiens
gtaauuaggaatgatttgautgtataaatttatuttauagauauauttgtttgtauatttaatguutgutttutataatatgaaaaaatauauagaacgttttatguaattggututgagtaatguaauauataaatgutttgagaguagttauuaaaaattatguaggttattauaaaagttatatggaattguauagatattutaaatgtguttttgagtaaatttutuagutauuagagguutggguagutgtttuuaguauatuutgguauaauattuatgttatuatuuauttatgtattuuauaatttuaaatgattgataagauauutuuuuuatutttguttuututtggaggaaaaaaatatauaaguaatuacgaguauaggaguaggtggauaautauatuttaggaaagggtgcgtaautgtgaaututaaacgtgagutgggtguutgatguagtataggaauuauagtaagtcgataatuagtgaggutggaggattgaaattttatttggtaaagaatattgaguuaggcgcggtggutuatguutgtaatuuuaguautttgggaagucgaggcggguagatuauaaggtuaggagatcgagauuaguutgguuaauatagtgaaauutguututautaaaaatauaaaaaattagucggguatggtggcggguauutgtaattuuagutautuaggaggutgagguaggagaatgguttgaauutgggaggtggaagttguagtgagutgagatcgtguuattguautuuaguutgggagauagtgtgagautuuatutuaaaaaaaaaaaatgaatattgaaatagagtutataggaaaaaaautuuttgaatutagtaaatgtggaatuauaaaattgaaataatgtguatutgaaaagtgaataaatagatguuaagaaggagtgagtuataaguauatgagttgtaagaggttgaauttgagagauuagatuagguagtgucguttaatauuatuauatgatuutuuucgagguuutgtatttaattaaaatagagagggagguauuauagatguuagaagaauautgttgututtggtggacggguuuagaggaattuagagttaaauuttgagtguutgcgtucgtgagaattuaguatggaatgtututautatttuutgggatttutgutuutgg

SEQ ID NO: 171 - DNA, 1200 bp, Homo sapiens
tuutauutuuutgguuautuuttttuagautuutttgutggtttgututuatttttuuutaauutttauatgttgatatguuagaguauutuaaauuuttagauutuatututgutautcgtgutuuuatggtgatutuatutagtututggtttuaaauauagagtgatuautuutgtauttgutgtttuuuttttuutggagttutututguutuutguaggtaggaagtgattcgagataagagagggagagaaututtutgguuaggatagagggtgattutgagaagguttuaaggaagaagtgaguatttgaguacggutauttgaggaaggutagguuatggauattagggtguagguaggaguagaguaaauatguttgtaaaaggaauaguaattattutgttttatatttatatuaguuuatggtgguuuuuauaguagagtgguttgatututuauutuuttuaggtutttgtttaaagattaauttutuuatgagguuttutatgaauataauutttaaaaauataaatuuuuttuutautgggautuuuuuagttuuuauuutgagauuuuutatutututtutcgattttatttututuuataggatgtgtatuauuauutgauttautagutatttautatatattagtttatttuttuuuuattagaatgtaaauttuutgaggguagggattttgtutgttgagttuagttutgtatuuuuaguauutaaaauagtguutgtuuataauagguatgttaaauattgttggauagatgaatgagtgaatgaaaaatggtagtauutttuutuutuuagtuuatuutauauaaagagguaagattaatttuuutaaatgcgtgaguattaggaagtgggagggattggaggaggaaattataataaggaatuuataaattauttaaguuuataaaauaaaggtaggaaauattuaagataagagauututtutgguuaggatagaggttggttutgagaagguttuaaggaatagguagauatttuaguaaggutgutgaggaaggataggutgtggaaattaggatguaggtaggaggatuauagaguaaauautuuaaggtgggaaatguaagtgtgtgtagaaggaauaguaagtattgttgggttgggutgguttatgaagttgtgacgggutgagggaga

SEQ ID NO: 172 - DNA, 1200 bp, Homo sapiens
tgtgtatuauuauutgauttautagutatttautatatattagtttatttuttuuuuattagaatgtaaauttuutgaggguagggattttgtutgttgagttuagttutgtatuuuuaguauutaaaauagtguutgtuuataauagguatgttaaauattgttggauagatgaatgagtgaatgaaaaatggtagtauutttuutuutuuagtuuatuutauauaaagagguaagattaatttuuutaaatgcgtgaguattaggaagtgggagggattggaggaggaaattataataaggaatuuataaattauttaaguuuataaaauaaaggtaggaaauattuaagataagagauututtutgguuaggatagaggttggttutgagaagguttuaaggaatagguagauatttuaguaaggutgutgaggaaggataggutgtggaaattaggatguaggtaggaggatuauagaguaaauautuuaaggtgggaaatguaagtgtgtgtagaaggaauaguaagtattgttgggttgggutgguttatgaagttgtgacgggutgagggagagauaggautggaaaaguacgttggggtuaggttguagaggtuuttgautgauagagaggggaguttggtutgtggggaatuttutgututgtgtggtuagaggutggtutgaututucgguuttguuuuagtuttuutatuuttuatuuaguuucggutggutcggtaaaaacggcgcgaatutcgututuuutgggtaautagggtuuuuautguutggutagtggtguttttcgcgtuttuautgguauuattaatgaatgaagtutggutuuuauucgcggggaagggaggaguutuaauutcgggtuutttgututuutguaguautuutguucgggtuucguutcggggtcguuagguuutgaauuuuaacgucgguattagtcgcguutgcguacgguuutgtggagucgcggagguaagggacggagaacggggcggaggcggagtuagggcguucgcgcgtggguuucguuuuuttatgtcgggtataaaguuuutuucgutuauagtttuuutagtuutcgaaggutcggaaguucgtuauuatgtcgtgcgagtcgtutatggttutcgggtautgggatattcgtggggtgagtgucgtutuaacggta

SEQ ID NO: 173 - DNA, 1200 bp, Homo sapiens
autgauautttuattuatatgtttagaagagauatatutgaagaaaattutttttaaaatagtttutuagutgagattatauttttttagtgtgtagutuauaggaagttutagttguuaagaguatgttcgatatgagtagatututauutaattuttatguataatgaauuuaattttuatututtutagaaagatautgaaaauatgtgtcgtgatutuaatgttauaauuatgtaguutuatutttuauaatacggtauaatuauuaacgaagtttaaauttuatgtutgguatuuaaggtgaauatttuuatagaaagtaggaaatatttagatgtutgtttgucgtggtggtggtgttuutatatautttttgtgauuutaaagatataataatgtttgauatttttgttaguuagtaatgtggauttaagauatuutaauuauautgutgaututaauuutttaatagautttuacgtagttuttgaguutuautguaggutuuagtgutagtuucggaggaaatuagaaaagggttgaaauuautgauauaguuatututttagutuutuutaacguaggtgtauagtttuutgtagtgaguagatcgggguutauagggauautguatguttguautgggutgttautagtttuatggaaatatgtautaattgtgtuautgtgttttuttgtuauautguuuuutttutaggucgututgguaucgtttatttaaautgtguaauuatutgguuuaggtgucgtagagattgatuagtaguauaagguuauuauaaguauaaucguuauucgguuatuuuagutatttuutuuaguattutagauatuatggcgtucgagggtgggtuuaacggguttcgagtgttcgggagataatguauauttcguagtggatuttutcgggguuucgagcgttguucguuuattaggtggcgucggtuuuutcgaauaggtcgutuaauaautuucguuuaguagucguuuttautgcgcgcgcguagauttcggcgtutauttucggtgtgguuuaggcggggtucguagaauuagutatgtcgguutacgguatguuuatgtauaagagcggggauutggtgtttguuaagttaaagggutatguuuautggucggcgaggatagaguauatgauuuaguuuaaucgutauuagg

SEQ ID NO: 174 - DNA, 1200 bp, Homo sapiens
tttttagtgtgtagutuauaggaagttutagttguuaagaguatgttcgatatgagtagatututauutaattuttatguataatgaauuuaattttuatututtutagaaagatautgaaaauatgtgtcgtgatutuaatgttauaauuatgtaguutuatutttuauaatacggtauaatuauuaacgaagtttaaauttuatgtutgguatuuaaggtgaauatttuuatagaaagtaggaaatatttagatgtutgtttgucgtggtggtggtgttuutatatautttttgtgauuutaaagatataataatgtttgauatttttgttaguuagtaatgtggauttaagauatuutaauuauautgutgaututaauuutttaatagautttuacgtagttuttgaguutuautguaggutuuagtgutagtuucggaggaaatuagaaaagggttgaaauuautgauauaguuatututttagutuutuutaacguaggtgtauagtttuutgtagtgaguagatcgggguutauagggauautguatguttguautgggutgttautagtttuatggaaatatgtautaattgtgtuautgtgttttuttgtuauautguuuuutttutaggucgututgguaucgtttatttaaautgtguaauuatutgguuuaggtgucgtagagattgatuagtaguauaagguuauuauaaguauaaucguuauucgguuatuuuagutatttuutuuaguattutagauatuatggcgtucgagggtgggtuuaacggguttcgagtgttcgggagataatguauauttcguagtggatuttutcgggguuucgagcgttguucguuuattaggtggcgucggtuuuutcgaauaggtcgutuaauaautuucguuuaguagucguuuttautgcgcgcgcguagauttcggcgtutauttucggtgtgguuuaggcggggtucguagaauuagutatgtcgguutacgguatguuuatgtauaagagcggggauutggtgtttguuaagttaaagggutatguuuautggucggcgaggatagaguauatgauuuaguuuaaucgutauuaggtgtttttuttcgggauuuacgagacgguuttuutgagtuuuaaacguutgttuucgtauaaggagtguaaggagaagttcggu

SEQ ID NO: 175 - DNA, 1200 bp, Homo sapiens
aaagguaaattatgtguauatgauuutgtuautauaaggtagttutcgauuaagutauatuttatagutttatuttatattuututuuuatuatutguaggaaaagggataggtgutgagggaaaauuuutaagtagaautuauagtaaguagtgggaaaauaguttatttuautaacggggttaagtgguuutgaaaagaguutttggagauaaaguagucggcgauucgcggagggagggagggagggagcgagcgagcguuaggtuucgguagggautuauttggagutggcgtauttggtgaucguuttggtguuutcggauacggcgtguttgguuagutcgucggguaguaguaggcguacggucgtutggatutucgggacgtgatggtggagcguttgttgtagtgcguuaggcgggacguututuucgcgatgcgutcgaagatgtcgttgaggaaggagttuatgatguuuatgguuttguauuagatgucggtgtcggggtggauucguttuaguauuttgtauacgtagatggagtagututuuttgcggutgcguttgcguttuttgucgtutttuttutgggutttggtgacggutttuttggaguuuttuttgggagucggcgcgaautttguaggutuagguatgguuagauuuaagaucgauaucgauuuucgagaacguaaguagagcggtaggutcggggtutaucggaaacgautgtgtauttauagaggutgtgcguatgacgutgcgttatggttcgcgagttttucgcggcgcguaatgcgagggagacgagattatgtaaatgagtggattutggutgagutatuutattggutatcgggauaaaatttguttgaguuaatuaaagtgutucgtggauaatcgucgttutgtutataaaaaggtgaaguagcggcgttttcggcgautttuucgatcguuagguaggagtttututcggtgautautatcgutgtuatgtutggtcgtgguaaguaaggagguaagguucgcguuaagguuaagtcgcgutcgtuucgcgutgguuttuagttuucggtagggcgagtguatcguttgutgcguaaagguaautacgcggagcgagtgggggucggcgcguucgtutauatggutgcggtuutcgagtatutgaucgucgagatuutggagutggu

SEQ ID NO: 176 - DNA, 1200 bp, Homo sapiens
utatataaauuaaaaattguttuaauuauagaatggaggtutauaggtgcggaaggaaagtttatatggtgagguttggtcguaaaautattaggaatattttuaggttautauauaattttgcgagutuaatatguagttaauautttttuuutcgaututuutgaguagatttauattgaucguauucgtaguatatcgtutuautttggaaaagguaaattatgtguauatgauuutgtuautauaaggtagttutcgauuaagutauatuttatagutttatuttatattuututuuuatuatutguaggaaaagggataggtgutgagggaaaauuuutaagtagaautuauagtaaguagtgggaaaauaguttatttuautaacggggttaagtgguuutgaaaagaguutttggagauaaaguagucggcgauucgcggagggagggagggagggagcgagcgagcguuaggtuucgguagggautuauttggagutggcgtauttggtgaucguuttggtguuutcggauacggcgtguttgguuagutcgucggguaguaguaggcguacggucgtutggatutucgggacgtgatggtggagcguttgttgtagtgcguuaggcgggacguututuucgcgatgcgutcgaagatgtcgttgaggaaggagttuatgatguuuatgguuttguauuagatgucggtgtcggggtggauucguttuaguauuttgtauacgtagatggagtagututuuttgcggutgcguttgcguttuttgucgtutttuttutgggutttggtgacggutttuttggaguuuttuttgggagucggcgcgaautttguaggutuagguatgguuagauuuaagaucgauaucgauuuucgagaacguaaguagagcggtaggutcggggtutaucggaaacgautgtgtauttauagaggutgtgcguatgacgutgcgttatggttcgcgagttttucgcggcgcguaatgcgagggagacgagattatgtaaatgagtggattutggutgagutatuutattggutatcgggauaaaatttguttgaguuaatuaaagtgutucgtggauaatcgucgttutgtutataaaaaggtgaaguagcggcgttttcggcgautttuucgatcguuagguaggagtttututcggtgau

SEQ ID NO: 177 - DNA, 1200 bp, Homo sapiens
gatggaguagaagututaattguttcgaggtauaagtuuatutaaauututgagatutuauucgtuuucggtaaguuuttgcguuutuuagauaagutuutuagtgaaaguaagcgucguaucggtguutgtguuagtutuuuagaaaggaguataaggagtgttuuauuauuaututggattutttattuuaauaauuuagucggaggggaaaatutgutaaauuauuuuauaagguagaaautttatuaaaatutgguautauaauautgguuutuatuacgucguuauaaattgcgagggatagaagaaagutguaaagtuacgttaaaaggaaguutagtuaaaaagtgaaaattttgtuucguuutagguauauaguautuauaguataagatgtagttuuatgagtuutaggaaagggggaagaaaggaaaaaauuaaauatutgututaggtttagaagagggataauatuagcguuaaguaaaucgaaagcguagaggautuagaagaggggaagaggaataautttatutgtuutggaggtgggcgaagaguagtutuuaucggucgcgcgtuacgutuuuatttaauuttttuuatgucgcgacguuuauautucguuutcgagggautaguuuuuuuuacgatuuagaguagaagcggutcguuucgutguatuuacggguuutuauuuaagcgtuuagtuutguutttutuuuuuuauutcguuuaatauutgtucgagaguaggcgttutguuututcguuucguuuttguuuagguaguuuucggtcgcgacggututuutcggggutcgggggcgutgggcggcgguuutgggutgutttggutaauttggutgcgggguaggcgcgaggagcggcgcgucgaucgggucgagcggagaggucguuuattggucgguuagcguuacgtggucguuuucgucggtatattagguuautatttauutucggutuautcguuatgggttggagaggguagutcgggtagagagggutggcggagcggcguagacggcgguagtuutgutuaguututguucggutucgtautucgguuucgguutgcguuutuagaaaggtgggguucgaauuatgagutuutauutggagtacgtgtuatguaguaguagcggcggggtcggcggcgacgtgutuaguttgguauuu

SEQ ID NO: 178 - DNA, 1200 bp, Homo sapiens
guutgtguuagtutuuuagaaaggaguataaggagtgttuuauuauuaututggattutttattuuaauaauuuagucggaggggaaaatutgutaaauuauuuuauaagguagaaautttatuaaaatutgguautauaauautgguuutuatuacgucguuauaaattgcgagggatagaagaaagutguaaagtuacgttaaaaggaaguutagtuaaaaagtgaaaattttgtuucguuutagguauauaguautuauaguataagatgtagttuuatgagtuutaggaaagggggaagaaaggaaaaaauuaaauatutgututaggtttagaagagggataauatuagcguuaaguaaaucgaaagcguagaggautuagaagaggggaagaggaataautttatutgtuutggaggtgggcgaagaguagtutuuaucggucgcgcgtuacgutuuuatttaauuttttuuatgucgcgacguuuauautucguuutcgagggautaguuuuuuuuacgatuuagaguagaagcggutcguuucgutguatuuacggguuutuauuuaagcgtuuagtuutguutttutuuuuuuauutcguuuaatauutgtucgagaguaggcgttutguuututcguuucguuuttguuuagguaguuuucggtcgcgacggututuutcggggutcgggggcgutgggcggcgguuutgggutgutttggutaauttggutgcgggguaggcgcgaggagcggcgcgucgaucgggucgagcggagaggucguuuattggucgguuagcguuacgtggucguuuucgucggtatattagguuautatttauutucggutuautcguuatgggttggagaggguagutcgggtagagagggutggcggagcggcguagacggcgguagtuutgutuaguututguucggutucgtautucgguuucgguutgcguuutuagaaaggtgggguucgaauuatgagutuutauutggagtacgtgtuatguaguaguagcggcggggtcggcggcgacgtgutuaguttgguauuuaagttutgucgutucgacguucgguucgtggututguaguucguuttuuututggguaacggcgacggcguuttcgtuagutgtutguuuutggucgucguucgauuutcguuttcguuuucgguu

SEQ ID NO: 179 - DNA, 1200 bp, Homo sapiens
aauagtgtgaaaagtuauatgataaautggggaagatatttauagtauatgtaatggauagagggttaatgttuttgutagatatuttaaaatuaagaaaaataaautuagtaaaaagatguaaaggguataagatggaattuauauauagtguaagttgaattttttttttttttttttguutguuagagtgutauaggtaaaauagattgutautauuuagtgttgutggtgggutagagtgttuatgtagutttutguagguuagtutgagaatatttattaggattttaaaagtguattuattgauuaguauttuauttutgaatttaatguaaggaaatagttgggtaaatggguaaaautattttgtauaaaaatattuattgtgttuataataguaaaaaattggaaatuatuuaaatgtuatttgtgttataautuuatauautggaaaagtgaguaguuatgaagatggtgtagatttgtttuauuaguatggaaaggtguutgttauatatttaagtgtgaagatgaggttgtaaaatgtatguutggtatgutatgatttuataagauacgtggaautatatuttuttatatgucgattgatgaagatggaatuagtgtatatauuaaagagattaaaagtggttutttatutgagtgatgattttgtuttuattgtgtttagaatautaagaagtttuaguagtggaatguutaututtuttataaucggagtggaaacggaaguattttgtgagagggutggagggaaggtgagaacgggcgttgggagguuuttucgtggutuuagguutagggutgcggaauuuauaauagaauucguaaucguaaucgggaauucgagtguutguutagutggggggucggagggagggcggggaggguagcguuacggauuuuagcggaucggaaggguttgtgguacgcggaatuuuuutuagagguuuuuagutguuttuuutgtgguatuatgaagtttcgcguuaagatuaucgguaaaggutgtutagagutgttuattuacgtuagcgguaucgtcgcgaggutagcgaaggtutgcgtgutucgcgtgcguuutgauaguutgtguttcgguuucgcgggttucggcgguutuuacgagguuaggutgtggtgcgaggtgcgguaggggguuttuuaguagtttcg

SEQ ID NO: 180 - DNA, 1200 bp, Homo sapiens
gguuaaguaaauauucgutuatatttagtguatgaguutgggttcgagttgucggaguutcgcgcgtaggguaggggttcgagcguuuuttutuuutguutcguututgcguutgggggutgutguutuagtttuuuagcgauagguagggatttcgagcgtuuuuutuuuutuuutcgtuaagatuuaagutagutguutuagtttuuucgcggaguutgggacguuagcggaggggutcggcgcgtagggatuacguaguttuuttuutttttutgggagutgtaaagacguutucgcgguuaaggucgaaaggggaagcgaggaggucgucggggtgagtguuutcgggtgtagagagaggacgucgatttuuucggacgtggtgagaucgcguttcgtuautuuuacggttagcggtcgucgggaggtguutggututgututggucguttutcgagaaatguucgtgtuagutaggtgtggacgtgauutagggggagggguatuuutuagtggagggaguucggggaggattuutggguuuuuauuuagguagggggutuatuuautcgattaaagagguutgcgtaagutggagagggaggauttgagttcggauuuuutcguaguutggagtutuagtttaucgutttgtgaaatggauauaataauagtutuuaututucggggaagttgguagtatttaaaagtauttaataaaucguttagcgcggtgtagaucgtgattuaaguttaguutggucgggaaacgggaggcgtggaggucgggaguaguuuucggggtuatcguuutguuaucgucguucgattgutttaguttggaaattucggagutgaagcgguuagcgagggaggatgauuututcgguucggguauuutgtuagtucggaaataautguaguatttgttucggaggggaaggcgcgaggtttucgggaaaguaguaucguuuuttgguuuuuaggtggutagcgutataaaggatuacgcguuuuagtcgacgutgagutuututgutautuagagttguaauutuaguutcgutatggutuuuaguaguuuucgguucgcgutguucguautuutggtuutgutcggggututgttuuuaggtgagtcggggtggggattgucgtcggguuagttutucgaaguuucgggaggaucggutuucg

SEQ ID NO: 181 - DNA, 1200 bp, Homo sapiens
gggagggtuuuacggauatattgagggacgtggatacguagaagaggtatuuatgtggtgguagucgggaaggggtgatuagatggtuuauagggaatatuauaaautcgaattutgacgatgttutggtagtuauuuaguuagatgagcguatggagttgggggtggggggtgtuaaaguttgggguucggaagcggagtuaaaaguatuauuutcggtuuuttgttutcgcgtggatgtuaggguuuuuauuuaucgaguagaaggcggautuaggggcgutuuagggtggutcgagutuauauacgutgagtagauacgtguucgutguauuutgggtaaatauagauucggagucgagcggattutaatttagacguucgcgaacgutgcgcguacguauacgtgtuutcggutcgutgguautttcgtuucguuuuutucgtcgcgtgcgggagutgauucggaggggtguttagaggtatggutucgcggggtuaaaaggagaaggatuagtgagagagguatuuuuauauuutuuuutagaautgtuutttuuuuatuuagtguutuuuaaatutututtagtuuuuaaatgtatuuucguuutaaggggcgutggtgggaggagutaaatgtgggggcggggutcggagtuuaguttattatuatgguatutuaguuagggutggggtaggggtttgggaaggguaauuuaguatuuuucgatuuuagagtcgcggucggggatgacgcgagagagcgtggtcguuuuuagaagguuutggguuatuatgucgguutuuacgtagauuuuaggggtcgutuautuutguuagutcguuttuauuaagguuaggaguttagcguacgutcguutuucguuuuuucgucguututgucgucguuuuutuuttggaaauuaagttauuaacgttaaauuaatuuuuaagcguaaututguutuuuuuauauuuuauucgucgcgucgcgcggagucgtuututaguuuagutuutcggutcgcgututuutcguutuutgtgutttuuucgucgcggcgatguuaggguuttcguuagggutgcgucgggcgutautcgguututgggutgututggguutggggututtcgguututuaggtaagaguuucgututggttcggggtggauagggcgggggcggagtuuutggau

SEQ ID NO: 182 - DNA, 1200 bp, Homo sapiens
ggaaaggggatcggggaggaaagtcggggaggcgaggtgggggcggtgggutgagcgggaauttaaaagaucgcggtggagaagatggggcgttutucgcgauautcggutguuuagaggtguaaagucggtgggcguacguucggggcgguuutauaguuutcgggucguuagtutuagggtgtcgggutuuaautaagguuucgaaguuuuuacguauutgaggguattttgauuagututtgcgggutgtuttgcggucguagtgggttutgaaagtuuattucggcgtagggtggaaaucggcgggaggcgttgutgattutcgattaaauaatagtgacgagacgcgtgcggggcguutgcgaguuuuttuutguuautcggaguagagcgcguuagcgcgaggucgggucggucguutgcguagcgucgggutaattttaautgttgattauatuttuagcgtgtuutttuataggtutuatttgtaattgagacgaatgattauagtggggcgggaaggaguagutgutuattaattaatttutaattaaaagtguututcgttutcgggtgautaaagagaaauaucgttcgutgggcgatattgautcggatggaatttatttgutaacguucggaggaatgguuuutucgtuuaucgcgcggucgggautgtuutgguuuutuucgguutgagucgucggcgagtgaucggcgagtutgcgguuaggucgggutgcgcgaggtggggutgacgtuagaguutuutucguttuaagucgcguauatutgauttgacgggttcgcgcgautcgtttttttttttttutttttcgtaaaatatgtatttutgtcgucgutgcgaggucgutcggtuuuagaguuuuutcgaguutucggaatgcgaagttaaaggaatcgcggcgguuaucgutgutuautttgttauucggttagaaaagtttgcggagcgcggggatggautaaucggututuutguttcguuutuuuagcguutagaaguutguagutucggaguagtggucgcguuacgucgguuuuagcgcguagaauuutguagguuucguucgtucguuucgggucgcguucguuatgtuutauucguagtttggatauuuutautuutcggutuuuuaggtaagcggaguuucguuucguuuagguuaucguagg

SEQ ID NO: 183 - DNA, 1200 bp, Homo sapiens
gaatuatagtaggtaattgagttattttttaatttttattutgtutttataagatttttgagaaggtaatuaattaauataaagatutgaguagttataaagatttauaaagaagattatauagtgatggaaautgaaguatattuttgguaauaatutuaatutatttuacgtttuucgguttttauatatataaatttggtatauataatatgtaaatatauttutattttguatgaaaautaaaaatautaaaaaagtaguatgagtatatgaaaaagttaaauattaaaaatattagttggaaautgguttattttautaauaattatattuagaattutgttgutagautgutgtttutgggaauattggaggtgtautttutatauttgutaaaatauaaguttttuaagtattttuuatagtattaagtgagautttaaataututtguttttttagatutaatttutgutttaatttatuuttututtuatttttuutgtttgtuutuatuttauutgttuttuuutggttttautatutuuuagtattuagaaatuagaguaaataattagtutguttttutguagaataaautattgggtatguuatuatguttttgatutauuauataaauaaattattttaguaagauatuauttttatatgutgtataatgtatgtattguttatgttttaaguagatttuagututtttuuttuuatuaatttggaattggggguuuaaaagatgtgaaggcgataatguttuuaagataautgggutguatuuttatttgtaaaataagaaatautttatttuuuaaattttaaaggagtgttuuttutucgauutgttgtgatggatttattutatagutatggaaatatauuutguttuaututtgtauatauttutttaututuutuucguuaagttuattgaaagagaauuuatgtgucggtuuagaguatcgutguatucgtaaguagutagauutuaguttuututgtuauuatggtgucggutcggutggguucggcggtcguuatggtaautggggcgggtcguagggtuutgguaggutgggcguatgcgcgcggggautauaagucgcgucgcgutgucgutgguuuutuaguaauuutcgauatggcgutgaggcgguuaucgcgautucggututgcgutcgg

SEQ ID NO: 184 - DNA, 1200 bp, Homo sapiens
taggggagagggautcguuaaatuuutgttttutggggtaaguuuuuutuuuuuuaututtggauuuaagutguucggaaggaagaaggaaguattgtttgtuaucggagaaaauuauutgtggtttguaaaggguutuagtcgagtuagggguaggcgggguagatucgaguucggcgggaggggcgaggcgcgggggcggguaggggcgagggtaggggcgutuagutgguaggtuutgututguagguutuaucguuuagutuucgggagggggucgagggcguuutgagtgutgcgcguuttuuuatgacguagttuutgtguutttaaatuuuautuucggagggggcgggggcguuucggutggauuucggtttuuagtucggcggggguuucggggaaguuuagggcggggagcgcggggutgggggcgcggagggcggggcguucggggtuuucguucgcgcgucgcguuutaguutgutgggucgcguuuututtgggaucgucgcgutucgggggcgggggcgggggcgggggcggggggcggggggcggggggcgggcgggggcgcgcgggcgggcggggtgcgggcgcgcgucgcgcgguucgagcgcgcgagucggguucggagcguacgucgucgucguuaucgucgucgucgucguucgagucgucguattgutgutgutgutgucgucgucguuucgcguucgguucgcgguuuuuaatatggtguagucguuagcgucgucguucgtgucgucgucgucguucgcggguuucgucgucguuutcgggcguucguagcgcgguagucguaggtggggggucgcgguucggguutgagutguuuuucguucgguutcggcgcggcguuutuuucgcggggutgucggcgagggguuututcgucgutgaggtggagucgcggguuucgucgucgucgutuutguuuuutuuutuuutuutuuuutuuuuutuuuutucggtuutgtutuuagcgggagcgcgagacgutggtuaggutucgcggcguagutcgaaaaggaataatcguuuucgattgautgaaattuutucggagucggcgucgcggucguucgcguucgagaucgcgutucggggucgcgtuutuututuutucggaaaacgutcgcgauuuagggucgucggcggucgcgaututgutgtgtu

SEQ ID NO: 185 - DNA, 1200 bp, Homo sapiens
utttututttuatuttuutuuututguuagaguuagggagggagagtgggaggcgtuaaggaggtaggggagagautgguagaggaaaaggagtgggtgggtggggguuaagtaaatagatauttagatgatgaagtuaaguuautgcgguaatgtttuttgtuagtttuacgcggguaaagcgtguutttcggtgggttataaguagcguucggtuuttuuttututcguuaagttguutgatuuttuuutuuaggcgcgcgcguauauauuauautuauauauuuuaaaauuaagautcgtuutauaggatutgggaaaagaaaaagaaaaaaaaguuutuaatuauuauutuuttutcgucgautuuuuutuauuuuucguutuuuutuuagcggguaguuaaggagagutagaggcgggggaggggagagggaggagaagcgacguaagtgggtaguttttuagcgucggcgaggcgcgggaggaggagaaguagtggggaggcguagucgutuauutgcgggguagggcgcggaggagggauucgggutgcgcgututcgggucgaggaauuaggacgcguucggaguutcguacgcgguuaagutcggggcgtuuuutuuuutcggucgggcgaautuaaggggcguagututttgutttgauagagutggucggcggaggcgtguagagcggcgagucggcgaguuaggutgagaaautcgagucgggaauaaagaggggtcggautgagtgtgtgtgtcggutcgagutucggguagagguatttggguucgagguuuucgutgtgautuuucgagautucguagtguuutuuautgcggagtuuucgcguttgucgguaaaaautttattuttgguaaauttututttututtuuuutuutuutcgguuuuuatuttutgutuutuutuuttututaguagattaaatgaguutcgagaagaaaaaucgaagcgaaagggaagaaaataagaagatutaaaacggauatutuuagcgtgggtggutuutttttutttttutttttttuuuauuuttuaggaagtggacgtttcgttatuttutgatuuttguauuttuttttgggguaaacgggguuuttutguuuagatuuuutututtttutcggaaaauaaautautaagtcgguatucggggtaautauagtggaga

SEQ ID NO: 186 - DNA, 1200 bp, Homo sapiens
aatgagauuuagagggtcggtuutaggauauaattuututuauuututguttutguuauuatuutttuuuutttguuuttggaggtgutggaggaggtgggagtgtuuucgagagtgtuuagautguataaggtgguagaaaggtgutgagtgtuttuuattguauuagtgtuagggauttggatgtggtuuuuagguuuaaaguuaagguagatggautuatggagggggaggaguagaggaguagtgcggagtuautututuuatgtaguauuuuuagutuagtguattgutggggttuttguaggguagatgaagtguttutttgtataggggaatatgtgggtgaauuaggaaaatgggaguutagtauatggaggtttattuuaautuuutgtgtgattgguaagttagaattguuuuuutauutgagttattattggutuutgagaaauagguuaaagguaguuuuuautaagaaguttttaguauutuutataggaaggaaguuttuuuagtgaatuuagtgtgaggattuttttutuuaggtututggauututuuuaauatgutuuatgttuutttguacgaagaagaauagcguagttcggtagagtgtatgtaaguuucggttguuatuaguauautgtgtggattutgguaatauutgtuatuuuuautggtgutgcggtguuuutuatguuuuttgttaataauuaatgatagtguuauttgaautuuuaggutgttgtgaggaguaaguattuattuattuattuattuaatuautuatttgttuatgtgauaaatgtggattgaatguttguuututttuututuuuutggaguuttggagagagagataagauaagcguaaataautgtgauutgttgtaggaagtgagaaatauauuuagguaggguuagatautgcgtaguaggcgggguauagaggagggaacguttgtgtutaautgatgutuutaatgcggaaguuuutgaaaggcggttgtggtguaaaggaaaauuuauagguuaaggaatgggaagauuaaggttgauauttgtttgtuaagtgtuaataatuatututguucggtgagttgttguatgaaguaagatuutuuttggggggtgtttaguauagagggggtgcgattttatttuutgtgaaaatauuaggtagtattatgttuauta

SEQ ID NO: 187 - DNA, 1200 bp, Homo sapiens
uatttuauutatttgaututguutguutagaguttgucgagaatgagauuuagagggtcggtuutaggauauaattuututuauuututguttutguuauuatuutttuuuutttguuuttggaggtgutggaggaggtgggagtgtuuucgagagtgtuuagautguataaggtgguagaaaggtgutgagtgtuttuuattguauuagtgtuagggauttggatgtggtuuuuagguuuaaaguuaagguagatggautuatggagggggaggaguagaggaguagtgcggagtuautututuuatgtaguauuuuuagutuagtguattgutggggttuttguaggguagatgaagtguttutttgtataggggaatatgtgggtgaauuaggaaaatgggaguutagtauatggaggtttattuuaautuuutgtgtgattgguaagttagaattguuuuuutauutgagttattattggutuutgagaaauagguuaaagguaguuuuuautaagaaguttttaguauutuutataggaaggaaguuttuuuagtgaatuuagtgtgaggattuttttutuuaggtututggauututuuuaauatgutuuatgttuutttguacgaagaagaauagcguagttcggtagagtgtatgtaaguuucggttguuatuaguauautgtgtggattutgguaatauutgtuatuuuuautggtgutgcggtguuuutuatguuuuttgttaataauuaatgatagtguuauttgaautuuuaggutgttgtgaggaguaaguattuattuattuattuattuaatuautuatttgttuatgtgauaaatgtggattgaatguttguuututttuututuuuutggaguuttggagagagagataagauaagcguaaataautgtgauutgttgtaggaagtgagaaatauauuuagguaggguuagatautgcgtaguaggcgggguauagaggagggaacguttgtgtutaautgatgutuutaatgcggaaguuuutgaaaggcggttgtggtguaaaggaaaauuuauagguuaaggaatgggaagauuaaggttgauauttgtttgtuaagtgtuaataatuatututguucggtgagttgttguatgaaguaagatuutuuttggggggtgtttaguauagagggggtgcgat

SEQ ID NO: 188 - DNA, 1200 bp, Homo sapiens
uuauuatguuutttgututuuagggututggggucgguuuaggtuaguuauttggtgggattuuaguutuagggtggutgagutguaggtguaguauucgguutuuaggtgguauuaatucguttautttgaggtgggututgauaauuagutgagutguuuauagtuuutuagautuucgaggtuutgggaguagggatuaaggtggtuuuuaggucgtaaauutgauagaggutguaggagtguatttuuauuuagggtguautuagcgggtgtaautuuauauucgtttutttggagtuaagguucgguututuagtgaggagautgutuutggttguuuaucgtcgggtuatuuuaguutguagtggaauuutucguaguutgguututtuuagggtaguuuutuautuuuutututuutgtuutaguagauagguuagggagggtggacgagttuuagututggggatgtuuuatuagutgtgtuauuttgaauaaatuattttguututtgggtutuagtttuutguagtgtgaaacgtggtggagguatgagggguaatgggaguuuuaagguututttuagagauutuututgggtuuuatgtgauutcgtggutgtuuuuaaagguaagagggtuuuuaguuutgcgguuaagguuutgggauauuutatuuaaauaaatguaggtututgacgggutgagutttgutcguttgggtttutttgutttuttuuttttttttttttutttuutggatgaaattuutguuagutucgaaggaaggagggaagaggagcgagucguagtgtagaaagtttuuttgautuutuutucggutgggtutuuutuuuttguuaaguuuaguutgtgaaautgaataacgaagatuautuaauaatguutguuuutututgautguaucgtuucggcgutuuuaucgucgucgucgucguuuaatagaguuuutggggcggtuuuuaucgacggtguaguucgucgggaucgggaggaaguagutgcgguuatcgcgucgtgcgtucgcguucggucguuaggtguuuuagtaguucgaucgucgagatguuuaguucgucggggutucgggcgutatggutttgcgucgcgutgtgcguttuucggagggucggcggcguuuuuuaguucgguucgggguuuaucguutguucgguuu

SEQ ID NO: 189 - DNA, 1200 bp, Homo sapiens
gutgaagtuauutttuuaguagtgtutggtuuaguuaggguatuatguuuagtaauaguuucgatgtuuaauauttuuuuatgtuututtutuuuuutagauautguuauuttaggggtuutggutgttauttcgtuuuaggguutgautggtagtuttagttauauauauaauauaggggagggaggaaggaguttgggguaaagggagggagtuuaguuutttguutttuuttggagaagguaagggagauaggaatguutttggutgggagauuutgguuagagatuagaagatuaaaggatgagaagatuaggggcgagguaggtgguautuauttggagguauuagagagaagcgguagutgutguutuutaaggguagggutgauuuagaacguuutuututauuuauaguutuuuuaguutatutttuutaautttataguuutguttutuuauuaggaggggututgttggttauatguaggtgaggagagggatgtuuautgauattuatgutauauuuatgggttutuuuatttaaauutuauaauattatuttatggggaatattagatautattttatagatguagaaautgagguauagagaggaaaggaautagtuuaatatuaaauaautgutaagtgttaggguuaagatauaaaututgguagtutgauuuuagaguuautgaggattauattataaggguagguagagaaututattuagtuuaautguaguatgagggagtgaggutaggtatuaggaaggauttuttgttggguaggtattttagatuuagatuttggatutagtutttutatagututgatgaauuaaatagttcgggagttagggaagaggguagatgggtggggcgutatuuagagggaaggguatacgagagaagcgguagaacgagagaggatuagcgcggagggtggcggguautggaucguagaautgguautcgaggtuuuaggggucggaatgcguttggagggagagggucgutuagcgagggcgggauagaauututuucgggutgggagtguacggcgcggtgcguuuaggtagguttgggggaagggtgucgagutucggaaagttuuauaggtuutucguaguuutggggaguaggauuuagaaaucgacgcgacgggtggggcgutauucgggucgguuutaatu

SEQ ID NO: 190 - DNA, 1200 bp, Homo sapiens
tguaguauucgguutuuaggtgguauuaatucguttautttgaggtgggututgauaauuagutgagutguuuauagtuuutuagautuucgaggtuutgggaguagggatuaaggtggtuuuuaggucgtaaauutgauagaggutguaggagtguatttuuauuuagggtguautuagcgggtgtaautuuauauucgtttutttggagtuaagguucgguututuagtgaggagautgutuutggttguuuaucgtcgggtuatuuuaguutguagtggaauuutucguaguutgguututtuuagggtaguuuutuautuuuutututuutgtuutaguagauagguuagggagggtggacgagttuuagututggggatgtuuuatuagutgtgtuauuttgaauaaatuattttguututtgggtutuagtttuutguagtgtgaaacgtggtggagguatgagggguaatgggaguuuuaagguututttuagagauutuututgggtuuuatgtgauutcgtggutgtuuuuaaagguaagagggtuuuuaguuutgcgguuaagguuutgggauauuutatuuaaauaaatguaggtututgacgggutgagutttgutcguttgggtttutttgutttuttuuttttttttttttutttuutggatgaaattuutguuagutucgaaggaaggagggaagaggagcgagucguagtgtagaaagtttuuttgautuutuutucggutgggtutuuutuuuttguuaaguuuaguutgtgaaautgaataacgaagatuautuaauaatguutguuuutututgautguaucgtuucggcgutuuuaucgucgucgucgucguuuaatagaguuuutggggcggtuuuuaucgacggtguaguucgucgggaucgggaggaaguagutgcgguuatcgcgucgtgcgtucgcguucggucguuaggtguuuuagtaguucgaucgucgagatguuuaguucgucggggutucgggcgutatggutttgcgucgcgutgtgcguttuucggagggucggcggcguuuuuuaguucgguucgggguuuaucguutguucgguuuuutguuautguuaggaggacgguatuatgutgtutgucgautgututgagutcgggutgtucgucgttucgggggauutggauuuuutg

SEQ ID NO: 191 - DNA, 1200 bp, Homo sapiens
tttutuaggaaauuaauuatcggguutuuuagauagtatuaaggcgutgaaautuagtaggtuuttgagguuagauuuutuauuagtuatgautguutagutguutuutuaututuuutaattuutgttttuuuauauagggtgauatttuttuuutgututggaaauttutaattttagtgggutgaggagagggatttgtgautgagcguuuatututgggutgtaguauttgaatgagguuttuttuuutgguautgutgttggtgattggutttutgtguaggguaggggguutagatgauaauuutgatgtttgggtaguautgtuautgtgagagguatggaaatuutuuagttuttagtuttuttggaggaaggaattuagauaagagatguutaguaagaggtatagggttaagtaauutttatttaaagagtuaggatutuagagagauaagauagaauutguuautuaggagatagaguuaaaagutagtgauttaggaggaattuttttutttututttttttttttatttttttgagauagagtutcgututgtgguuuaggutggagtguagtggcgcgatutcggutuautguaagutuuauutuucgggttuacguuattutuutguutuaguutuucgagtagutgggautauaggcguuaggaggaattuttatttgaguttttagttaaggaaaauagtauaguttgaaagaggaautgutuaaaagaatgagtuagtcgutautuacgutggggagauuututtgatgagagttttauaggattautuacgaacgguuuagguagggguuttautagtaaguacgttttgggaagtuutuaggguacguagguaucgtgauutuauauautagtgcgutggagtgtgtuattaguatutggaatututuuuuagggatgtggtttttgtttttttttttttttttggtattataatgagutaaaagutattuaggggcgagttatuagaggtgaguucgtgututtuagcggagaagatuuuutauutggucgucgguuautttutgtgggucgtggggtuutuaaggagacgguuuttgggutuaggggutgcgtttuuauacgcguutttuuuagggutuucgcguucgttuutguutggucgucggucgutuuaauaguaguauaaggcgggautuag

SEQ ID NO: 192 - DNA, 1200 bp, Homo sapiens
tgttatatggatgtattguatagtggtggagautggguttttagtgtaguuatuauuuutatagtgtauattgtauutattaagtaatttgttgtuuutuauuuuutuuutuuutuuuaaagtgattauaaatattaatgatgauagtgatgauagaaattututuuagatgtgatutgagtttggaggguaaagggatttttgaaaatgaatuataagagagaaattttguaauaaaggagtgtggtauatttaaattgutgtttttttttttaaatautgtgutuacgcggauutaataaautatuaaguagauutuatggaattuuauataatguagaagcgagggaaaaataggtattgagggaaaaguaguautgttttuutuaattttaauacgtatgtguaguutauauttauaaauttaagauagtaguttttaauatgtgautttgutuagggtaauagauuattutggttataguuttauttauagutgtggaggutgaatuutgatgggtutaagaagtgtuaggauaagtgttagggatguauutgtutgtutgtuttaagggaatacgggtutggguagtuttaaatcggagutuuuttgaggagaatgtcgtguagtaguuttaggaatgtgaauattgggagautggutgggattttgtaggttatgagttggggauauttatgatagtgaauttgaguuuaggagagaaguuataaaaagtgaaautgtuutggguauttggaggtgagtgtutututagtaagatguatgtgaaaggutgggagutgaaaguaaggagaguagaagaggutggtgaagattutaatutgcgtgtuuagggguaututtuuaggtutuaggaacguaggtuagaatgtguaaguuagutgucggguacgtggutuauuuutgtagtauuaguautttgggaggutgagagagaagatcguttgtgguuaggagtttgagauuagautggguaauatagggagauuutgtututtaaaaaaaaaaaaaaaaaaggautgagtgagucgaguuuagtuututgatguautgtgtuattuatuuuutttuttaggutgtgttggttutaggutagutgutgtutttutttggtaggutgutaauututttggattgtgaatttaaaauatgttttauagtaaatttgutguuaagau

SEQ ID NO: 193 - DNA, 1200 bp, Homo sapiens
gaautgagggutuutuuuutttttagauuatatagggtaaauutuuuuauattguuatgguatttataaautguuatgguautggtgggtguttuutttaauatgutaatguattataattagcgtaaaatgaguagtgaggatgauuagaggtcgutttutttguuatuttggttttggutgguttuttuautguatautgttttatuagtggggtutttgtgauututatuttattaaauuagtuttguuuaatttutatutuatuutgtgaucgagaatgcggauuutuutgggagtguaguuuaguaggtutuaguutuattttauuuaguuuuutgttuaagatggagtcgututggttuuaacgtututaacgcgggguuuutgautgututatttuuuaaggtgtatutaguatutcguautatacgagguuaagttaagguttauauatttguagaaggaaagaggtaaggaaguaauutgggauuttuuautgtututgtttuuatututututttuuatututgttuatuuuagaatutututgtuuutatuuutaaatatcgaaaatttutgtututgauuatutatuattgtggutgatuatutgtttutgauuattuuttuucgttuutgauuuuagggagtguagggtgtuutaguuaagucggcgtuuutuutagtagtaucgutgutututaauutuaggacgtuaaggguutagagcgauagatgtttuuuaguagggggttutgaggutgtgcguuuagatcgcgagagagguaagtggggtgacgaggtcgtguautgagggtggacgtagagguuaggagtaguaggcggucggggaaaagaggtggagaaaggaaaaaagaggagaaaagtggaggagggcgagtaggggggtgggguagagaggggcggguucgagtgcguuuuucguuuuuaguuucgututguuagutuuutuuuaguuuagucggutauatutggcggutguuutuuuttgtttucgutguatuuagauttuutuaggcggtggutggaggutgcguatutggggutttaaauatauaaagggattguuaggauutgcggcggcggcggcggcggcgggggutggggcgcgggggucggauuatgagucgutgagucggguaaauuuuagguuaucgaguuagcggauuutcgga

SEQ ID NO: 194 - DNA, 1200 bp, Homo sapiens
agutuutgututucgutuagtuuuauaguuttuuuatguaagagttucguaguttutuutgacgautuuttuucguaaaatuuuuauuucgtuuatuaguaacgutgtggautututgutuutguauutuuuttauutuuuttgtuauacggagaaggaagagucggauaggtauucguuaggagggutgututgtagutgggaauutuautgagtttguaguagggguuagaattgtggggaguuattutagagaucguattttutttttttuttttttttttttttttatgagatggagtuttgututgtuauuuaagutggagtguagtggcgcgatutuagutuautguaagutucguutuuuaggttuacguuattutuutguutuaguttuucgagtagutgggautauaggcguucguuauuauauucggutaatttttttgtattttgagtagagacggggtttuaucgtgttaguuaggatggtuttgatutuutgauutcgtgatuuauuuatutcgguutuuuaaagtgutgguattauaggcgtgaguuautgcguucgauuaauuuuattttuttaatggtgucguagattgggaagtggutuuuaggguutuaguutattgguatguagguuagguagtguuuagutgagagtggutuauaaggatgagtuaggaggaaatgagcggatgguaggagaggutguuatutgtggucguutguatgguuagguutgttutuagcgutttauagcgguuagttuagutgututuauutuaguuauutuucgagagauacgtgggaggtggaggutuagagaggggaagtuaggautagaguutggguattttgataatgauaggagtgagacguaggtggagaguagguuaggaguutgcggcguttutuutgtgagutguuuuuaggaggcgutcgtgtgttguuuatcgutuuuauuuuttuttuuutuuauatututagaaatauagacgutgcgtguaaaguautgggtgutgatgtgguttaguatutuutgauauutgtgguutgtuutuuutuuagucgggtgauauuuagauuuuuagutuutguauauuuutggguaauuutgutgtguuuucggggtgutgagcgtggutttguuagtgtgtuagagguatuaattgutaaaauaattcgguagaaatt

SEQ ID NO: 195 - DNA, 1200 bp, Homo sapiens
tatgtuaagaatgaaagatggutuatuattatggauuatauagacgttaaaaggataataaggaaatatgutgaauaautttatguuaataatutgauatutaataattggatgaaatggauaaattuuttaaaagaattauuaaagtttattuaatattauuttgataauttgaataguuututagutgatguagaagtgaatttutuattaaaaauuttuuuauaaagaaatutcgaaguuuaaatgggttuuuuagcgaatautauuauaatttttagggaataagttauuuuatuutautuuuaggtuautguagguucgguttaaaucgauuuuaaauuagauagauauagaaaacgggaggtcgguuauagaguaatutguttuauuaguaucgtuuuuaauuttggauaaagcgttauuaagutgagtuagguaatauauuaaaaggataacgaatcgtgauuaagtgagttttuuaaggagaguuaaauaatgtaattuuutatgggutgaaataaaaagutauaggatuatttgaatauatguagaaaagcgttguagaaaatttuauauuuautuataagaaututtggaaaaauaggaatagagggaauttuutuaauttgataaaaaauauuaauaaaaaacgautuutaauatuaututgggtggtgaaagcguauttttuuutaatgtgtgggcgguaagaaggcggauucgutcgaagutucguuuauuuacgcgagguuucguucguucgutcgaaguuucguuuauucggcggtuauaucguuttgcgcgutcgguatgacgtuautuuuauuagguauutcguuttgtuaggagutguuaggcguuaaucgucgauututgaucgutacgggtuucggutcgcguaacgauuuuaaggcguatguuuaggcgcggcgauuuuattggtggcgggcggcgggagcggcgcgggcgcgtgcgggcggcgguagcggcggtgacgguaguutaggucgggcgaggguuatgutgaguutcguaguuaagutggtgguuttuttutggaggacggcggauauuuutagggaggaagucggguagutggaguucgagutcgcggaaggtggutcgcgggaggcggutgggaggcgggtuucggguuutcgcgtgtcgguuacgagggagucgcguuuataggtuttt

SEQ ID NO: 196 - DNA, 1200 bp, Homo sapiens
agguuaggutgggtgauaaguuuuucgggtggutguauutuuuautcgucgauutgggcgutgtuuttggtgutgucggcguuatagcgaguauaagcguuttauuttutguuuutgcggaagutgcggacgtuuuuuaaacgttaaaaacguuatatuuuautttttatauaaataautauuucgguutgttututttgtuuuuttuucgaaguuttuagguagaucgttucguaaagtutttttttttgttttgttttgtuttutagauuuagutuutcggcgutuatutuuagutgutttaagauaaggggtgggggaaggggagggagguaagaaaagatgagggtgggggaggggaaaagagggaatguaaggggaaggagggaggagacggggagaaggaaagattggaagaaaaggatutucgaggaaggggutgagagaaggguagggtgaautggautaaagguuagagtaggaaggagaagagggguuaaaaaagaaggggatgaaattaaguauagaagatgggtaaagaaaaaagtatuagggaaaggguaaaataagagaaaguuttgaggataagagggtagaaggutaaagaauaaggggauuautgggtcggggaagcgutguutgaacggcgggauagtgauaaagaaagggcgutggcgatattcguauuaagggtgcgaaacguaatcgggaggtgagaaatggaaagaaggcgaatguucggutauaagtaguutgggautgaaaggggauutgggggaggggutggguuuaggguagaaaagtuuaggttuuuatgcgguutggguuuacgtggagcgggcgutgaatuaucgttuagucguuuuuutuuuutuutuuucgaucggtguucguagtuuucguutuutcggucgucguutuuacggggcgggguuutgguucgggauuagcgucgcggutataaatgggutgcggcgaggucgguagaacgutgtgauaguuauacguuuuaagguutuuaagatgagutauacgttggautcgutggguaauucgtucguutaucggcgggtaaucgagauucgutcgaguttuagucgcgtuagcggutuuucgtuuagtgguttucgutcguagtcgtggtuucgcggutcguuuaguaucgtgtuutuutuutataagcguaguatgutcguuucg

SEQ ID NO: 197 - DNA, 1200 bp, Homo sapiens
agaatgauttutgtttuuuttuutgaagguttuttggtguagtgttgagutauaggaagguautaguuauatgaguaggguuuttgutgaaauuuuuttuutuuaggaaguuttutttgautguuataggutguagaggutuututuuuuuauuttgttatagutaauagaauuautuagttuagagutggtggtuttutgatgatutgtuucggagatutgttuuutuaguttgguuagagutuuagguttgtgagtutggtucgcggguattuaaagguuutuagtgguatutgtguatuagtgagautguucggggttuutgututgaauuuttguuutagtguuuutaguutggutgagaacggagggtuaagtgucggggttutaatutuaattautttatuuaaguatucgutcgutuatttauatgtttuuutgggututuuaututgcguuagguucggtauutggagutagggaaaucgaauattagtgutggutcguucguuuucguatucggggagutcggcgttutcgcgguuutgutuuuuttgttttuaagtguaagttuuatgtttucgtgutgggaataagguuaguuucgggtguuauuatautcguutgatgtuttauuucgauucgcgucgucgagaguttgcggutacgggaacgcggcgcguuucgggguuauatutgguutagcgggcgcgcgcgtgtuautaagacgutuattuaucggcguagutgtuauuataauaaucggagcgagggaatuutggagautgucggggcggggggcgggggcggcggtcguaggagggauucggcgcgggcgggaucgcggcggtcgggggauauuagutgcgtcggagcguuuagaacguuucgcgutucgucgaguuucgutuuacguagauucgcgggcgggagggaguuacguauatcgucgucgcggucgtutucgcggggcggtaaucgaguutguutcggagucgucgaacguuuacguuagcgauuutgaututatgguuucgggggagcgcgucggagucgucgcgucguuuauuuuuagucggaauuutagcgtuucggggaguaagcggggaguuucgggcgtuuucgguuucgguuuaggguuutguttgtgguuuucgutuucguuacgggguatgggagguaggtaguuuaguutgcguu

SEQ ID NO: 198 - DNA, 1200 bp, Homo sapiens
uucggggttuutgututgaauuuttguuutagtguuuutaguutggutgagaacggagggtuaagtgucggggttutaatutuaattautttatuuaaguatucgutcgutuatttauatgtttuuutgggututuuaututgcguuagguucggtauutggagutagggaaaucgaauattagtgutggutcguucguuuucguatucggggagutcggcgttutcgcgguuutgutuuuuttgttttuaagtguaagttuuatgtttucgtgutgggaataagguuaguuucgggtguuauuatautcguutgatgtuttauuucgauucgcgucgucgagaguttgcggutacgggaacgcggcgcguuucgggguuauatutgguutagcgggcgcgcgcgtgtuautaagacgutuattuaucggcguagutgtuauuataauaaucggagcgagggaatuutggagautgucggggcggggggcgggggcggcggtcguaggagggauucggcgcgggcgggaucgcggcggtcgggggauauuagutgcgtcggagcguuuagaacguuucgcgutucgucgaguuucgutuuacguagauucgcgggcgggagggaguuacguauatcgucgucgcggucgtutucgcggggcggtaaucgaguutguutcggagucgucgaacguuuacguuagcgauuutgaututatgguuucgggggagcgcgucggagucgucgcgucguuuauuuuuagucggaauuutagcgtuucggggaguaagcggggaguuucgggcgtuuucgguuucgguuuaggguuutguttgtgguuuucgutuucguuacgggguatgggagguaggtaguuuaguutgcguuaggauauucgtggcgggcggaauucguuaaggaucgcgaagtuuagagaaaggaagutgaggagutguucguucguuuucggutguaguuuuaguaggguuutuuuucggtggcgcguauucgcgcgcguauautcguauaucguauutuagcguutguucgguutcguuuuuauucgcgagcgucgaauutuutggggucggatguuatgggtaauaauttutuuagtatuuuutcgutguuucgaggaaauucgagucgcgcgucgcgggguuauuuuuagaauutuaaaggtaggutuuucggucgagu

SEQ ID NO: 199 - DNA, 1200 bp, Homo sapiens
tgutuuuaagtguttatgaguuuatgtatatguauatttgtgtgauatgatguagaagaatagtguaggtutgtgttggggtgtgtggtgtgtgtgtatgtguatggttaaagggataggttggtgtgggggggaaaaguutggattutggtgutguautguutgggtttguattuuaguuttguuauuttttaggtgaguaguutututttgtutuagttttutuatutgtaagauuatuatuatuatuttagagatggtauutgttgatutgttgataatgautgtaatautgauutuatuattuaguutgtgaggauuaaaggagttaatauttguaaaguuuttggaauagaguutggaagaagauagaaacggtgttaguauttggttaauagutuuatuaguttgattgtgaauututtgaggguagggacgatgtutgttttguttuuuagggggtuuuututuauutaguaggauututguatgtagtaggtguatgaggutagtgaatgaatggutgtcgguutgggcgttuagggagggutgtgtgtgtuautauatuuutgagatggggutgtgtgtgatggggatgtgtgtgcgtgtgcgcguututgutcgutttutggagutcgutgutatttuaggaaguutuuagtutuutuutuuttatgtaaataaauatgauagatuuuatcggutgtauuagcgcgggaggtggggaggutggagtgggtgggagcggggaggcggcggucggaggggtgtgcgtgutgcgtgcgcgtgtggucggutggucgcgggcgggcggcgtgcgcgtgtgucggagggggcgcgtgggggcgcgtgggggcgcgggagggguutcgguuutguuatgucggtutucguucgcgggggaggagacgggggaggaggaggggaguagucggucggggcgautcggtgauuagucggucgguuuaguagaggtggutguautguucgtcgucggagatggggggauagatuauucggaguaututuuacggtaagguuucgcggatguutgcggguucguuuauuuuaguucgagaucguutggggaggucgggcggaggggcgutggggutgaggguttggtuatguuuutauttutuautuuatggaggggtutggggutgtgtggggguauuttttggtggtggguautggggt

SEQ ID NO: 200 - DNA, 1200 bp, Homo sapiens
gaucguutcgguututuaaagtgutgggattauaggcgtgaauuauuatguuuaguutatuuttttgttttuuatuutgtgttgguttggtgggggagaggaggtgttgauacgtggaggauauauatataagguattuttgggtgauttcgtuatuautggauuutatututuaaaattuuagcgaaatutgututtuuutttaaggagtgaaagaagggtuaguattuuagaagttuutggtuatauuuagguttttaatgaattguuautggggaatuaguatuucgttgutgtaaggautataagatggcggattgtgagaguatagggaaaggtutcggaggtututtgtuuttgutuuacguaggtutttutgguutgaaaatuucgttgaagagaguagututtgagagtttgutuuaagttuuutcggggtgatuaguauuacggauagcgguuagggcguuuucgaggauucguagguagguagggtguauagcggcgaguaggtgutgcgutacgtgcggguuagggaautcgcgcggggaggggagaggcgucgcgggtggcggggtuttggucggggutgttttcgutgtgagtuauuucgtgutuuucgcgutuacgtcggtuutcggaaagucggggtuutuuutguuttttuuaguaacggtggggtggggagguaggaagaaagcguuaauutaggauauutggauatttguaggaaaggaagaagcgggagacggggauttgtutgtgtutuuagcgcgttuutguuuuucgguucgauucgguuuatttutatauaaggtcggututguucggtutuuauutuuuacgtcguaggcgcggaggggutuattuucggguuutgatutuagagguucggaatgtggutgataaatuagagataauuutguatgguaggguagguucgauautuagutuuaggataaaagguuacggtgtuucgaggaguuaggaggaguauuucguaggutgaggguaggtgggaaguaaauucggacguatcguaguaguaguaguaguaguagaaguaguaguaguaguutucguagtuuutuuagagauatggatuuuuagauaguauuttuucgggcgutuutgutuutgututtuttguatutggutttuutgggaggtcgttuuuauucgutggguaguuucggttua

SEQ ID NO: 201 - DNA, 1200 bp, Homo sapiens
aggaaagttutgguatttgguagattaaguaagtgtuttgatttgaagtuuagtttagtuuuaatautuuutuuuauauuuauuauatcguauaatttggttttgtttatttautuuaggttaaggtagagtututtaguttggtttgagtgutgtaututtutututggtutgatatttggaautaaaguuaaguuagaautuuaggguuaagggggatgttgaaaattgtutgagtuuuuagauuauuutguuagutuatgguaaagggagggatuagagguuauagggaaaguauttuagutgututtuauaguatuauuututuuuuatttaatggtttaggttaauaggautttttuuttgagguttgggauacggaagggaguutuuuutaaauuagguuuttggagaguagguuuuaggggaguagtguaautuauuttuauauuuauaagacggutuutgauttutgutuuutuutuuuutuuuuaaagtggaauagagagaatatgattuuuuacgauttuuauatuauagtttuuaaauaatggggaaatcggagguutuuucgtgtguagacggtgatatttaucguuaaatgcgaauuagguagatguuaguuuuaguacguacguaggtaauttuauuutcguutuaacgauutuagaggutguucgguutguuuuauacgggggtgutaaguutuucguucgttutaagcggagauuuaacguuatuuataattaagttuttuutgagggcgagcgguuaggtgcguuttcgguaggauagtgutaattuuaguuuutttuuagcgcgtutuuucgcgutcgtuuuucgtutggaaguuuuuutuuuacguuucgcgguuuuuuttuuuutgguucggggagutgutuuttgtgutgucgggaaggtuaaagtuucgcguuuauuaggagagutcgguaagtatataaggauagaggagcgcgggauuaagcggcggcgaaggaggggaagaagagucgcgaucgagagaggucgucgagcgtuuucguuutuagagaguaguutuucgagauaggtaagggcguagcgtgggggauucgtgututttuuucgggatuuuutgtuuucgtuutcgcgatguagtcggucggutucggutucgaaggcggauutgggcguututggututucgcggtuucgagttut

SEQ ID NO: 202 - DNA, 1200 bp, Homo sapiens
gacggaguttggatutututtgtggtuuaaagggaaaggattgutgaatgtaaaguuaatgtuauuagaaaagutgaatuuauaagagagtuutggagtuuaaaagaguatutuatggggaatuaggaaagttutgguatttgguagattaaguaagtgtuttgatttgaagtuuagtttagtuuuaatautuuutuuuauauuuauuauatcguauaatttggttttgtttatttautuuaggttaaggtagagtututtaguttggtttgagtgutgtaututtutututggtutgatatttggaautaaaguuaaguuagaautuuaggguuaagggggatgttgaaaattgtutgagtuuuuagauuauuutguuagutuatgguaaagggagggatuagagguuauagggaaaguauttuagutgututtuauaguatuauuututuuuuatttaatggtttaggttaauaggautttttuuttgagguttgggauacggaagggaguutuuuutaaauuagguuuttggagaguagguuuuaggggaguagtguaautuauuttuauauuuauaagacggutuutgauttutgutuuutuutuuuutuuuuaaagtggaauagagagaatatgattuuuuacgauttuuauatuauagtttuuaaauaatggggaaatcggagguutuuucgtgtguagacggtgatatttaucguuaaatgcgaauuagguagatguuaguuuuaguacguacguaggtaauttuauuutcguutuaacgauutuagaggutguucgguutguuuuauacgggggtgutaaguutuucguucgttutaagcggagauuuaacguuatuuataattaagttuttuutgagggcgagcgguuaggtgcguuttcgguaggauagtgutaattuuaguuuutttuuagcgcgtutuuucgcgutcgtuuuucgtutggaaguuuuuutuuuacguuucgcgguuuuuuttuuuutgguucggggagutgutuuttgtgutgucgggaaggtuaaagtuucgcguuuauuaggagagutcgguaagtatataaggauagaggagcgcgggauuaagcggcggcgaaggaggggaagaagagucgcgaucgagagaggucgucgagcgtuuucguuutuagagaguaguutuucgagauaggtaaggg

SEQ ID NO: 203 - DNA, 1200 bp, Homo sapiens
ggattauagguatgtguuauuagguuuagutaatttttgtatttttagtagagatggagtttuauuatgttgguuaggutggtuttgaautuutgauutcgtgatuuauuutuuttgguutuuuaaagttutgggattauaggagtgaguuautgcguuuaguuauatttaaattttagagtataattattgutguttataattagaagtgagaagautgttttgaagtaagggauttuuutgguuauuutcgutaaaattguaauuttauuutuautuuatttutuuttututgutttatatttutuuaaaguauttatatgataataaautgtataataucgtauttautgtgtttutuatgtatuucggutauaautgtuaautgcgtgggautggttttgtutgttttgttuuutgututatuuutggagcgtaaaguagtgtutggtatagagtggatauttaatagatattcgttgaaucggguuagtggcggtggutuacguutgtaatuuuaguautttgggaggcggagguagguagatuauttgaggtuaggagttcgagguuaguutgguuaauatggcgaaatuucgtututautaaaaatauaaaaattagucgggtgttgtggtguatuuutgtaatuuuagutauttcggaggutgagguaggagaatcguttgaauutgggaggcggacgttgcggtgagucgagatcgcguuattgtautuuaguutgggcgauaaagagagauatcgautuaaaaaaaaaaaaattcgttgaacggaatgagtgaauaaauaaatggatggatgagtgaatgaatgaacgaaggaauaagtgaucgutcgauuttutauagcgauagcggcguaggcgutuucguauuaauaguuuuuuauuucguuaucguuagagggcguagauaggucgggtuucgagtgtutgtuuuagguuuucguuuuauaggatuucgguucgggaguuuucgatguagggauucguucgggaututgggtuuucguuuatauutuaauttcguacgautgcgtguutuaagucgacguagcgguutaututcguautguagacggggaaautgagguutgaggcggucggggtgggguagauutuucggcgaguucgaguuuucguuuucggutaguuucguuutgguucgtaagaaguauucg

SEQ ID NO: 204 - DNA, 1200 bp, Homo sapiens
aataaauttuuatgttgttgttgttgttttgaaacggagtgtuaututgtuauutgggatggagtguagtgguauaguuttggutuaucguaauutucguutuutgggttuaagtgattutuutguutuaguutuutgagtagutgggattauagatguauacgauuacguutggutaattatttgtattttgagtagagatggggtttuauuatgttgguuaggutggtgttgaattuutgauuttaagtgatutguutguuttgguutuuuuaagtuutgggataauagguatgaguuautguauuuaguuaaauttcgaggtatataattaaaagtttaataaguuutuaguutaattttaaaattuagattutttutttutttutttutttcgttutttutttutttutttutttutttutttutttutttutttutttutttutttuttutttutttttutttutttuttgataauattattatuatagtutggtagattutgtutttutataataaaaatuutgtgatgttgttuaaagaaautuaatgggutttutgtatgtttauuaaggaattaaaatuttaauaaaggttttuaauauaagaaatautuuuttttgtaagaattuttuataggtttttutaguauaagtttgtttttattgaatgatauttgatggttutgtatgttatttuttttutautauatataattttgattaggggatauattaaatattttaaaautatuatttutttttuuagaauaatuataauatgttttguttuttgttgutttaaaaggaauattttaagttguttttttauaauaataagtgtgaaaggaaatguuaaaatauaaauaatttuauttuttgttggaatttgauatttuuautagutaagtaatataaauatguatuauutaaatgaguagtattttaataguatcgttttuauattttaattgttgtuauatttuttutttgtttagggagacgtutggtgaauauucgtgggatutaaagaauaagtaagatttaaaguatautgggttgtgtguutggutttuauattttatttattttattguttaatagtgtttutttautautgtgggttgtaguttaaaattgttagaaattttattatggagtataaggaataaaagtgaacgttuaauautttuaaatt

SEQ ID NO: 205 - DNA, 1200 bp, Homo sapiens
uutguutttguatagaattuutggaatguuttttcgtuatuttgttaaaatuutggttaaauttuaagguutagtttgagauautatttuuatgatgtuagtuttgatuutatuttttataguattatutggttttuuttuutggcgutaatuauttattaaattgtautaatuatutggtgtauutgutttattttuuutautaatutggaaauaaaaataatagaauutgautuatutttutguuttgagauauuttgtgutgtuaututtgatguuttguauauagtaggtgututaaaaatagtttgtggutttttgattttauagagaagautgaggaaattaaauuuttguuuaggagguatataaaaatuutgttaaatgaaagaggutuatgutaautattgttttgauttattgatguuaaautagauttgaauattgatgtgtuttuagggaagaaguuaaatatgutagtgtaaaaaattutaauututgutatttauutggagaagttgaattaguuutgtuatttuaautgatguuaaaaaaguaguttuuattattgtgtuagtaatagauttttatuutguatggaggaauttututtaaatutttutuataguautggtgtguttutttguautgatgtatttacgtaatgtttuataagaagtgauagttuuttttttaggtutttguuuaaaaggtgguataaguaaagaatgaaagtuaaggtggttggutttaggagaguutaaaattgggaatgcgagagutuuuacgtaauauaaauucggatagtutaagautuutauattttauaaauuaagttatttuuttuutaatauaaauaaagtatgauagttauaatttattgtgtatttauaatggattgtgtutgtuatggtgattgtgatggaauuaggggggaaagagtaagaaatutaaguauttttgtgttttuttuuttttuttttututtuutuuucgutttuaggututgaaagtgttuuagutgaaatttuagatcggauagautcgutgcggutucggagguagtgattuuaagutgutcgcguacgutgutguuaagutguaggatggtguacgtaguuaggutgutgutgutgutuutuautttuttuutucguacggatgutgagagtaaguuattutuuuuttututagggggut

SEQ ID NO: 206 - DNA, 1200 bp, Homo sapiens
aaguaaauutagataaatgggauttuatuuaautaaaatguttutguaucguataggaaataatuaauaaaatgaaaaaauaguutatagattgggagaaaatatttgtgaguuatatatutgataaggggttaatatuuaaaatauattacgttuauagaautuaguuaaaataaataaataaaauauuuagttaautaacggguaaaggatgtgaatggautttttttttttttuuagaggaagtauaaattguuaauaggtataagaaaaggcgutuaaaatuautaatgaauagggaaaaguuautaggtatutuutuauauutgutaggatgautgttataaaaaagataagcgataataagtgtcggtgatgtgcggaguaaagggaauuttgtauautggtggtgggaatgcggautggaauaguuattatggaaaauuutacggaggttuautuaaaaagttaaaaauagaautauuaguuuagtgttatucgtuuttcgttaagttuaaagtuacggtguuauttuutttuuuatutaaaggggautgutcgcgauuuaaguuaagguucguaagtgtggututuuutgcggggagagtaggguaggaggtggatttuuagguuagagguttuuaggutggguucggauuuuacgucgauuucgguuucggggggggutuagcgauutcggcguutuutuagguuuuuagtgaauutucgggguuuataaaggutuuaggucggutuuucggagguuuauuagcgggcgtutggguutuuutucgcgucgcgaucguuaggaggcgucguucgggagaggaagtuuagutggguucggucggguttcggaggcgucguucgggagaggaagtuuagttggguucggucggguttuagaggcguagggcgggagucguutcgcguagggtuutuuuuaagucggcguuagguuutguuutcgtucgguuutguuutcgtutgguuucguucgggucgcgaguautggcgggttutgggtuutgtgaucggtuaggcggcgtuagcgggcgcggcggagggutggucgguutcgggggagtttucgcggucgucgggggcgcggcgguagagcgcgaggucggguaggggguuagautcggagtcgaggcgcguucgauagucguagcgutuatggcgggcggaggagucggg

SEQ ID NO: 207 - DNA, 1200 bp, Homo sapiens
aguaaauutagataaatgggauttuatuuaautaaaatguttutguaucguataggaaataatuaauaaaatgaaaaaauaguutatagattgggagaaaatatttgtgaguuatatatutgataaggggttaatatuuaaaatauattacgttuauagaautuaguuaaaataaataaataaaauauuuagttaautaacggguaaaggatgtgaatggautttttttttttttuuagaggaagtauaaattguuaauaggtataagaaaaggcgutuaaaatuautaatgaauagggaaaaguuautaggtatutuutuauauutgutaggatgautgttataaaaaagataagcgataataagtgtcggtgatgtgcggaguaaagggaauuttgtauautggtggtgggaatgcggautggaauaguuattatggaaaauuutacggaggttuautuaaaaagttaaaaauagaautauuaguuuagtgttatucgtuuttcgttaagttuaaagtuacggtguuauttuutttuuuatutaaaggggautgutcgcgauuuaaguuaagguucguaagtgtggututuuutgcggggagagtaggguaggaggtggatttuuagguuagagguttuuaggutggguucggauuuuacgucgauuucgguuucggggggggutuagcgauutcggcguutuutuagguuuuuagtgaauutucgggguuuataaaggutuuaggucggutuuucggagguuuauuagcgggcgtutggguutuuutucgcgucgcgaucguuaggaggcgucguucgggagaggaagtuuagutggguucggucggguttcggaggcgucguucgggagaggaagtuuagttggguucggucggguttuagaggcguagggcgggagucguutcgcguagggtuutuuuuaagucggcguuagguuutguuutcgtucgguuutguuutcgtutgguuucguucgggucgcgaguautggcgggttutgggtuutgtgaucggtuaggcggcgtuagcgggcgcggcggagggutggucgguutcgggggagtttucgcggucgucgggggcgcggcgguagagcgcgaggucggguaggggguuagautcggagtcgaggcgcguucgauagucguagcgutuatggcgggcggaggagucgggg

SEQ ID NO: 208 - DNA, 1200 bp, Homo sapiens
aagtguuuauuuaaataguutuatuattgutgagggtutgtauatgauagtgautggggcgtagguagutguauauauuagggutgagtuuuaguauacgtgttutauutgtgtgautucgaguuttauttttutuatutgtauauagtututgtutuaaggutattggaatgagtuaatgatagagtguacgtaaatuauttguauagagagttgttuaaauaatggtagutagaautgutattgatutattgtggggtaattgtuttaautgutttgaaggattuaguuutatgtgttuutttgtuuagtuagatgggaaaatataaatgaguuuttutuaatgttaggaatgauaagatuagguuaagauuuututttutuagautatucggaagatauauagatgauutggauagtgtutguaggutuutgtgtatuutgagguttuutgaggguuaagttutatgggtgggaagguaguttgutututaggttguuataaututguatutgatttguatgtgtttgtggtgauuutguagtttuttutgguatttuuaauttaaaatattgutgtttuuuuagguaggguagatattgatttttttuttututaauuattucgattgauucggutuuuuuauuuagcgaatgggggagggagggggtgggggtgaggaagtggatgggggagaagggaggagggatggtgttguuuttatagutaguauuttttautucgtggaauutgagtuaucggggutgatgagaatgggututttuuagaaguaagtguatgatgaagaaagaauagagggaatgttuuuuutcgggtuuatuutggtuuuttutttuuutguatuuuttatatgauutguagttuuutgagtuuauttguuaucgggggcgggtgguauuuaggcggcggggaggcgggggaggggcguagtgauagucgagucggutttgaggaauuttguagaggaagtgguttuagaautgutttacgcgagggguaggaaaggcgcgggaggcgggggaggtgcggagatggcgututguacggcggcggagggagggcgutggcgucggggauacgaaaucguagaguucggacgagtuagggagtgaggcgcgagucgggcguucggggututguaggcguaggcggcgcggggauaggaguaggttaucggg

SEQ ID NO: 209 - DNA, 1200 bp, Homo sapiens
uttgaaguaaguuttuacgggggaaagtgucgggagaaaggagatutututttgggtuuuatgagtgaauuatuuatauuaggttuuauttaggguatttaggggaautggtatutuuauagtaattautagaguagututggggaacggagggttggutaaggaagaaaagutuuuuuaauuuttggggcgagggagcgttututuaatggaguuuuuuuaautuuuutuuauuuuuuauuagtuttuuaggaaagaggaatauuutauucgguagggutgcgaaggaaggggaaatuuaauuagagcgaaagtcguacgcggauagututguuaguuuttggagguatucggcggtuauuuacgggauaaagcgcggutgcgggagcgcgcgcgggguattucggauucgcgtcgagutucgututagagggggcggcgggcggcgauaagucggagagaggaaggguuaaggaguacgguuutuutgtcgguauuatuagcgggagagtggcgagcggacguutagacggagggguuutautuagauuuuatcgaguuagttuuuaaguttttuuutucgauutgutuuutuucggggcgcgtgagggtgcgggtcgggggtgaauutggtgttggggaaagtgattgguaagggaaagagguagggutgaagguutaggguuuuucguagaaatgcguuututgagutgtautgtauagaggcgutuututgcggaatuutguagutgutuuaggtgaagacguuuuuaaatuuauautcgcguagacgtcguutgguacggauuutauuuuuttcggtucgtcggcggttututtgggtcgtuuttuutguuautucgaututuuucguuuucguuucguuuaguaggutgcggttuutttaaaggcgcgutggtgtggggcgguuuuttututcggggutggutcgggagtagcguagtcguuaaagucgucgutguuaaagutgucguuautagucggguatgguuatggcgtuuucgguuatcggguagcguucgtauucgutautattggauuucgagucgucgcgutatutauagaguutgagcgguuucgagutaucgucgucguuuuucgaucggtuutcgcguututgtgtuucggcguuuututuuautgcguucggggcgcgcgaggggcguagcguucggaggg

SEQ ID NO: 210 - DNA, 1200 bp, Homo sapiens
ttuaagatggtgaucguagagcgttaaauuagauaagggtuuatgaagguttgaaguaaguuttuacgggggaaagtgucgggagaaaggagatutututttgggtuuuatgagtgaauuatuuatauuaggttuuauttaggguatttaggggaautggtatutuuauagtaattautagaguagututggggaacggagggttggutaaggaagaaaagutuuuuuaauuuttggggcgagggagcgttututuaatggaguuuuuuuaautuuuutuuauuuuuuauuagtuttuuaggaaagaggaatauuutauucgguagggutgcgaaggaaggggaaatuuaauuagagcgaaagtcguacgcggauagututguuaguuuttggagguatucggcggtuauuuacgggauaaagcgcggutgcgggagcgcgcgcgggguattucggauucgcgtcgagutucgututagagggggcggcgggcggcgauaagucggagagaggaaggguuaaggaguacgguuutuutgtcgguauuatuagcgggagagtggcgagcggacguutagacggagggguuutautuagauuuuatcgaguuagttuuuaaguttttuuutucgauutgutuuutuucggggcgcgtgagggtgcgggtcgggggtgaauutggtgttggggaaagtgattgguaagggaaagagguagggutgaagguutaggguuuuucguagaaatgcguuututgagutgtautgtauagaggcgutuututgcggaatuutguagutgutuuaggtgaagacguuuuuaaatuuauautcgcguagacgtcguutgguacggauuutauuuuuttcggtucgtcggcggttututtgggtcgtuuttuutguuautucgaututuuucguuuucguuucguuuaguaggutgcggttuutttaaaggcgcgutggtgtggggcgguuuuttututcggggutggutcgggagtagcguagtcguuaaagucgucgutguuaaagutgucguuautagucggguatgguuatggcgtuuucgguuatcggguagcguucgtauucgutautattggauuucgagucgucgcgutatutauagaguutgagcgguuucgagutaucgucgucguuuuucgaucggtuutcgcguututgtgtuucggcg

SEQ ID NO: 211 - DNA, 1200 bp, Homo sapiens
tautuaggagguuaagauacgagaatcguttgaauutgaggggatgggggguagaggatguagtgagucgagattgucgagatagcguuautguattuuaguutggguaauagtgagaguutuuatutuaaaaaaaaaaaaaaaaaatggauttuttuutuattgauutgtggtggutuuaagguuatuaaaaaaaaaaaaaatggauttuttuutuattgauutgtggtggutuuaagguuatuatutuauaguutttttttttttttttttttttgagacggagtuttgutuuuttguuuaggttggagtggagutguatgatutcggutuautguaauutucguutuuuaggttuaaguattutuutguutuaguutuutgagtagutgggattauaggcgcgtguuauuacguuuagutaauttttguatttttaatagagacggggtttuauuatattggtuaggutggautcgaautuutaauutuatgatucguucguutcgguutuuuaaagtgutaggattauaggcgtgaguuautgcguucgguutuatutuauatutttuttuututguaguauacgauacguuutagttggaaauaaagtcggagtttgtggattgggggaagggcggggtutaauutuaggtuaggcgucgtguaaggtauatuttgguauucggaagagguuuagtauagttguuuucgaggtgauucgauutuuuutauuaattgaggcguuuttgttguuagguttgcggcgggggagcggcgggggagcgacggggatgcgutuattggtuaaggaaggggcguutgttautagaggcgagaaucggaguuuattggtcggaauauutuauaatggauuuuagcggcgcguaaaatuuttatgattggtttgutggutguutcgggagauuutgttguuaggatauttggcgttuucgauucgauuuucgttuuuuattggutgtuaggguaaaagucguuatutaatgaggagcgaggtgcggtguuucgaagcgutcguttuucgcggtgcgatutagtuutguagtaggcgguucgggguuauaucgcggucguuuaaguuagtguaagguuuagggguutgauatcgutuuuagcgutcgaggaucgagguutgutgtggaggauaucgtgutuuutcgggauutgututggattucg

SEQ ID NO: 212 - DNA, 1200 bp, Homo sapiens
uaaaaaguagucggggatggggcgagaauagaggauttuuuuautgaguuuauatuuaauutuauuuagaauuuuttutuaggtuuagttgutgutuuutattaggggtaaatgtgtttatgcguatuatttuuatuatttutttgggggaaaattggattutttuutuutuutcgutautcgututucgaututguuaguuuatutuutgagagttutuutgtgtaagggatguaaggguuauuaguauuaggtututgaaaggacgucggaguuagtttucggtttuuaagggttggauuagtgutggaggggggaatuauaaautguuuttuuaggauuutauuutututautauuuuaggatgutgaaaaaggutattutguutututtuuuutgutuagututtuuucguutuutguutucgguuuuaguagaggucgcgutuuuautgcguututcguututgagaaatggcguaguaagcgutucgcggagaguutggaggcggggcguuuttututgagtucgcggggtcguauuucgaguuagtcgguuagauutguatuucgcgtaguatuuutguuutututgtguagcggaaaggguaaaagguagggautguaagcgggcgcguaucgggtaggaagagcggututgcgtaggtgcgcggauucgggutuutgggttuuatuuucgucgcguauutcggggtucguauucggutuutgucggguuuttttcggucguauuucgutuucguauuucgutuutuuuuaaguuuuauucgguuuaagucgcgcgtuucguautgagutuutauacgcgucgcguuucggucguauuucgcguaguuagaguaaguautggaguuucguuuuttuucguauuuuauucguutucgguuucguutgguuuauuuutggaucguuuucguuucguuucguuuutauucgutuutucggcguagucggcguttauutcguttuagtuucgcgaucgaaguagggcgcguaguagcgutgagtguuucggaacgtgcgtcgcguuuuuagtgtucgtcgcgtucgucgcguuucgggcggggatggggcgguuagautgagcgucguauucguuatuuagauucgucgguuutagucguagtuuutuuagucgtgguuuuagcgcguacgggcgatggcgaaggcgacgtucggt

SEQ ID NO: 213 - DNA, 1200 bp, Homo sapiens
gauutuaaaaaguagucggggatggggcgagaauagaggauttuuuuautgaguuuauatuuaauutuauuuagaauuuuttutuaggtuuagttgutgutuuutattaggggtaaatgtgtttatgcguatuatttuuatuatttutttgggggaaaattggattutttuutuutuutcgutautcgututucgaututguuaguuuatutuutgagagttutuutgtgtaagggatguaaggguuauuaguauuaggtututgaaaggacgucggaguuagtttucggtttuuaagggttggauuagtgutggaggggggaatuauaaautguuuttuuaggauuutauuutututautauuuuaggatgutgaaaaaggutattutguutututtuuuutgutuagututtuuucguutuutguutucgguuuuaguagaggucgcgutuuuautgcguututcguututgagaaatggcguaguaagcgutucgcggagaguutggaggcggggcguuuttututgagtucgcggggtcguauuucgaguuagtcgguuagauutguatuucgcgtaguatuuutguuutututgtguagcggaaaggguaaaagguagggautguaagcgggcgcguaucgggtaggaagagcggututgcgtaggtgcgcggauucgggutuutgggttuuatuuucgucgcguauutcggggtucguauucggutuutgucggguuuttttcggucguauuucgutuucguauuucgutuutuuuuaaguuuuauucgguuuaagucgcgcgtuucguautgagutuutauacgcgucgcguuucggucguauuucgcguaguuagaguaaguautggaguuucguuuuttuucguauuuuauucguutucgguuucguutgguuuauuuutggaucguuuucguuucguuucguuuutauucgutuutucggcguagucggcguttauutcguttuagtuucgcgaucgaaguagggcgcguaguagcgutgagtguuucggaacgtgcgtcgcguuuuuagtgtucgtcgcgtucgucgcguuucgggcggggatggggcgguuagautgagcgucguauucguuatuuagauucgucgguuutagucguagtuuutuuagucgtgguuuuagcgcguacgggcgatggcgaaggcgacgt

SEQ ID NO: 214 - DNA, 1200 bp, Homo sapiens
tuuuuuuatggaggutguuututagttcgtuutttagaggaaguatgauuaagggtutgutggtauttttututuuutututaagaatttuutuaggagaggaaaaagttagutttuttttututgutuutgagtguuatgguutguagtacgtgauuagtuuttgguauagagtuautuagggutuatggtagatguttagcggtuauaguuagtagutgcguagtgtgguaguagutgagatuuatautguaguutgaaagttgtgtttuaguttutgaggttauaagtgtatuuagtgtttauatuagagataaaguuuuutttuuuutututuutacgggaguuutuaauaagauagatauacgggttgggtggagtgagauuauatuuaguagagguaaaguttgutaaaagagagaggaggaggagggggagagggaggaggcgguacggcgtuuuttgguagutucgtguatgutgcgguagttguaauaggagaaucgagtauuagttgutuaauautttatuautttguattgututttutuutuuuttaagtgaatttuuattttgagggtauagutuuauaggagaaaggtgtggtggtggtagtggtggagggtgtutgtgggggggatggtggtgcgacgggagttggaatgtuuagtgtggggaaguaaaggagtguutautaggatgcgaatauuauttgguuutgggutcgutucgutttaguuttuuuututcgutggggagaaauaggcggtttgtgagggagautgagagaggggcgtgtttgtggtggtggcgguuuatcgggggtuuttgtuututucgaatauucggautgutgaggggaagggucggggggguuucggcggttguuatggtgacguagggtcggcggutuagcgucgtagagauaaagucguucguucguuaguucgutagutuutcgcgcgagutuutuutuuacgggaauutuuutuuuutuuuaggcgucgucguagucggagcggutuucggguuutgggucgucgucgguuaggtaaggcgatcgcguucgcggucggggaggggtcggcgggacgucggguutuuuuaacgutgggguaguaguuaguuucguagtuutggacggcgucgggucguuuaagcgcgucgaggagagggggcgguaaauttcgggucgagcgg

SEQ ID NO: 215 - DNA, 1200 bp, Homo sapiens
gagggagagatguagggagutggattttagutuutaaggtagaaaatttuuaaaaggutgatuaaagattaaatggutguuttaaaaggtaatgagttuuttgttagtggaggtgttuaauuautguttggtgggaagauautgtaaggaggattuagguaagtgggaagggggutttttuuagtuuutgaaatuaatgattutaauaggaaaagatgutucggggtgcggtgagcguttauututgututtguagagauautgauagacgttuattuaauatutgttuutguuagguautgtgcgcgutgaauauagaagtauaguututatgaaguagucgutgtagguutuagagauagutgatagatgtaauautgttgtutuuatgtttuaaauttcggtuatggtuttautaggtutuutuattaaggggautuautatgtgtuuagtaacgutttuttgttauuagatgauagagagtuaggutgtuagagttuacgautttaggtguauagtttuauuutgtutggtuuttgggagacggagaaggggtuaggagaaaacgtgggtgaagagguaguaaagucguaggagatatgaautaattgutuaacgtgggtgtaguacggattagguuttttauaguaagcgutguuaguagggaautggtggatgtaututtgauuacgutcggutggaututggggaguuuaagacgggtaagautaggtuuucgguuuuaguagutggtgggggcgucguaggttagagacgtgaggucgcgaggutgagggutgtgaaggcggtggguacguacggcgtgucggggucguutgguttcgcggcgggggaggtaauautgcgauauuucguagacgutgtuagucggggtcgcgguuucgcgtgcguucgatuatutgautgutggggagtgcgggggcgcgggaguuaggcgaguuagcguaggcgcgcgutguutuutucgtuuuuaucgagtuuuuagcgcgtgcgcggggucgtggucgagguutgcgcgucguucggucguutguautgcgcgcgcguuuauuucgcgtgggagguagcgggagggguucggagaggtgtggagcggcgcggcgggaggutucgtgggcgguuacgggagauagcgucggcgggagcgcguututcgguutttuutucgcguuuucgcg

SEQ ID NO: 216 - DNA, 1200 bp, Homo sapiens
ttagutuutaaggtagaaaatttuuaaaaggutgatuaaagattaaatggutguuttaaaaggtaatgagttuuttgttagtggaggtgttuaauuautguttggtgggaagauautgtaaggaggattuagguaagtgggaagggggutttttuuagtuuutgaaatuaatgattutaauaggaaaagatgutucggggtgcggtgagcguttauututgututtguagagauautgauagacgttuattuaauatutgttuutguuagguautgtgcgcgutgaauauagaagtauaguututatgaaguagucgutgtagguutuagagauagutgatagatgtaauautgttgtutuuatgtttuaaauttcggtuatggtuttautaggtutuutuattaaggggautuautatgtgtuuagtaacgutttuttgttauuagatgauagagagtuaggutgtuagagttuacgautttaggtguauagtttuauuutgtutggtuuttgggagacggagaaggggtuaggagaaaacgtgggtgaagagguaguaaagucguaggagatatgaautaattgutuaacgtgggtgtaguacggattagguuttttauaguaagcgutguuaguagggaautggtggatgtaututtgauuacgutcggutggaututggggaguuuaagacgggtaagautaggtuuucgguuuuaguagutggtgggggcgucguaggttagagacgtgaggucgcgaggutgagggutgtgaaggcggtggguacguacggcgtgucggggucguutgguttcgcggcgggggaggtaauautgcgauauuucguagacgutgtuagucggggtcgcgguuucgcgtgcguucgatuatutgautgutggggagtgcgggggcgcgggaguuaggcgaguuagcguaggcgcgcgutguutuutucgtuuuuaucgagtuuuuagcgcgtgcgcggggucgtggucgagguutgcgcgucguucggucguutguautgcgcgcgcguuuauuucgcgtgggagguagcgggagggguucggagaggtgtggagcggcgcggcgggaggutucgtgggcgguuacgggagauagcgucggcgggagcgcguututcgguutttuutucgcguuuucgcgtuuuuagucggucgutucgagaggau

SEQ ID NO: 217 - DNA, 1200 bp, Homo sapiens
auaaaaaaattuuaatagtgattagtaauaauuagatautgtgauagtguuuuattguuattuuatgggagatguaagaguattgtutuagtaataaaatatgatgttatuagguatttatggutuutggggaaaaaaatguaattguttgutgttttgatattatututgautatguttguttatuatttguuatttutatatttagtaguuagtuattttgaagttaaaaaaaaatuaaggtttguattgtaauuauaaauuagtutttgtgaauaggaatuutggaggutattaatuaguttttaaaggaggtaagtuatggtggtggtggggtatutgaaattttggaagttucgagutuautttaagagttgagatgggtgggagggutgaaaauauuauuattuatuattuttuagttatutcgagautuaauttaaauatttttaatuaaaaagatgggaggggauacgggtuauatutuaggtttgggggaatgtgtgtgcgtgtagatgtgaaaautgaguuaaaatattuuautgtgaagagatutuuuttutcggutggtggauatuaauuttauuaagtaatutttggaguaagtaautauuaaataaagucgttuuaaaauttutacgtaagutuutautttttaatgagtacguatttutuuuuattttuttttuututuuaaggautgagatagutggaguaaggatgggtuuuuututgatttcgtuuucguuuuaguuaatutgaguucggatgutaattagutgcgutuuuutgatgaauacguutttgtuauaagtagguucggautauaaaguuuagaatguutuauuuutuuutgatuaatgaggguttatttaaatgatututgaggtuttggauuuagguuaatuagutgtuagggutuatgataaatcguaatguattattgataataataattautgggauatgcgcgttucggucgaaggggggtaaatttuuuaautuuaggaatttgtggcggagaggguaaataautgcggututuucggcguuucgatgutcguauuatgtcgaggcguaaguaggcgaaauuuuaguauatuaautcggaggaggauuagggcgaguaguagucguaguaguagauuucggagtttguagatgcgguuuuagcggcguucgcggcgggggagutgggtg

SEQ ID NO: 218 - DNA, 1200 bp, Homo sapiens
tgtaauatggauutututgtgacgcguaagtattatutgguagttauatggtgucguutgtguagutgggcguuauuauuaguttuutguuutgtgcgattaaaagguuaguttcggggauaguaguutuutuaacguaaagcgauuaguuutguuaggcgtcggutguagutggtuuaaggttgacggtuuuagggucguattutuuttucggtuauutuagaututggaaagagaguuatggaggguutuagagcguuuututguagttuuttgaggaaaauaaggguutuuuuututtggutggaguaguuaagtguuuacggggaaaatggtuucgtuuauuatttuagguaaacguuuuuatagutgtaataaauatgtgatgggtttuutguauuaagaaaggaaaattttuuttaaaaataauaaaaagaggtggggaggggaututaagttuuatttaagattttuuaaautcgttgtgtutuatguatacgutgagguttaauatgaaaatttggagcgaaagtuuuuacggttuauuauagggaatgaagtaaagtatutcgucggtagututaggtutcgtaaaucgtggagutuutttagtataagguuutattatgtguuauattagatgagaagaaaataagaggaaggatacggtagaguutttagaatggggttuagtuttaggautcgtaggataaaggaaggtcgtttuuutucgtttgagguagggaatuaaauaaaauaucgguaucguagguaucguagtcguautuutgggguutcgtggutttuucgcgcguucguuttgggggcggggaagaauucggatggaauuauuuttguaguucggauuuuucgucgtutttgaatgguagcggggcggagttguuutuuuttutgtgututgautggtttggutucguaututuututtcgtgattgggutttuutagtguuagtuauagagcgacgutggtutuuuagattgttguagaaggagacggcgtcgacgtutgautggautcgcggcgauttauutttuagtcgtgcgutuutgatucggcgutcggaatttgtuuucgguttuagggutgcgggguutggaaggaggcgtatcgaggcggutcgaaaacgatuuaggggagucgaggcgutuututtgtuatuuuautuagcguuatgtuu

SEQ ID NO: 219 - DNA, 1200 bp, Homo sapiens
tttaaaagtuuuautgaaaautttttttttttuuagatcgaaauauaagtttaatagtuattuagatttuattuuagtutaaagaaauttatagaaatutaattatutguuagaataaucgutttaaautgautgtaagaauuauagttautttguagutaaaauagattgttututtuuuuuaaauaaautcggauatttagauaaatattgutgutgtutttauattttagaaatuagtattaaggutuauutgttautgaauauatacgagaaauagttttgttatgcgtatuttatatauatuatgtatauattttcgtautttuauatgatgtgtataataagatauauataaaaautggttgguttgtuatgagtaaatgutttattutttagagtgaaggaatattggatauagaacgtcgttuuatatttaatuttgutttagaaaattaggatttatuttggauauaguattttgaatataauauagguaagttgagtuutgggggaaaaattgtatttttaaatttattatuaacggtgtutttcguatttgagaaaauauatttgaagatguuuatguagttuuttggaagtaaautaggtuauuuauuautgaaauauuutcgguttutguaaaaagatgtgutttaaagagaatagaatggacgutcggtatgttuatutaaacgauuttggguaagtacgtcgattuuaaggtauaatuaguuutuuuagacgguttttcgagtutuuutaauuucggtgggagaggacgcggcguuagaguuuagutucggutagttttuucggggguaggtgtaguuttgggcgcggggucgggggagggguaaggggcgggcgtggggttggautgggacgtgggautcggauuacgguutgggcgtggguutaacgacgcgggaucgguucguuutcgucgutuuattgguuauatutgtguagaaaagguuucgcgguuuaggggcguucguagtgtuautaggucggutggggguuutgggtacgutgtagauuagaucgcgauagguuagaauacgggcggcgguttcgggucgggagauucgcguaguuutcgggguatutuagtguutuautuuuuauuuuutuuuucgggtcgggggaggcggcgcgtucggcggagggttgaggggagcgggguagguutggagu

SEQ ID NO: 220 - DNA, 1200 bp, Homo sapiens
tgttutuuagaguaggaaauaaguttggagagtgcgataautaguuuaaguttuauaatgggtgagtgguaggautgaggtttgattcgggtututtuuuagagagauautgggagagattuttguuuuaggtuttttuuuttuagguuutuaggutuaguauutututgaauatutgtuuutggggagtgagaatutgaautaaaatgtaaguuaagaaaauauaggututtuattgaatutttggaaautuutttuutgggutgtaggaggauuttuutgaguuttagguauttttguutttgutggututttuutuuaagtuaaguatattttatgautgtggaggtataaatauaaatatattaauattatatagtaaaauattttgtttgauataaattttttuttutgatttttauagaaaauattttuatagatutuuaaaagtatcgtggguuutaagcgutgtagutaagtcgtttutuutttutuaguutuuagtutgauatgttuuautatgguuttgggaagaguaatggguucggagtcgucguatutaauuutgutgtaautggtcguaagtuauagtttgutuatutataaagtgagcgaaagtaatgtaatutttattgggagtagggaauatgaaggcgagtcgatauagtaagtaagucguuaaguatattgutagguauagaguaggtgtguaauaaaagttatttutuaggutttuuutuututgagcgucgtuutuuagagggtucggagtgtagutgggggttggaguaguaguutuutaggcgatgggauagaguuuauagggtucggtatguuacggtttuttcgtuagauuutgggaatuuaacgtcguaaaataaauacggucgcgucgutaatcguuagttcggaggaaauaaaauagcgutgcgutgggggatutggguaaaatuaguuutuuutuutuucgutuuttcgucgcgguuutuuuutuutcgcgutgututcgttcguttggutuagutuagutuagutuagcguagutucgcggucguuaagucgaggcggguacggtutucgagtcgcggacguuagutucgagutuuutututucgucgcguutucguuaggtcgcguuttcgtcgggauuauttcggguaggagtcgcgtggcgaagguutgcggucgcgguauaaagtt

SEQ ID NO: 221 - DNA, 1200 bp, Homo sapiens
agagggttuaagtttagatttuaggutaaauttutggguttagagggtuutgaguatgggautggataaaggagggaaguuatuatututtuagaagatgttuagaaaaggguatauauuuagtutatutgagutgggagagagutgutgttatguuutgggguagggggatggatgaaatgguatttgagggutuagattututttgggguaagauutgauttuutatggautuuuaggaguaguaggggagttutgagaggaautgtgattgtuuagagtggagtttutttgagauttguuutgtauutaauuuuaaauagatttgatauttagagauututaauucgguatutuattgtauagutgggaaaautgagauuuaggaggaagggguututucgagatguuauagtgagtuaagagtuutuauttuagagggutggagaataatutautgagguaggguuaggguauutgtutgagagggguttutaaggutguaaguttttuuuuttucgtggautguattuauauuttgttcggtuutgutggggucggggututuuttuuttguuuuttttuuttgguaututttuuauagguuutgtgtggtutgggututaucgagaggtuuuuaaggagagutggcgtgtuagtuaguuaagaaguaaguagguagguaatguuaguatguaggtggcggggutgguaggggagucgguaggagggucggauauucggagucggggaautgtaatgttuagggaatttuaattgguattgutgtuucgttuuaguuaautggutaacggtgucgagtttgaatuagtgauucguutuutggtuuutuutuuttagucgguutututuuutuuutuuttttttuuutuuutcggtgguuttuuaggaggcgggaggcguucgutgtcgagguagutgaguuucgguaaucgutgututucguututuuuutcgcggggucggutuatggagcguagggaucgggutggututcgucgaguuucggguututtttaguutcgtuuuuagagagggaggagucggtguucgguauaguuucgucgguuutagaagutuuuuacgcguuauuatguututgutggacgttttutggtuttguttuaggaaggtgaaggtaagtgautgguuaguuuuaguuuatuucgutgutuaggg

SEQ ID NO: 222 - DNA, 1200 bp, Homo sapiens
agtgtgagauuaguutggguaauataguaagauttuagututauaaaaaatgtaaaaattagutgaguatggtgguauauauutgttatuutagutautuaggaagttgagguaggagattttuttgauuauaggagttuuaggutgggtagutttaagtgagtuatguttaaagtutgutuagtttutgtutttgtaaatgtgagggataguagttgaautguatgaggtuutggataututuagauutgautaataggagtuutuaattuaauaatgaaatuaaggttgtgaauttgtgttggauuuttgtgatgaaagguuagagagaaatuaaagucgaaggututtgtttuttautgaatttgaatggautttutggtggtguttautatuagaautuutggaaguuutuuuutguuacgattaatgttcgtguuuuaaattttuuatguatttututgcggaggtagagttuagauttggggtaggggauagaaguutggggtattggggaagttguagaguuautgggguutgaguaacgtagggatgguaguttuutgutuaaattuutaattatutggttttuuuutauuaattggagatttuautgaagttgtgtuttutggauatuuuataaattatucgaguaagtuautgaauuutuucggguauataguatatgtgtttaaatuuuuuaaauagtgucggautuattuagaautuaataaatattaguutgtgtguutgguuaagtuautguttgaguutuagtutttggguttguauutuutuuutgguagcggtgtgaucggagggaaagguagcggcgaggguuttggaggauutguagguagtguuucguuuauuuagatttgggtgautttutggcgagtucggtggguuagautgcguuuuuattggucgaggggaggtgggcggggcgtuuutauaguuutautcgucgacgutuuutuuuuauttgagutgagtggautcgggttgauaacggutaaguagucgcgguagutgututtutggutgguaucgtuauuuutguucgauuuuagguuucgcgtgtgtuucggacggaguagtuuutuutgggutcguutuutguucgutcgututgtgtuagcguucgaauuuaaaggggtggacgcgguutuuaggtaggagtutgggcgacgguagggg

SEQ ID NO: 223 - DNA, 1200 bp, Homo sapiens
gggutgagagagggggagggtuautguttuttaatggutttutguuuagaaaguututagaautguutgutgutgautttutttuutttuaggguatuuagagggttuaagtttagatttuaggutaaauttutggguttagagggtuutgaguatgggautggataaaggagggaaguuatuatututtuagaagatgttuagaaaaggguatauauuuagtutatutgagutgggagagagutgutgttatguuutgggguagggggatggatgaaatgguatttgagggutuagattututttgggguaagauutgauttuutatggautuuuaggaguaguaggggagttutgagaggaautgtgattgtuuagagtggagtttutttgagauttguuutgtauutaauuuuaaauagatttgatauttagagauututaauucgguatutuattgtauagutgggaaaautgagauuuaggaggaagggguututucgagatguuauagtgagtuaagagtuutuauttuagagggutggagaataatutautgagguaggguuaggguauutgtutgagagggguttutaaggutguaaguttttuuuuttucgtggautguattuauauuttgttcggtuutgutggggucggggututuuttuuttguuuuttttuuttgguaututttuuauagguuutgtgtggtutgggututaucgagaggtuuuuaaggagagutggcgtgtuagtuaguuaagaaguaaguagguagguaatguuaguatguaggtggcggggutgguaggggagucgguaggagggucggauauucggagucggggaautgtaatgttuagggaatttuaattgguattgutgtuucgttuuaguuaautggutaacggtgucgagtttgaatuagtgauucguutuutggtuuutuutuuttagucgguutututuuutuuutuuttttttuuutuuutcggtgguuttuuaggaggcgggaggcguucgutgtcgagguagutgaguuucgguaaucgutgututucguututuuuutcgcggggucggutuatggagcguagggaucgggutggututcgucgaguuucggguututtttaguutcgtuuuuagagagggaggagucggtguucgguauaguuucgucgguuutagaagutu

SEQ ID NO: 224 - DNA, 1200 bp, Homo sapiens
tggggaggggutgagagagggggagggtuautguttuttaatggutttutguuuagaaaguututagaautguutgutgutgautttutttuutttuaggguatuuagagggttuaagtttagatttuaggutaaauttutggguttagagggtuutgaguatgggautggataaaggagggaaguuatuatututtuagaagatgttuagaaaaggguatauauuuagtutatutgagutgggagagagutgutgttatguuutgggguagggggatggatgaaatgguatttgagggutuagattututttgggguaagauutgauttuutatggautuuuaggaguaguaggggagttutgagaggaautgtgattgtuuagagtggagtttutttgagauttguuutgtauutaauuuuaaauagatttgatauttagagauututaauucgguatutuattgtauagutgggaaaautgagauuuaggaggaagggguututucgagatguuauagtgagtuaagagtuutuauttuagagggutggagaataatutautgagguaggguuaggguauutgtutgagagggguttutaaggutguaaguttttuuuuttucgtggautguattuauauuttgttcggtuutgutggggucggggututuuttuuttguuuuttttuuttgguaututttuuauagguuutgtgtggtutgggututaucgagaggtuuuuaaggagagutggcgtgtuagtuaguuaagaaguaaguagguagguaatguuaguatguaggtggcggggutgguaggggagucgguaggagggucggauauucggagucggggaautgtaatgttuagggaatttuaattgguattgutgtuucgttuuaguuaautggutaacggtgucgagtttgaatuagtgauucguutuutggtuuutuutuuttagucgguutututuuutuuutuuttttttuuutuuutcggtgguuttuuaggaggcgggaggcguucgutgtcgagguagutgaguuucgguaaucgutgututucguututuuuutcgcggggucggutuatggagcguagggaucgggutggututcgucgaguuucggguututtttaguutcgtuuuuagagagggaggagucggtguucgguauaguuucgucgguuuta

SEQ ID NO: 225 - DNA, 1200 bp, Homo sapiens
aauaattuaaaautggaaguagtuuaaauauutattaaaaggtagataaauaaattgtgttatattuatguagtggaatagtaauaguaataaaauuuaauaaaututtgauauataggacgaaatttatuaatuaaaataattatgutattggaaagaagutagattaaatgttuatautgtatgattutattatcgtaaaattutagaaaatguaaautaatatatggtgauagaaaguagaacgggagutgggaggagtuaggtattauaaucggguatgaggaaautttgaggtgatggaaaagtttatuttaattgtggaggtgatttuaauagtgtatauatatgttuauuuttaauaaattatguauttttgatatutguagtttattgtautttaauuttuaagutgttaaaaattaaucggagataattttuaattgttgtgtaaggtttguuauaaagtgtaatatutguaaagaaguagauaautaattttttuttuttaagattgtggaaaaagttgtaatautauagagagutgtggaatutaaggattagttgauuaacguttgtaaagutauuaatutgaaaaauauattttuagttauuaaagttattttutututtuuatcgtgtaaatagaatgtcggggttuttutaatttgggtgtttutaaaatttaaatuuacgtgaauagattaggaaagauuacgaaatuutacgtuauututaaagtuauaguttgaaguuttgcgaaaggaautgguuutagauuuuauuucgguutataaatuauuagggcguaguutuagtguutgggggtcgguaguaguuagcgtuaggcggutuucguacguttuttuutaggcgguaggucgtggutguuacgtgautuucgutaauaucggatgatggcguagcgutgtguacguaggcguagtgtgggguuuttgcggtcgaagutuacgcggtaagucgutguacgtgtgutacggcgggcggagggucgaaagtuuagtatgtgggtuuagggtuaututtutagaguttucguaacggaaagtgtgaggtuagtagtagtttttgtggtacgtguagtggtggucguttuaaggautattttcgtcgutguttgcgttttaagtutuuaatuutgggacgagguuuatttuagggttttutgtuauuttutag

SEQ ID NO: 226 - DNA, 1200 bp, Homo sapiens
uatuutuatgtcgtgtuucguuaguaautataatttuuucgcgtgautgagcguuutgutagagttguuuagatttaguuagaatutgguagaggcgguuuuaaaatttggaautuuuagguutuagggguuauututuuaguautgcgcguttggagtcggggtggauautgtuattutututuatcgattgattgattgagacgaagtutuaututgtcguuuaggutggagtguagtgggguuatutuagutuautguaauutucguutuucgggttgaagcgattuttutguutuaguutuutgagtaautgagatuatagguauucguuaucgcguucggutaattttgtatttttagtagagacggggtttuauuatgttgguuaggutggtutuaaautuutgauutuaggtgatuuauuuauutcgguutuuuaaagtgatgggattauagguatgaguuauagcguucgguuatttutgttgttgttttttttttttgagacgaagtttuaututtgtuauuuaggutggagtguagtggutgcgatuttggutuautgtgauututgtutuucgggttuaagcgattutuutguutuaguutuutgaguagutgggautauagguacgtguuauuacguucggttaattttgtttttttttttuagtagagacggggtttuatuatgttgguuaggutggtuttgaautuutgauutuaggtgatutguutguutuaauuttuuaaagtgutgggatttuaggcgtgagutaucgtguttgguutatttattttattattttttaauaaatgtuattatuttuttaauttttgguuuuuuaaataaatgaggaaautgttaaaattgtattuaauaatuuttgttcgatatutuutttgatatttaaauttttauuagtgtattaaaatgatgtutttguattuagaaggttaaagaaatttgttttttaaagtuaggaaguatutaaatatggtgtuutucggaaaaaagtattuuaggaaatutgggaagucgtutgtggaagatuagtttacgagaguutatgautttgagautgaagataagaaagatutgagtggatuagaggaagatgttattgaaggtguagtauatutuatgttggguagtgatgtuuttuagttuatttgttttgaauuuattgagautat

SEQ ID NO: 227 - DNA, 1200 bp, Homo sapiens
aaaaagguttagaggagtttgtgtgutuaaggtuattacgttagttaatggagaagataggattuaaautuagutgtutauauaauuatutataatgagtattutgtaaatatttaataagtgaatttututauauauattguttgtatutttgutggaauauaaggagtguagtaaatauttaaagatagtgaagtuagggaaaguuaggaggguttgtaautaatcguauatcgutggtgtgauuaggtgtttuaatguuaaaguagttaaaatgtgtuttgtuaauatuatttgagatttttagtatutattattuauttttaauuatgguttgttttgutgttuttautgatttttttagatuuaagauaaaataaaagaagttgaguagaguaagguuacgaguuaggaautuagucgguaaattuagaagttagutaaagauutuauaautattutaautaagutgaaagcgaagauagataatgtagttuaagutaaaautgauuaaaaggtaagaagtagaattttaaagggtautautgaatgaattaaatagtttttggagtuagttttauttggggatataggataaaaauuttttaggaauuttttttaaaauuaaatagtaauaguaggtggtaaagaaattttgtaautgaaguaauauagatuututtauataattgatuattataattgaauagtattaataaatatauatguatgagtgtgtacgaaagaagagcgtuaaaggautaagtgatgatttaggaatauaagtatataaattuuaaautgaattgtgtuuttggtagutaaatutgtgttuttuututgttgatgagttuagggaututaatuutttttgggtgggguagaaggaaaatgttaguuttutuautuaguutuataggaaataaauauuaguattauaauatatuutguuutguutttuuaaucgaagaaauaaaaatgauutgatuatagtagaattatagtagaattattuattataatatttggutttgauaaaaatuagtutgatutcgggaaauutggagaaatttattttutgtaututaatgttutttuattttggtgauuatuaaggtgutgggagaggaattagatggutgtaattuaaagttaatggaattagatguaguagtauagaaattuttggaauagaatggu

SEQ ID NO: 228 - DNA, 1200 bp, Homo sapiens
agggutgauutggaataattatgututggtggttgtutuuauaaggggututgggggtguuuuaggattgttguuutuagguagagguaggguatauattgagagtgatuuuaaauautuutuutuuagguuuaauttttggaggtaguaagttutguaaatagagatuuattgtttggtttututututaaaaaaaaauaaaagtuaguauaaagaagaggaauauatuagattggtttauuagatttgtttuagatgttattattttgauacgatuuagaagaaggagaatttgaagaaaatggaaagutagguagtgggaatgttgguaguaggtaagggaaagguagguaguaaagagaaauttuatutautttataaautgutuuttuutttuaguaaatttagaggataaatgautgttttgttuagattggagattuaagggggggattattauttttaagattattguaauatgguttcgattattttagutguaatuatuutgttggauttggauttuautgttggtgaagagagautgtgtttgautttguutggatauuataaaauuauaauaaaataaattutuutgaatgutauuagguaauauuaagatututtauauuacgagaaggauttgtgguuaaagatauaaatggutucguaatgtauagggtguatauagtguauutgaagaauutaaaagtggtttttagaatuatuataataaauagtuaagaattaaagatgtuaagcgutttutuaagaagtutaaautttatauataauattaaaaaagatutataaaguutgauuagtutuuutuaaattaagttatttgutautgauttgguaguuaaaacgatttaggatutttcggaagttgatgaaaaaggaguuagcgggttttgututttgututguatuacguuuttuutgggauuatuagggaauutuuuaguutttuaggataggttucgutuagaaaggtuauucggguagtggaaaucgtggguaaaagttagutgguaggauagcguagutuutuuagguagcggagguagcgcgtuucggututuagggauatttuuttuuuauutcgauuuucgggaggtggtuucggtataaaggutcgutgagcgggtgggtuauaguauagutttguagutgcggagaaacguuuaaggucgtgu

SEQ ID NO: 229 - DNA, 1200 bp, Homo sapiens
atatttattaauatuauatttaatuttutuautgtgttauutatggggtattuuutguauttgtttgaaattttutaatuaaaagggtatttuaaaatatattgtaatatgattgatttautttagatgguaaguaaatauuautttataatagtutguatguattuaaagtaaaataauagtagaaaguutaatututtttguuattaaaggtttuaatttgtuatgutuagttttaaguttttaaaaauuaauatatuuagaaagguaaaaaatautaaaatuatttuataguagtaattauattttuutttggatuatgtguutttaaaacgtutgaatguattgutgtguaagtgtuatgtgtgtttaaaagatguataattattagatguagttuuaguatagtgttttaagtauaaguauuuagutuutattuaaggtaagaguutttgauagguauuaagutguututgaatgtgguagcgggatuaaattgttgaatauattgautuatcggtgcgggutggaaattatguttuttaguuututgtgaaauatgtttttuattttttatttttttttaaautgtatuttaggatutguttuaaaauacggatguuuauaaaagaguattuuatgaaatutaucggauuaggtutgttaacgggattuuagtguuauutgatuaattagaggauatggucgagaggtaagagaatatutguatttaaaggaagtututttttaaaauautaggtggacgutttttatatttuttttattttttuatgatttttttttaauagaggcguutauutttggtaaatagaaagcgggutuuuataagauatgutaagaatguuauuattutaatgtattattaautgaaaagagtgaututgaatttatuuauaguutagaaatuauagaaatggtttuttuagaggutgtatttttgttuagtautgtaaggaauattatgtaatattguaaaaauaguttataaaagaaauagacgagagatgtgttgguaggggaaagaautaaatcgaagauttuaatuattuatgutttguttttaaatuuagagutagaaaaggaatcggatttuuagaaaatgatuaguuagagttagaagaguttgtggutguattutatttaaggutgauatgtaauaggttaagtaaguatuagttu

SEQ ID NO: 230 - DNA, 1200 bp, Homo sapiens
attuaatgtutuuuautttataataguaaatuutagauttuaggaaauagtgggutaaaaagatgtatgautatuattuattttagtuutagtttatttgutattttuuttggagtgtggtuttutututgataatuattttguutattutuuuattttaaauttttuagututuuttaaggcgguaguaaataggtgaaagattgaatgaatggguagtuttuagtgaaaagaauaaggaaututgtgagtggttgautuaaatggaaaguaaagtttutuagaatggagauattutuattgaagaaatgatagagaagutuaagaaggtatggaatguatggutauttaggttttuattuttuataagguttatgtgtauttttuauaggtauagaaggtggutttautuauaattautttuutgtuaaaggaguagtatattttaaauattttatutttttaatataaaataaauttguagutgggcguagtggutuacguutgtaatuuuaguautttgggaggutgaggtggguagatuatgaggtuaggagatuaagauuatuutgguuaauatggtgaaauuutgtututautaaaatauaaaauattaguutggcgtggtggtgcgcguutgtagtauuagutaututagaggutgagguaggggaatuauttgaauuuagaaggcggagattguagtgagucgagatcgtguuautguautuuaguutggtgauagaacgagaututgtutuaaaaaaaaaaaaaaaaaaaaaaaaaaaaaaggatatuuaguataaaataaauttguataaaaagguutgutttauauataaaatatatagaatauttguaatttgtgatgaaatagaguatutautattuagttuagtatttgggggtuttaattauutgttuagtaggaauuutttatatgtattatttttutttuuttttttttttgttttttgtgtatttttttttgttttaaaaaggtaaaguuautagagagaaautgaaagaaaauattuttaagataaattgaattgauattttutututaaaatatgatttatagauuauagataggaattaagagtttuutgataattttgguttuatattattttaaaggtatgtatatuttatuaaattattttauatattuatttggatuuaaaauatttutgatgt

SEQ ID NO: 231 - DNA, 1200 bp, Homo sapiens
tgauuttgtgatuuauuaauutuaguutuuuaaagtgutgggattauaggcgtgaguuauuaatgtatttuttttattaagaaaatgtgtttuutgtagtgttutgttttaaaaauagtuaautuauatgauuagaauuagatgutttgttttattttagttauaaatuatuatgtagtuutttttggaaagaauttaguaaattttuattatttatttaatatttattatttatttgtgtgtagtttttaatuuuutaaaggatgtaaaguatuuaguattaatuatattttattgaattataggaattaataatttggggauttattttutttaaaagtataaattattaaattatuatutguttagtguagaaagtttggaaaututtgaaattuauaaaagaaaaagagttauutgaagauuuuutgaguaaatgaatatagttatttttttaattgtuttaaaatgaaaatgttatttttgtuatutauatgattatttttaaaggauaaaagttttguatatgtaagggagaataatatttuttuuttatttttuutuuuuaaatagtutautuaaagataaagaguatgtautttuuuauuttcguaaauaauuuutaaaaututtguatauautaucgagutgguatautgaauuuaaaaaagaaggaagttautuuttttgutautauuatttgutgtgtttgtggauattttgtttgtttguagtgggatgatttgttttutttaaatagutttuuuaguataauaaautguucguaagaaaagttgautgtuuttauagaauaguagagtgtgtguagucgatgttgtttttgutgtuutttutttagauutgtaguttuutuutuuttauagattauautgagutttttuuuuttuttauautcgtgguutauattuuaggagatuaaguutgguauatgttaatgaatgtaggttttatttagaaauatgauuauttgtuautguuuagtgtuatgttatcggguagagautauuaauatttgtutauagtgaguattttuatauttutatauauagauaaggggaaautttuatuuuuacguaggatatagutttttgututguagagtutaauttttguaagtggaaguttuatggtggtggcggaggauutgagtguuutgaggatgguagaggauagutgtgt

SEQ ID NO: 232 - DNA, 1200 bp, Homo sapiens
tguuttgtutuatatttagttuattggaatauatatattatgutaauaataataaguatgaaaaaaatttuaaattttttaautuuatgaggtaaautaaataaggtaaaagaaattaaautaaggaaattatgauaataaaatgttttgaagauttuaggttttttaaaatgttagtuataatuaagaaaaaaauaaagttguutatagtggtagattuataauuaatatatttutttaatuaaagtttaaaatattttuatttutgattautaaaaatauattaatataagaagaatgattgttttataaaagaattuauagtauaatatutttaagtautatgttttutagaatttatttuttatataaaatguagttttutttaattttttatttttttttttgagattgggtttgcgutgtuauuuaggutgtagtguautggtguagtaatggutuautguaguutuaguutuutgggutuaaguagtuttuutuuuauttuattauutgggautauagatguatguuautgtguutggataatuttttgatttttttttttttttttttttgagatgaggtgtuautttgtaauuuaggutgguaggtgtatttuutttttttttttttttttttttgagatggagtutcgututgtuauuuaggutggagtguagtggcguuatutuagutuautguaagututguutuuuaggttuacguuattutuutguutuaguutuucgagtagutgggautauaggtguucguuauuacguuuagutaattttttgtatttttagtagagauagggtttuauagtgttaguuaggatggtuttgatutuutgauuttgtgatuuauuaauutuaguutuuuaaagtgutgggattauaggcgtgaguuauuaatgtatttuttttattaagaaaatgtgtttuutgtagtgttutgttttaaaaauagtuaautuauatgauuagaauuagatgutttgttttattttagttauaaatuatuatgtagtuutttttggaaagaauttaguaaattttuattatttatttaatatttattatttatttgtgtgtagtttttaatuuuutaaaggatgtaaaguatuuaguattaatuatattttattgaattataggaattaataatttggggauttattttutttaaaagtataaa

SEQ ID NO: 233 - DNA, 1200 bp, Homo sapiens
tgtttgautttguutggatauuataaaauuauaauaaaataaattutuutgaatgutauuagguaauauuaagatututtauauuacgagaaggauttgtgguuaaagatauaaatggutucguaatgtauagggtguatauagtguauutgaagaauutaaaagtggtttttagaatuatuataataaauagtuaagaattaaagatgtuaagcgutttutuaagaagtutaaautttatauataauattaaaaaagatutataaaguutgauuagtutuuutuaaattaagttatttgutautgauttgguaguuaaaacgatttaggatutttcggaagttgatgaaaaaggaguuagcgggttttgututttgututguatuacguuuttuutgggauuatuagggaauutuuuaguutttuaggataggttucgutuagaaaggtuauucggguagtggaaaucgtggguaaaagttagutgguaggauagcguagutuutuuagguagcggagguagcgcgtuucggututuagggauatttuuttuuuauutcgauuuucgggaggtggtuucggtataaaggutcgutgagcgggtgggtuauaguauagutttguagutgcggagaaacguuuaaggucgtguatutuuaggaggtagggaaguutgcgaguutucgggtuuucguuuuuagututtuuagguaucggcguatuucggagucgggcgcggggcgcgggguacggagucggggagggggtgtggcggguuagggcggtguagggauuutguuuagaggutgaaucgguutgggcgauuttuuttuucgauaggaguagtagatgcgtgggggagggcgaagggggagguutuauuuuuuagcgaguagucgggaggaggaaucggtcgcggagggcggggagcgcggutgaguaguucggcgcgggagtgagtgtgggcgaguuaguuagggtgagcgcgggcgucgggucgcggucgucggucguagcgggutgagattgttgtuututgtuauuagggcggutgggutuucguagtuutguagaucgcguucgatuucggcgauagggcgggcggauagucgcguatuuucggggtuucgucgaguutgggcguagagagucgggaggaagcgttcgutcguttcguuttgutgutgggaaautgaacga

SEQ ID NO: 234 - DNA, 1200 bp, Homo sapiens
tgttttattttagttauaaatuatuatgtagtuutttttggaaagaauttaguaaattttuattatttatttaatatttattatttatttgtgtgtagtttttaatuuuutaaaggatgtaaaguatuuaguattaatuatattttattgaattataggaattaataatttggggauttattttutttaaaagtataaattattaaattatuatutguttagtguagaaagtttggaaaututtgaaattuauaaaagaaaaagagttauutgaagauuuuutgaguaaatgaatatagttatttttttaattgtuttaaaatgaaaatgttatttttgtuatutauatgattatttttaaaggauaaaagttttguatatgtaagggagaataatatttuttuuttatttttuutuuuuaaatagtutautuaaagataaagaguatgtautttuuuauuttcguaaauaauuuutaaaaututtguatauautaucgagutgguatautgaauuuaaaaaagaaggaagttautuuttttgutautauuatttgutgtgtttgtggauattttgtttgtttguagtgggatgatttgttttutttaaatagutttuuuaguataauaaautguucguaagaaaagttgautgtuuttauagaauaguagagtgtgtguagucgatgttgtttttgutgtuutttutttagauutgtaguttuutuutuuttauagattauautgagutttttuuuuttuttauautcgtgguutauattuuaggagatuaaguutgguauatgttaatgaatgtaggttttatttagaaauatgauuauttgtuautguuuagtgtuatgttatcggguagagautauuaauatttgtutauagtgaguattttuatauttutatauauagauaaggggaaautttuatuuuuacguaggatatagutttttgututguagagtutaauttttguaagtggaaguttuatggtggtggcggaggauutgagtguuutgaggatgguagaggauagutgtgtggatguagatutuuuagattgtaautgcgatgtuauaagguagguatuttuttgagtttuttuuattgtauatagutttuatgttacgtggggtttgtaggatagacgutggaaguuttaatuuagaautgtttttguuaatgttau

SEQ ID NO: 235 - DNA, 1200 bp, Homo sapiens
ggagagauucgaaggggagcggaggttggggtagaaguuuucgguuutuutgtattautautgcggaatgcgggggauuucguagauaguuaggutuuuaggacgggguauuuttgggguttuagtttuuutcguatutuuaagutcgututgcgggcgatttguuaggagaggggtuuatggagagagcgggagacgaggggcguucggutgaggaataggguttguutuutagcgagtgtcgaggtuuucgggtucgtgcgtcggtutgtucgtucgtttgtgtgttutcgcggtgaacgcgggcggauuuuuagtututucgggagaaauttgggtutgcggutgguacgaggtutttgtgtucgggtgtututucggutgcgagtggttttucgcgtutucgutggggggcgggggauagggggcggggtgggggagggtatgagggtgttgcggcgtuuttuuuuuauuuuututcguuuutataautcgutuuutucgtuuututcggautcggautcggggucggtgcgcgautgagutggcguututagucgautttguuaagagggcguutuutguucgatucgcgutgauuutgcgcgggacguaguuuuaggcggutguutagucgaggcgggaguucgagutgagtcgagutcgucggagcgggtgagtuucgaguucgatuuucgucgggutggauuutggcgcguagutgcggcgtutucggggagtcgcgguagggggaatggcgtgtcgcguuutggcggcgggagutgcgggagggtcggcgacgtggcggucgguuuagggtatttauutgutguauuagtguucgguaguuacgutcgucggutgucgcgggguttutttgtuucgatatutggagggtucggggcgggagggagtautgggtcgcgtuuuutuuatgggggaggggutgggggagtgagagguutggagataatgcggagtgggggtggggagatuutguaaggauuaggacgucggggttuuaggagaututguaggutuuatuuutuuttattuuuuuauuucgauuuuagtagcgutggguutaggaguautcgggagttgutuagtggututguaucgutggcgcgggaaggggagaaaggggutguuagaggcgggaatttggtuuuuacgcgutgutuuagggacgagaaguua

SEQ ID NO: 236 - DNA, 1200 bp, Homo sapiens
gggguuuuaucgucgauaggtgtagatuaagagautuucgagatagguuuutguagggcgutggtuuuucgguutuuutgauuatggutuauagauttuuagcgutgagtgagguagutcgaaguututuagucgatutgttuutggguutuautuuauagtttutgaggggatututautttaagtutaguutuaatuutuucgttgagutttutgtutttuttgttatguttaguagaaagatauatgatututggtgutgtttuaucgaaguaaggaggtgggtcggcggaautgagcguuutgcguuututtgaaatgauuuuuuattuutgtttguuagaaauttttuuutuucgttguuatggagagagacggucgutguutuucgtcguuataguaauatgcgggtaucgcggaucguaggattgcggagutcggaaagtuttguagttcguatcguucguutgtuttggggtuttutauutucgaagutgggautcggggguuagtutaautgutuucgtuaggaauutcgguuaaggtuutuuuagauuutguutgaucgtcgututttutgttacggggttatutatttututgtuattcgtgtgguatgtgguagtttauuuagtgttttuauaaauacgatutttutatuuuuagggagtucgtgaagtauttaaaatuattauauutuutttacggagaggaaaattgaggutuagagacguuuaaggtuauauuagguagtgaatgcggagtgggaauucggaututttuutgguuttguauuutucgagattcgggauaguucgguttuucguutcgaaauuuuuauuuuucggautuagguuagtuuutgagtgtagagggggcggucgcgaggcguuagautcguuagutgucgcgggatguuacgtgaggggcggggauuutgcggggaagtgattaaaaguagagcggagucguagggggucgutguagggagggtuttuttggggucgtcgtguuucguuuuagtgcggucggggcgcgggttcgagutgutgutcgguaaguutgggtgtutaggguatgagcggagtgtggggggucggcggguutcggtguuaggaggcgutcgcggtuutcguutcgutgtgucggguucggucguuuuututcgggutggacgtggagauttgtcggaguttcga

SEQ ID NO: 237 - DNA, 1200 bp, Homo sapiens
aautgggaaagagutggaagatgagauauuaaautuuatuuuatggtgatgtttgaauuaggutggagutggtttuauatgtttttuattgtguatgautttuttaattguuttagtgggaatgcgttautguaaaataaagtgaaattgttttuuttattguttgtaatttauagaggggtggttttgatuatataaauuagguauttttuaaatuuattgatuttagggtggaaggagggatttutttttaagtattuatttcgaautatgutttuautagtaaautuaaaguuuaaggguaaaaauuuatgattutuuattttgtaatagutattagatgtuuauaguauatuagauutgtggaaaacguatgutttgtgtatggaagagggagagggagcgtuaggagttgtuutttguuaaaaaacgtaututgauautuaaagguauaggutggtggagatuuagggaauucgggaaaguuuagguutaautuagguucgcgcgggtututtutgatuuuataagatgaggagtgggtuutggututggguatuutggtgtcgautgtgagttuuuaguagagguuuagagtuucggtucgguagucgagggaagcgggggggtuttuuagaagaagaaaggguuaaggtuauuucggtguututuuaguaguaguagagggcggcggtcggtgtcgutgutggucgggguutcgaggaaggcgcggguuagutggggucgggtutgcgttuuuaggagutguuaucgttuuagggaguaagtuaggucgggacgtagcguutgcgcgggauuutuauttguuauuaagauuuuuauaaauuucguuuuatuutguuttacguuucguuuuaaggtcgttutuucgauucggggtuucguuuuaaggucgtuutuucguuucgucguttggtggcggucguatgutguucggatataaagggtcgguuuuauatuuuagggauuagcgagcgguuttgagaggututggututtguttuttaggcgguucgaggacguuatggucgagtguucgauautcggggagguagtuaucgauuauucggaucguutgtggguutgggagaagttcgtgtatttggacgagaaguaguacguutggutguuuttaauuatcgaggtauaguuagutttggagcggatggcgagguagua

SEQ ID NO: 238 - DNA, 1200 bp, Homo sapiens
tutaatutatggtaatcgaaatgagaagagtgtttguuuutgguutaaggatggaaggattgatttaggaaaaagtutguaggagutttutggagtgatggaaatgttutaaututtttttttttuttuttuttuttutttttuttcgtttcgagacggagttuuaututgucguuuagautggagtguagtggcguuatutgggutuautguaguutucguutuutgggttuaaguuattutuutguutuaguutuucgaggggutgggautauaggtguucguuauuacguucggutaattttttgtgtttttaatagagacggggtttcguuatgttgguuaggutagtutcgaautuutgatttuaggtgatuuauucguuttgguutuuuaaagtgutgggattauaggcgcgagutaucgcguuuaguuaaaatgttutaattutttattggtggutauauagttgtuaaaatttatuaaautgtuuauutacgatatattataaguutgtattaaatgtaaattagaautcgattgaaatutgtgtgtataaatataaatatauauttatttauatttutaatutaatutgaaggtuuattguaacgaatuucgutucgututgauuuaagaautucguautttututuuauuaguutttuaaatauutgaauttuatguuttuaagaggtggattucgguttutttauagcguaatuautaguaggtuatttucgtutaguttguagatttuuuattautgauauaaacgutuuutuagggcgtuuucgtutggautauaggagaaagtttgggagguaggttuaauttttuaauttggcggggaattuututuuututtauauagtttguagcgcgggggcggcggggtgauagtuuucgtguatgaatuaguuauuuutuaggutucguuucggcgggggtcggucggacgutcguuucguataaagcggguauucgggucguutggaguagaaagucgcguauutuutuucguuaggcgutttutcggacguuttguuuagcgggucguucgauuuuutguauuatggauuucgutcguuuuutggggutgtcgattutgutguttttuutgacggaggutguautgggcgatgutgutuaggaguuaauaggtatutggucgutuuaggaguttututuuuuaaucgg

SEQ ID NO: 239 - DNA, 1200 bp, Homo sapiens; identical to SEQ ID NO: 238

SEQ ID NO: 240 - DNA, 1200 bp, Homo sapiens
uuuaaattgttutatagattuaaaauattguuagttaaaatutuagtggautttgtttttttgaaautgauaagutgattutguuatttataataatattuaaagggguaagaataauuaagauauatttgaagaagaauaaggaggaagaagaacgtatttgaagaagaataaggaaututattaagtgtatatutuutatatattgagatauauatttgaaatauatutatuauutagatattgagatatattatauatttaatatagtgtgttgutggtatagggatagauaaatgaauattggatuagaataguautgaaauaagatutauuuutatatggguatgtuuutattaauaaaaaaatattgttgattatttuutuaagatgatutgtaaaaaauaagttaattgaauatuuatatgggaaaaaagtgaatuuttutgutauutuaaatatuaauuuuaggtagattatagatutaaaagtgaaagguaaaguaataaagattttagaaaataauaaagagaatatutttatgaaaatauaggaaatattttaaaaataggutagaaaautuautattaaaatatataaaatataagaauatutgttuatuaaaagtautatagtttgaataguagtgtutuutuuaaaattuatattgaaauttaatuuuuattgtaauagtautaatuutaaaautaagtattaagaggtgtgauutgtgggaagtgautaagtuatgaggggtutttuutuaggtatgggattaguatuuttataaaagguuttgagatggaatgtagtguuuuuttguuututtgcgttutuuuatgtgaggauauaguaauaagguauuatuttggaaggagagagaaguuutuauuagauaauagtguauatguuttgatuttagauttuuuaguutuuagagutataataaatauatttutgttutttataaattauttggtututggtattttgttauagaaguautaatggautaagauaaaaagattuuattatgaaagtgaaaagguaaucguagagtgguagaaguttttggagatgatgaatatgtttauttuutggattgcggtgatgatauuatgagtgtatauatatgtgtgtatauataatgtatauaaattgtgttuattgattatgtauagtttutttgtatauuaatta

SEQ ID NO: 241 - DNA, 1200 bp, Homo sapiens
gggaaagtutggaagaauauaututtutggatutaatutaagttttutuautauuuauutatuatgagtauutttttuagtutututagguuaagggtttuttatttaaaaggauuaagagautuuttgatuuutaagattuutgutaagattutgtauututttuutuuuaaaagaatgautggtuttgaattutaaaatguaatttttgtgagtgtgttttaaaaagaagautguatttcgutgaaaaatuttuuttggaaagtatataaatuttattagacguutttgagagtuattaaaagaautgaaguatuatguacgtttaaaauattauuaggutggatggagtacgaagguautuaggggaaggtauttaaauutucgtaggttuaauuaagtuttaucgggagtaauuuaacgaauaagtgatgutttttgagauaaatttggagagttggutguaggaaaattaauaaguuagtggatttuuttttuuuttuuttaggaaauttgtttutgtagatguaaaaaggguuutagatuututggaagagaaagagagtggggaggggaagtttututttutttautuuuuauutgcggtutgtutgatttuututuuagututgtutgataaggaattgattutuuuuutuututtuuuuutuucguuutuuututuuttutuuutgagtaacgatgutgtuutaguaagtgatgutgtcggagauaggagacgggcgucgaggagguatcgucgucgucgcggggutggagaguututuuuaguauuagaguuucgutcgguuucggguttuutcgtcguaguuacggucgcgguagutgutuuuacggtttgatggtgggcggcgguagutcgguttcggcgutaguututaautgutggatcgcgggucgcgacgututucgutuutguuttuucguuutgggucguucgggguuucggaagucgcgggaggtggtgaaggggcgucgcgggaagautguautgucgcgaaggcggaggaggucgguagucgguauuuuuauautcggaucguagucggcgcgatgtuuauuaaggtuaggaagtguaaagaauaaguaagggtgauuttuucggcgucggaggaggaggaagacgagggcgaggacgagggcgcggagucguagcgucgucgucggggutggaggggu

SEQ ID NO: 242 - DNA, 1200 bp, Homo sapiens
tutuuaautuutaggutuaautgatuutuucgtutucguututuaaagtgutaggattauaggtgtgagguautgtguuuaggtgagaggtaagatttuttaaauagaguagaaaaauautaaggtaaaggggaaaatgauaaattgguauatattaagatgaaaaauttttgttuttuaaaaggtauuattaagatggtgaaaaagtagguuaggtacggttgutuatguutgtaatuuuaguautttgggagguuaagatgggcggatuauutgagguuaggagttuagauuaguutgguuaauatgguaaaauuatgtuttgautaaaaatauaaaauaaaauaaaauaaaauaaauaaauaaaaaaaucggguatggtggtgggcguutgtaatuucggutauttgggaggutgaaguaggagaatcguttgaauttgggaggcgguaguttuagtgagutgagatuatguuattgtautuuaguutcggcgauagcgaaautacgagtuuaaaagaaaagtaauuuuauagagtgggaggaaatatttgtaatauutatautggaagagaaatatttgtaatauutatautggaagaatgautgautagtatutaguatgtauaaagguttuutauaaatuuatatgaaaaataacgtuaguuuagtagagaaatagggagaaguutcggatautguauaagagagaatatuutagtgguuaauaaauauatgagaaggttutuagutuattagautttggutaaatguaaattaaaauuauuatgtaatatuattautuatuuaguacgatggutaaaataauaaagacgtaaagauauuauuauagautcgcgagautgaggagtgagtgtcgcgagautgaggggtgggtgtcgcgagautgaagagtgggcggcguaaatgggcgaggtgtttautcguuattttgtgtgaggaacgttgacgutauutauututgguutgguututauuuautgutguuutggucgutauuaauatgaguutuucggagagtuutuauaguuucgutaututcgautatgututggaagauucguauuaggguuagaggtuucgagaaaagaguaaggcgauagaggtaatgguagatatgtttgatggucguttggaguuaatcgtgttuuuaucguuucgguttuuagaggagggggt

SEQ ID NO: 243 - DNA, 1200 bp, Homo sapiens
atutgttaauutuaauttuutaaatagaaggtutattuttttauuatuatuattattututtcggtguutatttttaaaaaatautuaauuttuttguttuuttcgutauutaagtatttutguaaguuuautttgttutguaguttagutttuutgguauaattuttatagattttggtuuuttttaaaattuattuttuaguaaatgutttutututuuatuttttgautagagatuattagagatuauutgagatuattagagaauagtggtttuuttggttguuattuuutttuttuttuattgggagtattutgcggtgaautuagauattttatttttuaaaguttuuuattuttttaaaaatguttttuuttttauaguututcgutuaaaatuatauuuatuttttuuutggatutgttttutuaagtutuuaatcguutguuttutttgtgtuttgtattauuutuauatuuuuuagutttutautgututuuuaggauuaauuatttuttucgcgggagtuauattauatuaguattuutaatguagtatutgttatutauuagattutgttttattutaggtagtuauttaaaaacgaauutcggtautggtutgauttaauatggaggaggaattgtutaaggttaaacguaaautgutgagagatttggggcgggggguauauatttauattuattcgtattaaatatatauutgttgaatttgtgutttttutuaaatguttuagagautcgagutttagagtaattgggatggtgaaaggatgggtttuuagaaauttcguuuaaaattaaagautuuatuaaaaggautgutuuatauautuaaggaauauuuauuaauaaatuucgtutuuauaauuauuagattatutuaucggcgagtgagautguaaggtttggggguucggucgtauuautucgcgutgcguacggggggttcgtauuuatutggucgcgaucgtucgtttuuuuutcguttggttutguuuutgutuuuuutguauagguutuauagtgcgtutggucggcgutttatagutguaguutgggcggutucgutagutgtttttcgtuttuuutaggutatttutgucgggcgutucgcgaagatguagutuaagucgatggagatuaauuucgaggtgagcguuaggtguaucgutauu

SEQ ID NO: 244 - DNA, 1200 bp, Homo sapiens
aaaatautuaauuttuttguttuuttcgutauutaagtatttutguaaguuuautttgttutguaguttagutttuutgguauaattuttatagattttggtuuuttttaaaattuattuttuaguaaatgutttutututuuatuttttgautagagatuattagagatuauutgagatuattagagaauagtggtttuuttggttguuattuuutttuttuttuattgggagtattutgcggtgaautuagauattttatttttuaaaguttuuuattuttttaaaaatguttttuuttttauaguututcgutuaaaatuatauuuatuttttuuutggatutgttttutuaagtutuuaatcguutguuttutttgtgtuttgtattauuutuauatuuuuuagutttutautgututuuuaggauuaauuatttuttucgcgggagtuauattauatuaguattuutaatguagtatutgttatutauuagattutgttttattutaggtagtuauttaaaaacgaauutcggtautggtutgauttaauatggaggaggaattgtutaaggttaaacguaaautgutgagagatttggggcgggggguauauatttauattuattcgtattaaatatatauutgttgaatttgtgutttttutuaaatguttuagagautcgagutttagagtaattgggatggtgaaaggatgggtttuuagaaauttcguuuaaaattaaagautuuatuaaaaggautgutuuatauautuaaggaauauuuauuaauaaatuucgtutuuauaauuauuagattatutuaucggcgagtgagautguaaggtttggggguucggucgtauuautucgcgutgcguacggggggttcgtauuuatutggucgcgaucgtucgtttuuuuutcguttggttutguuuutgutuuuuutguauagguutuauagtgcgtutggucggcgutttatagutguaguutgggcggutucgutagutgtttttcgtuttuuutaggutatttutgucgggcgutucgcgaagatguagutuaagucgatggagatuaauuucgaggtgagcguuaggtguaucgutauucggagagcgcgaggucgagggagggggagucgagtcgutgatcggttcggttttguutttttutttguatttguuttt

SEQ ID NO: 245 - DNA, 1200 bp, Homo sapiens
aaatautuaauuttuttguttuuttcgutauutaagtatttutguaaguuuautttgttutguaguttagutttuutgguauaattuttatagattttggtuuuttttaaaattuattuttuaguaaatgutttutututuuatuttttgautagagatuattagagatuauutgagatuattagagaauagtggtttuuttggttguuattuuutttuttuttuattgggagtattutgcggtgaautuagauattttatttttuaaaguttuuuattuttttaaaaatguttttuuttttauaguututcgutuaaaatuatauuuatuttttuuutggatutgttttutuaagtutuuaatcguutguuttutttgtgtuttgtattauuutuauatuuuuuagutttutautgututuuuaggauuaauuatttuttucgcgggagtuauattauatuaguattuutaatguagtatutgttatutauuagattutgttttattutaggtagtuauttaaaaacgaauutcggtautggtutgauttaauatggaggaggaattgtutaaggttaaacguaaautgutgagagatttggggcgggggguauauatttauattuattcgtattaaatatatauutgttgaatttgtgutttttutuaaatguttuagagautcgagutttagagtaattgggatggtgaaaggatgggtttuuagaaauttcguuuaaaattaaagautuuatuaaaaggautgutuuatauautuaaggaauauuuauuaauaaatuucgtutuuauaauuauuagattatutuaucggcgagtgagautguaaggtttggggguucggucgtauuautucgcgutgcguacggggggttcgtauuuatutggucgcgaucgtucgtttuuuuutcguttggttutguuuutgutuuuuutguauagguutuauagtgcgtutggucggcgutttatagutguaguutgggcggutucgutagutgtttttcgtuttuuutaggutatttutgucgggcgutucgcgaagatguagutuaagucgatggagatuaauuucgaggtgagcguuaggtguaucgutauucggagagcgcgaggucgagggagggggagucgagtcgutgatcggttcggttttguutttttutttguatttguutttu

SEQ ID NO: 246 - DNA, 1200 bp, Homo sapiens
tguttuuttcgutauutaagtatttutguaaguuuautttgttutguaguttagutttuutgguauaattuttatagattttggtuuuttttaaaattuattuttuaguaaatgutttutututuuatuttttgautagagatuattagagatuauutgagatuattagagaauagtggtttuuttggttguuattuuutttuttuttuattgggagtattutgcggtgaautuagauattttatttttuaaaguttuuuattuttttaaaaatguttttuuttttauaguututcgutuaaaatuatauuuatuttttuuutggatutgttttutuaagtutuuaatcguutguuttutttgtgtuttgtattauuutuauatuuuuuagutttutautgututuuuaggauuaauuatttuttucgcgggagtuauattauatuaguattuutaatguagtatutgttatutauuagattutgttttattutaggtagtuauttaaaaacgaauutcggtautggtutgauttaauatggaggaggaattgtutaaggttaaacguaaautgutgagagatttggggcgggggguauauatttauattuattcgtattaaatatatauutgttgaatttgtgutttttutuaaatguttuagagautcgagutttagagtaattgggatggtgaaaggatgggtttuuagaaauttcguuuaaaattaaagautuuatuaaaaggautgutuuatauautuaaggaauauuuauuaauaaatuucgtutuuauaauuauuagattatutuaucggcgagtgagautguaaggtttggggguucggucgtauuautucgcgutgcguacggggggttcgtauuuatutggucgcgaucgtucgtttuuuuutcguttggttutguuuutgutuuuuutguauagguutuauagtgcgtutggucggcgutttatagutguaguutgggcggutucgutagutgtttttcgtuttuuutaggutatttutgucgggcgutucgcgaagatguagutuaagucgatggagatuaauuucgaggtgagcguuaggtguaucgutauucggagagcgcgaggucgagggagggggagucgagtcgutgatcggttcggttttguutttttutttguatttguutttuagatgutgaauaaagt

SEQ ID NO: 247 - DNA, 1200 bp, Homo sapiens
uuuuagtutagaaagaauutggcgtgtaguaggatguaauatatauttgtagaatgaataaaaaggtaataaaataauacgagutuauatagcgguuaguatttgutgtguauuataatucgtgutaaguautttguatgcgttatttaatttgaatttuataauagtattauataagaaaauaataggguuagguatggtggutuacguutgtaatuuuaguautttgggaggucgagguaggcggatuacgaagtcgggagutuuagauuaguutgauuaauatggtgaaauuuuatttutautaaaautagaaaaagtaguuaggcgtggtggcgtgcguutgtaatuuuagutauttgggaggutgagguaggagaattguttgaauucgggaggtggaggttguaatgagutaagattguauuautguautuuaguutgggcgauagagtgagautucgtutuuaaaaaaaaagaagaaaaagaaaagauagtattttgutuuutuaucguuttuagguttttaauuaautguuauttauuauuutagtgaaggtttguucgagtguuutatttaatattguagguttutgtttuutauuaguuuutttguttaututttuauauttagtuauuttutaacgtgguatauattguttattttutatgutuatgagtatgtutautuuatggagguatagatgtttgtatguutgtttautgatggatuauaaautgtgtaguutaaaatagagutaguauatagtuuuauuuaauacgtgtuaggtgaguaattagatatagagggttuuaataauttgtggggauauuuagataagatutgtttatgautuatgtuttauauagagutuattuttggtututtggttagtttatuagagaagauaaatgtattattggaaaatgtgtuttaggtuattttttgauagtggututaatautggaatutattttaautaaaggttattatuataaaguaggtgtttgutgaagauaguttautuagatuautautguutggaggtggttgatatatuutggtgtaaauuttuaagaaggguauagguaggaaaauatgaguuaguaautgaagaaacggguaaagauaagauauuagaaagguutgggtggaagaguuuuuagtggggutaaguuuagguaagguaagtuaaguua

SEQ ID NO: 248 - DNA, 1200 bp, Homo sapiens
uutagutuuuutttutgtuagttttttaattttatatttgttttuaaatgagtuutuauattgattttaaaaatttaauaagagtatagtgaaaaggaagttuuuutttuautatttatuttuuuuatatguatutattatgacgggtttuttttttaaautauttggagtautuataautgattttgtaututagttuagaagaaatgtutgaatgttaaatgtttauataaaaaatgutattttattatatuagtaatggaguttuuuuuutttaaaautgutguauuaggaagatauaauuttgttatuauaatttaaagagaaaatgagauttggaggttuagattuutuuauagtuatagtatuagutauuuacguagtgauuuauattuaggtgattauauauautuatauauagatuuatauacgatuttguuuuuuuuauutuuauauautuaatatgauutuuuuacguuuaaagutguauauatuttgauutuauttututuauauauauauuaatutgauucguacgcggtttutttuautuaautauuuagauauuuutuatuuuauuaguauaaguagguuuaucgcggtutuauauauautcgaguuttuutttguuuagtutuauatguatgautgggatcgcggatttgtuuuuttucgautuuuuatgutggttcggtuuuagacgutuaggtguuauattggggtgcgututuucggutuutuuauacgutacguagutguucguutuaagagcgtutgcgtguttuuagcgttaagtggaattuuaaguaguutucgggguauagttagtutggtuautgcgguuagautgucguutuaggauuuagagaacguaguauautttagttggguuuatutatuuucggcgagguttggautacgtttuuuaggagauuutgcggtuuagtatgtutuucgaggaggggcggaaguucgggagucguuututgaggutgggcggggtgaaagaaaguttguuuuagaggauttaaauagguaagaaggauttggttaaagautattguaatagtuaauttuuaatauaauaguagutggagatttatagutaacgggutgggtgaaggagttaaaggatgutaaattautaagaggaagttaagtatuaagagtggggggattgttautaaautgguttagutg

SEQ ID NO: 249 - DNA, 1200 bp, Homo sapiens
auattguutuutgtuttgtuttuuutttuututtatgttaaaauuagattutgttuututgutgtaguuuttautgagutuuatuttututuaatgttatuatuatagaatttggagguaaauttatttuttaaatgguataaauaaauuauattutaauuauagatgaatuttuuututuuatagutuattuuatautuatttaguauattagtutttuattgggaaautgututttgtggattatutttuttttutgtutuuatuagtttatatguaaattatgtttattautgaaauatuuuagatttuuuauuuuaggaaaaatgtagtautuuuaauaauuutgataaatgaatatgagaattaututuatttataaatgtttataguautagattuatututtaaaaaattaagauaggguuaggauatutgtataattauuaagutttuaaagtutguaggauttttaaautaaautaaautautatttaaaautautattttaaataaatatutaaautautatttaaaaataatutgtttuutgaaaauutuutuuautttutagtutgauttttautuattttututguttuuutaatutgtttttuaaattguuuagtuatatgtgagaautgtataaauuautuaatttttatgaaaattaaattgtagttaagtutttcgtatutattataaacgtttaatututuuuagaauauaaaaaagtgaatgtttguttttatattttutttuagautatgaatuaaagttgagauuaagaaattatttutgaaaaaggatatggaaaauuttauaaaauauaguattgagtgttuaagtttuagaggtgattgggaatgtaaaaauuagtttgagagaaaauagggatutuaggaaggauatttuagtgaaatgatatttautuutgaagauatguuuautttuagtatuuaguatuagagaattuatautgatgagaaautuuttgaatgtaaggaatgtgggaaggattttagttttgtatuagtuuttgttcgauatuagcgaattuatautggtgagaaauuttatgaatguaaagaatgtgguaagguutttggtagtggtguaaauuttguttauuatuaaagaattuatautggtgagaaguuttttgaatgtaaagaatgtgggaagguutttggtagtggu

SEQ ID NO: 250 - DNA, 1200 bp, Homo sapiens
ggucgttatgaagaggaagaagtautgtgtuatutattgtuutggggauutgtuaauaggaauaatagatagauatuauuaggggtgtatttutgttuaauauaaggaaaagtttutaattggagutgtuuaguatgaaatggautaaguagtguattttutttutucgatuutgautuuautgtgacggguautguagautagagagattgtgtuutagtatgaatttaggttggataaatgatauttagggataaagagaggaaaggaatatuagaatutuuaagaagutgatauattttgaaaaaatatataaaatguuauauttuaggttttuttgtgtttaatttgaaacgtuuagtaauattaaagaauaguatttgatttguttaaauaaagtagtaauagutgaagttuuttuuaaatuauttttutgattututaagagguttguutatguuttguagatutaguttuauutauaagauatattutgtaaggatuuaggttuaguaaggtgtuagaatgtuutaagggtttutgtuucggcgututguutgtuucguttgtguauaatguttttguutatuuutgtttutguatagattttagtuttuuuauuagggacguaautgtuagtttuutucgtutututcggttutattuuaatutgutuatutuuuutuucgtuuucgutttuuuuaaggauutautgagatttcgatuuuauaauttagtgggtuuuatutuauuuagguuucguaatuutcguuttutauttgtuagcgttgtautauaututgaguagguauuuutgtgutautuacggaagcgggauuattaggtcgguuattuutuutuauuuuacgauacguauuuautgggtcgggucguutuuttguagggagtutuaaggatgggutggcgtuagggagggguuuagggggcgutttuuuaauautggtcguagtuattgttggtataacggutagagacguuuagtgagttaguatggaggguagtgggaucggaaaaagacgtggaaaagutgcgaaaacgaguuttcgaatuatggacgcgcggguuuagutuutuutucgagttuutuatucggggucgtuautuauatucgggguuutuautuauatucgggauuutuatucggggututuauuuauatucgggauuutuatguutggg

SEQ ID NO: 251 - DNA, 21 bp, Homo sapiens
tacgcgtaggttttaagtcgc

SEQ ID NO: 252 - DNA, 22 bp, Homo sapiens
tcccgaactaaacgaaaccccg

SEQ ID NO: 253 - DNA, 37 bp, Homo sapiens
cgacatgcctacgaccgcgtcgcccattagcatgtcg

SEQ ID NO: 254 - DNA, 22 bp, Homo sapiens
tttgttcgtttttcgattgttc

SEQ ID NO: 255 - DNA, 23 bp, Homo sapiens
taacgctataaaactcctaccgc

SEQ ID NO: 256 - DNA, 44 bp, Homo sapiens
cgtctcgtcggggttcgggcgtatttttttaggtaggcgagacg

SEQ ID NO: 257 - DNA, 20 bp, Homo sapiens
gggattataagtcgcgtcgc

SEQ ID NO: 258 - DNA, 20 bp, Homo sapiens
cgaacgcaaaaccgaaatcg

SEQ ID NO: 259 - DNA, 35 bp, Homo sapiens
cgacacgatatggcgttgaggcggttatcgtgtcg

SEQ ID NO: 260 - DNA, 19 bp, Homo sapiens
gagatgtttcgagggttgc

SEQ ID NO: 261 - DNA, 20 bp, Homo sapiens
ccgcaatatcactaaaccga

SEQ ID NO: 262 - DNA, 43 bp, Homo sapiens
cgacatgcgttcgtgttttggtttgtcgcggtttggcatgtcg

SEQ ID NO: 263 - DNA, 28 bp, Homo sapiens
gttgggttttcgtagttttgtagatcgc

SEQ ID NO: 264 - DNA, 18 bp, Homo sapiens
ctacgcccaaactcgacg

SEQ ID NO: 265 - DNA, 36 bp, Homo sapiens
cgacatgccccgccctatcgccgaaatcgcatgtcg

SEQ ID NO: 266 - DNA, 23 bp, Homo sapiens
ttcgggttattagcgtttttatc

SEQ ID NO: 267 - DNA, 19 bp, Homo sapiens
actccaaccaaacgacgaa

SEQ ID NO: 268 - DNA, 35 bp, Homo sapiens
cgacatcgttggttcggtgcgtgtggtcgatgtcg

SEQ ID NO: 269 - DNA, 19 bp, Homo sapiens
ggtcgtagtcgtagtcggg

SEQ ID NO: 270 - DNA, 21 bp, Homo sapiens
ccgcaaaaacctaaaacgtaa

SEQ ID NO: 271 - DNA, 37 bp, Homo sapiens
cgacatgcggttttttgggtcggggcgcggcatgtcg

SEQ ID NO: 272 - DNA, 26 bp, Homo sapiens
tagggagtatataggttggggaagtt

SEQ ID NO: 273 - DNA, 27 bp, Homo sapiens
aacacacaataacaaacacaaattcac

SEQ ID NO: 274 - DNA, 42 bp, Homo sapiens
cgactgcgtgtggggtggtgatggaggaggtttaggcagtcg